(19) United States
(12) Patent Application Publication
(10) Pub. No.: US 2025/0099481 A1
(43) Pub. Date: Mar. 27, 2025

(54) BCL-2 INHIBITORS

(71) Applicant: Newave Pharmaceutical Inc., Pleasanton, CA (US)

(72) Inventor: Yi Chen, Pleasanton, CA (US)

(21) Appl. No.: 18/722,153

(22) PCT Filed: Jun. 22, 2022
(86) PCT No.: PCT/US2022/034518

§ 371 Date: Jun. 20, 2024

Related U.S. Application Data
(60) Provisional application No. 63/298,726, filed on Jan. 12, 2022.

(51) Int. Cl. A61K 31/551; C07D 498/22; C07D 519/00

(52) U.S. Cl. CPC A61K 31/551; C07D 498/22; C07D 519/00

(57) ABSTRACT

The disclosure includes compounds of Formulae (1) wherein Q₁, Q₂, Q₃, Q₄, Q₅, Q₆, Q₇, Q₈, R₁, R₂, R₃, R₄, R₅, R₆, R₇, R₉, R₁₀, R₁₁, a, b, c, g, j, k, m, n, L, Z₁, Z₂, and Z₅ are defined herein. Also disclosed is a method for treating a neoplastic disease and autoimmune disease with these compounds.

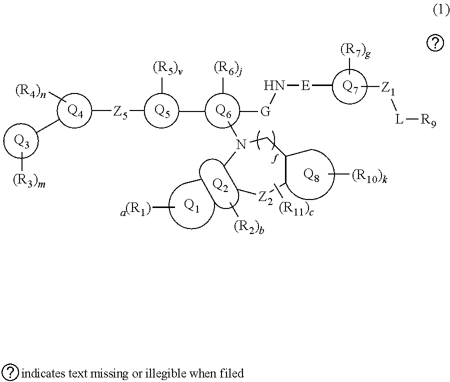

### REFERENCE TO RELATED APPLICATIONS

This international patent application claims priority to International Patent Application No. PCT/US2021/064278, filed on Dec. 20, 2021, and to U.S. Provisional Patent Application No. 63/298,726, filed on Jan. 12, 2022, the entire contents of each of the above-referenced applications are hereby incorporated herein by reference.

### BACKGROUND OF THE INVENTION

Apoptosis, or programmed cell death, is a conserved and regulated process that is the primary mechanism for the removal of aged, damaged and unnecessary cells. The ability to block apoptotic signaling is a key hallmark of cancer and is thus important for oncogenesis, tumor maintenance and chemoresistance [Hanahan, D. & Weinberg, R. A. The hallmarks of cancer. Cell 100, 57-70 (2000).]. Dynamic binding interactions between prodeath (for example, BCL-2-associated X protein (BAX), BCL-2 antagonist/killer 1 (BAK), BCL-2-associated agonist of cell death (BAD), BCL-2-like 11 (BIM), NOXA and BCL-2 binding component 3 (PUMA)) and prosurvival (BCL-2, BCL-XL, BCL-2-like 2 (BCL-W), myeloid cell leukemia sequence 1 (MCL-1) and BCL-2-related protein A1 (BFL-1)) proteins in the BCL-2 family control commitment to programmed cell death. Altering the balance among these opposing factions provides one means by which cancer cells undermine normal apoptosis and gain a survival advantage [Youle, R. J. & Strasser, A. The BCL-2 protein family: opposing activities that mediate cell death. Nat. Rev. Mol. Cell Biol. 9, 47-59 (2008)].

BCL-2, the first identified apoptotic regulator, was originally cloned from the breakpoint of a t(14; 18) translocation present in human B cell lymphomas [Tsujimoto, Y., et al. Science 228, 1440-1443 (1985); Cleary, M. L., et al Cell 47, 19-28 (1986); Boise, L. H. et al. Cell 74, 597-608 (1993)]. This protein has since been shown to have a dominant role in the survival of multiple lymphoid malignancies [Vaux, D. L., et al pre-B cells. Nature 335, 440-442 (1988)]. Overexpression of Bcl-2 proteins correlates with resistance to chemotherapy, clinical outcome, disease progression, overall prognosis or a combination thereof in various cancers and disorders of the immune system. Involvement of Bcl-2 proteins in bladder cancer, brain cancer, breast cancer, bone marrow cancer, cervical cancer, chronic lymphocytic leukemia, colorectal cancer, esophageal cancer, hepatocellular cancer, lymphoblastic leukemia, follicular lymphoma, lymphoid malignancies of T-cell or B-cell origin, melanoma, myelogenous leukemia, myeloma, oral cancer, ovarian cancer, non-small cell lung cancer, prostate cancer, small cell lung cancer, spleen cancer, and the like is described in PCT/US2004/36770, published as WO 2005/049593, and PCT/US2004/37911, published as WO/2005/049594. Involvement of Bcl-2 proteins in immune and autoimmune diseases is described in Current Allergy and Asthma Reports 2003, 3, 378-384; British Journal of Hematology 2000, 110(3), 584-90; Blood 2000, 95(4), 1283-92; and New England Journal of Medicine 2004, 351(14), 1409-1418. Involvement of Bcl-2 proteins in arthritis is disclosed in WO 2009/064938. Involvement of Bcl-2 proteins in bone marrow transplant rejection is disclosed in US 2008-0182845 A1. All incorporated herein by reference.

In the last decade, several Bcl-2 inhibitors such as ABT-737, ABT-263, and ABT-199 as shown below have been identified and entered human clinical trials for cancers treatment.

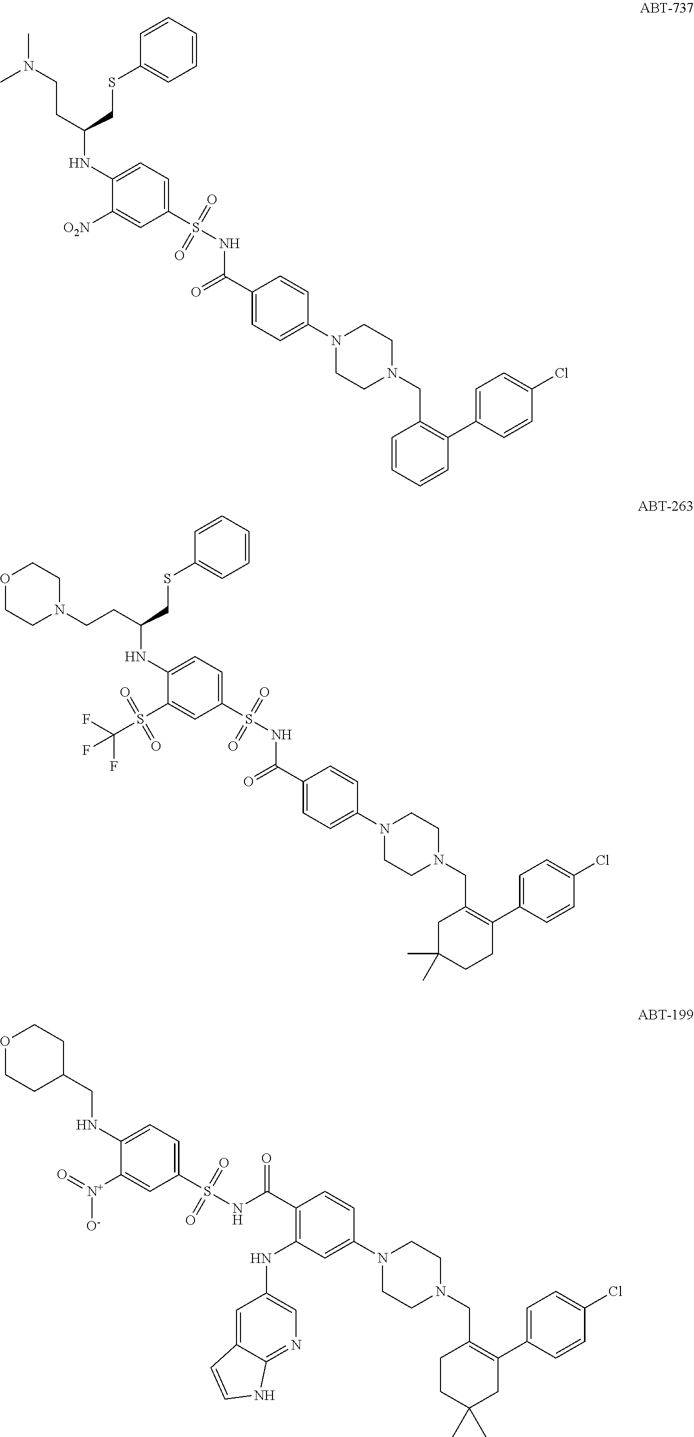 [SMILES: CN(C)CC[C@@H](CSc1ccccc1)Nc1ccc(S(=O)(=O)NC(=O)c2ccc(N3CCN(Cc4ccccc4-c4ccc(Cl)cc4)CC3)cc2)cc1[N+](=O)[O-] ; CC1(C)CCC(c2ccc(Cl)cc2)=C(CN2CCN(c3ccc(C(=O)NS(=O)(=O)c4ccc(N[C@@H](CCN5CCOCC5)CSc5ccccc5)c(S(=O)(=O)C(F)(F)F)c4)cc3)CC2)C1 ; CC1(C)CCC(CN2CCN(c3ccc(C(=O)NS(=O)(=O)c4ccc(NCC5CCOCC5)c([N+](=O)[O-])c4)c(Nc4cnc5[nH]ccc5c4)c3)CC2)=C(c2ccc(Cl)cc2)C1]

ABT-737 is discovered by nuclear magnetic resonance (NMR)-based screening, parallel synthesis and structure based fragment drug design [Tillman Oltersdorf, et al, Nature, Vol 435, 2005, p 677]. ABT-737 a small-molecule inhibitor of the anti-apoptotic proteins Bcl-2, Bcl-XL and Bcl-w, with an affinity two to three orders of magnitude more potent than previously reported compounds. Mechanistic studies reveal that ABT-737 does not directly initiate the apoptotic process, but enhances the effects of death signals, displaying synergistic cytotoxicity with chemotherapeutics and radiation. ABT-737 exhibits single-agent-mechanism-based killing of cells from lymphoma and small-cell lung carcinoma lines, as well as primary patient-derived cells, and in animal models, ABT-737 improves survival, causes regression of established tumors, and produces cures in a high percentage of the mice. Unfortunately, ABT-737 is not orally bioavailable, and its formulation for intravenous delivery is hampered by its low aqueous solubility.

After extensive MedChem effort, an orally bioavailable Bcl-2 inhibitor ABT-263 (Navitoclax) has been developed [Cheol-Min Park, et al J. Med. Chem. 2008, 51, 6902-6915]. ABT-263 is a potent inhibitor of Bcl-xL, Bcl-2 and Bcl-w with Ki of 0.5 nM, <1 nM and 1 nM. ABT-263 has an IC₅₀ of 110 nM against SCLC H146 cell line.

When ABT-263 is administered at 100 mg/kg/day in the H345 xenograft model, significant antitumor efficacy is observed with 80% TGI and 20% of treated tumors indicating at least a 50% reduction in tumor volume. Oral administration of ABT-263 alone causes complete tumor regressions in xenograft models of small-cell lung cancer and acute lymphoblastic leukemia [Tse C, et al. Cancer Res. 2008, 68(9), 3421-3428]. In the clinical trial, however, the inhibition of BCL-XL by ABT-263 (navitoclax) induces a rapid, concentration-dependent decrease in the number of circulating platelets. This mechanism-based thrombocytopenia is the dose-limiting toxicity of single-agent navitoclax treatment in patients and limits the ability to drive drug concentrations into a highly efficacious range.

Thus, a BCL-2 selective (BCL-XL sparing) inhibitor would culminate in substantially reduced thrombocytopenia while maintaining efficacy in lymphoid malignancies. The resulting increase in the therapeutic window should allow for greater BCL-2 suppression and clinical efficacy in BCL-2-dependent tumor types. After extensive MedChem, ABT-199 (GDC-0199) has been successfully developed [Andrew J Souers, et al, Nature Medicine, Volume 19, 22, p202, 2013]. ABT-199 is a Bcl-2-selective inhibitor with Ki of <0.01 nM, >4800-fold more selective versus Bcl-xL and Bcl-w, and no activity to Mcl-1. ABT-199 potently inhibits RS4;11 cells with EC₅₀ of 8 nM. In addition, ABT-199 induces a rapid apoptosis in RS4;11 cells with cytochrome c release, caspase activation, and the accumulation of sub-G0/G1 DNA. Quantitative immunoblotting reveals that sensitivity to ABT-199 correlated strongly with the expression of Bcl-2, including NHL, DLBCL, MCL, AML and ALL cell lines. ABT-199 also induces apoptosis in CLL with an average EC₅₀ of 3.0 nM. A single dose of 100 mg/kg of ABT-199 causes a maximal tumor growth inhibition of 95% and tumor growth delay of 152% in RS4;11 xenografts. ABT-199 also inhibits xenograft growth (DoHH2, Granta-519) as a single agent or in combination with Bendamustine and other agents. Human Phase I and ∥ data showed that ABT-199 is highly efficacious for CLL who have 17p deletion, and was approved by FDA in 2016.

WO/2017/132474, WO/2019/040550, and WO/2019/040573 disclosed a novel class of BCL-2 inhibitors. However, there is still a strong need for continuing search in this field of art for more potent BCL-2 inhibitor.

### SUMMARY OF THE INVENTION

In a first embodiment, this invention provides compounds of the Formula (1) or an N-oxide thereof, or a pharmaceutically acceptable salt, solvate, polymorph, tautomer, stereoisomer, an isotopic form, or a prodrug of said compound of Formula (1) or N-oxide thereof:

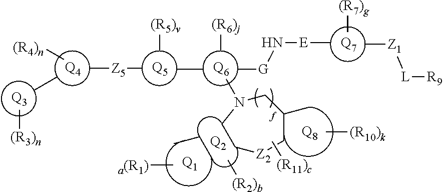

wherein
   G is C(O), S(O₂), P(O)(Rₐ), or S(O)(NRₐ);
   E is S(O₂), P(O)(Rₐ), S(O)(NRₐ) or C(O);
   each of Q₁, Q₂, Q₃, Q₄, Q₅, Q₆, Q₇, and Q₈, independently, is cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is independently optionally substituted with one or more R_{d};
   each of R₁, R₂, R₃, R₄, R₅, R₆, R₇, R₉, R₁₀, and R₁₁, independently, is absent, H, D, alkyl, alkenyl, alkynyl, halo, nitro, oxo, cyano, ORₐ, SRₐ, alkyl-Rₐ, NH(CH₂)ₚRₐ, C(O)Rₐ, S(O)Rₐ, SO₂Rₐ, C(O)ORₐ, OC(O)Rₐ, NR_{b}R_{c}, C(O)N(R_{b})R_{c}, N(R_{b})C(O)R_{c}, —P(O)R_{b}R_{c}, -alkyl-P(O)R_{b}R_{c}, —S(O)(=N(R_{b}))R_{c}, —N═S(O)R_{b}R_{c}, ═NR_{b}, SO₂N(R_{b})R_{c}, N(R_{b})SO₂R_{c}, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is independently optionally substituted with one or more R_{d};
   each of Z₁, Z₂, and Z₅, independently, is a bond, (CRₐR_{b})ₚ, a bivalent alkenyl group, or a bivalent alkynyl group, N(Rₐ), O, S, C(O), S(O₂), —O(CRₐR_{b})ₚ—, —N(Rₐ)(CRₐR_{b})ₚ—, OC(O), C(O)O, OSO₂, S(O₂)O, C(O)S, SC(O), C(O)C(O), C(O)N(Rₐ), N(Rₐ)C(O), S(O₂)N(Rₐ), N(Rₐ)S(O₂), OC(O)O, OC(O)S, OC(O)N(Rₐ), N(Rₐ)C(O)O, N(Rₐ)C(O)S, N(Rₐ)C(O)N(Rₐ), (CRₐR_{b})ₚN(Rₐ)(CRₐR_{b})_{q}, (CRₐR_{b})ₚN(Rₐ)C(O)(CRₐR_{b})_{q}, OC(O)N(R_{b})(CRₐR_{b})ₚ₊₁N(R_{b})(CRₐR_{b})_{q}, or (CRₐR_{b})_{P}C(O)N(Rₐ)(CRₐR_{b})_{q};
   L is absent, a bond, (CRₐR_{b})ₚ, N(R_{c}), O, S, C(O), S(O₂), —O(CRₐR_{b})ₚ—, —N(R_{c})(CRₐR_{b})ₚ—, OC(O), C(O)O, OSO₂, S(O₂)O, C(O)S, SC(O), C(O)C(O), C(O)N(R_{c}), N(R_{c})C(O), S(O₂)N(R_{c}), N(R_{c})S(O₂), OC(O)O, OC(O)S, OC(O)N(R_{c}), N(R_{c})C(O)O, N(R_{c})C(O)S, N(R_{c})C(O)N(R_{c}), (CRₐR_{b})ₚN(R_{c})(CRₐR_{b})_{q}, (CRₐR_{b})_{P}N(R_{c})C(O)(CRₐR_{b})_{q}, OC(O)N(R_{c})(CRₐR_{b})ₚ₊₁N(R_{c})(CRₐR_{b})_{q}, (CRₐR_{b})PC(O)N(R_{c})(CRₐR_{b})_{q}, bivalent alkenyl, bivalent alkynyl, bivalent cycloalkyl, bivalent cycloalkenyl, bivalent spirocycloalkyl, bivalent fused-carbocyclic, bivalent bridged-carbocyclic, bivalent heterocycloalkyl, bivalent heterocycloalkenyl, bivalent spiro-heterocyclic, bivalent fused-heterocyclic, bivalent bridged-heterocyclic, bivalent aryl, or bivalent heteroaryl, each of the aforementioned is independently optionally substituted with one or more R_{d};
   each of Rₐ, R_{b}, R_{c} and R_{d}, independently, is H, D, alkyl, spiroalkyl, alkenyl, alkynyl, halo, cyano, amine, nitro, hydroxy, ═O, -alkyl-O—P(O)(OH)(OH), C(O)NHOH, C(O)OH, C(O)NH₂, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is independently optionally substituted with one or more Rₑ;
   each Rₑ is independently H, D, alkyl, alkenyl, alkynyl, halo, cyano, amine, nitro, hydroxy, ═O, -alkyl-O—P(O)(OH)(OH), C(O)NHOH, alkoxy, alkoxyalkyl, haloalkyl, hydroxyalkyl, aminoalkyl, alkylcarbonyl, alkoxycarbonyl, alkylcarbonylamino, alkylamino, halo-alkylamino, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is independently optionally substituted with one or more R_{f};
   R_{f} is absent, H, D, alkyl, alkenyl, alkynyl, halo, cyano, amine, nitro, hydroxy, ═O, -alkyl-O—P(O)(OH)(OH), C(O)NHOH, alkoxy, alkoxyalkyl, haloalkyl, hydroxyalkyl, aminoalkyl, alkylcarbonyl, alkoxycarbonyl, alkylcarbonylamino, alkylamino, halo-alkylamino, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is independently optionally substituted with one or more R_{g};
   R_{g} is absent, H, D, alkyl, alkenyl, alkynyl, halo, cyano, amine, nitro, hydroxy, ═O, -alkyl-O—P(O)(OH)(OH), C(O)NHOH, alkoxy, alkoxyalkyl, haloalkyl, hydroxyalkyl, aminoalkyl, alkylcarbonyl, alkoxycarbonyl, alkylcarbonylamino, alkylamino, halo-alkylamino, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl;
   Rₐ and R_{b}, groups, taken together with the atom to which they are attached, may optionally form a cycloalkyl, heterocycloalkyl, aryl, or heteroaryl, each of the aforementioned is independently optionally substituted with one or more R_{d};
   R_{b} and R_{c} groups, taken together with the atom to which they are attached, may optionally form a cycloalkyl, heterocycloalkyl, aryl, or heteroaryl, each of the aforementioned is independently optionally substituted with one or more R_{d};
   two of R_{d} group, taken together with the atom to which they are attached, may optionally form a cycloalkyl, heterocycloalkyl, aryl, or heteroaryl, each of the aforementioned is independently optionally substituted with one or more Rₑ;
   two of Rₑ groups, taken together with the atom to which they are attached, may optionally form a cycloalkyl, heterocycloalkyl, aryl, or heteroaryl, each of the aforementioned is independently optionally substituted with one or more R_{f};
   two of R_{f} groups, taken together with the atom to which they are attached, may optionally form a cycloalkyl, heterocycloalkyl, aryl, or heteroaryl, each of the aforementioned is independently optionally substituted with one or more R_{g};
   two of R₁ group, taken together with the atom to which they are attached, may optionally form a cycloalkyl, heterocycloalkyl, aryl, or heteroaryl, each of the aforementioned is independently optionally substituted with one or more R_{d};
   two of R₂ group, taken together with the atom to which they are attached, may optionally form a cycloalkyl, heterocycloalkyl, aryl, or heteroaryl, each of the aforementioned is independently optionally substituted with one or more R_{d};
   two of R₃ group, taken together with the atom to which they are attached, may optionally form a cycloalkyl, heterocycloalkyl, aryl, or heteroaryl, each of the aforementioned is independently optionally substituted with one or more R_{d};
   two of R₄ group, taken together with the atom to which they are attached, may optionally form a cycloalkyl, heterocycloalkyl, aryl, or heteroaryl, each of the aforementioned is independently optionally substituted with one or more R_{d};
   two of R₅ group, taken together with the atom to which they are attached, may optionally form a cycloalkyl, heterocycloalkyl, aryl, or heteroaryl, each of the aforementioned is independently optionally substituted with one or more R_{d};
   two of R₆ group, taken together with the atom to which they are attached, may optionally form a cycloalkyl, heterocycloalkyl, aryl, or heteroaryl, each of the aforementioned is independently optionally substituted with one or more R_{d};
   two of R₇ group, taken together with the atom to which they are attached, may optionally form a cycloalkyl, heterocycloalkyl, aryl, or heteroaryl, each of the aforementioned is independently optionally substituted with one or more R_{d};
   two of R₁₀ group, taken together with the atom to which they are attached, may optionally form a cycloalkyl, heterocycloalkyl, aryl, or heteroaryl, each of the aforementioned is independently optionally substituted with one or more R_{d};
   two of R₁₁ group, taken together with the atom to which they are attached, may optionally form a cycloalkyl, heterocycloalkyl, aryl, or heteroaryl, each of the aforementioned is independently optionally substituted with one or more R_{d};
   R₃ and R₄ group, taken together with the atom to which they are attached, may optionally form a cycloalkyl, heterocycloalkyl, aryl, or heteroaryl, each of the aforementioned is independently optionally substituted with one or more R_{d};
   R₄ and R₅ group, taken together with the atom to which they are attached, may optionally form a cycloalkyl, heterocycloalkyl, aryl, or heteroaryl, each of the aforementioned is independently optionally substituted with one or more R_{d};
   R₅ and R₆ group, taken together with the atom to which they are attached, may optionally form a cycloalkyl, heterocycloalkyl, aryl, or heteroaryl, each of the aforementioned is independently optionally substituted with one or more R_{d};
   R₁₀ and R₁₁ group, taken together with the atom to which they are attached, may optionally form a cycloalkyl, heterocycloalkyl, aryl, or heteroaryl, each of the aforementioned is independently optionally substituted with one or more R_{d};
   R₇ and —Z₁-L-R₉ group, taken together with the atom to which they are attached, may optionally form a cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is independently optionally substituted with one or more R_{d};
   one or more (e.g., both) border ring atom(s) between ring Q₈ and the ring with Z₂ can be carbon or heteroatom(s);
   each of a, b, c, g, j, k, m, n, and v is, independently, 0, 1, 2, 3, 4, 5, 6, 7, or 8;
   f is 0, 1, or 2; and
   each of p, and q is, independently, 0, 1, 2, 3, 4, or 5.

In certain embodiments, the invention provides a compound represented by Formula (2):

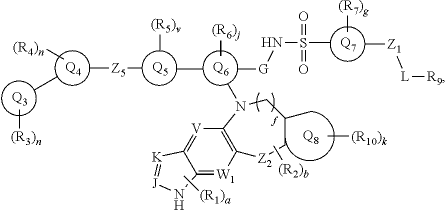

wherein
   each of W₁, V, K, J, independently, is C(Rₐ) or N. The remaining groups are as defined in Formula (1).

In certain embodiments, the invention provides a compound represented by Formula (3):

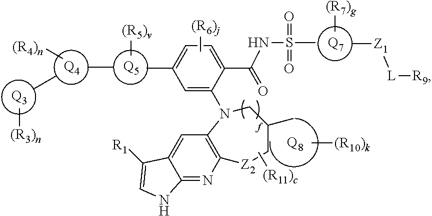

The remaining groups are as defined in Formula (1).

In certain embodiments, the invention provides a compound represented by Formula (4):

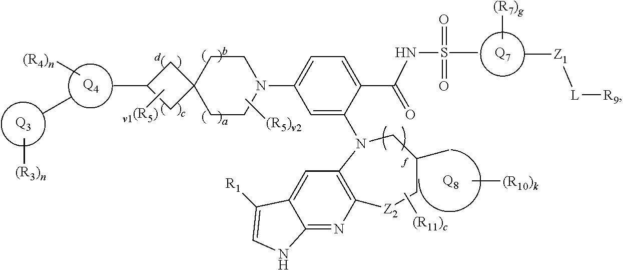

wherein
   each of a, b, c, and d, independently, is 0, 1, or 2; and
   v1 and v2 are both integers including 0, and v1+v2=v.
      The remaining groups are as defined in Formula (1).

In certain embodiments, the invention provides a compound represented by Formula (5):

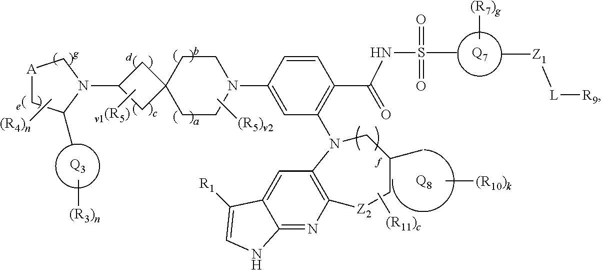

wherein
   each of e and g, independently is 0, 1, or 2; and
   A is O, S, SO₂, N(Rₐ), or C(RₐR^{b}).
   The remaining groups are as defined in Formula (1).

In certain embodiments, the invention provides a compound represented by Formula (6):

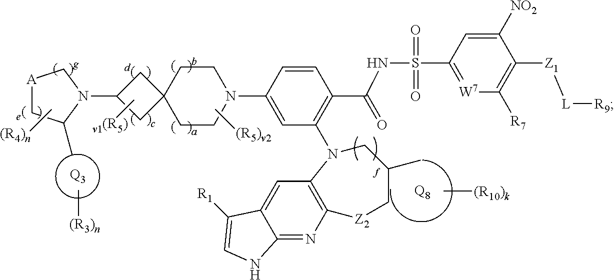

wherein
   W⁷ is N, or C(Rₐ).
   The remaining groups are as defined in Formula (1).

In certain embodiments, the invention provides a compound represented by Formula (7):

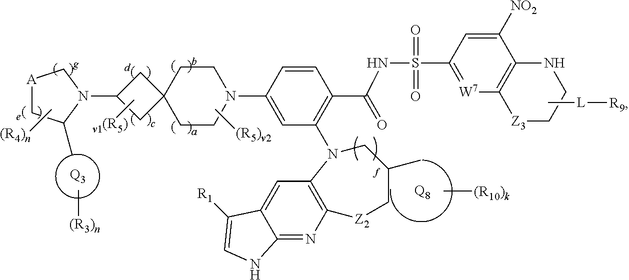

wherein
   W⁷ is N, or C(Rₐ); and
   Z₃ is a bond, (CH₂)ₚ, N(H), O, S, C(O), S(O₂), OC(O), C(O)O, OSO₂, S(O₂)O, C(O)S, SC(O), C(O)C(O), C(O)N(H), N(H)C(O), S(O₂)N(H), N(H)S(O₂), OC(O)O, OC(O)S, OC(O)N(H), N(H)C(O)O, N(H)C(O)S, N(H)C(O)N(H), (CH₂)ₚN(H)(CH₂)_{q}, (CH₂)ₚN(H)C(O)(CH₂)_{q}, (CH₂)PC(O)N(H)(CH₂)_{q}, or OC(O)N(H)(CH₂)ₚ₊₁N(H)(CH₂)_{q}.

In certain embodiments, the invention provides a compound represented by Formula (A):

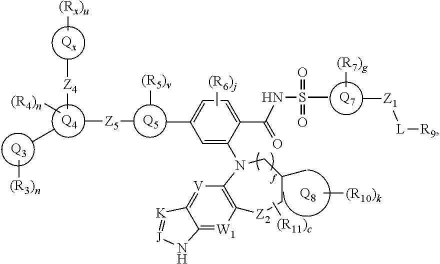

wherein
   each of W₁, V, K, J, independently, is C(Rₐ) or N;
   u is 0, 1, 2, 3, 4, or 5;
   Z₄ is a bond, (CRₐR_{b})ₚ, a bivalent alkenyl group, or a bivalent alkynyl group, N(Rₐ), O, S, C(O), S(O₂), —O(CRₐR_{b})ₚ—, —N(Rₐ)(CRₐR_{b})ₚ—, OC(O), C(O)O, OSO₂, S(O₂)O, C(O)S, SC(O), C(O)C(O), C(O)N(Rₐ), N(Rₐ)C(O), S(O₂)N(Rₐ), N(Rₐ)S(O₂), OC(O)O, OC(O)S, OC(O)N(Rₐ), N(Rₐ)C(O)O, N(Rₐ)C(O)S, N(Rₐ)C(O)N(Rₐ), (CRₐR_{b})ₚN(Rₐ)(CRₐR_{b})_{q}, (CRₐR_{b})ₚN(Rₐ)C(O)(CRₐR_{b})_{q}, OC(O)N(R_{b})(CRₐR_{b})ₚ₊₁N(R_{b})(CRₐR_{b})_{q}, or (CRₐR_{b})PC(O)N(Rₐ)(CRₐR_{b})_{q};
   Qₓ is cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is independently optionally substituted with one or more R_{d}; and
   Rₓ is absent, H, D, alkyl, alkenyl, alkynyl, halo, nitro, oxo, cyano, ORₐ, SRₐ, alkyl-Rₐ, NH(CH₂)ₚRₐ, C(O)Rₐ, S(O)Rₐ, SO₂Rₐ, C(O)ORₐ, OC(O)Rₐ, NR_{b}R_{c}, C(O)N(R_{b})R_{c}, N(R_{b})C(O)R_{c}, —P(O)R_{b}R_{c}, -alkyl-P(O)R_{b}R_{c}, —S(O)(=N(R_{b}))R_{c}, —N═S(O)R_{b}R_{c}, ═NR_{b}, SO₂N(R_{b})R_{c}, or N(R_{b})SO₂R_{c}, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is independently optionally substituted with one or more R_{d}.
   The remaining groups are as defined in Formula (1).

In certain embodiments, the invention provides a compound represented by Formula (IV), wherein R₃ and R₄ group, taken together with the atom to which they are attached, form a cycloalkyl or heterocycloalkyl, in which said cycloalkyl or heterocycloalkyl of R₃ or R₄, is optionally substituted with one or more R_{d}.

In certain embodiments, the invention provides a compound represented by Formula (B):

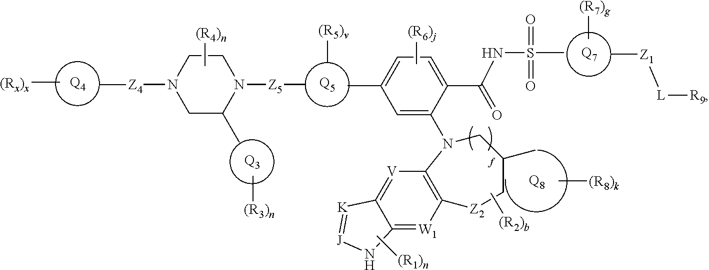

The remaining groups are as defined in Formula (1).

In certain embodiments, the invention provides a compound represented by Formula (C):

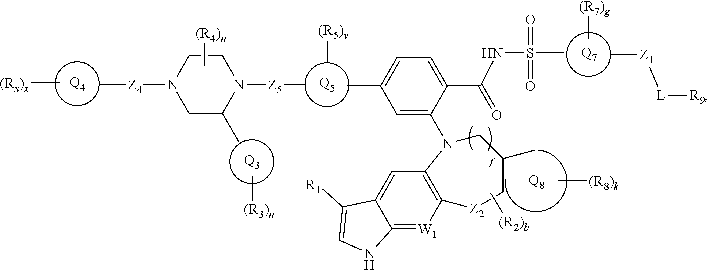

The remaining groups are as defined in Formula (1).

In certain embodiments, the invention provides a compound represented by Formula (D):

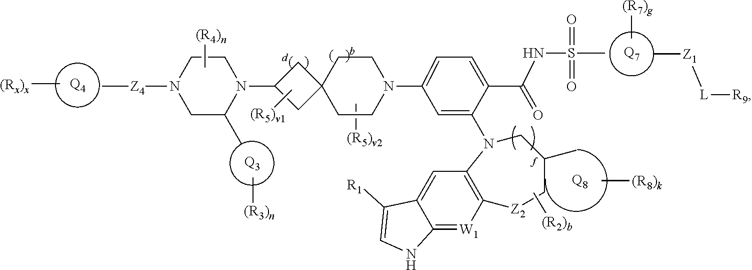

wherein
   each of b, and d, independently, is 0, 1, or 2; and
   v1 and v2 are both integers including 0, and v1+v2=v.
      The remaining groups are as defined in Formula (1).

In certain embodiments, the invention provides a compound represented by Formula (E):

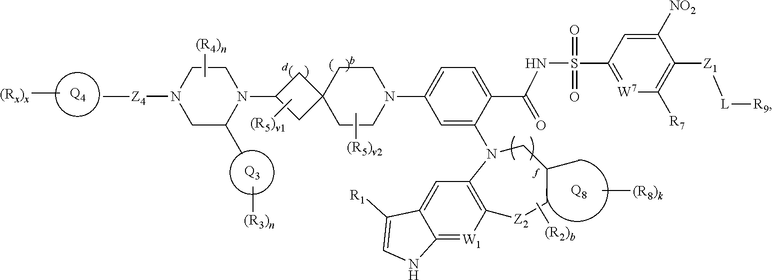

wherein
W⁷ is N, or C(Rₐ).

The remaining groups are as defined in Formula (1).

In certain embodiments, the invention provides a compound represented by Formula (F):

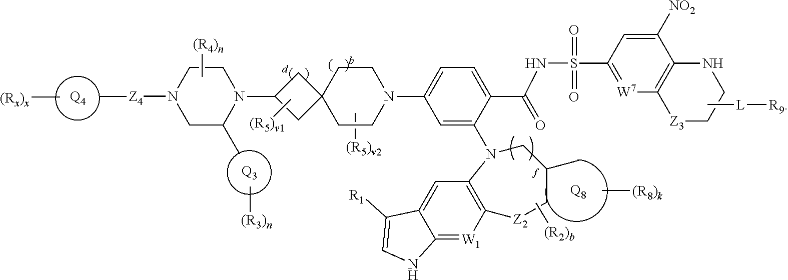

wherein
   W⁷ is N, or C(Rₐ); and
   Z₃ is a bond, (CH₂)ₚ, N(H), O, S, C(O), S(O₂), OC(O), C(O)O, OSO₂, S(O₂)O, C(O)S, SC(O), C(O)C(O), C(O)N(H), N(H)C(O), S(O₂)N(H), N(H)S(O₂), OC(O)O, OC(O)S, OC(O)N(H), N(H)C(O)O, N(H)C(O)S, N(H)C(O)N(H), (CH₂)ₚN(H)(CH₂)_{q}, (CH₂)ₚN(H)C(O)(CH₂)_{q}, (CH₂)PC(O)N(H)(CH₂)_{q}, or OC(O)N(H)(CH₂)ₚ₊₁N(H)(CH₂)_{q}.

In certain embodiments, the invention provides a compound represented by Formula (I):

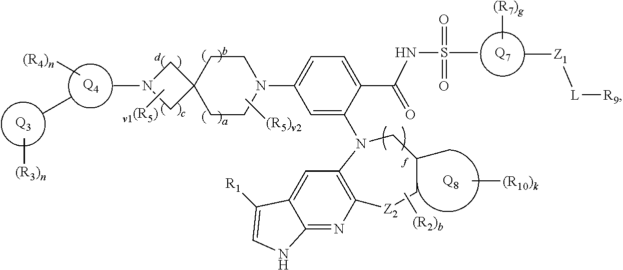

wherein each of a, b, c, and d, independently, is 0, 1, or 2; wherein v1 and v2 are both integers including 0, and v1+v2=v.

The remaining groups are as defined in Formula (1).

In certain embodiments, the invention provides a compound represented by Formula (II):

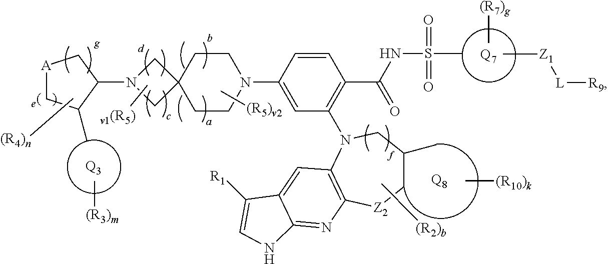

wherein each of e and g, independently is 0, 1, or 2; A is O, S, SO₂, N(Rₐ), or C(RₐR_{b}).

The remaining groups are as defined in Formula (1).

In certain embodiments, the invention provides a compound represented by Formula (III):

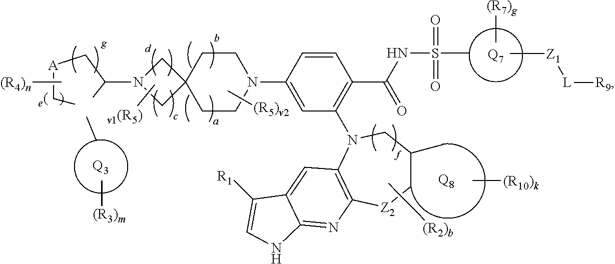

wherein each of e and g, independently is 0, 1, or 2; A is O, S, SO₂, N(Rₐ), or C(RₐR_{b}).

The remaining groups are as defined in Formula (1).

In certain embodiments, the invention provides a compound represented by Formula (IV):

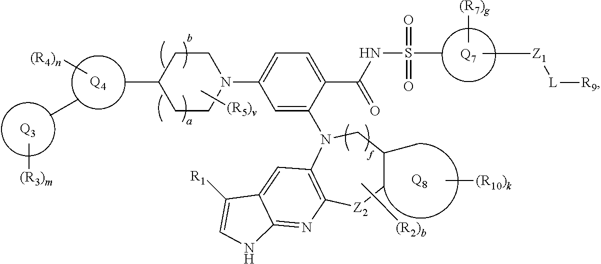

wherein each of a and b, independently, is 0, 1, or 2.

The remaining groups are as defined in Formula (1).

In certain embodiments, the invention provides a compound represented by Formula (V):

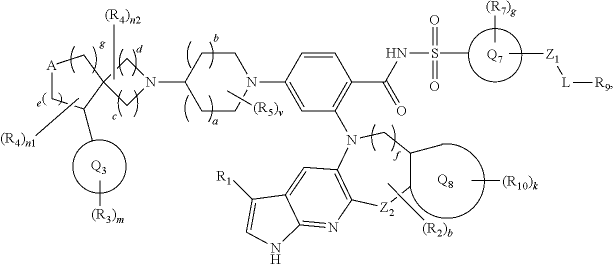

wherein each of a, b, c, d, e and g, independently is 0, 1, or 2; A is O, S, SO₂, N(Rₐ), or C(RₐR_{b}); n1 and n2 are both integers including 0, and n1+n2=n.

The remaining groups are as defined in Formula (1).

In certain embodiments, the invention provides a compound represented by Formula (VI):

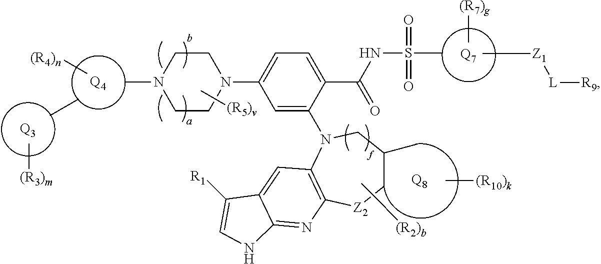

wherein each of a and b, independently, is 0, 1, or 2.

The remaining groups are as defined in Formula (1).

In certain embodiments, the invention provides a compound represented by Formula (VII):

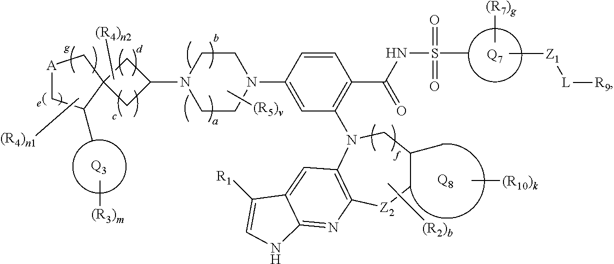

wherein each of a, b, c, d, e and g, independently is 0, 1, or 2; A is O, S, SO₂, N(Rₐ), or C(RₐR_{b}); n1 and n2 are both integers including 0, and n1+n2=n.

The remaining groups are as defined in Formula (1).

In certain embodiments, the invention provides a compound represented by Formula (VIII):

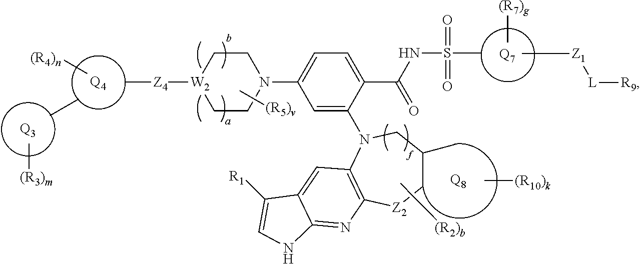

wherein
each of a and b, independently, is 0, 1, or 2;
W₂ is N or C(Rₐ); and
Z₄ is a cycloalkyl, cycloalkenyl, heterocycloalkyl, heterocycloalkenyl, spiroheterocyclyl, fused heterocyclyl, bridged heterocyclyl, aryl, or heteroaryl, in which said cycloalkyl, cycloalkenyl, heterocycloalkyl, heterocycloalkenyl, aryl, or heteroaryl is optionally substituted with one or more R_{d}.

The remaining groups are as defined in Formula (1).

A modified compound of any one of such compounds including a modification having an improved (e.g., enhanced, greater) pharmaceutical solubility, stability, bioavailability, and/or therapeutic index as compared to the unmodified compound is also contemplated. Exemplary modifications include (but are not limited to) applicable prodrug derivatives, and deuterium-enriched compounds.

Also within the scope of this invention is a pharmaceutical composition containing one or more of the compounds (such as any one of those in Formulae (1-7), (A-E), (I-VIII), or a pharmaceutically acceptable salt, solvate, polymorph, tautomer, stereoisomer, an isotopic form, or a prodrug thereof or an N-oxide thereof), modifications, and/or salts thereof described herein, and a pharmaceutically acceptable diluent or carrier, for use in treating a neoplastic disease, therapeutic uses thereof, and use of the compounds for the manufacture of a medicament for treating the disease/disorder.

This invention also relates to a method of treating a neoplastic disease, an autoimmune disease, or a neurodegenerative disease, comprising administering to a subject in need thereof an effective amount of one or more compounds of the invention (such as any one of those in Formulae (1-7), (A-E), (I-VII), or a pharmaceutically acceptable salt, solvate, polymorph, tautomer, stereoisomer, an isotopic form, or a prodrug thereof or an N-oxide thereof), modifications, and/or salts thereof described herein, or a pharmaceutical composition comprising the compound(s) of the invention.

In certain embodiments, the neoplastic disease, autoimmune disease, or neurodegenerative disease is characterized by abnormal (e.g., enhanced or increased) Bcl-2 activity. For example, the neoplastic disease can be a hematological malignancy or cancer including solid tumor; the autoimmune disease can be type I diabetes; and the neurodegenerative disease can be schizophrenia.

In certain embodiments, the neoplastic disease is myeloma, multiple myeloma, lymphoma, follicular lymphoma (FL), non-Hodgkin's lymphoma, leukemia, acute leukemia, acute lymphoblastic leukemia (ALL) (such as BCL-2-dependent ALL and pediatric ALL), chronic lymphoblastic leukemia (CLL) (such as relapsed/refractory CLL, del(17p) CLL), chronic myeloid leukemia (CML) (such as blast-crisis CML), mantle cell lymphoma (MCL), diffuse large B-cell lymphoma, lung cancer such as small cell lung cancer (SCLC), melanoma, breast cancer, or prostate cancer, including drug-resistant cancer thereof.

In certain embodiments, the method further comprises administering one or more further treatment(s) effective to treat the neoplastic disease, such as surgery, radiation therapy, a chemotherapeutic agent (such as bendamustine, NL-101 (7-(5-(bis(2-chloroethyl)amino)-1-methyl-1H-benzo[d]imidazol-2-yl)-N-hydroxyheptanamide), cisplatin, carboplatin, etoposide, topotecan), a target therapy (e.g., an anti-CD20 antibody such as rituximab, a Bruton's tyrosine kinase inhibitor such as ibrutinib and acalabrutinib (ACP-196), a PI3Kδ inhibitor such as idelalisib); an antibody-drug conjugate or ADC (such as anti-CD30 ADC brentuximab vedotin), an immunotherapy (such as an anti-PD-1 antibody including pembrolizumab and nivolumab, or an anti-PD-L1 antibody including atezolizumab, durvalumab, and avelumab), or a CAR-T therapy (such as tisagenlecleucel, axicabtagene ciloleucel).

Also provided herein is the use of one or more compounds of the invention, or a pharmaceutically acceptable salt thereof, or a pharmaceutical composition comprising one or more compounds of the invention, for the preparation of a medicament for the treatment of the above-referenced diseases or conditions.

In another embodiment, provided herein the compounds of the invention, or a pharmaceutically acceptable salt thereof, or a pharmaceutical composition comprising one or more of the disclosed compounds are for use in treating the above-referenced diseases or conditions.

The details of one or more embodiments of the invention are set forth in the description below. Other features, objects, and advantages of the invention will be apparent from the description and from the claims. It should be understood that all embodiments/features of the invention (compounds, pharmaceutical compositions, methods of make/use, etc) described herein, including any specific features described in the examples and original claims, can combine with one another unless not applicable or explicitly disclaimed.

### DETAILED DESCRIPTION OF THE INVENTION

Exemplary compounds described herein include, but are not limited to, the following:
2-((5aS,9aR)-3-fluoro-6,7,9,9a-tetrahydro-1H-pyrano[3,4-b]pyrrolo[3′,2′:5,6]pyrido[3,2-e][1,4]oxazin-5(5aH)-yl)-N-((4-((((1r,4r)-4-hydroxy-4-methylcyclohexyl)methyl)amino)-3-nitrophenyl)sulfonyl)-4-(2-((S)-2-(2-isopropoxyphenyl)-4-methylpiperazin-1-yl)-7-azaspiro[3.5]nonan-7-yl)benzamide,
N-((4-((((1r,4r)-4-hydroxy-4-methylcyclohexyl)methyl)amino)-3-nitrophenyl)sulfonyl)-4-(2-((S)-2-(2-isopropoxyphenyl)-4-methylpiperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((R)-4-methyl-2-(o-tolyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((2S,6R)-2,4-dimethyl-6-(o-tolyl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((2R,6R)-2,4-dimethyl-6-(o-tolyl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((2R,5R)-4,5-dimethyl-2-(o-tolyl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((2R,5S)-4,5-dimethyl-2-(o-tolyl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
2-((5aS,9aR)-3-fluoro-6,7,9,9a-tetrahydro-1H-pyrano[3,4-b]pyrrolo[3′,2′:5,6]pyrido[3,2-e][1,4]oxazin-5(5aH)-yl)-N-((4-((((1r,4r)-4-hydroxy-4-methylcyclohexyl)methyl)amino)-3-nitrophenyl)sulfonyl)-4-(2-((S)-2-(2-isopropoxyphenyl)-4-methylpiperazin-1-yl)-1-methyl-7-azaspiro[3.5]nonan-7-yl)benzamide,
4-(2-((S)-2-(2-(tert-butoxy)phenyl)-4-methylpiperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((S)-4-methyl-2-(2-(trifluoromethoxy)phenyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((S)-2-(2-(2-hydroxypropan-2-yl)phenyl)-4-methylpiperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((S)-2-(2-isopropylphenyl)piperidin-1-yl)-1,1,3,3-tetramethyl-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((R)-2-(2-isopropylphenyl)piperidin-1-yl)-1,1,3,3-tetramethyl-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-2-(o-tolyl)piperidin-1-yl)-7-azaspiro[3.5]nonan-7-yl)benzamide,
N-((4-(((4-fluorotetrahydro-2H-pyran-4-yl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-2-(o-tolyl)piperidin-1-yl)-7-azaspiro[3.5]nonan-7-yl)benzamide,
N-((3-(4-hydroxy-4-methylcyclohexyl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-2-(o- tolyl)piperidin-1-yl)-7-azaspiro[3.5]nonan-7-yl)benzamide,
N-((3-(1-fluoro-4-hydroxy-4-methylcyclohexyl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-2-(o- tolyl)piperidin-1-yl)-7-azaspiro[3.5]nonan-7-yl)benzamide,
N-((4-((((1r,4r)-4-hydroxy-4-methylcyclohexyl)methyl)amino)-3-nitrophenyl)sulfonyl)-4-(2-((R)-2-(2-isopropylphenyl)piperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((S)-2-(2-isopropoxyphenyl)piperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((S)-2-(3-isopropylthiophen-2-yl)piperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((S)-2-(4-isopropylthiophen-3-yl)piperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((S)-2-(2-isopropylthiophen-3-yl)piperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
2-((5aS,9aR)-3-fluoro-6,7,9,9a-tetrahydro-1H-pyrano[3,4-b]pyrrolo[3′,2′:5,6]pyrido[3,2-e][1,4]oxazin-5(5aH)-yl)-N-((4-((((1r,4r)-4-hydroxy-4-methylcyclohexyl)methyl)amino)-3-nitrophenyl)sulfonyl)-4-(2-((R)-2-(2-isopropylphenyl)piperidin-1-yl)-7-azaspiro[3.5]nonan-7-yl)benzamide,
N-(((S)-3-((1r,4S)-4-hydroxy-4-methylcyclohexyl)-5-nitro-3,4-dihydro-2H-benzo[b][1,4]oxazin-7-yl)sulfonyl)-4-(2-((R)-2-(2-isopropylphenyl)piperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-(((S)-3-((1s,4R)-4-hydroxy-4-methylcyclohexyl)-5-nitro-3,4-dihydro-2H-benzo[b][1,4]oxazin-7-yl)sulfonyl)-4-(2-((R)-2-(2-isopropylphenyl)piperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-3-((1r,4R)-4-hydroxy-4-methylcyclohexyl)-5-nitro-3,4-dihydro-2H-benzo[b][1,4]oxazin-7-yl)sulfonyl)-4-(2-((R)-2-(2-isopropylphenyl)piperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-3-((1s,4S)-4-hydroxy-4-methylcyclohexyl)-5-nitro-3,4-dihydro-2H-benzo[b][1,4]oxazin-7-yl)sulfonyl)-4-(2-((R)-2-(2-isopropylphenyl)piperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-3-((1s,4S)-1-fluoro-4-hydroxy-4-methylcyclohexyl)-5-nitro-3,4-dihydro-2H-benzo[b][1,4]oxazin-7-yl)sulfonyl)-4-(2-((R)-2-(2-isopropylphenyl)piperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-(((S)-3-((1s,4R)-1-fluoro-4-hydroxy-4-methylcyclohexyl)-5-nitro-3,4-dihydro-2H-benzo[b][1,4]oxazin-7-yl)sulfonyl)-4-(2-((R)-2-(2-isopropylphenyl)piperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-3-((1r,4R)-1-fluoro-4-hydroxy-4-methylcyclohexyl)-5-nitro-3,4-dihydro-2H-benzo[b][1,4]oxazin-7-yl)sulfonyl)-4-(2-((R)-2-(2-isopropylphenyl)piperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-(((S)-3-((1r,4S)-1-fluoro-4-hydroxy-4-methylcyclohexyl)-5-nitro-3,4-dihydro-2H-benzo[b][1,4]oxazin-7-yl)sulfonyl)-4-(2-((R)-2-(2-isopropylphenyl)piperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-2-(2-isopropylphenyl)piperidin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-(((4-fluorotetrahydro-2H-pyran-4-yl)methyl)amino)-3-nitrophenyl)sulfonyl)-4-(2-((R)-2-(2-isopropylphenyl)piperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-2-(2-cyclopropoxyphenyl)piperidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-2-(2-cyclobutoxyphenyl)piperidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-((1S,2S,4S,6R)-2-((S)-2-(2-isopropylphenyl)piperidin-1-yl)-1,6-dimethyl-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-((1R,2R,4S,6S)-2-((R)-2-(2-isopropylphenyl)piperidin-1-yl)-1,6-dimethyl-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((S)-2-(2-isopropoxyphenyl)piperidin-1-yl)-1-methyl-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((R)-2-(2-isopropylphenyl)piperidin-1-yl)-1-methyl-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-(((S)-3-((1r,4S)-4-hydroxy-4-methylcyclohexyl)-5-nitro-3,4-dihydro-2H-benzo[b][1,4]oxazin-7-yl)sulfonyl)-4-(2-((S)-2-(2-isopropoxyphenyl)piperidin-1-yl)-1-methyl-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-(((S)-3-((1r,4S)-4-hydroxy-4-methylcyclohexyl)-5-nitro-3,4-dihydro-2H-benzo[b][1,4]oxazin-7-yl)sulfonyl)-4-(2-((R)-2-(2-isopropylphenyl)piperidin-1-yl)-1-methyl-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
2-((5aS,9aR)-3-fluoro-6,7,9,9a-tetrahydro-1H-pyrano[3,4-b]pyrrolo[3′,2′:5,6]pyrido[3,2-e][1,4]oxazin-5(5aH)-yl)-N-((4-((((1 r,4r)-4-hydroxy-4-methylcyclohexyl)methyl)amino)-3-nitrophenyl)sulfonyl)-4-(2-((R)-2-(o-tolyl)piperidin-1-yl)-7-azaspiro[3.5]nonan-7-yl)benzamide,
2-((5aS,9aR)-3-fluoro-6,7,9,9a-tetrahydro-1H-pyrano[3,4-b]pyrrolo[3′,2′:5,6]pyrido[3,2-e][1,4]oxazin-5(5aH)-yl)-N-((4-((((1 r,4r)-4-hydroxy-4-methylcyclohexyl)methyl)amino)-3-nitrophenyl)sulfonyl)-4-(6-((R)-2-(o-tolyl)piperidin-1-yl)-2-azaspiro[3.3]heptan-2-yl)benzamide,
4-(2-((2S,4R)-4-hydroxy-2-(2-isopropylphenyl)-4-methylpiperidin-1-yl)-7-azaspiro[3.5]nonan-7-yl)-N-((4-((((1 r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((2S,4S)-4-hydroxy-2-(2-isopropylphenyl)-4-methylpiperidin-1-yl)-7-azaspiro[3.5]nonan-7-yl)-N-((4-((((1 r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((2R,4R)-4-hydroxy-2-(2-isopropylphenyl)-4-methylpiperidin-1-yl)-7-azaspiro[3.5]nonan-7-yl)-N-((4-((((1 r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((2R,4S)-4-hydroxy-2-(2-isopropylphenyl)-4-methylpiperidin-1-yl)-7-azaspiro[3.5]nonan-7-yl)-N-((4-((((1 r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((2S,4R)-4-hydroxy-4-methyl-2-(o-tolyl)piperidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((2S,4S)-4-hydroxy-4-methyl-2-(o-tolyl)piperidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((2R,4R)-4-hydroxy-4-methyl-2-(o-tolyl)piperidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((2R,4S)-4-hydroxy-4-methyl-2-(o-tolyl)piperidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((2S,5S)-5-hydroxy-2-(2-isopropylphenyl)-5-methylpiperidin-1-yl)-7-azaspiro[3.5]nonan-7-yl)-N-((4-((((1 r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((2S,5R)-5-hydroxy-2-(2-isopropylphenyl)-5-methylpiperidin-1-yl)-7-azaspiro[3.5]nonan-7-yl)-N-((4-((((1 r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((2R,5S)-5-hydroxy-2-(2-isopropylphenyl)-5-methylpiperidin-1-yl)-7-azaspiro[3.5]nonan-7-yl)-N-((4-((((1 r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((2R,5R)-5-hydroxy-2-(2-isopropylphenyl)-5-methylpiperidin-1-yl)-7-azaspiro[3.5]nonan-7-yl)-N-((4-((((1 r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((2S,5S)-5-hydroxy-5-methyl-2-(o-tolyl)piperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((2S,5R)-5-hydroxy-5-methyl-2-(o-tolyl)piperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((2R,5S)-5-hydroxy-5-methyl-2-(o-tolyl)piperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((2R,5R)-5-hydroxy-5-methyl-2-(o-tolyl)piperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((S)-2-(2-isopropoxyphenyl)-4,4-dimethylpiperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((2S,5S)-5-hydroxy-2-(2-isopropoxyphenyl)-5-methylpiperidin-1-yl)-7-azaspiro[3.5]nonan-7-yl)-N-((4-((((1 r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((2S,5R)-5-hydroxy-2-(2-isopropoxyphenyl)-5-methylpiperidin-1-yl)-7-azaspiro[3.5]nonan-7-yl)-N-((4-((((1 r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((2S,4R)-4-hydroxy-2-(2-isopropoxyphenyl)-4-methylpiperidin-1-yl)-7-azaspiro[3.5]nonan-7-yl)-N-((4-((((1 r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((2S,4S)-4-hydroxy-2-(2-isopropoxyphenyl)-4-methylpiperidin-1-yl)-7-azaspiro[3.5]nonan-7-yl)-N-((4-((((1 r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((S)-2-(2-isopropoxyphenyl)-5,5-dimethylpiperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
(1R,4s)-4-fluoro-4-((S)-7-(N-(2-((5aS,9aR)-3-fluoro-7,7-dimethyl-6,7,9,9a-tetrahydro-1H-pyrano[3,4-b]pyrrolo[3′,2′:5,6]pyrido[3,2-e][1,4]oxazin-5(5aH)-yl)-4-((R)-2-((R)-2-(2-isopropylphenyl)-5,5-dimethylpiperidin-1-yl)-6-methyl-7-azaspiro[3.5]nonan-7-yl)benzoyl)sulfamoyl)-5-nitro-3,4-dihydro-2H-benzo[b][1,4]oxazin-3-yl)-1-methylcyclohexyl dihydrogen phosphate,
N-((4-((((1r,4r)-4-hydroxy-4-methylcyclohexyl)methyl)amino)-3-nitrophenyl)sulfonyl)-4-(2-((R)-2-(2-isopropylphenyl)-4,4-dimethylpiperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((S)-2-(2-isopropylphenyl)-4,4-dimethylpiperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-(4-hydroxy-2-(2-isopropylphenyl)-4-methylpiperidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((R)-2-(2-isopropylphenyl)-5,5-dimethylpiperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((S)-2-(2-isopropylphenyl)-5,5-dimethylpiperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-(5-hydroxy-2-(2-isopropylphenyl)-5-methylpiperidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-3-(2-(2-aminopropan-2-yl)phenyl)morpholino)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(2-((S)-1,1,1-trifluoro-2-hydroxypropan-2-yl)phenyl)morpholino)-7-azaspiro[3.5]nonan-7-yl)benzamide,
4-(2-((R)-3-(2-(1,1,1,3,3,3-hexafluoro-2-hydroxypropan-2-yl)phenyl)morpholino)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-3-(2-(tert-butyl)phenyl)morpholino)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-3-(2-ethynylphenyl)morpholino)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(2-(1,1,1-trifluoro-2-methylpropan-2-yl)phenyl)morpholino)-7-azaspiro[3.5]nonan-7-yl)benzamide,
N-((4-((((1r,4r)-4-hydroxy-4-methylcyclohexyl)methyl)amino)-3-nitrophenyl)sulfonyl)-4-(2-((R)-3-(2-isopropoxyphenyl)morpholino)-7-azaspiro[3.5]nonan-7-yl)-2-((5aS,7S,9aR)-7-methyl-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((R)-3-(2-isopropoxyphenyl)morpholino)-7-azaspiro[3.5]nonan-7-yl)-2-((5aS,7R,9aR)-7-methyl-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(6-((R)-3-(2-isopropoxyphenyl)morpholino)-2-azaspiro[3.3]heptan-2-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
2-((5aS,9aR)-3-fluoro-6,7,9,9a-tetrahydro-1H-pyrano[3,4-b]pyrrolo[3′,2′:5,6]pyrido[3,2-e][1,4]oxazin-5(5aH)-yl)-N-((4-((((1r,4r)-4-hydroxy-4-methylcyclohexyl)methyl)amino)-3-nitrophenyl)sulfonyl)-4-(6-((R)-3-(2-isopropoxyphenyl)morpholino)-2-azaspiro[3.3]heptan-2-yl)benzamide,
4-(2-((R)-3-(2-isopropoxyphenyl)morpholino)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-(((4-fluorotetrahydro-2H-pyran-4-yl)methyl)amino)-3-nitrophenyl)sulfonyl)-4-(2-((R)-3-(2-isopropoxyphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-3-((1r,4R)-4-hydroxy-4-methylcyclohexyl)-5-nitro-3,4-dihydro-2H-benzo[b][1,4]oxazin-7-yl)sulfonyl)-4-(2-((R)-3-(2-isopropoxyphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-(((S)-3-((1r,4S)-4-hydroxy-4-methylcyclohexyl)-5-nitro-3,4-dihydro-2H-benzo[b][1,4]oxazin-7-yl)sulfonyl)-4-(2-((R)-3-(2-isopropoxyphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-(((S)-3-((1s,4R)-4-hydroxy-4-methylcyclohexyl)-5-nitro-3,4-dihydro-2H-benzo[b][1,4]oxazin-7-yl)sulfonyl)-4-(2-((R)-3-(2-isopropoxyphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-3-((1s,4S)-4-hydroxy-4-methylcyclohexyl)-5-nitro-3,4-dihydro-2H-benzo[b][1,4]oxazin-7-yl)sulfonyl)-4-(2-((R)-3-(2-isopropoxyphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-3-((1s,4S)-1-fluoro-4-hydroxy-4-methylcyclohexyl)-5-nitro-3,4-dihydro-2H-benzo[b][1,4]oxazin-7-yl)sulfonyl)-4-(2-((R)-3-(2-isopropoxyphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-(((S)-3-((1s,4R)-1-fluoro-4-hydroxy-4-methylcyclohexyl)-5-nitro-3,4-dihydro-2H-benzo[b][1,4]oxazin-7-yl)sulfonyl)-4-(2-((R)-3-(2-isopropoxyphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-3-((1r,4R)-1-fluoro-4-hydroxy-4-methylcyclohexyl)-5-nitro-3,4-dihydro-2H-benzo[b][1,4]oxazin-7-yl)sulfonyl)-4-(2-((R)-3-(2-isopropoxyphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-(((S)-3-((1r,4S)-1-fluoro-4-hydroxy-4-methylcyclohexyl)-5-nitro-3,4-dihydro-2H-benzo[b][1,4]oxazin-7-yl)sulfonyl)-4-(2-((R)-3-(2-isopropoxyphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-3-(2-(tert-butoxy)phenyl)morpholino)-7-azaspiro[3.5]nonan-7-yl)-N-(((S)-3-((1r,4S)-1-fluoro-4-hydroxy-4-methylcyclohexyl)-5-nitro-3,4-dihydro-2H-benzo[b][1,4]oxazin-7-yl)sulfonyl)-2-((5aS,9aR)-3-fluoro-6,7,9,9a-tetrahydro-1H-pyrano[3,4-b]pyrrolo[3′,2′:5,6]pyrido[3,2-e][1,4]oxazin-5(5aH)-yl)benzamide,
N-(((S)-3-((1r,4S)-1-fluoro-4-hydroxy-4-methylcyclohexyl)-5-nitro-3,4-dihydro-2H-benzo[b][1,4]oxazin-7-yl)sulfonyl)-2-((5aS,9aR)-3-fluoro-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(2-(2-hydroxypropan-2-yl)phenyl)morpholino)-7-azaspiro[3.5]nonan-7-yl)benzamide,
N-(((S)-3-((1r,4S)-1-fluoro-4-hydroxy-4-methylcyclohexyl)-5-nitro-3,4-dihydro-2H-benzo[b][1,4]oxazin-7-yl)sulfonyl)-2-((5aS,9aR)-3-fluoro-6,7,9,9a-tetrahydro-1H-pyrano[3,4-b]pyrrolo[3′,2′:5,6]pyrido[3,2-e][1,4]oxazin-5(5aH)-yl)-4-(2-((R)-3-(2-(trifluoromethoxy)phenyl)morpholino)-7-azaspiro[3.5]nonan-7-yl)benzamide,
N-(((S)-3-((1r,4S)-1-fluoro-4-hydroxy-4-methylcyclohexyl)-5-nitro-3,4-dihydro-2H-benzo[b][1,4]oxazin-7-yl)sulfonyl)-2-((5aS,9aR)-3-fluoro-6,7,9,9a-tetrahydro-1H-pyrano[3,4-b]pyrrolo[3′,2′:5,6]pyrido[3,2-e][1,4]oxazin-5(5aH)-yl)-4-(2-((R)-5-(2-isopropoxyphenyl)-2,2-dimethylmorpholino)-7-azaspiro[3.5]nonan-7-yl)benzamide,
N-(((S)-3-((1r,4S)-1-fluoro-4-hydroxy-4-methylcyclohexyl)-5-nitro-3,4-dihydro-2H-benzo[b][1,4]oxazin-7-yl)sulfonyl)-2-((5aS,9aR)-3-fluoro-6,7,9,9a-tetrahydro-1H-pyrano[3,4-b]pyrrolo[3′,2′:5,6]pyrido[3,2-e][1,4]oxazin-5(5aH)-yl)-4-(2-((2S,5R)-5-(2-isopropoxyphenyl)-2-methylmorpholino)-7-azaspiro[3.5]nonan-7-yl)benzamide,
N-(((S)-3-((1r,4S)-1-fluoro-4-hydroxy-4-methylcyclohexyl)-5-nitro-3,4-dihydro-2H-benzo[b][1,4]oxazin-7-yl)sulfonyl)-2-((5aS,9aR)-3-fluoro-6,7,9,9a-tetrahydro-1H-pyrano[3,4-b]pyrrolo[3′,2′:5,6]pyrido[3,2-e][1,4]oxazin-5(5aH)-yl)-4-(2-((2R,5R)-5-(2-isopropoxyphenyl)-2-methylmorpholino)-7-azaspiro[3.5]nonan-7-yl)benzamide,
(1S,4r)-4-fluoro-4-((3S)-7-(N-(2-((5aS,9aR)-3-fluoro-7,7-dimethyl-6,7,9,9a-tetrahydro-1H-pyrano[3,4-b]pyrrolo[3′,2′:5,6]pyrido[3,2-e][1,4]oxazin-5(5aH)-yl)-4-((1R,4R,6R)-2-((R)-3-(2-isopropoxyphenyl)morpholino)-1,6-dimethyl-7-azaspiro[3.5]nonan-7-yl)benzoyl)sulfamoyl)-5-nitro-3,4-dihydro-2H-benzo[b][1,4]oxazin-3-yl)-1-methylcyclohexyl dihydrogen phosphate
N-((4-((((1r,4r)-4-hydroxy-4-methylcyclohexyl)methyl)amino)-3-nitrophenyl)sulfonyl)-4-(2-((R)-3-(2-isopropoxyphenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((S)-2-(2-(trifluoromethoxy)phenyl)piperidin-1-yl)-7-azaspiro[3.5]nonan-7-yl)benzamide,
4-(2-((S)-2-(2-(tert-butoxy)phenyl)piperidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((R)-3-(2-(2-hydroxypropan-2-yl)phenyl)morpholino)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-4-((tetrahydro-2H-pyran-4-yl)methyl)-2-(o-tolyl)piperazin-1-yl)-7-azaspiro[3.5]nonan-7-yl)benzamide,
4-(2-((R)-2-(2-isopropylphenyl)-4-((tetrahydro-2H-pyran-4-yl)methyl)piperazin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-2-(2-isopropoxyphenyl)-4-((tetrahydro-2H-pyran-4-yl)methyl)piperazin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((R)-2-(2-isopropylphenyl)-4-((tetrahydro-2H-pyran-4-yl)methyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-4-((tetrahydro-2H-pyran-4-yl)methyl)-2-(o-tolyl)piperazin-1-yl)-7-azaspiro[3.5]nonan-7-yl)benzamide,
N-((4-((((1r,4r)-4-hydroxy-4-methylcyclohexyl)methyl)amino)-3-nitrophenyl)sulfonyl)-4-(2-((S)-2-(2-isopropoxyphenyl)-4-((tetrahydro-2H-pyran-4-yl)methyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((4-hydroxy-4-methylcyclohexyl)methyl)-2-(2-isopropylphenyl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((4-hydroxy-4-methylcyclohexyl)methyl)-2-(o-tolyl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-4-((4-hydroxy-4-methylcyclohexyl)methyl)-2-(2-isopropoxyphenyl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((S)-2-(2-isopropoxyphenyl)-4-((tetrahydro-2H-pyran-4-yl)methyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((R)-2-(2-isopropylphenyl)-4-(4-methoxybenzyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(3,4-difluorobenzyl)-2-(2-isopropylphenyl)piperazin-1-yl)-7-azaspiro[3.5]nonan-7-yl)-N-((4-((((1 r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(4-cyclopropyl-3-methoxybenzyl)-2-(2-isopropylphenyl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((R)-2-(2-isopropylphenyl)-4-((7-methoxy-2-methylbenzofuran-5-yl)methyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
2-((5aS,9aR)-3-fluoro-6,7,9,9a-tetrahydro-1H-pyrano[3,4-b]pyrrolo[3′,2′:5,6]pyrido[3,2-e][1,4]oxazin-5(5aH)-yl)-N-((4-((((1r,4r)-4-hydroxy-4-methylcyclohexyl)methyl)amino)-3-nitrophenyl)sulfonyl)-4-(2-((R)-2-(2-isopropylphenyl)-4-(4-methoxybenzyl)piperazin-1-yl)-7-azaspiro[3.5]nonan-7-yl)benzamide,
4-(2-((R)-4-(3,4-difluorobenzyl)-2-(2-isopropylphenyl)piperazin-1-yl)-7-azaspiro[3.5]nonan-7-yl)-2-((5aS,9aR)-3-fluoro-6,7,9,9a-tetrahydro-1H-pyrano[3,4-b]pyrrolo[3′,2′:5,6]pyrido[3,2-e][1,4]oxazin-5(5aH)-yl)-N-((4-((((1r,4r)-4-hydroxy-4-methylcyclohexyl)methyl)amino)-3-nitrophenyl)sulfonyl)benzamide,
4-(2-((R)-4-(4-cyclopropyl-3-methoxybenzyl)-2-(2-isopropylphenyl)piperazin-1-yl)-7-azaspiro[3.5]nonan-7-yl)-2-((5aS,9aR)-3-fluoro-6,7,9,9a-tetrahydro-1H-pyrano[3,4-b]pyrrolo[3′,2′:5,6]pyrido[3,2-e][1,4]oxazin-5(5aH)-yl)-N-((4-((((1r,4r)-4-hydroxy-4-methylcyclohexyl)methyl)amino)-3-nitrophenyl)sulfonyl)benzamide,
2-((5aS,9aR)-3-fluoro-6,7,9,9a-tetrahydro-1H-pyrano[3,4-b]pyrrolo[3′,2′:5,6]pyrido[3,2-e][1,4]oxazin-5(5aH)-yl)-N-((4-((((1r,4r)-4-hydroxy-4-methylcyclohexyl)methyl)amino)-3-nitrophenyl)sulfonyl)-4-(2-((R)-2-(2-isopropylphenyl)-4-((7-methoxy-2-methylbenzofuran-5-yl)methyl)piperazin-1-yl)-7-azaspiro[3.5]nonan-7-yl)benzamide,
N-((4-((((1r,4r)-4-hydroxy-4-methylcyclohexyl)methyl)amino)-3-nitrophenyl)sulfonyl)-4-(6-((R)-2-(2-isopropylphenyl)-4-(4-methoxybenzyl)piperazin-1-yl)-2-azaspiro[3.3]heptan-2-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(6-((R)-4-(3,4-difluorobenzyl)-2-(2-isopropylphenyl)piperazin-1-yl)-2-azaspiro[3.3]heptan-2-yl)-N-((4-((((1 r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(6-((R)-4-(4-cyclopropyl-3-methoxybenzyl)-2-(2-isopropylphenyl)piperazin-1-yl)-2-azaspiro[3.3]heptan-2-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(6-((R)-2-(2-isopropylphenyl)-4-((7-methoxy-2-methylbenzofuran-5-yl)methyl)piperazin-1-yl)-2-azaspiro[3.3]heptan-2-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
2-((5aS,9aR)-3-fluoro-6,7,9,9a-tetrahydro-1H-pyrano[3,4-b]pyrrolo[3′,2′:5,6]pyrido[3,2-e][1,4]oxazin-5(5aH)-yl)-N-((4-((((1r,4r)-4-hydroxy-4-methylcyclohexyl)methyl)amino)-3-nitrophenyl)sulfonyl)-4-(6-((R)-2-(2-isopropylphenyl)-4-(4-methoxybenzyl)piperazin-1-yl)-2-azaspiro[3.3]heptan-2-yl)benzamide,
4-(6-((R)-4-(3,4-difluorobenzyl)-2-(2-isopropylphenyl)piperazin-1-yl)-2-azaspiro[3.3]heptan-2-yl)-2-((5aS,9aR)-3-fluoro-6,7,9,9a-tetrahydro-1H-pyrano[3,4-b]pyrrolo[3′,2′:5,6]pyrido[3,2-e][1,4]oxazin-5(5aH)-yl)-N-((4-((((1r,4r)-4-hydroxy-4-methylcyclohexyl)methyl)amino)-3-nitrophenyl)sulfonyl)benzamide,
4-(6-((R)-4-(4-cyclopropyl-3-methoxybenzyl)-2-(2-isopropylphenyl)piperazin-1-yl)-2-azaspiro[3.3]heptan-2-yl)-2-((5aS,9aR)-3-fluoro-6,7,9,9a-tetrahydro-1H-pyrano[3,4-b]pyrrolo[3′,2′:5,6]pyrido[3,2-e][1,4]oxazin-5(5aH)-yl)-N-((4-((((1r,4r)-4-hydroxy-4-methylcyclohexyl)methyl)amino)-3-nitrophenyl)sulfonyl)benzamide,
2-((5aS,9aR)-3-fluoro-6,7,9,9a-tetrahydro-1H-pyrano[3,4-b]pyrrolo[3′,2′:5,6]pyrido[3,2-e][1,4]oxazin-5(5aH)-yl)-N-((4-((((1r,4r)-4-hydroxy-4-methylcyclohexyl)methyl)amino)-3-nitrophenyl)sulfonyl)-4-(6-((R)-2-(2-isopropylphenyl)-4-((7-methoxy-2-methylbenzofuran-5-yl)methyl)piperazin-1-yl)-2-azaspiro[3.3]heptan-2-yl)benzamide,
4-(2-((R)-4-(3,4-difluorobenzyl)-2-(2-isopropylphenyl)piperazin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-4-(3,4-difluorobenzyl)-2-(2-isopropylphenyl)piperazin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-4-(4-cyclopropyl-3-methoxybenzyl)-2-(2-isopropoxyphenyl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((S)-2-(2-isopropoxyphenyl)-4-((7-methoxy-2-methylbenzofuran-5-yl)methyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((S)-2-(2-isopropoxyphenyl)-4-(4-methoxybenzyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-4-(3,4-difluorobenzyl)-2-(2-isopropoxyphenyl)piperazin-1-yl)-7-azaspiro[3.5]nonan-7-yl)-N-((4-((((1 r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-4-(3,4-difluorobenzyl)-2-(2-isopropoxyphenyl)piperazin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-4-(3,4-difluorobenzyl)-2-(2-isopropoxyphenyl)piperazin-1-yl)-7-azaspiro[3.5]nonan-7-yl)-N—(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-4-(3,4-difluorobenzyl)-2-(2-isopropoxyphenyl)piperazin-1-yl)-7-azaspiro[3.5]nonan-7-yl)-N—(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-4-(3,4-difluorobenzyl)-2-(2-isopropoxyphenyl)piperazin-1-yl)-7-azaspiro[3.5]nonan-7-yl)-N-(((S)-3-((1r,4S)-4-hydroxy-4-methylcyclohexyl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((S)-2-(2-isopropoxyphenyl)-4-(4-((R)-3-methylmorpholino)benzyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-4-(3-fluoro-4-((R)-3-methylmorpholino)benzyl)-2-(2-isopropoxyphenyl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(4-cyclopropyl-3-methoxybenzyl)-2-(o-tolyl)piperazin-1-yl)-7-azaspiro[3.5]nonan-7-yl)-N-((4-((((1 r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((R)-4-((7-methoxy-2-methylbenzofuran-5-yl)methyl)-2-(o-tolyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((R)-4-(4-methoxybenzyl)-2-(o-tolyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(3,4-difluorobenzyl)-2-(o-tolyl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((R)-4-(4-((R)-3-methylmorpholino)benzyl)-2-(o-tolyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(3-fluoro-4-((R)-3-methylmorpholino)benzyl)-2-(o-tolyl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-((1R,2S)-2-((R)-4-(3,4-difluorobenzyl)-2-(o-tolyl)piperazin-1-yl)-1-methyl-7-azaspiro[3.5]nonan-7-yl)-N-((4-((((1 r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-((1S,2S)-2-((R)-4-(3,4-difluorobenzyl)-2-(o-tolyl)piperazin-1-yl)-1-methyl-7-azaspiro[3.5]nonan-7-yl)-N-((4-((((1 r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(3,4-difluorobenzyl)-2-(o-tolyl)piperazin-1-yl)-7-azaspiro[3.5]nonan-7-yl)-2-((5aS,9aR)-3-fluoro-6,7,9,9a-tetrahydro-1H-pyrano[3,4-b]pyrrolo[3′,2′:5,6]pyrido[3,2-e][1,4]oxazin-5(5aH)-yl)-N-((4-((((1r,4r)-4-hydroxy-4-methylcyclohexyl)methyl)amino)-3-nitrophenyl)sulfonyl)benzamide,
4-(2-((R)-4-(3,4-difluorobenzyl)-2-(o-tolyl)piperazin-1-yl)-7-azaspiro[3.5]nonan-7-yl)-N—(((R)-3-((1r,4R)-4-hydroxy-4-methylcyclohexyl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(3,4-difluorobenzyl)-2-(o-tolyl)piperazin-1-yl)-7-azaspiro[3.5]nonan-7-yl)-N-(((S)-3-((1r,4S)-4-hydroxy-4-methylcyclohexyl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(3,4-difluorobenzyl)-2-(o-tolyl)piperazin-1-yl)-7-azaspiro[3.5]nonan-7-yl)-N-(((S)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(3,4-difluorobenzyl)-2-(o-tolyl)piperazin-1-yl)-7-azaspiro[3.5]nonan-7-yl)-N—(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(3,4-difluorobenzyl)-2-(o-tolyl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aR,9aS)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(3,4-difluorobenzyl)-2-(o-tolyl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aR,9aR)-5a,8,9,9a-tetrahydro-6H-pyrano[4,3-b]pyrrolo[3′,2′:5,6]pyrido[3,2-e][1,4]oxazin-5(1H)-yl)benzamide,
4-(2-((R)-4-(3,4-difluorobenzyl)-2-(o-tolyl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aS)-5a,8,9,9a-tetrahydro-6H-pyrano[4,3-b]pyrrolo[3′,2′:5,6]pyrido[3,2-e][1,4]oxazin-5(1H)-yl)benzamide,
4-(2-((R)-4-(3,4-difluorobenzyl)-2-(o-tolyl)piperazin-1-yl)-7-azaspiro[3.5]nonan-7-yl)-2-((5aS,9aR)-7,7-dimethyl-6,7,9,9a-tetrahydro-1H-pyrano[3,4-b]pyrrolo[3′,2′:5,6]pyrido[3,2-e][1,4]oxazin-5(5aH)-yl)-N-((4-((((1r,4r)-4-hydroxy-4-methylcyclohexyl)methyl)amino)-3-nitrophenyl)sulfonyl)benzamide,
4-(2-((R)-4-((S)-1-(3,4-difluorophenyl)ethyl)-2-(o-tolyl)piperazin-1-yl)-7-azaspiro[3.5]nonan-7-yl)-N-((4-((((1 r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((R)-1-(3,4-difluorophenyl)ethyl)-2-(o-tolyl)piperazin-1-yl)-7-azaspiro[3.5]nonan-7-yl)-N-((4-((((1 r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(3,4-difluorobenzyl)-2-(o-tolyl)piperazin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-4-(3,4-difluorobenzyl)-2-(2-isopropoxyphenyl)piperazin-1-yl)-7-azaspiro[3.5]nonan-7-yl)-2-((5aS,9aR)-3-fluoro-6,7,9,9a-tetrahydro-1H-pyrano[3,4-b]pyrrolo[3′,2′:5,6]pyrido[3,2-e][1,4]oxazin-5(5aH)-yl)-N-((3-nitro-4-(((tetrahydro-2H-pyran-4-yl)methyl)amino)phenyl)sulfonyl)benzamide,
4-(2-((S)-4-(3,4-difluorobenzyl)-2-(2-isopropoxyphenyl)piperazin-1-yl)-1-methyl-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(3,4-difluorobenzyl)-2-(o-tolyl)piperazin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-4-(3,4-difluorobenzyl)-2-(o-tolyl)piperazin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-2-(2-chlorophenyl)-4-(3,4-difluorobenzyl)piperazin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-2-(2-chlorophenyl)-4-(3,4-difluorobenzyl)piperazin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(3,4-difluorobenzyl)-2-(2-isopropoxyphenyl)piperazin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((S)-2-(2-isopropoxyphenyl)-4-((5-methoxypyridin-2-yl)methyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-4-((5,6-dimethoxypyridin-2-yl)methyl)-2-(2-isopropoxyphenyl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((S)-2-(2-isopropoxyphenyl)-4-((6-methoxy-5-methylpyridin-2-yl)methyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((S)-2-(2-isopropoxyphenyl)-4-((8-methoxy-4,4-dimethyl-3,4-dihydro-2H-pyrano[2,3-c]pyridin-6-yl)methyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-4-((4,4-dimethyl-8-((S)-3-methylmorpholino)-3,4-dihydro-2H-pyrano[2,3-c]pyridin-6-yl)methyl)-2-(2-isopropoxyphenyl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((S)-2-(2-isopropoxyphenyl)-4-((7-methoxy-2-methylfuro[2,3-c]pyridin-5-yl)methyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((S)-2-(2-isopropoxyphenyl)-4-((2-methyl-7-((R)-3-methylmorpholino)furo[2,3-c]pyridin-5-yl)methyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((S)-2-(2-isopropoxyphenyl)-4-((5-morpholinopyridin-2-yl)methyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((S)-2-(2-isopropoxyphenyl)-4-((6-methoxy-5-morpholinopyridin-2-yl)methyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((S)-2-(2-isopropoxyphenyl)-4-((6-methoxypyridin-3-yl)methyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((S)-2-(2-isopropoxyphenyl)-4-((6-morpholinopyridin-3-yl)methyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((S)-2-(2-isopropoxyphenyl)-4-((6-methoxy-5-methylpyridin-3-yl)methyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-4-((5-cyclopropyl-6-methoxypyridin-3-yl)methyl)-2-(2-isopropoxyphenyl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((S)-2-(2-isopropoxyphenyl)-4-((5-methoxy-6-methylpyridin-3-yl)methyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-4-((6-cyclopropyl-5-methoxypyridin-3-yl)methyl)-2-(2-isopropoxyphenyl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-4-((5,6-dimethoxypyridin-3-yl)methyl)-2-(2-isopropoxyphenyl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-4-((2,3-dihydro-[1,4]dioxino[2,3-b]pyridin-7-yl)methyl)-2-(2-isopropoxyphenyl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((S)-2-(2-isopropoxyphenyl)-4-((7-methoxy-2-methylfuro[2,3-c]pyridin-4-yl)methyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((3,3-dimethyl-2,3-dihydro-[1,4]dioxino[2,3-b]pyridin-7-yl)methyl)-2-(o-tolyl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((2,2-dimethyl-2,3-dihydro-[1,4]dioxino[2,3-b]pyridin-7-yl)methyl)-2-(o-tolyl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((R)-4-((7-methoxy-2-methylfuro[2,3-c]pyridin-4-yl)methyl)-2-(o-tolyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((5,6-dimethoxypyridin-3-yl)methyl)-2-(o-tolyl)piperazin-1-yl)-7-azaspiro[3.5]nonan-7-yl)-N-(((S)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((5,6-dimethoxypyridin-3-yl)methyl)-2-(o-tolyl)piperazin-1-yl)-7-azaspiro[3.5]nonan-7-yl)-N—(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((5,6-dimethoxypyridin-3-yl)methyl)-2-(o-tolyl)piperazin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-((1S,2S)-2-((R)-4-((5,6-dimethoxypyridin-3-yl)methyl)-2-(o-tolyl)piperazin-1-yl)-1-methyl-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-1-(5,6-dimethoxypyridin-3-yl)ethyl)-2-(o-tolyl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-((1R,2S)-2-((R)-4-((5,6-dimethoxypyridin-3-yl)methyl)-2-(o-tolyl)piperazin-1-yl)-1-methyl-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((R)-1-(5,6-dimethoxypyridin-3-yl)ethyl)-2-(o-tolyl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((S)-2-(2-isopropoxyphenyl)-4-((6-methoxy-5-morpholinopyridin-3-yl)methyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((S)-2-(2-isopropoxyphenyl)-4-((7-methoxy-2-methylfuro[2,3-c]pyridin-4-yl)methyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((2,3-dihydro-[1,4]dioxino[2,3-b]pyridin-7-yl)methyl)-2-(o-tolyl)piperazin-1-yl)-7-azaspiro[3.5]nonan-7-yl)-N-(((S)-3-((1r,4S)-4-hydroxy-4-methylcyclohexyl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((2,3-dihydro-[1,4]dioxino[2,3-b]pyridin-7-yl)methyl)-2-(o-tolyl)piperazin-1-yl)-7-azaspiro[3.5]nonan-7-yl)-2-((5aS,9aR)-3-fluoro-6,7,9,9a-tetrahydro-1H-pyrano[3,4-b]pyrrolo[3′,2′:5,6]pyrido[3,2-e][1,4]oxazin-5(5aH)-yl)-N-((4-((((1r,4r)-4-hydroxy-4-methylcyclohexyl)methyl)amino)-3-nitrophenyl)sulfonyl)benzamide,
N-((4-((((1r,4r)-4-hydroxy-4-methylcyclohexyl)methyl)amino)-3-nitrophenyl)sulfonyl)-4-(2-((S)-2-(2-isopropoxyphenyl)-4-((5-methoxy-6-morpholinopyridin-3-yl)methyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
2-((5aS,9aR)-3-fluoro-6,7,9,9a-tetrahydro-1H-pyrano[3,4-b]pyrrolo[3′,2′:5,6]pyrido[3,2-e][1,4]oxazin-5(5aH)-yl)-N-((4-((((1r,4r)-4-hydroxy-4-methylcyclohexyl)methyl)amino)-3-nitrophenyl)sulfonyl)-4-(2-((S)-2-(2-isopropoxyphenyl)-4-((5-methoxy-6-morpholinopyridin-3-yl)methyl)piperazin-1-yl)-7-azaspiro[3.5]nonan-7-yl)benzamide,
N-((4-((((1r,4r)-4-hydroxy-4-methylcyclohexyl)methyl)amino)-3-nitrophenyl)sulfonyl)-4-((1S,2R)-2-((S)-2-(2-isopropoxyphenyl)-4-((5-methoxy-6-morpholinopyridin-3-yl)methyl)piperazin-1-yl)-1-methyl-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-((1R,2R)-2-((S)-2-(2-isopropoxyphenyl)-4-((5-methoxy-6-morpholinopyridin-3-yl)methyl)piperazin-1-yl)-1-methyl-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((S)-2-(2-isopropoxyphenyl)-4-((5-methoxy-6-((R)-3-methylmorpholino)pyridin-3-yl)methyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((S)-2-(2-isopropoxyphenyl)-4-((5-methoxy-6-((S)-3-methylmorpholino)pyridin-3-yl)methyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-(((S)-3-((1r,4S)-4-hydroxy-4-methylcyclohexyl)-5-nitro-3,4-dihydro-2H-benzo[b][1,4]oxazin-7-yl)sulfonyl)-4-(2-((S)-2-(2-isopropoxyphenyl)-4-((5-methoxy-6-morpholinopyridin-3-yl)methyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-(((S)-3-((1s,4R)-4-hydroxy-4-methylcyclohexyl)-5-nitro-3,4-dihydro-2H-benzo[b][1,4]oxazin-7-yl)sulfonyl)-4-(2-((S)-2-(2-isopropoxyphenyl)-4-((5-methoxy-6-morpholinopyridin-3-yl)methyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-2-(2-isopropoxyphenyl)-4-((5-methoxy-6-morpholinopyridin-3-yl)methyl)piperazin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-2-(2-isopropoxyphenyl)-4-((5-methoxy-6-morpholinopyridin-3-yl)methyl)piperazin-1-yl)-7-azaspiro[3.5]nonan-7-yl)-N-(((S)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-2-(2-isopropoxyphenyl)-4-((5-methoxy-6-morpholinopyridin-3-yl)methyl)piperazin-1-yl)-7-azaspiro[3.5]nonan-7-yl)-N—(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((S)-2-(2-isopropoxyphenyl)-4-((5-(methoxy-d3)-6-morpholinopyridin-3-yl)methyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((S)-4-((5-isopropoxy-6-morpholinopyridin-3-yl)methyl)-2-(2-isopropoxyphenyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-4-((4,4-dimethyl-8-morpholino-3,4-dihydro-2H-pyrano[2,3-c]pyridin-5-yl)methyl)-2-(2-isopropoxyphenyl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((S)-2-(2-isopropoxyphenyl)-4-((2-methyl-7-morpholinofuro[2,3-c]pyridin-4-yl)methyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((S)-2-(2-isopropoxyphenyl)-4-((R)-1-(5-methoxy-6-morpholinopyridin-3-yl)ethyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((S)-2-(2-isopropoxyphenyl)-4-((S)-1-(5-methoxy-6-morpholinopyridin-3-yl)ethyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-4-((6-((2S,4S)-4-hydroxy-2,4-dimethylpiperidin-1-yl)-5-methoxypyridin-3-yl)methyl)-2-(2-isopropoxyphenyl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((R)-4-((7-methoxy-2-methylfuro[2,3-c]pyridin-4-yl)methyl)-2-(o-tolyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((2,3-dihydro-[1,4]dioxino[2,3-b]pyridin-7-yl)methyl)-2-(o-tolyl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((5,6-dimethoxypyridin-3-yl)methyl)-2-(o-tolyl)piperazin-1-yl)-7-azaspiro[3.5]nonan-7-yl)-N-((4-((((1 r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((R)-4-((5-methoxy-6-((R)-3-methylmorpholino)pyridin-3-yl)methyl)-2-(o-tolyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((R)-4-((5-methoxy-6-((S)-3-methylmorpholino)pyridin-3-yl)methyl)-2-(o-tolyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((4,4-dimethyl-8-morpholino-3,4-dihydro-2H-pyrano[2,3-c]pyridin-5-yl)methyl)-2-(o-tolyl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((R)-4-((2-methyl-7-morpholinofuro[2,3-c]pyridin-4-yl)methyl)-2-(o-tolyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((R)-4-((5-isopropoxy-6-morpholinopyridin-3-yl)methyl)-2-(o-tolyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((R)-4-((5-(methoxy-d3)-6-morpholinopyridin-3-yl)methyl)-2-(o-tolyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-((1R,2S)-2-((R)-4-((5-methoxy-6-morpholinopyridin-3-yl)methyl)-2-(o-tolyl)piperazin-1-yl)-1-methyl-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-((1S,2S)-2-((R)-4-((5-methoxy-6-morpholinopyridin-3-yl)methyl)-2-(o-tolyl)piperazin-1-yl)-1-methyl-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-(((S)-3-((1r,4S)-4-hydroxy-4-methylcyclohexyl)-5-nitro-3,4-dihydro-2H-benzo[b][1,4]oxazin-7-yl)sulfonyl)-4-(2-((R)-4-((5-methoxy-6-morpholinopyridin-3-yl)methyl)-2-(o-tolyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((5-methoxy-6-morpholinopyridin-3-yl)methyl)-2-(o-tolyl)piperazin-1-yl)-7-azaspiro[3.5]nonan-7-yl)-N—(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((5-methoxy-6-morpholinopyridin-3-yl)methyl)-2-(o-tolyl)piperazin-1-yl)-7-azaspiro[3.5]nonan-7-yl)-N-(((S)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((5-methoxy-6-morpholinopyridin-3-yl)methyl)-2-(o-tolyl)piperazin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((R)-4-((5-methoxy-6-morpholinopyridin-3-yl)methyl)-2-(o-tolyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((R)-4-((S)-1-(5-methoxy-6-morpholinopyridin-3-yl)ethyl)-2-(o-tolyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((R)-4-((R)-1-(5-methoxy-6-morpholinopyridin-3-yl)ethyl)-2-(o-tolyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((R)-4-((5-methoxy-6-morpholinopyridin-3-yl)methyl)-2-(o-tolyl)piperazin-1-yl)-1,1,3,3-tetramethyl-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((S)-2-(2-isopropoxyphenyl)-4-((5-methoxy-6-morpholinopyridin-3-yl)methyl)piperazin-1-yl)-1,1,3,3-tetramethyl-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((R)-4-(5-methoxy-6-morpholinonicotinoyl)-2-(o-tolyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((R)-4-((5-methoxy-6-morpholinopyridin-3-yl)sulfonyl)-2-(o-tolyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
2-((5aS,9aR)-3-fluoro-6,7,9,9a-tetrahydro-1H-pyrano[3,4-b]pyrrolo[3′,2′:5,6]pyrido[3,2-e][1,4]oxazin-5(5aH)-yl)-N-((4-((((1r,4r)-4-hydroxy-4-methylcyclohexyl)methyl)amino)-3-nitrophenyl)sulfonyl)-4-(2-((R)-4-((5-methoxy-6-morpholinopyridin-3-yl)methyl)-2-(o-tolyl)piperazin-1-yl)-7-azaspiro[3.5]nonan-7-yl)benzamide,
2-((5aS,9aR)-3-fluoro-6,7,9,9a-tetrahydro-1H-pyrano[3,4-b]pyrrolo[3′,2′:5,6]pyrido[3,2-e][1,4]oxazin-5(5aH)-yl)-N-((4-((((1r,4r)-4-hydroxy-4-methylcyclohexyl)methyl)amino)-3-nitrophenyl)sulfonyl)-4-(6-((R)-4-((5-methoxy-6-morpholinopyridin-3-yl)methyl)-2-(o-tolyl)piperazin-1-yl)-2-azaspiro[3.3]heptan-2-yl)benzamide,
4-(2-((S)-4-((5,6-dimethoxypyrazin-2-yl)methyl)-2-(2-isopropoxyphenyl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-4-((4,5-dimethoxypyrimidin-2-yl)methyl)-2-(2-isopropoxyphenyl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-4-((5,6-dimethoxypyrimidin-4-yl)methyl)-2-(2-isopropoxyphenyl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-4-((3,3-dimethyl-2,3-dihydro-[1,4]dioxino[2,3-b]pyridin-8-yl)methyl)-2-(2-isopropoxyphenyl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((3,3-dimethyl-2,3-dihydro-[1,4]dioxino[2,3-b]pyridin-8-yl)methyl)-2-(o-tolyl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-4-((3,3-dimethyl-2,3-dihydro-[1,4]dioxino[2,3-b]pyridin-8-yl)methyl)-2-(2-isopropoxyphenyl)piperazin-1-yl)-1-methyl-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((3,3-dimethyl-2,3-dihydro-[1,4]dioxino[2,3-b]pyridin-8-yl)methyl)-2-(o-tolyl)piperazin-1-yl)-1-methyl-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-4-((3,3-dimethyl-2,3-dihydro-[1,4]dioxino[2,3-b]pyridin-8-yl)methyl)-2-(2-isopropoxyphenyl)piperazin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((3,3-dimethyl-2,3-dihydro-[1,4]dioxino[2,3-b]pyridin-8-yl)methyl)-2-(o-tolyl)piperazin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-4-((3,3-dimethyl-2,3-dihydro-[1,4]dioxino[2,3-b]pyridin-8-yl)methyl)-2-(2-isopropoxyphenyl)piperazin-1-yl)-7-azaspiro[3.5]nonan-7-yl)-N-(((S)-3-((1r,4S)-4-hydroxy-4-methylcyclohexyl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((3,3-dimethyl-2,3-dihydro-[1,4]dioxino[2,3-b]pyridin-8-yl)methyl)-2-(o-tolyl)piperazin-1-yl)-7-azaspiro[3.5]nonan-7-yl)-N-(((S)-3-((1r,4S)-4-hydroxy-4-methylcyclohexyl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-4-((3,3-dimethyl-2,3-dihydro-[1,4]dioxino[2,3-b]pyridin-8-yl)methyl)-2-(2-isopropoxyphenyl)piperazin-1-yl)-7-azaspiro[3.5]nonan-7-yl)-2-((5aS,9aR)-3-fluoro-6,7,9,9a-tetrahydro-1H-pyrano[3,4-b]pyrrolo[3′,2′:5,6]pyrido[3,2-e][1,4]oxazin-5(5aH)-yl)-N-((4-((((1r,4r)-4-hydroxy-4-methylcyclohexyl)methyl)amino)-3-nitrophenyl)sulfonyl)benzamide,
4-(2-((R)-4-((3,3-dimethyl-2,3-dihydro-[1,4]dioxino[2,3-b]pyridin-8-yl)methyl)-2-(o-tolyl)piperazin-1-yl)-7-azaspiro[3.5]nonan-7-yl)-2-((5aS,9aR)-3-fluoro-6,7,9,9a-tetrahydro-1H-pyrano[3,4-b]pyrrolo[3′,2′:5,6]pyrido[3,2-e][1,4]oxazin-5(5aH)-yl)-N-((4-((((1r,4r)-4-hydroxy-4-methylcyclohexyl)methyl)amino)-3-nitrophenyl)sulfonyl)benzamide,
4-(2-((S)-4-((2,3-dimethoxypyridin-4-yl)methyl)-2-(2-isopropoxyphenyl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((S)-2-(2-isopropoxyphenyl)-4-((3-methoxy-2-morpholinopyridin-4-yl)methyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((S)-2-(2-isopropoxyphenyl)-4-(tetrahydro-2H-pyran-4-yl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-4-(tetrahydro-2H-pyran-4-yl)-2-(2-(trifluoromethoxy)phenyl)piperazin-1-yl)-7-azaspiro[3.5]nonan-7-yl)benzamide,
4-(2-((R)-2-(2-(tert-butoxy)phenyl)-4-(tetrahydro-2H-pyran-4-yl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((R)-2-(2-(2-hydroxypropan-2-yl)phenyl)-4-(tetrahydro-2H-pyran-4-yl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((S)-2-(2-isopropoxyphenyl)-4-(tetrahydro-2H-pyran-4-yl)piperazin-1-yl)-1,1,3,3-tetramethyl-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((S)-2-(2-isopropoxyphenyl)-4-(tetrahydro-2H-pyran-4-yl)piperazin-1-yl)-7-azaspiro[3.5]nonan-7-yl)-2-((5aR,9aS)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((S)-2-(2-isopropoxyphenyl)-4-(tetrahydro-2H-pyran-4-yl)piperazin-1-yl)-7-azaspiro[3.5]nonan-7-yl)-2-((5aR,9aR)-5a,8,9,9a-tetrahydro-6H-pyrano[4,3-b]pyrrolo[3′,2′:5,6]pyrido[3,2-e][1,4]oxazin-5(1H)-yl)benzamide,
N-((4-((((1r,4r)-4-hydroxy-4-methylcyclohexyl)methyl)amino)-3-nitrophenyl)sulfonyl)-4-(2-((S)-2-(2-isopropoxyphenyl)-4-(tetrahydro-2H-pyran-4-yl)piperazin-1-yl)-7-azaspiro[3.5]nonan-7-yl)-2-((5aS,9aS)-5a,8,9,9a-tetrahydro-6H-pyrano[4,3-b]pyrrolo[3′,2′:5,6]pyrido[3,2-e][1,4]oxazin-5(1H)-yl)benzamide,
4-(2-((S)-4-((1r,4S)-4-hydroxy-4-methylcyclohexyl)-2-(2-isopropoxyphenyl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-4-((1s,4R)-4-hydroxy-4-methylcyclohexyl)-2-(2-isopropoxyphenyl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-2-(2-isopropoxyphenyl)-4-(tetrahydro-2H-pyran-4-yl)piperazin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-4-(tetrahydro-2H-pyran-4-yl)-2-(o-tolyl)piperazin-1-yl)-7-azaspiro[3.5]nonan-7-yl)benzamide,
4-(2-((R)-4-((1r,4R)-4-hydroxy-4-methylcyclohexyl)-2-(o-tolyl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((1s,4S)-4-hydroxy-4-methylcyclohexyl)-2-(o-tolyl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(3-oxaspiro[5.5]undecan-9-yl)-2-(o-tolyl)piperazin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((R)-2-(2-isopropoxyphenyl)-4-((3S,4R)-3-methyltetrahydro-2H-pyran-4-yl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((R)-2-(2-isopropoxyphenyl)-4-((3R,4S)-3-methyltetrahydro-2H-pyran-4-yl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((1R,4R)-4-((R)-3-methylmorpholino)cyclohexyl)-2-(o-tolyl)piperazin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((1S,4S)-4-((R)-3-methylmorpholino)cyclohexyl)-2-(o-tolyl)piperazin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-4-(1-(tetrahydro-2H-pyran-4-yl)piperidin-4-yl)-2-(o-tolyl)piperazin-1-yl)-7-azaspiro[3.5]nonan-7-yl)benzamide,
N-((4-((((1r,4r)-4-hydroxy-4-methylcyclohexyl)methyl)amino)-3-nitrophenyl)sulfonyl)-4-((2S,4R,6S)-2-((R)-2-(2-isopropoxyphenyl)-4-(tetrahydro-2H-pyran-4-yl)piperazin-1-yl)-6-methyl-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-((2R,4R,6R)-2-((R)-2-(2-isopropoxyphenyl)-4-(tetrahydro-2H-pyran-4-yl)piperazin-1-yl)-6-methyl-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-((1S,2S,4S,6S)-2-((R)-2-(2-isopropoxyphenyl)-4-(tetrahydro-2H-pyran-4-yl)piperazin-1-yl)-1,6-dimethyl-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((R)-2-(2-isopropoxyphenyl)-4-(tetrahydro-2H-pyran-4-yl)piperazin-1-yl)-1-methyl-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((R)-2-(2-isopropoxyphenyl)-4-(tetrahydro-2H-pyran-4-yl)piperazin-1-yl)-1,1,3,3-tetramethyl-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
2-((5aS,9aR)-7,7-dimethyl-6,7,9,9a-tetrahydro-1H-pyrano[3,4-b]pyrrolo[3′,2′:5,6]pyrido[3,2-e][1,4]oxazin-5(5aH)-yl)-N-((4-((((1r,4r)-4-hydroxy-4-methylcyclohexyl)methyl)amino)-3-nitrophenyl)sulfonyl)-4-(2-((R)-2-(2-isopropoxyphenyl)-4-(tetrahydro-2H-pyran-4-yl)piperazin-1-yl)-7-azaspiro[3.5]nonan-7-yl)benzamide,
N-((4-((((1r,4r)-4-hydroxy-4-methylcyclohexyl)methyl)amino)-3-nitrophenyl)sulfonyl)-4-(2-((S)-2-(2-isopropoxyphenyl)-4-(3-oxaspiro[5.5]undecan-9-yl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((S)-2-(2-isopropoxyphenyl)-4-((1S,4R)-4-((R)-3-methylmorpholino)cyclohexyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((S)-2-(2-isopropoxyphenyl)-4-((1R,4S)-4-((R)-3-methylmorpholino)cyclohexyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((S)-2-(2-isopropoxyphenyl)-4-(1-(tetrahydro-2H-pyran-4-yl)piperidin-4-yl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(4-chloro-3-fluorophenyl)-2-(o-tolyl)piperazin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((3-nitro-4-(((tetrahydro-2H-pyran-4-yl)methyl)amino)phenyl)sulfonyl)-4-(2-((R)-4-(pyridin-4-yl)-2-(o-tolyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-2-(o-tolyl)-4-(2-(trifluoromethyl)pyridin-4-yl)piperazin-1-yl)-7-azaspiro[3.5]nonan-7-yl)benzamide,
4-(2-((R)-4-(2-((R)-3-methylmorpholino)pyridin-4-yl)-2-(o-tolyl)piperazin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-2-(o-tolyl)-4-(4-(trifluoromethyl)phenyl)piperazin-1-yl)-7-azaspiro[3.5]nonan-7-yl)benzamide,
4-(2-((R)-4-(4-(isopropylsulfonyl)phenyl)-2-(o-tolyl)piperazin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((3-nitro-4-(((tetrahydro-2H-pyran-4-yl)methyl)amino)phenyl)sulfonyl)-4-(2-((R)-4-(pyridin-3-yl)-2-(o-tolyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-2-(o-tolyl)-4-(2-(trifluoromethyl)pyridin-3-yl)piperazin-1-yl)-7-azaspiro[3.5]nonan-7-yl)benzamide,
N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-2-(o-tolyl)-4-(6-(trifluoromethyl)pyridin-3-yl)piperazin-1-yl)-7-azaspiro[3.5]nonan-7-yl)benzamide,
4-(2-((R)-4-(6-((R)-3-methylmorpholino)pyridin-3-yl)-2-(o-tolyl)piperazin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(2-((S)-3-methylmorpholino)pyridin-3-yl)-2-(o-tolyl)piperazin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((3-nitro-4-(((tetrahydro-2H-pyran-4-yl)methyl)amino)phenyl)sulfonyl)-4-(2-((R)-4-(pyridin-2-yl)-2-(o-tolyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-2-(o-tolyl)-4-(6-(trifluoromethyl)pyridin-2-yl)piperazin-1-yl)-7-azaspiro[3.5]nonan-7-yl)benzamide,
4-(2-((R)-4-(6-((R)-3-methylmorpholino)pyridin-2-yl)-2-(o-tolyl)piperazin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((3-nitro-4-(((tetrahydro-2H-pyran-4-yl)methyl)amino)phenyl)sulfonyl)-4-(2-((R)-4-(pyrimidin-4-yl)-2-(o-tolyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((3-nitro-4-(((tetrahydro-2H-pyran-4-yl)methyl)amino)phenyl)sulfonyl)-4-(2-((R)-4-(pyrimidin-5-yl)-2-(o-tolyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((3-nitro-4-(((tetrahydro-2H-pyran-4-yl)methyl)amino)phenyl)sulfonyl)-4-(2-((R)-4-(pyrazin-2-yl)-2-(o-tolyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((3-nitro-4-(((tetrahydro-2H-pyran-4-yl)methyl)amino)phenyl)sulfonyl)-4-(2-((R)-4-(pyrimidin-2-yl)-2-(o-tolyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-4-(4-chloro-3-fluorophenyl)-2-(2-isopropoxyphenyl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((S)-2-(2-isopropoxyphenyl)-4-(pyridin-4-yl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((S)-2-(2-isopropoxyphenyl)-4-(2-(trifluoromethyl)pyridin-4-yl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((S)-2-(2-isopropoxyphenyl)-4-(2-((R)-3-methylmorpholino)pyridin-4-yl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-4-(4-chloro-3-fluorophenyl)-2-(2-isopropoxyphenyl)piperazin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-2-(2-isopropoxyphenyl)-4-(pyridin-4-yl)piperazin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-2-(2-isopropoxyphenyl)-4-(2-(trifluoromethyl)pyridin-4-yl)piperazin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-2-(2-isopropoxyphenyl)-4-(2-((R)-3-methylmorpholino)pyridin-4-yl)piperazin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((R)-4-methoxy-3-morpholino-5,6,7,8-tetrahydroisoquinolin-8-yl)-2-(o-tolyl)piperazin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-4-methoxy-3-morpholino-5,6,7,8-tetrahydroisoquinolin-8-yl)-2-(o-tolyl)piperazin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((R)-3-methoxy-2-morpholino-5,6,7,8-tetrahydroquinolin-5-yl)-2-(o-tolyl)piperazin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-3-methoxy-2-morpholino-5,6,7,8-tetrahydroquinolin-5-yl)-2-(o-tolyl)piperazin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-7-morpholino-3,4-dihydro-2H-pyrano[3,2-c]pyridin-4-yl)-2-(o-tolyl)piperazin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((R)-7-morpholino-3,4-dihydro-2H-pyrano[3,2-c]pyridin-4-yl)-2-(o-tolyl)piperazin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((R)-7-morpholino-3,4-dihydro-2H-pyrano[2,3-b]pyridin-4-yl)-2-(o-tolyl)piperazin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-7-morpholino-3,4-dihydro-2H-pyrano[2,3-b]pyridin-4-yl)-2-(o-tolyl)piperazin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-8-methoxy-7-morpholino-3,4-dihydro-1H-pyrano[4,3-c]pyridin-4-yl)-2-(o-tolyl)piperazin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((R)-8-methoxy-7-morpholino-3,4-dihydro-1H-pyrano[4,3-c]pyridin-4-yl)-2-(o-tolyl)piperazin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-3-methoxy-2-morpholino-5,8-dihydro-6H-pyrano[3,4-b]pyridin-5-yl)-2-(o-tolyl)piperazin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((R)-3-methoxy-2-morpholino-5,8-dihydro-6H-pyrano[3,4-b]pyridin-5-yl)-2-(o-tolyl)piperazin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-6-fluoro-7-((S)-3-methylmorpholino)-3,4-dihydro-2H-pyrano[2,3-b]pyridin-4-yl)-2-(o-tolyl)piperazin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-8-fluoro-7-((S)-3-methylmorpholino)-3,4-dihydro-2H-pyrano[3,2-c]pyridin-4-yl)-2-(o-tolyl)piperazin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-5-fluoro-8-((S)-3-methylmorpholino)-3,4-dihydro-2H-pyrano[2,3-c]pyridin-4-yl)-2-(o-tolyl)piperazin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-6,7-difluorochroman-4-yl)-2-(o-tolyl)piperazin-1-yl)-7-azaspiro[3.5]nonan-7-yl)-N-((4-((((1 r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((R)-6,7-difluorochroman-4-yl)-2-(o-tolyl)piperazin-1-yl)-7-azaspiro[3.5]nonan-7-yl)-N-((4-((((1 r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((R)-7,8-difluorochroman-4-yl)-2-(o-tolyl)piperazin-1-yl)-7-azaspiro[3.5]nonan-7-yl)-N-((4-((((1 r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-7,8-difluorochroman-4-yl)-2-(o-tolyl)piperazin-1-yl)-7-azaspiro[3.5]nonan-7-yl)-N-((4-((((1 r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-6,8-difluorochroman-4-yl)-2-(o-tolyl)piperazin-1-yl)-7-azaspiro[3.5]nonan-7-yl)-N-((4-((((1 r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((R)-6,8-difluorochroman-4-yl)-2-(o-tolyl)piperazin-1-yl)-7-azaspiro[3.5]nonan-7-yl)-N-((4-((((1 r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-6,7-difluorochroman-4-yl)-2-(o-tolyl)piperazin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((R)-6,7-difluorochroman-4-yl)-2-(o-tolyl)piperazin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((R)-7,8-difluorochroman-4-yl)-2-(o-tolyl)piperazin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-7,8-difluorochroman-4-yl)-2-(o-tolyl)piperazin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-6,8-difluorochroman-4-yl)-2-(o-tolyl)piperazin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((R)-6,8-difluorochroman-4-yl)-2-(o-tolyl)piperazin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((2R)-4-(2-(1-methyl-2,6-dioxopiperidin-3-yl)-1,3-dioxoisoindolin-5-yl)-2-(o-tolyl)piperazin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-4-((S)-1-(tetrahydro-2H-pyran-4-yl)-1,2,3,4-tetrahydro-1,8-naphthyridin-3-yl)-2-(o-tolyl)piperazin-1-yl)-7-azaspiro[3.5]nonan-7-yl)benzamide,
4-(2-((R)-4-((R)-7-((S)-3-methylmorpholino)-3,4-dihydro-2H-pyrano[2,3-b]pyridin-3-yl)-2-(o-tolyl)piperazin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-4-((S)-6,7-difluorochroman-4-yl)-2-(2-isopropoxyphenyl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-4-((R)-6,7-difluorochroman-4-yl)-2-(2-isopropoxyphenyl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-4-((R)-7,8-difluorochroman-4-yl)-2-(2-isopropoxyphenyl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-4-((S)-7,8-difluorochroman-4-yl)-2-(2-isopropoxyphenyl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-4-((S)-6,8-difluorochroman-4-yl)-2-(2-isopropoxyphenyl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-4-((R)-6,8-difluorochroman-4-yl)-2-(2-isopropoxyphenyl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-4-((S)-6,7-difluorochroman-4-yl)-2-(2-isopropoxyphenyl)piperazin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-4-((R)-6,7-difluorochroman-4-yl)-2-(2-isopropoxyphenyl)piperazin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-4-((R)-7,8-difluorochroman-4-yl)-2-(2-isopropoxyphenyl)piperazin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-4-((S)-7,8-difluorochroman-4-yl)-2-(2-isopropoxyphenyl)piperazin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-4-((S)-6,8-difluorochroman-4-yl)-2-(2-isopropoxyphenyl)piperazin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-4-((R)-6,8-difluorochroman-4-yl)-2-(2-isopropoxyphenyl)piperazin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((S)-2-(2-isopropoxyphenyl)-4-((S)-8-methoxy-7-morpholino-3,4-dihydro-1H-pyrano[4,3-c]pyridin-4-yl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((S)-2-(2-isopropoxyphenyl)-4-((R)-8-methoxy-7-morpholino-3,4-dihydro-1H-pyrano[4,3-c]pyridin-4-yl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((S)-2-(2-isopropoxyphenyl)-4-((S)-3-methoxy-2-morpholino-5,8-dihydro-6H-pyrano[3,4-b]pyridin-5-yl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((S)-2-(2-isopropoxyphenyl)-4-((R)-3-methoxy-2-morpholino-5,8-dihydro-6H-pyrano[3,4-b]pyridin-5-yl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((S)-2-(2-isopropoxyphenyl)-4-((R)-7-morpholino-3,4-dihydro-2H-pyrano[2,3-b]pyridin-4-yl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((S)-2-(2-isopropoxyphenyl)-4-((S)-7-morpholino-3,4-dihydro-2H-pyrano[2,3-b]pyridin-4-yl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((S)-2-(2-isopropoxyphenyl)-4-((R)-8-morpholino-3,4-dihydro-2H-pyrano[2,3-c]pyridin-4-yl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((S)-2-(2-isopropoxyphenyl)-4-((S)-8-morpholino-3,4-dihydro-2H-pyrano[2,3-c]pyridin-4-yl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((S)-2-(2-isopropoxyphenyl)-4-((S)-8-methoxy-3,4-dihydro-2H-pyrano[2,3-c]pyridin-4-yl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((S)-2-(2-isopropoxyphenyl)-4-((R)-8-methoxy-3,4-dihydro-2H-pyrano[2,3-c]pyridin-4-yl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((S)-2-(2-isopropoxyphenyl)-4-((R)-7-morpholino-3,4-dihydro-2H-pyrano[3,2-c]pyridin-4-yl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((S)-2-(2-isopropoxyphenyl)-4-((S)-7-morpholino-3,4-dihydro-2H-pyrano[3,2-c]pyridin-4-yl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-4-((S)-6-fluoro-7-((S)-3-methylmorpholino)-3,4-dihydro-2H-pyrano[2,3-b]pyridin-4-yl)-2-(2-isopropoxyphenyl)piperazin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-4-((S)-8-fluoro-7-((S)-3-methylmorpholino)-3,4-dihydro-2H-pyrano[3,2-c]pyridin-4-yl)-2-(2-isopropoxyphenyl)piperazin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-4-((S)-5-fluoro-8-((S)-3-methylmorpholino)-3,4-dihydro-2H-pyrano[2,3-c]pyridin-4-yl)-2-(2-isopropoxyphenyl)piperazin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-2-(2-isopropoxyphenyl)-4-((R)-4-methoxy-3-morpholino-5,6,7,8-tetrahydroisoquinolin-8-yl)piperazin-1-yl)-7-azaspiro[3.5]nonan-7-yl)-N-((3-nitro-4-(((tetrahydro-2H-pyran-4-yl)methyl)amino)phenyl)sulfonyl-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-2-(2-isopropoxyphenyl)-4-((S)-4-methoxy-3-morpholino-5,6,7,8-tetrahydroisoquinolin-8-yl)piperazin-1-yl)-7-azaspiro[3.5]nonan-7-yl)-N-((3-nitro-4-(((tetrahydro-2H-pyran-4-yl)methyl)amino)phenyl)sulfonyl-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-2-(2-isopropoxyphenyl)-4-((R)-3-methoxy-2-morpholino-5,6,7,8-tetrahydroquinolin-5-yl)piperazin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-2-(2-isopropoxyphenyl)-4-((S)-3-methoxy-2-morpholino-5,6,7,8-tetrahydroquinolin-5-yl)piperazin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((2S,4R)-2-(2-isopropylphenyl)-4-morpholinopiperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((2S,4S)-2-(2-isopropylphenyl)-4-morpholinopiperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((2R,4R)-2-(2-isopropylphenyl)-4-morpholinopiperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((2R,4S)-2-(2-isopropylphenyl)-4-morpholinopiperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((2S,4R)-4-morpholino-2-(o-tolyl)piperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((2S,4S)-4-morpholino-2-(o-tolyl)piperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((2R,4R)-4-morpholino-2-(o-tolyl)piperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((2R,4S)-4-morpholino-2-(o-tolyl)piperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((2S,5S)-2-(2-isopropylphenyl)-5-morpholinopiperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((2S,5S)-2-(2-isopropylphenyl)-2-methyl-5-morpholinopiperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((2S,5R)-2-(2-isopropylphenyl)-5-morpholinopiperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((2R,5S)-2-(2-isopropylphenyl)-5-morpholinopiperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((2R,5R)-2-(2-isopropylphenyl)-5-morpholinopiperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((2S,5S)-5-morpholino-2-(o-tolyl)piperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((2S,5R)-5-morpholino-2-(o-tolyl)piperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((2R,5S)-5-morpholino-2-(o-tolyl)piperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((2R,5R)-5-morpholino-2-(o-tolyl)piperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((2S,4R)-2-(2-isopropylphenyl)-4-((5-methoxy-6-morpholinopyridin-3-yl)methyl)piperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((2S,4S)-2-(2-isopropylphenyl)-4-((5-methoxy-6-morpholinopyridin-3-yl)methyl)piperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((2R,4R)-2-(2-isopropylphenyl)-4-((5-methoxy-6-morpholinopyridin-3-yl)methyl)piperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((2R,4S)-2-(2-isopropylphenyl)-4-((5-methoxy-6-morpholinopyridin-3-yl)methyl)piperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((2S,4R)-4-((5-methoxy-6-morpholinopyridin-3-yl)methyl)-2-(o-tolyl)piperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((2S,4S)-4-((5-methoxy-6-morpholinopyridin-3-yl)methyl)-2-(o-tolyl)piperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((2R,4R)-4-((5-methoxy-6-morpholinopyridin-3-yl)methyl)-2-(o-tolyl)piperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((2R,4S)-4-((5-methoxy-6-morpholinopyridin-3-yl)methyl)-2-(o-tolyl)piperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((2S,4S)-4-fluoro-4-((5-methoxy-6-morpholinopyridin-3-yl)methyl)-2-(o-tolyl)piperidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((2S,4R)-4-((5-methoxy-6-morpholinopyridin-3-yl)methyl)-4-methyl-2-(o-tolyl)piperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((2S,4S)-4-hydroxy-4-((5-methoxy-6-morpholinopyridin-3-yl)methyl)-2-(o-tolyl)piperidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((2S,4S)-4-cyano-4-((5-methoxy-6-morpholinopyridin-3-yl)methyl)-2-(o-tolyl)piperidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((2S,4R)-4-hydroxy-4-methyl-2-(o-tolyl)piperidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((2S,4S)-4-hydroxy-4-methyl-2-(o-tolyl)piperidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((2R,4R)-4-hydroxy-4-methyl-2-(o-tolyl)piperidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((2R,4S)-4-hydroxy-4-methyl-2-(o-tolyl)piperidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((2S,5S)-5-hydroxy-5-methyl-2-(o-tolyl)piperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((2S,5R)-5-hydroxy-5-methyl-2-(o-tolyl)piperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((2R,5S)-5-hydroxy-5-methyl-2-(o-tolyl)piperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((2R,5R)-5-hydroxy-5-methyl-2-(o-tolyl)piperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((2S,4R)-4-hydroxy-2-(2-isopropylphenyl)-4-methylpiperidin-1-yl)-7-azaspiro[3.5]nonan-7-yl)-N-((4-((((1 r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((2S,4S)-4-hydroxy-2-(2-isopropylphenyl)-4-methylpiperidin-1-yl)-7-azaspiro[3.5]nonan-7-yl)-N-((4-((((1 r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((2R,4R)-4-hydroxy-2-(2-isopropylphenyl)-4-methylpiperidin-1-yl)-7-azaspiro[3.5]nonan-7-yl)-N-((4-((((1 r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((2R,4S)-4-hydroxy-2-(2-isopropylphenyl)-4-methylpiperidin-1-yl)-7-azaspiro[3.5]nonan-7-yl)-N-((4-((((1 r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((2S,5S)-5-hydroxy-2-(2-isopropylphenyl)-5-methylpiperidin-1-yl)-7-azaspiro[3.5]nonan-7-yl)-N-((4-((((1 r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((2S,5R)-5-hydroxy-2-(2-isopropylphenyl)-5-methylpiperidin-1-yl)-7-azaspiro[3.5]nonan-7-yl)-N-((4-((((1 r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((2R,5S)-5-hydroxy-2-(2-isopropylphenyl)-5-methylpiperidin-1-yl)-7-azaspiro[3.5]nonan-7-yl)-N-((4-((((1 r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((2R,5R)-5-hydroxy-2-(2-isopropylphenyl)-5-methylpiperidin-1-yl)-7-azaspiro[3.5]nonan-7-yl)-N-((4-((((1 r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((2S,4R)-4-hydroxy-2-(2-isopropylphenyl)-4-methylpiperidin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((cyclohexylmethyl)amino)-3-nitrophenyl)sulfonyl)-4-(2-((2S,4S)-4-hydroxy-2-(2-isopropylphenyl)-4-methylpiperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((2R,4R)-4-hydroxy-2-(2-isopropylphenyl)-4-methylpiperidin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((2R,4S)-4-hydroxy-2-(2-isopropylphenyl)-4-methylpiperidin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((2S,5S)-5-hydroxy-2-(2-isopropylphenyl)-5-methylpiperidin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((2S,5R)-5-hydroxy-2-(2-isopropylphenyl)-5-methylpiperidin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((2R,5S)-5-hydroxy-2-(2-isopropylphenyl)-5-methylpiperidin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((2R,5R)-5-hydroxy-2-(2-isopropylphenyl)-5-methylpiperidin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((2S,4R)-4-(3,4-difluorobenzyl)-2-(2-isopropylphenyl)piperidin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((2S,4S)-4-(3,4-difluorobenzyl)-2-(2-isopropylphenyl)piperidin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((2R,4R)-4-(3,4-difluorobenzyl)-2-(2-isopropylphenyl)piperidin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((2R,4S)-4-(3,4-difluorobenzyl)-2-(2-isopropylphenyl)piperidin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((2S,4R)-4-hydroxy-4-methyl-2-(o-tolyl)piperidin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((cyclohexylmethyl)amino)-3-nitrophenyl)sulfonyl)-4-(2-((2S,4S)-4-hydroxy-4-methyl-2-(o-tolyl)piperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((2R,4R)-4-hydroxy-4-methyl-2-(o-tolyl)piperidin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((2R,4S)-4-hydroxy-4-methyl-2-(o-tolyl)piperidin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((2S,5S)-5-hydroxy-5-methyl-2-(o-tolyl)piperidin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((2S,5R)-5-hydroxy-5-methyl-2-(o-tolyl)piperidin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((2R,5S)-5-hydroxy-5-methyl-2-(o-tolyl)piperidin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((2R,5R)-5-hydroxy-5-methyl-2-(o-tolyl)piperidin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((2S,4R)-4-(3,4-difluorobenzyl)-2-(o-tolyl)piperidin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((2S,4S)-4-(3,4-difluorobenzyl)-2-(o-tolyl)piperidin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((2R,4R)-4-(3,4-difluorobenzyl)-2-(o-tolyl)piperidin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((2R,4S)-4-(3,4-difluorobenzyl)-2-(o-tolyl)piperidin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(4-(((R)-2-(2-isopropoxyphenyl)-4-(tetrahydro-2H-pyran-4-yl)piperazin-1-yl)methyl)piperidin-1-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(4-(((S)-2-(2-isopropoxyphenyl)-4-(tetrahydro-2H-pyran-4-yl)piperazin-1-yl)methyl)piperidin-1-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(4-(((R)-4-(tetrahydro-2H-pyran-4-yl)-2-(o-tolyl)piperazin-1-yl)methyl)piperidin-1-yl)benzamide,
N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(4-(((S)-4-(tetrahydro-2H-pyran-4-yl)-2-(o-tolyl)piperazin-1-yl)methyl)piperidin-1-yl)benzamide,
N-((4-((((1r,4r)-4-hydroxy-4-methylcyclohexyl)methyl)amino)-3-nitrophenyl)sulfonyl)-4-(4-(((R)-2-(2-isopropylphenyl)-4-(tetrahydro-2H-pyran-4-yl)piperazin-1-yl)methyl)piperidin-1-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(4-(((S)-2-(2-isopropylphenyl)-4-(tetrahydro-2H-pyran-4-yl)piperazin-1-yl)methyl)piperidin-1-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(4-(((S)-2-(2-isopropoxyphenyl)piperidin-1-yl)methyl)piperidin-1-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(4-(((R)-2-(2-isopropoxyphenyl)piperidin-1-yl)methyl)piperidin-1-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(4-(((S)-2-(2-isopropylphenyl)piperidin-1-yl)methyl)piperidin-1-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(4-(((R)-2-(2-isopropylphenyl)piperidin-1-yl)methyl)piperidin-1-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(4-(((S)-2-(o-tolyl)piperidin-1-yl)methyl)piperidin-1-yl)benzamide,
N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(4-(((R)-2-(o-tolyl)piperidin-1-yl)methyl)piperidin-1-yl)benzamide,
N-((4-((((1r,4r)-4-hydroxy-4-methylcyclohexyl)methyl)amino)-3-nitrophenyl)sulfonyl)-4-(2-((S)-2-(3-isopropylthiophen-2-yl)-4-((5-methoxy-6-morpholinopyridin-3-yl)methyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((R)-2-(4-isopropylthiophen-3-yl)-4-((5-methoxy-6-morpholinopyridin-3-yl)methyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((R)-2-(2-isopropylthiophen-3-yl)-4-((5-methoxy-6-morpholinopyridin-3-yl)methyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
2-((5aS,9aR)-3-fluoro-6,7,9,9a-tetrahydro-1H-pyrano[3,4-b]pyrrolo[3′,2′:5,6]pyrido[3,2-e][1,4]oxazin-5(5aH)-yl)-N-((4-((((1r,4r)-4-hydroxy-4-methylcyclohexyl)methyl)amino)-3-nitrophenyl)sulfonyl)-4-(2-((S)-2-(3-isopropylthiophen-2-yl)-4-((5-methoxy-6-morpholinopyridin-3-yl)methyl)piperazin-1-yl)-7-azaspiro[3.5]nonan-7-yl)benzamide,
2-((5aS,9aR)-3-fluoro-6,7,9,9a-tetrahydro-1H-pyrano[3,4-b]pyrrolo[3′,2′:5,6]pyrido[3,2-e][1,4]oxazin-5(5aH)-yl)-N-((4-((((1r,4r)-4-hydroxy-4-methylcyclohexyl)methyl)amino)-3-nitrophenyl)sulfonyl)-4-(2-((R)-2-(4-isopropylthiophen-3-yl)-4-((5-methoxy-6-morpholinopyridin-3-yl)methyl)piperazin-1-yl)-7-azaspiro[3.5]nonan-7-yl)benzamide,
2-((5aS,9aR)-3-fluoro-6,7,9,9a-tetrahydro-1H-pyrano[3,4-b]pyrrolo[3′,2′:5,6]pyrido[3,2-e][1,4]oxazin-5(5aH)-yl)-N-((4-((((1r,4r)-4-hydroxy-4-methylcyclohexyl)methyl)amino)-3-nitrophenyl)sulfonyl)-4-(2-((R)-2-(2-isopropylthiophen-3-yl)-4-((5-methoxy-6-morpholinopyridin-3-yl)methyl)piperazin-1-yl)-7-azaspiro[3.5]nonan-7-yl)benzamide,
N-((4-((((1r,4r)-4-hydroxy-4-methylcyclohexyl)methyl)amino)-3-nitrophenyl)sulfonyl)-4-(2-((S)-2-(3-isopropylthiophen-2-yl)-4-((5-methoxy-6-morpholinopyridin-3-yl)methyl)piperazin-1-yl)-1-methyl-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((R)-2-(4-isopropylthiophen-3-yl)-4-((5-methoxy-6-morpholinopyridin-3-yl)methyl)piperazin-1-yl)-1-methyl-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((R)-2-(2-isopropylthiophen-3-yl)-4-((5-methoxy-6-morpholinopyridin-3-yl)methyl)piperazin-1-yl)-1-methyl-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-4-((5,6-dimethoxypyridin-3-yl)methyl)-2-(2-isopropylthiophen-3-yl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((5,6-dimethoxypyridin-3-yl)methyl)-2-(2-isopropylthiophen-3-yl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-4-((5,6-dimethoxypyridin-3-yl)methyl)-2-(2-methylthiophen-3-yl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((5,6-dimethoxypyridin-3-yl)methyl)-2-(2-methylthiophen-3-yl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-4-((5,6-dimethoxypyridin-3-yl)methyl)-2-(4-isopropylthiophen-3-yl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((5,6-dimethoxypyridin-3-yl)methyl)-2-(4-isopropylthiophen-3-yl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-4-((5,6-dimethoxypyridin-3-yl)methyl)-2-(4-methylthiophen-3-yl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((5,6-dimethoxypyridin-3-yl)methyl)-2-(4-methylthiophen-3-yl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((5,6-dimethoxypyridin-3-yl)methyl)-2-(3-isopropylthiophen-2-yl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-4-((5,6-dimethoxypyridin-3-yl)methyl)-2-(3-isopropylthiophen-2-yl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((5,6-dimethoxypyridin-3-yl)methyl)-2-(3-methylthiophen-2-yl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-4-((5,6-dimethoxypyridin-3-yl)methyl)-2-(3-methylthiophen-2-yl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((R)-4-(1-((5-methoxy-6-morpholinopyridin-3-yl)methyl)piperidin-4-yl)-2-(2-(trifluoromethoxy)phenyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-2-(2-(tert-butoxy)phenyl)-4-(1-((5-methoxy-6-morpholinopyridin-3-yl)methyl)piperidin-4-yl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((R)-2-(2-(2-hydroxypropan-2-yl)phenyl)-4-(1-((5-methoxy-6-morpholinopyridin-3-yl)methyl)piperidin-4-yl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-2-(2-chlorophenyl)-4-((5,6-dimethoxypyridin-3-yl)methyl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-2-(2-chlorophenyl)-4-((5,6-dimethoxypyridin-3-yl)methyl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-4-((5,6-dimethoxypyridin-3-yl)methyl)-2-(2-isopropylphenyl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((5,6-dimethoxypyridin-3-yl)methyl)-2-(2-isopropylphenyl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
2-((6aR,10aR)-6,6a,7,8,10,10a-hexahydropyrano[4,3-f]pyrrolo[3′,2′:5,6]pyrido[2,3-b][1,4]oxazepin-5(1H)-yl)-N-((4-((((1r,4r)-4-hydroxy-4-methylcyclohexyl)methyl)amino)-3-nitrophenyl)sulfonyl)-4-(2-((R)-4-((5-methoxy-6-morpholinopyridin-3-yl)methyl)-2-(o-tolyl)piperazin-1-yl)-7-azaspiro[3.5]nonan-7-yl)benzamide,
2-((6aS,10aS)-6,6a,7,8,10,10a-hexahydropyrano[4,3-f]pyrrolo[3′,2′:5,6]pyrido[2,3-b][1,4]oxazepin-5(1H)-yl)-N-((4-((((1r,4r)-4-hydroxy-4-methylcyclohexyl)methyl)amino)-3-nitrophenyl)sulfonyl)-4-(2-((R)-4-((5-methoxy-6-morpholinopyridin-3-yl)methyl)-2-(o-tolyl)piperazin-1-yl)-7-azaspiro[3.5]nonan-7-yl)benzamide,
N-((4-((((1r,4r)-4-hydroxy-4-methylcyclohexyl)methyl)amino)-3-nitrophenyl)sulfonyl)-4-(2-((R)-4-((5-methoxy-6-morpholinopyridin-3-yl)methyl)-2-(o-tolyl)piperazin-1-yl)-7-azaspiro[3.5]nonan-7-yl)-2-((6aS,10aS)-6a,9,10,10a-tetrahydro-1H,7H-pyrano[3,4-f]pyrrolo[3′,2′:5,6]pyrido[2,3-b][1,4]oxazepin-5(6H)-yl)benzamide,
2-((6aR,10aR)-6,6a,7,8,10,10a-hexahydropyrano[4,3-f]pyrrolo[3′,2′:5,6]pyrido[2,3-b][1,4]oxazepin-5(1H)-yl)-N-((4-((((1r,4r)-4-hydroxy-4-methylcyclohexyl)methyl)amino)-3-nitrophenyl)sulfonyl)-4-(2-((R)-4-((5-methoxy-6-morpholinopyridin-3-yl)methyl)-2-(o-tolyl)piperazin-1-yl)-7-azaspiro[3.5]nonan-7-yl)benzamide,
N-((4-((((1r,4r)-4-hydroxy-4-methylcyclohexyl)methyl)amino)-3-nitrophenyl)sulfonyl)-4-(2-((R)-4-((5-methoxy-6-morpholinopyridin-3-yl)methyl)-2-(o-tolyl)piperazin-1-yl)-7-azaspiro[3.5]nonan-7-yl)-2-((6aR,10aR)-6a,9,10,10a-tetrahydro-1H,7H-pyrano[3,4-f]pyrrolo[3′,2′:5,6]pyrido[2,3-b][1,4]oxazepin-5(6H)-yl)benzamide,
2-((6aS,10aS)-6,6a,7,8,10,10a-hexahydropyrano[4,3-f]pyrrolo[3′,2′:5,6]pyrido[2,3-b][1,4]oxazepin-5(1H)-yl)-N-((4-((((1r,4r)-4-hydroxy-4-methylcyclohexyl)methyl)amino)-3-nitrophenyl)sulfonyl)-4-(2-((R)-4-((5-methoxy-6-morpholinopyridin-3-yl)methyl)-2-(o-tolyl)piperazin-1-yl)-7-azaspiro[3.5]nonan-7-yl)benzamide,
N-((4-((((1r,4r)-4-hydroxy-4-methylcyclohexyl)methyl)amino)-3-nitrophenyl)sulfonyl)-4-(2-((R)-4-((5-methoxy-6-morpholinopyridin-3-yl)methyl)-2-(o-tolyl)piperazin-1-yl)-7-azaspiro[3.5]nonan-7-yl)-2-((6aS,10aS)-6a,9,10,10a-tetrahydro-1H,7H-pyrano[3,4-f]pyrrolo[3′,2′:5,6]pyrido[2,3-b][1,4]oxazepin-5(6H)-yl)benzamide,
N-((4-((((1r,4r)-4-hydroxy-4-methylcyclohexyl)methyl)amino)-3-nitrophenyl)sulfonyl)-4-(2-((R)-4-((5-methoxy-6-morpholinopyridin-3-yl)methyl)-2-(o-tolyl)piperazin-1-yl)-7-azaspiro[3.5]nonan-7-yl)-2-((6aR,10aR)-6a,9,10,10a-tetrahydro-1H,7H-pyrano[3,4-f]pyrrolo[3′,2′:5,6]pyrido[2,3-b][1,4]oxazepin-5(6H)-yl)benzamide,
2-((S)-1,2,4,4a,5,6-hexahydro-[1,4]oxazino[4,3-d]pyrrolo[3′,2′:5,6]pyrido[3,2-b][1,4]diazepin-7(11H)-yl)-N-((4-((((1r,4r)-4-hydroxy-4-methylcyclohexyl)methyl)amino)-3-nitrophenyl)sulfonyl)-4-(2-((R)-4-((5-methoxy-6-morpholinopyridin-3-yl)methyl)-2-(o-tolyl)piperazin-1-yl)-7-azaspiro[3.5]nonan-7-yl)benzamide,
2-((R)-1,2,4,4a,5,6-hexahydro-[1,4]oxazino[4,3-d]pyrrolo[3′,2′:5,6]pyrido[3,2-b][1,4]diazepin-7(11H)-yl)-N-((4-((((1r,4r)-4-hydroxy-4-methylcyclohexyl)methyl)amino)-3-nitrophenyl)sulfonyl)-4-(2-((R)-4-((5-methoxy-6-morpholinopyridin-3-yl)methyl)-2-(o-tolyl)piperazin-1-yl)-7-azaspiro[3.5]nonan-7-yl)benzamide,
2-((S)-1,6a,7,9,10,12-hexahydro-[1,4]oxazino[4,3-a]pyrrolo[3′,2′:5,6]pyrido[3,2-e][1,4]diazepin-5(6H)-yl)-N-((4-((((1r,4r)-4-hydroxy-4-methylcyclohexyl)methyl)amino)-3-nitrophenyl)sulfonyl)-4-(2-((R)-4-((5-methoxy-6-morpholinopyridin-3-yl)methyl)-2-(o-tolyl)piperazin-1-yl)-7-azaspiro[3.5]nonan-7-yl)benzamide,
2-((R)-1,6a,7,9,10,12-hexahydro-[1,4]oxazino[4,3-a]pyrrolo[3′,2′:5,6]pyrido[3,2-e][1,4]diazepin-5(6H)-yl)-N-((4-((((1r,4r)-4-hydroxy-4-methylcyclohexyl)methyl)amino)-3-nitrophenyl)sulfonyl)-4-(2-((R)-4-((5-methoxy-6-morpholinopyridin-3-yl)methyl)-2-(o-tolyl)piperazin-1-yl)-7-azaspiro[3.5]nonan-7-yl)benzamide,
2-((R)-2,3,5,5a,6,7-hexahydro-1H-pyrrolo[3″,2″:5′,6′]pyrido[2′,3′:2,3][1,4]diazepino[7,1-c][1,4]oxazepin-8(12H)-yl)-N-((4-((((1r,4r)-4-hydroxy-4-methylcyclohexyl)methyl)amino)-3-nitrophenyl)sulfonyl)-4-(2-((R)-4-((5-methoxy-6-morpholinopyridin-3-yl)methyl)-2-(o-tolyl)piperazin-1-yl)-7-azaspiro[3.5]nonan-7-yl)benzamide,
2-((S)-2,3,5,5a,6,7-hexahydro-1H-pyrrolo[3″,2″:5′,6′]pyrido[2′,3′:2,3][1,4]diazepino[7,1-c][1,4]oxazepin-8(12H)-yl)-N-((4-((((1r,4r)-4-hydroxy-4-methylcyclohexyl)methyl)amino)-3-nitrophenyl)sulfonyl)-4-(2-((R)-4-((5-methoxy-6-morpholinopyridin-3-yl)methyl)-2-(o-tolyl)piperazin-1-yl)-7-azaspiro[3.5]nonan-7-yl)benzamide,
2-((5aR,13aS)-2,3,5,5a,13,13a-hexahydro-[1,4]dioxepino[5,6-f]pyrrolo[3′,2′:5,6]pyrido[2,3-b][1,4]oxazepin-12(8H)-yl)-N-((4-((((1r,4r)-4-hydroxy-4-methylcyclohexyl)methyl)amino)-3-nitrophenyl)sulfonyl)-4-(2-((R)-4-((5-methoxy-6-morpholinopyridin-3-yl)methyl)-2-(o-tolyl)piperazin-1-yl)-7-azaspiro[3.5]nonan-7-yl)benzamide,
2-((5aS,13aR)-2,3,5,5a,13,13a-hexahydro-[1,4]dioxepino[5,6-f]pyrrolo[3′,2′:5,6]pyrido[2,3-b][1,4]oxazepin-12(8H)-yl)-N-((4-((((1r,4r)-4-hydroxy-4-methylcyclohexyl)methyl)amino)-3-nitrophenyl)sulfonyl)-4-(2-((R)-4-((5-methoxy-6-morpholinopyridin-3-yl)methyl)-2-(o-tolyl)piperazin-1-yl)-7-azaspiro[3.5]nonan-7-yl)benzamide,
N-((4-((((1r,4r)-4-hydroxy-4-methylcyclohexyl)methyl)amino)-3-nitrophenyl)sulfonyl)-4-(2-((R)-4-((5-methoxy-6-morpholinopyridin-3-yl)methyl)-2-(o-tolyl)piperazin-1-yl)-7-azaspiro[3.5]nonan-7-yl)-2-((S)-1,2,4a,5-tetrahydro-4H-pyrrolo[3″,2″:5′,6′]pyrido[3′,2′:5,6]pyrazino[2,1-c][1,4]oxazin-6(10H)-yl)benzamide,
N-((4-((((1r,4r)-4-hydroxy-4-methylcyclohexyl)methyl)amino)-3-nitrophenyl)sulfonyl)-4-(2-((R)-4-((5-methoxy-6-morpholinopyridin-3-yl)methyl)-2-(o-tolyl)piperazin-1-yl)-7-azaspiro[3.5]nonan-7-yl)-2-((R)-1,2,4a,5-tetrahydro-4H-pyrrolo[3″,2″:5′,6′]pyrido[3′,2′:5,6]pyrazino[2,1-c][1,4]oxazin-6(10H)-yl)benzamide,
N-((4-((((1r,4r)-4-hydroxy-4-methylcyclohexyl)methyl)amino)-3-nitrophenyl)sulfonyl)-4-(2-((R)-4-((5-methoxy-6-morpholinopyridin-3-yl)methyl)-2-(o-tolyl)piperazin-1-yl)-7-azaspiro[3.5]nonan-7-yl)-2-((S)-2,3,5a,6-tetrahydro-1H,5H-pyrrolo[3″,2″:5′,6′]pyrido[3′,2′:5,6]pyrazino[2,1-c][1,4]oxazepin-7(11H)-yl)benzamide,
N-((4-((((1r,4r)-4-hydroxy-4-methylcyclohexyl)methyl)amino)-3-nitrophenyl)sulfonyl)-4-(2-((R)-4-((5-methoxy-6-morpholinopyridin-3-yl)methyl)-2-(o-tolyl)piperazin-1-yl)-7-azaspiro[3.5]nonan-7-yl)-2-((R)-2,3,5a,6-tetrahydro-1H,5H-pyrrolo[3″,2″:5′,6′]pyrido[3′,2′:5,6]pyrazino[2,1-c][1,4]oxazepin-7(11H)-yl)benzamide,
2-((5aR,10aR)-5a,6,7,9,10,10a-hexahydrooxepino[4,5-b]pyrrolo[3′,2′:5,6]pyrido[3,2-e][1,4]oxazin-5(1H)-yl)-N-((4-((((1r,4r)-4-hydroxy-4-methylcyclohexyl)methyl)amino)-3-nitrophenyl)sulfonyl)-4-(2-((R)-4-((5-methoxy-6-morpholinopyridin-3-yl)methyl)-2-(o-tolyl)piperazin-1-yl)-7-azaspiro[3.5]nonan-7-yl)benzamide,
2-((5aS,10aS)-5a,6,7,9,10,10a-hexahydrooxepino[4,5-b]pyrrolo[3′,2′:5,6]pyrido[3,2-e][1,4]oxazin-5(1H)-yl)-N-((4-((((1r,4r)-4-hydroxy-4-methylcyclohexyl)methyl)amino)-3-nitrophenyl)sulfonyl)-4-(2-((R)-4-((5-methoxy-6-morpholinopyridin-3-yl)methyl)-2-(o-tolyl)piperazin-1-yl)-7-azaspiro[3.5]nonan-7-yl)benzamide,
N-((4-((((1r,4r)-4-hydroxy-4-methylcyclohexyl)methyl)amino)-3-nitrophenyl)sulfonyl)-4-(4-((1S,3R)-3-((R)-2-(2-isopropylphenyl)-4-((5-methoxy-6-morpholinopyridin-3-yl)methyl)piperazin-1-yl)cyclobutyl)piperazin-1-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(4-((1R,3S)-3-((R)-2-(2-isopropylphenyl)-4-((5-methoxy-6-morpholinopyridin-3-yl)methyl)piperazin-1-yl)cyclobutyl)piperazin-1-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(4-(3-((R)-2-(2-isopropylphenyl)-4-((5-methoxy-6-morpholinopyridin-3-yl)methyl)piperazin-1-yl)azetidin-1-yl)piperidin-1-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(4-((1S,4R)-4-((R)-2-(2-isopropylphenyl)-4-((5-methoxy-6-morpholinopyridin-3-yl)methyl)piperazin-1-yl)cyclohexyl)piperazin-1-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(4-((1R,4S)-4-((R)-2-(2-isopropylphenyl)-4-((5-methoxy-6-morpholinopyridin-3-yl)methyl)piperazin-1-yl)cyclohexyl)piperazin-1-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(4-((R)-2-(2-isopropylphenyl)-4-((5-methoxy-6-morpholinopyridin-3-yl)methyl)piperazin-1-yl)-[1,4′-bipiperidin]-1′-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(9-((R)-2-(2-isopropylphenyl)-4-((5-methoxy-6-morpholinopyridin-3-yl)methyl)piperazin-1-yl)-3-azaspiro[5.5]undecan-3-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-((R)-8-((R)-2-(2-isopropylphenyl)-4-((5-methoxy-6-morpholinopyridin-3-yl)methyl)piperazin-1-yl)-3-azaspiro[5.5]undecan-3-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-((S)-8-((R)-2-(2-isopropylphenyl)-4-((5-methoxy-6-morpholinopyridin-3-yl)methyl)piperazin-1-yl)-3-azaspiro[5.5]undecan-3-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-((S)-2-((R)-2-(2-isopropylphenyl)-4-((5-methoxy-6-morpholinopyridin-3-yl)methyl)piperazin-1-yl)-8-azaspiro[4.5]decan-8-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-((R)-2-((R)-2-(2-isopropylphenyl)-4-((5-methoxy-6-morpholinopyridin-3-yl)methyl)piperazin-1-yl)-8-azaspiro[4.5]decan-8-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(7-((R)-2-(2-isopropylphenyl)-4-((5-methoxy-6-morpholinopyridin-3-yl)methyl)piperazin-1-yl)-2-azaspiro[3.5]nonan-2-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(9-((3R,4S)-3-(2-isopropylphenyl)-1-((5-methoxy-6-morpholinopyridin-3-yl)methyl)piperidin-4-yl)-3,9-diazaspiro[5.5]undecan-3-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(9-((3R,4R)-3-(2-isopropylphenyl)-1-((5-methoxy-6-morpholinopyridin-3-yl)methyl)piperidin-4-yl)-3,9-diazaspiro[5.5]undecan-3-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((3R,4S)-3-(2-isopropylphenyl)-1-((5-methoxy-6-morpholinopyridin-3-yl)methyl)piperidin-4-yl)-2,7-diazaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((3R,4R)-3-(2-isopropylphenyl)-1-((5-methoxy-6-morpholinopyridin-3-yl)methyl)piperidin-4-yl)-2,7-diazaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(4-((R)-1-(2-isopropylphenyl)-3-((5-methoxy-6-morpholinopyridin-3-yl)methyl)-3-azaspiro[5.5]undecan-9-yl)piperazin-1-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(4-((R)-7-(2-isopropylphenyl)-9-((5-methoxy-6-morpholinopyridin-3-yl)methyl)-3,9-diazaspiro[5.5]undecan-3-yl)piperidin-1-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(4-((R)-5-(2-isopropylphenyl)-7-((5-methoxy-6-morpholinopyridin-3-yl)methyl)-7-azaspiro[3.5]nonan-2-yl)piperazin-1-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(4-((S)-5-(2-isopropylphenyl)-7-((5-methoxy-6-morpholinopyridin-3-yl)methyl)-2,7-diazaspiro[3.5]nonan-2-yl)piperidin-1-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(9-((S)-1-(2-isopropylphenyl)-4-((5-methoxy-6-morpholinopyridin-3-yl)methyl)piperazin-2-yl)-3-azaspiro[5.5]undecan-3-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(9-((R)-1-(2-isopropylphenyl)-4-((5-methoxy-6-morpholinopyridin-3-yl)methyl)piperazin-2-yl)-3-azaspiro[5.5]undecan-3-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((S)-1-(2-isopropylphenyl)-4-((5-methoxy-6-morpholinopyridin-3-yl)methyl)piperazin-2-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((R)-1-(2-isopropylphenyl)-4-((5-methoxy-6-morpholinopyridin-3-yl)methyl)piperazin-2-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4′-((5,6-dimethoxypyridin-3-yl)methyl)-3,3-dimethyl-2,3-dihydrospiro[indene-1,2′-piperazin]-1′-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4′-((5,6-dimethoxypyridin-3-yl)methyl)-1,1-dimethylspiro[isochromane-4,2′-piperazin]-1′-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((1R,3S)-4′-((5-methoxy-6-morpholinopyridin-3-yl)methyl)-3-methyl-2,3-dihydrospiro[indene-1,2′-piperazin]-1′-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((1R,3R)-4′-((5-methoxy-6-morpholinopyridin-3-yl)methyl)-3-methyl-2,3-dihydrospiro[indene-1,2′-piperazin]-1′-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((1S,3S)-4′-((5-methoxy-6-morpholinopyridin-3-yl)methyl)-3-methyl-2,3-dihydrospiro[indene-1,2′-piperazin]-1′-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((1S,3R)-4′-((5-methoxy-6-morpholinopyridin-3-yl)methyl)-3-methyl-2,3-dihydrospiro[indene-1,2′-piperazin]-1′-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-3-(((1r,4R)-4-hydroxy-4-methylcyclohexyl)methyl)-5-nitro-3,4-dihydro-2H-benzo[b][1,4]oxazin-7-yl)sulfonyl)-4-(2-((R)-4-((5-methoxy-6-morpholinopyridin-3-yl)methyl)-2-(o-tolyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-3-(((1s,4S)-4-hydroxy-4-methylcyclohexyl)methyl)-5-nitro-3,4-dihydro-2H-benzo[b][1,4]oxazin-7-yl)sulfonyl)-4-(2-((R)-4-((5-methoxy-6-morpholinopyridin-3-yl)methyl)-2-(o-tolyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-(((S)-3-(((1r,4S)-4-hydroxy-4-methylcyclohexyl)methyl)-5-nitro-3,4-dihydro-2H-benzo[b][1,4]oxazin-7-yl)sulfonyl)-4-(2-((R)-4-((5-methoxy-6-morpholinopyridin-3-yl)methyl)-2-(o-tolyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-(((S)-3-(((1s,4R)-4-hydroxy-4-methylcyclohexyl)methyl)-5-nitro-3,4-dihydro-2H-benzo[b][1,4]oxazin-7-yl)sulfonyl)-4-(2-((R)-4-((5-methoxy-6-morpholinopyridin-3-yl)methyl)-2-(o-tolyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-3-(((1s,4S)-4-hydroxycyclohexyl)methyl)-5-nitro-3,4-dihydro-2H-benzo[b][1,4]oxazin-7-yl)sulfonyl)-4-(2-((R)-4-((5-methoxy-6-morpholinopyridin-3-yl)methyl)-2-(o-tolyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-3-(((1r,4R)-4-hydroxycyclohexyl)methyl)-5-nitro-3,4-dihydro-2H-benzo[b][1,4]oxazin-7-yl)sulfonyl)-4-(2-((R)-4-((5-methoxy-6-morpholinopyridin-3-yl)methyl)-2-(o-tolyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-(((S)-3-(((1s,4R)-4-hydroxycyclohexyl)methyl)-5-nitro-3,4-dihydro-2H-benzo[b][1,4]oxazin-7-yl)sulfonyl)-4-(2-((R)-4-((5-methoxy-6-morpholinopyridin-3-yl)methyl)-2-(o-tolyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-(((S)-3-(((1s,4R)-4-hydroxycyclohexyl)methyl)-5-nitro-3,4-dihydro-2H-benzo[b][1,4]oxazin-7-yl)sulfonyl)-4-(2-((R)-4-((5-methoxy-6-morpholinopyridin-3-yl)methyl)-2-(o-tolyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-3-((4-hydroxy-4-methylpiperidin-1-yl)methyl)-5-nitro-3,4-dihydro-2H-benzo[b][1,4]oxazin-7-yl)sulfonyl)-4-(2-((R)-4-((5-methoxy-6-morpholinopyridin-3-yl)methyl)-2-(o-tolyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-3-((4-hydroxy-4-methylpiperidin-1-yl)methyl)-5-nitro-3,4-dihydro-2H-benzo[b][1,4]oxazin-7-yl)sulfonyl)-4-(2-((R)-4-((5-methoxy-6-morpholinopyridin-3-yl)methyl)-2-(o-tolyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((5-methoxy-6-morpholinopyridin-3-yl)methyl)-2-(o-tolyl)piperazin-1-yl)-7-azaspiro[3.5]nonan-7-yl)-N—(((R)-3-(morpholinomethyl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((5-methoxy-6-morpholinopyridin-3-yl)methyl)-2-(o-tolyl)piperazin-1-yl)-7-azaspiro[3.5]nonan-7-yl)-N-(((S)-3-(morpholinomethyl)-5-nitro-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-3-(((1S,4S)-2-oxa-5-azabicyclo[2.2.1]heptan-5-yl)methyl)-5-nitro-3,4-dihydro-2H-benzo[b][1,4]oxazin-7-yl)sulfonyl)-4-(2-((R)-4-((5-methoxy-6-morpholinopyridin-3-yl)methyl)-2-(o-tolyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-(((S)-3-(((1S,4S)-2-oxa-5-azabicyclo[2.2.1]heptan-5-yl)methyl)-5-nitro-3,4-dihydro-2H-benzo[b][1,4]oxazin-7-yl)sulfonyl)-4-(2-((R)-4-((5-methoxy-6-morpholinopyridin-3-yl)methyl)-2-(o-tolyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-3-(((1R,4R)-2-oxa-5-azabicyclo[2.2.1]heptan-5-yl)methyl)-5-nitro-3,4-dihydro-2H-benzo[b][1,4]oxazin-7-yl)sulfonyl)-4-(2-((R)-4-((5-methoxy-6-morpholinopyridin-3-yl)methyl)-2-(o-tolyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-(((S)-3-(((1R,4R)-2-oxa-5-azabicyclo[2.2.1]heptan-5-yl)methyl)-5-nitro-3,4-dihydro-2H-benzo[b][1,4]oxazin-7-yl)sulfonyl)-4-(2-((R)-4-((5-methoxy-6-morpholinopyridin-3-yl)methyl)-2-(o-tolyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-3-(isopropoxymethyl)-5-nitro-3,4-dihydro-2H-benzo[b][1,4]oxazin-7-yl)sulfonyl)-4-(2-((R)-4-((5-methoxy-6-morpholinopyridin-3-yl)methyl)-2-(o-tolyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-(((S)-3-(isopropoxymethyl)-5-nitro-3,4-dihydro-2H-benzo[b][1,4]oxazin-7-yl)sulfonyl)-4-(2-((R)-4-((5-methoxy-6-morpholinopyridin-3-yl)methyl)-2-(o-tolyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-3-((4-isopropylpiperazin-1-yl)methyl)-5-nitro-3,4-dihydro-2H-benzo[b][1,4]oxazin-7-yl)sulfonyl)-4-(2-((R)-4-((5-methoxy-6-morpholinopyridin-3-yl)methyl)-2-(o-tolyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-(((S)-3-((4-isopropylpiperazin-1-yl)methyl)-5-nitro-3,4-dihydro-2H-benzo[b][1,4]oxazin-7-yl)sulfonyl)-4-(2-((R)-4-((5-methoxy-6-morpholinopyridin-3-yl)methyl)-2-(o-tolyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-3-((4,4-difluoropiperidin-1-yl)methyl)-5-nitro-3,4-dihydro-2H-benzo[b][1,4]oxazin-7-yl)sulfonyl)-4-(2-((R)-4-((5-methoxy-6-morpholinopyridin-3-yl)methyl)-2-(o-tolyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-(((S)-3-((4,4-difluoropiperidin-1-yl)methyl)-5-nitro-3,4-dihydro-2H-benzo[b][1,4]oxazin-7-yl)sulfonyl)-4-(2-((R)-4-((5-methoxy-6-morpholinopyridin-3-yl)methyl)-2-(o-tolyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-3-((4-(isopropylsulfonyl)piperazin-1-yl)methyl)-5-nitro-3,4-dihydro-2H-benzo[b][1,4]oxazin-7-yl)sulfonyl)-4-(2-((R)-4-((5-methoxy-6-morpholinopyridin-3-yl)methyl)-2-(o-tolyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-(((S)-3-((4-(isopropylsulfonyl)piperazin-1-yl)methyl)-5-nitro-3,4-dihydro-2H-benzo[b][1,4]oxazin-7-yl)sulfonyl)-4-(2-((R)-4-((5-methoxy-6-morpholinopyridin-3-yl)methyl)-2-(o-tolyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((5-methoxy-6-morpholinopyridin-3-yl)methyl)-2-(o-tolyl)piperazin-1-yl)-7-azaspiro[3.5]nonan-7-yl)-N—(((R)-5-nitro-3-(pyridin-2-ylmethyl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((5-methoxy-6-morpholinopyridin-3-yl)methyl)-2-(o-tolyl)piperazin-1-yl)-7-azaspiro[3.5]nonan-7-yl)-N-(((S)-5-nitro-3-(pyridin-2-ylmethyl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-(((S)-2-((1r,4S)-4-hydroxy-4-methylcyclohexyl)-8-nitro-2,3-dihydro-1H-pyrido[2,3-b][1,4]oxazin-6-yl)sulfonyl)-4-(2-((R)-4-((5-methoxy-6-morpholinopyridin-3-yl)methyl)-2-(o-tolyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((5-methoxy-6-morpholinopyridin-3-yl)methyl)-2-(o-tolyl)piperazin-1-yl)-7-azaspiro[3.5]nonan-7-yl)-N—(((R)-8-nitro-2-(tetrahydro-2H-pyran-4-yl)-2,3-dihydro-1H-pyrido[2,3-b][1,4]oxazin-6-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((5-methoxy-6-morpholinopyridin-3-yl)methyl)-2-(o-tolyl)piperazin-1-yl)-7-azaspiro[3.5]nonan-7-yl)-N-(((S)-8-nitro-2-(tetrahydro-2H-pyran-4-yl)-2,3-dihydro-1H-pyrido[2,3-b][1,4]oxazin-6-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((5-methoxy-6-morpholinopyridin-3-yl)methyl)-2-(o-tolyl)piperazin-1-yl)-7-azaspiro[3.5]nonan-7-yl)-N-((4-nitro-5-(((tetrahydro-2H-pyran-4-yl)methyl)amino)pyridin-2-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-(((S)-2-((1r,4S)-4-hydroxy-4-methylcyclohexyl)-8-nitro-2,3-dihydro-1H-pyrido[2,3-b][1,4]oxazin-6-yl)sulfonyl)-4-(2-((R)-4-((5-methoxy-6-morpholinopyridin-3-yl)methyl)-2-(o-tolyl)piperazin-1-yl)-1-methyl-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((5-methoxy-6-morpholinopyridin-3-yl)methyl)-2-(o-tolyl)piperazin-1-yl)-1-methyl-7-azaspiro[3.5]nonan-7-yl)-N—(((R)-8-nitro-2-(tetrahydro-2H-pyran-4-yl)-2,3-dihydro-1H-pyrido[2,3-b][1,4]oxazin-6-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((5-methoxy-6-morpholinopyridin-3-yl)methyl)-2-(o-tolyl)piperazin-1-yl)-1-methyl-7-azaspiro[3.5]nonan-7-yl)-N-(((S)-8-nitro-2-(tetrahydro-2H-pyran-4-yl)-2,3-dihydro-1H-pyrido[2,3-b][1,4]oxazin-6-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((5-methoxy-6-morpholinopyridin-3-yl)methyl)-2-(o-tolyl)piperazin-1-yl)-1-methyl-7-azaspiro[3.5]nonan-7-yl)-N-((4-nitro-5-(((tetrahydro-2H-pyran-4-yl)methyl)amino)pyridin-2-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-(((S)-2-((1r,4S)-4-hydroxy-4-methylcyclohexyl)-8-nitro-2,3-dihydro-1H-pyrido[2,3-b][1,4]oxazin-6-yl)sulfonyl)-4-(2-((R)-4-((5-methoxy-6-((R)-3-methylmorpholino)pyridin-3-yl)methyl)-2-(o-tolyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((5-methoxy-6-((R)-3-methylmorpholino)pyridin-3-yl)methyl)-2-(o-tolyl)piperazin-1-yl)-7-azaspiro[3.5]nonan-7-yl)-N—(((R)-8-nitro-2-(tetrahydro-2H-pyran-4-yl)-2,3-dihydro-1H-pyrido[2,3-b][1,4]oxazin-6-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((5-methoxy-6-((R)-3-methylmorpholino)pyridin-3-yl)methyl)-2-(o-tolyl)piperazin-1-yl)-7-azaspiro[3.5]nonan-7-yl)-N-(((S)-8-nitro-2-(tetrahydro-2H-pyran-4-yl)-2,3-dihydro-1H-pyrido[2,3-b][1,4]oxazin-6-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((5-methoxy-6-((R)-3-methylmorpholino)pyridin-3-yl)methyl)-2-(o-tolyl)piperazin-1-yl)-7-azaspiro[3.5]nonan-7-yl)-N-((4-nitro-5-(((tetrahydro-2H-pyran-4-yl)methyl)amino)pyridin-2-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-(((S)-2-((1r,4S)-4-hydroxy-4-methylcyclohexyl)-8-nitro-2,3-dihydro-1H-pyrido[2,3-b][1,4]oxazin-6-yl)sulfonyl)-4-(2-((S)-2-(2-isopropoxyphenyl)-4-((5-methoxy-6-morpholinopyridin-3-yl)methyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-2-(2-isopropoxyphenyl)-4-((5-methoxy-6-morpholinopyridin-3-yl)methyl)piperazin-1-yl)-7-azaspiro[3.5]nonan-7-yl)-N—(((R)-8-nitro-2-(tetrahydro-2H-pyran-4-yl)-2,3-dihydro-1H-pyrido[2,3-b][1,4]oxazin-6-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-2-(2-isopropoxyphenyl)-4-((5-methoxy-6-morpholinopyridin-3-yl)methyl)piperazin-1-yl)-7-azaspiro[3.5]nonan-7-yl)-N-(((S)-8-nitro-2-(tetrahydro-2H-pyran-4-yl)-2,3-dihydro-1H-pyrido[2,3-b][1,4]oxazin-6-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-2-(2-isopropoxyphenyl)-4-((5-methoxy-6-morpholinopyridin-3-yl)methyl)piperazin-1-yl)-7-azaspiro[3.5]nonan-7-yl)-N-((4-nitro-5-(((tetrahydro-2H-pyran-4-yl)methyl)amino)pyridin-2-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-(((S)-2-((1r,4S)-4-hydroxy-4-methylcyclohexyl)-8-nitro-2,3-dihydro-1H-pyrido[2,3-b][1,4]oxazin-6-yl)sulfonyl)-4-(2-((S)-2-(2-isopropoxyphenyl)-4-((5-methoxy-6-morpholi nopyridin-3-yl)methyl)piperazin-1-yl)-1-methyl-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-2-(2-isopropoxyphenyl)-4-((5-methoxy-6-morpholinopyridin-3-yl)methyl)piperazin-1-yl)-1-methyl-7-azaspiro[3.5]nonan-7-yl)-N—(((R)-8-nitro-2-(tetrahydro-2H-pyran-4-yl)-2,3-dihydro-1H-pyrido[2,3-b][1,4]oxazin-6-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-2-(2-isopropoxyphenyl)-4-((5-methoxy-6-morpholinopyridin-3-yl)methyl)piperazin-1-yl)-1-methyl-7-azaspiro[3.5]nonan-7-yl)-N-(((S)-8-nitro-2-(tetrahydro-2H-pyran-4-yl)-2,3-dihydro-1H-pyrido[2,3-b][1,4]oxazin-6-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-2-(2-isopropoxyphenyl)-4-((5-methoxy-6-morpholinopyridin-3-yl)methyl)piperazin-1-yl)-1-methyl-7-azaspiro[3.5]nonan-7-yl)-N-((4-nitro-5-(((tetrahydro-2H-pyran-4-yl)methyl)amino)pyridin-2-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-(((S)-2-((1r,4S)-4-hydroxy-4-methylcyclohexyl)-8-nitro-2,3-dihydro-1H-pyrido[2,3-b][1,4]oxazin-6-yl)sulfonyl)-4-(2-((S)-2-(2-isopropoxyphenyl)-4-((5-methoxy-6-((R)-3-methylmorpholino)pyridin-3-yl)methyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-2-(2-isopropoxyphenyl)-4-((5-methoxy-6-((R)-3-methylmorpholino)pyridin-3-yl)methyl)piperazin-1-yl)-7-azaspiro[3.5]nonan-7-yl)-N—(((R)-8-nitro-2-(tetrahydro-2H-pyran-4-yl)-2,3-dihydro-1H-pyrido[2,3-b][1,4]oxazin-6-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-2-(2-isopropoxyphenyl)-4-((5-methoxy-6-((R)-3-methylmorpholino)pyridin-3-yl)methyl)piperazin-1-yl)-7-azaspiro[3.5]nonan-7-yl)-N-(((S)-8-nitro-2-(tetrahydro-2H-pyran-4-yl)-2,3-dihydro-1H-pyrido[2,3-b][1,4]oxazin-6-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((S)-2-(2-isopropoxyphenyl)-4-((5-methoxy-6-((R)-3-methylmorpholino)pyridin-3-yl)methyl)piperazin-1-yl)-7-azaspiro[3.5]nonan-7-yl)-N-((4-nitro-5-(((tetrahydro-2H-pyran-4-yl)methyl)amino)pyridin-2-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((5-methoxy-6-morpholinopyridin-3-yl)methyl)-2-(o-tolyl)piperazin-1-yl)-7-azaspiro[3.5]nonan-7-yl)-2-((R)-3-methyl-2,3-dihydropyrrolo[3′,2′:5,6]pyrido[2,3-b][1,4]oxazin-1(6H)-yl)-N-(((S)-8-nitro-2-(tetrahydro-2H-pyran-4-yl)-2,3-dihydro-1H-pyrido[2,3-b][1,4]oxazin-6-yl)sulfonyl)benzamide,
4-(2-((R)-4-((5-methoxy-6-morpholinopyridin-3-yl)methyl)-2-(o-tolyl)piperazin-1-yl)-7-azaspiro[3.5]nonan-7-yl)-2-((R)-3-methyl-2,3-dihydropyrrolo[3′,2′:5,6]pyrido[2,3-b][1,4]oxazin-1(6H)-yl)-N—(((R)-8-nitro-2-(tetrahydro-2H-pyran-4-yl)-2,3-dihydro-1H-pyrido[2,3-b][1,4]oxazin-6-yl)sulfonyl)benzamide,
4-(2-((R)-4-((5-methoxy-6-morpholinopyridin-3-yl)methyl)-2-(o-tolyl)piperazin-1-yl)-7-azaspiro[3.5]nonan-7-yl)-2-((S)-3-methyl-2,3-dihydropyrrolo[3′,2′:5,6]pyrido[2,3-b][1,4]oxazin-1(6H)-yl)-N-(((S)-8-nitro-2-(tetrahydro-2H-pyran-4-yl)-2,3-dihydro-1H-pyrido[2,3-b][1,4]oxazin-6-yl)sulfonyl)benzamide,
4-(2-((R)-4-((5-methoxy-6-morpholinopyridin-3-yl)methyl)-2-(o-tolyl)piperazin-1-yl)-7-azaspiro[3.5]nonan-7-yl)-2-((S)-3-methyl-2,3-dihydropyrrolo[3′,2′:5,6]pyrido[2,3-b][1,4]oxazin-1(6H)-yl)-N—(((R)-8-nitro-2-(tetrahydro-2H-pyran-4-yl)-2,3-dihydro-1H-pyrido[2,3-b][1,4]oxazin-6-yl)sulfonyl)benzamide,
2-(2,3-dihydropyrrolo[3′,2′:5,6]pyrido[2,3-b][1,4]oxazin-1(6H)-yl)-4-(2-((R)-2-(2-isopropylphenyl)-4-((5-methoxy-6-morpholinopyridin-3-yl)methyl)piperazin-1-yl)-7-azaspiro[3.5]nonan-7-yl)-N—(((R)-8-nitro-2-(tetrahydro-2H-pyran-4-yl)-2,3-dihydro-1H-pyrido[2,3-b][1,4]oxazin-6-yl)sulfonyl)benzamide,
2-(2,3-dihydropyrrolo[3′,2′:5,6]pyrido[2,3-b][1,4]oxazin-1(6H)-yl)-4-(2-((R)-2-(2-isopropoxyphenyl)-4-((5-methoxy-6-morpholinopyridin-3-yl)methyl)piperazin-1-yl)-7-azaspiro[3.5]nonan-7-yl)-N—(((R)-8-nitro-2-(tetrahydro-2H-pyran-4-yl)-2,3-dihydro-1H-pyrido[2,3-b][1,4]oxazin-6-yl)sulfonyl)benzamide,
4-(2-((S)-4-((5-methoxy-6-morpholinopyridin-3-yl)methyl)-2-(o-tolyl)piperazin-1-yl)-7-azaspiro[3.5]nonan-7-yl)-2-((S)-3-methyl-2,3-dihydropyrrolo[3′,2′:5,6]pyrido[2,3-b][1,4]oxazin-1(6H)-yl)-N—(((R)-8-nitro-2-(tetrahydro-2H-pyran-4-yl)-2,3-dihydro-1H-pyrido[2,3-b][1,4]oxazin-6-yl)sulfonyl)benzamide,
2-(2,3-dihydropyrrolo[3′,2′:5,6]pyrido[2,3-b][1,4]oxazin-1(6H)-yl)-4-(2-((S)-2-(2-isopropylphenyl)-4-((5-methoxy-6-morpholinopyridin-3-yl)methyl)piperazin-1-yl)-7-azaspiro[3.5]nonan-7-yl)-N—(((R)-8-nitro-2-(tetrahydro-2H-pyran-4-yl)-2,3-dihydro-1H-pyrido[2,3-b][1,4]oxazin-6-yl)sulfonyl)benzamide,
2-(2,3-dihydropyrrolo[3′,2′:5,6]pyrido[2,3-b][1,4]oxazin-1(6H)-yl)-4-(2-((S)-2-(2-isopropoxyphenyl)-4-((5-methoxy-6-morpholinopyridin-3-yl)methyl)piperazin-1-yl)-7-azaspiro[3.5]nonan-7-yl)-N—(((R)-8-nitro-2-(tetrahydro-2H-pyran-4-yl)-2,3-dihydro-1H-pyrido[2,3-b][1,4]oxazin-6-yl)sulfonyl)benzamide,
4-(2-((R)-4-(3,4-difluorobenzyl)-2-(o-tolyl)piperazin-1-yl)-7-azaspiro[3.5]nonan-7-yl)-2-(2,3-dihydropyrrolo[3′,2′:5,6]pyrido[2,3-b][1,4]oxazin-1(6H)-yl)-N—(((R)-8-nitro-2-(tetrahydro-2H-pyran-4-yl)-2,3-dihydro-1H-pyrido[2,3-b][1,4]oxazin-6-yl)sulfonyl)benzamide,
4-(2-((R)-4-(3,4-difluorobenzyl)-2-(2-isopropylphenyl)piperazin-1-yl)-7-azaspiro[3.5]nonan-7-yl)-2-(2,3-dihydropyrrolo[3′,2′:5,6]pyrido[2,3-b][1,4]oxazin-1(6H)-yl)-N—(((R)-8-nitro-2-(tetrahydro-2H-pyran-4-yl)-2,3-dihydro-1H-pyrido[2,3-b][1,4]oxazin-6-yl)sulfonyl)benzamide,
4-(2-((R)-4-(3,4-difluorobenzyl)-2-(2-isopropoxyphenyl)piperazin-1-yl)-7-azaspiro[3.5]nonan-7-yl)-2-(2,3-dihydropyrrolo[3′,2′:5,6]pyrido[2,3-b][1,4]oxazin-1(6H)-yl)-N—(((R)-8-nitro-2-(tetrahydro-2H-pyran-4-yl)-2,3-dihydro-1H-pyrido[2,3-b][1,4]oxazin-6-yl)sulfonyl)benzamide,
2-(2,3-dihydropyrrolo[3′,2′:5,6]pyrido[2,3-b][1,4]oxazin-1(6H)-yl)-4-(2-((R)-4-methyl-2-(o-tolyl)piperazin-1-yl)-7-azaspiro[3.5]nonan-7-yl)-N—(((R)-8-nitro-2-(tetrahydro-2H-pyran-4-yl)-2,3-dihydro-1H-pyrido[2,3-b][1,4]oxazin-6-yl)sulfonyl)benzamide,
2-(2,3-dihydropyrrolo[3′,2′:5,6]pyrido[2,3-b][1,4]oxazin-1(6H)-yl)-4-(2-((R)-2-(2-isopropylphenyl)-4-methylpiperazin-1-yl)-7-azaspiro[3.5]nonan-7-yl)-N—(((R)-8-nitro-2-(tetrahydro-2H-pyran-4-yl)-2,3-dihydro-1H-pyrido[2,3-b][1,4]oxazin-6-yl)sulfonyl)benzamide,
2-(2,3-dihydropyrrolo[3′,2′:5,6]pyrido[2,3-b][1,4]oxazin-1(6H)-yl)-4-(2-((R)-2-(2-isopropoxyphenyl)-4-methylpiperazin-1-yl)-7-azaspiro[3.5]nonan-7-yl)-N—(((R)-8-nitro-2-(tetrahydro-2H-pyran-4-yl)-2,3-dihydro-1H-pyrido[2,3-b][1,4]oxazin-6-yl)sulfonyl)benzamide,
2-(2,3-dihydropyrrolo[3′,2′:5,6]pyrido[2,3-b][1,4]oxazin-1(6H)-yl)-N—(((R)-8-nitro-2-(tetrahydro-2H-pyran-4-yl)-2,3-dihydro-1H-pyrido[2,3-b][1,4]oxazin-6-yl)sulfonyl)-4-(2-((R)-3-(o-tolyl)morpholino)-7-azaspiro[3.5]nonan-7-yl)benzamide,
2-(2,3-dihydropyrrolo[3′,2′:5,6]pyrido[2,3-b][1,4]oxazin-1(6H)-yl)-4-(2-((R)-3-(2-isopropylphenyl)morpholino)-7-azaspiro[3.5]nonan-7-yl)-N—(((R)-8-nitro-2-(tetrahydro-2H-pyran-4-yl)-2,3-dihydro-1H-pyrido[2,3-b][1,4]oxazin-6-yl)sulfonyl)benzamide,
2-(2,3-dihydropyrrolo[3′,2′:5,6]pyrido[2,3-b][1,4]oxazin-1(6H)-yl)-4-(2-((R)-3-(2-isopropoxyphenyl)morpholino)-7-azaspiro[3.5]nonan-7-yl)-N—(((R)-8-nitro-2-(tetrahydro-2H-pyran-4-yl)-2,3-dihydro-1H-pyrido[2,3-b][1,4]oxazin-6-yl)sulfonyl)benzamide,
(R)-2-(2,3-dihydropyrrolo[3′,2′:5,6]pyrido[2,3-b][1,4]oxazin-1(6H)-yl)-4-(2-(4-methyl-2-(o-tolyl)piperazin-1-yl)-7-azaspiro[3.5]nonan-7-yl)-N-((4-nitro-5-(((tetrahydro-2H-pyran-4-yl)methyl)amino)pyridin-2-yl)sulfonyl)benzamide,
(R)-2-(2,3-dihydropyrrolo[3′,2′:5,6]pyrido[2,3-b][1,4]oxazin-1(6H)-yl)-4-(2-(2-(2-isopropylphenyl)-4-methylpiperazin-1-yl)-7-azaspiro[3.5]nonan-7-yl)-N-((4-nitro-5-(((tetrahydro-2H-pyran-4-yl)methyl)amino)pyridin-2-yl)sulfonyl)benzamide,
(R)-2-(2,3-dihydropyrrolo[3′,2′:5,6]pyrido[2,3-b][1,4]oxazin-1(6H)-yl)-4-(2-(2-(2-isopropoxyphenyl)-4-methylpiperazin-1-yl)-7-azaspiro[3.5]nonan-7-yl)-N-((4-nitro-5-(((tetrahydro-2H-pyran-4-yl)methyl)amino)pyridin-2-yl)sulfonyl)benzamide,
(R)-2-(2,3-dihydropyrrolo[3′,2′:5,6]pyrido[2,3-b][1,4]oxazin-1(6H)-yl)-4-(2-(4-methyl-2-(o-tolyl)piperazin-1-yl)-7-azaspiro[3.5]nonan-7-yl)-N-((5-nitro-6-(((tetrahydro-2H-pyran-4-yl)methyl)amino)pyridin-3-yl)sulfonyl)benzamide,
(R)-2-(2,3-dihydropyrrolo[3′,2′:5,6]pyrido[2,3-b][1,4]oxazin-1(6H)-yl)-4-(2-(2-(2-isopropylphenyl)-4-methylpiperazin-1-yl)-7-azaspiro[3.5]nonan-7-yl)-N-((6-nitro-5-(((tetrahydro-2H-pyran-4-yl)methyl)amino)pyridin-2-yl)sulfonyl)benzamide,
(R)-2-(2,3-dihydropyrrolo[3′,2′:5,6]pyrido[2,3-b][1,4]oxazin-1(6H)-yl)-4-(2-(2-(2-isopropoxyphenyl)-4-methylpiperazin-1-yl)-7-azaspiro[3.5]nonan-7-yl)-N-((5-nitro-6-(((tetrahydro-2H-pyran-4-yl)methyl)amino)pyridazin-3-yl)sulfonyl)benzamide,
2-(2,3-dihydropyrrolo[3′,2′:5,6]pyrido[2,3-b][1,4]oxazin-1(6H)-yl)-5-fluoro-4-(2-((R)-4-((5-methoxy-6-morpholinopyridin-3-yl)methyl)-2-(o-tolyl)piperazin-1-yl)-7-azaspiro[3.5]nonan-7-yl)-N—(((R)-8-nitro-2-(tetrahydro-2H-pyran-4-yl)-2,3-dihydro-1H-pyrido[2,3-b][1,4]oxazin-6-yl)sulfonyl)benzamide,
2-(2,3-dihydropyrrolo[3′,2′:5,6]pyrido[2,3-b][1,4]oxazin-1(6H)-yl)-5-fluoro-6-(2-((R)-2-(2-isopropylphenyl)-4-((5-methoxy-6-morpholinopyridin-3-yl)methyl)piperazin-1-yl)-7-azaspiro[3.5]nonan-7-yl)-N—(((R)-8-nitro-2-(tetrahydro-2H-pyran-4-yl)-2,3-dihydro-1H-pyrido[2,3-b][1,4]oxazin-6-yl)sulfonyl)nicotinamide,
4-(2,3-dihydropyrrolo[3′,2′:5,6]pyrido[2,3-b][1,4]oxazin-1(6H)-yl)-5-fluoro-6-(2-((R)-2-(2-isopropoxyphenyl)-4-((5-methoxy-6-morpholinopyridin-3-yl)methyl)piperazin-1-yl)-7-azaspiro[3.5]nonan-7-yl)-N—(((R)-8-nitro-2-(tetrahydro-2H-pyran-4-yl)-2,3-dihydro-1H-pyrido[2,3-b][1,4]oxazin-6-yl)sulfonyl)nicotinamide,
2-(8-fluoro-2,3-dihydropyrrolo[3′,2′:5,6]pyrido[2,3-b][1,4]oxazin-1(6H)-yl)-4-(2-((R)-4-((5-methoxy-6-morpholinopyridin-3-yl)methyl)-2-(o-tolyl)piperazin-1-yl)-7-azaspiro[3.5]nonan-7-yl)-N—(((R)-8-nitro-2-(tetrahydro-2H-pyran-4-yl)-2,3-dihydro-1H-pyrido[2,3-b][1,4]oxazin-6-yl)sulfonyl)benzamide,
2-(8-fluoro-2,3-dihydropyrrolo[3′,2′:5,6]pyrido[2,3-b][1,4]oxazin-1(6H)-yl)-4-(2-((R)-2-(2-isopropylphenyl)-4-((5-methoxy-6-morpholinopyridin-3-yl)methyl)piperazin-1-yl)-7-azaspiro[3.5]nonan-7-yl)-N—(((R)-8-nitro-2-(tetrahydro-2H-pyran-4-yl)-2,3-dihydro-1H-pyrido[2,3-b][1,4]oxazin-6-yl)sulfonyl)benzamide,
2-(8-fluoro-2,3-dihydropyrrolo[3′,2′:5,6]pyrido[2,3-b][1,4]oxazin-1(6H)-yl)-4-(2-((R)-2-(2-isopropoxyphenyl)-4-((5-methoxy-6-morpholinopyridin-3-yl)methyl)piperazin-1-yl)-7-azaspiro[3.5]nonan-7-yl)-N—(((R)-8-nitro-2-(tetrahydro-2H-pyran-4-yl)-2,3-dihydro-1H-pyrido[2,3-b][1,4]oxazin-6-yl)sulfonyl)benzamide,
N-((4-((((1r,4r)-4-hydroxy-4-methylcyclohexyl)methyl)amino)-3-nitrophenyl)sulfonyl)-6-(2-((R)-4-((5-methoxy-6-morpholinopyridin-3-yl)methyl)-2-(o-tolyl)piperazin-1-yl)-7-azaspiro[3.5]nonan-7-yl)-4-((5aS,9aR)-6,7,9,9a-tetrahydro-1H-pyrano[3,4-b]pyrrolo[3′,2′:5,6]pyrido[3,2-e][1,4]oxazin-5(5aH)-yl)nicotinamide,
N-((4-((((1r,4r)-4-hydroxy-4-methylcyclohexyl)methyl)amino)-3-nitrophenyl)sulfonyl)-6-(2-((R)-4-((5-methoxy-6-morpholinopyridin-3-yl)methyl)-2-(o-tolyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)nicotinamide,
N-((4-((((1r,4r)-4-hydroxy-4-methylcyclohexyl)methyl)amino)-3-nitrophenyl)sulfonyl)-2-(2-((R)-4-((5-methoxy-6-morpholinopyridin-3-yl)methyl)-2-(o-tolyl)piperazin-1-yl)-7-azaspiro[3.5]nonan-7-yl)-4-((5aS,9aR)-6,7,9,9a-tetrahydro-1H-pyrano[3,4-b]pyrrolo[3′,2′:5,6]pyrido[3,2-e][1,4]oxazin-5(5aH)-yl)pyrimidine-5-carboxamide,
N-((4-((((1r,4r)-4-hydroxy-4-methylcyclohexyl)methyl)amino)-3-nitrophenyl)sulfonyl)-5-(2-((R)-4-((5-methoxy-6-morpholinopyridin-3-yl)methyl)-2-(o-tolyl)piperazin-1-yl)-7-azaspiro[3.5]nonan-7-yl)-3-((5aS,9aR)-6,7,9,9a-tetrahydro-1H-pyrano[3,4-b]pyrrolo[3′,2′:5,6]pyrido[3,2-e][1,4]oxazin-5(5aH)-yl)picolinamide,
5-fluoro-N-((4-((((1r,4r)-4-hydroxy-4-methylcyclohexyl)methyl)amino)-3-nitrophenyl)sulfonyl)-4-(2-((R)-4-((5-methoxy-6-morpholinopyridin-3-yl)methyl)-2-(o-tolyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
6-(2-((R)-4-((5-methoxy-6-morpholinopyridin-3-yl)methyl)-2-(o-tolyl)piperazin-1-yl)-7-azaspiro[3.5]nonan-7-yl)-N-((3-nitro-4-(((tetrahydro-2H-pyran-4-yl)methyl)amino)phenyl)sulfonyl)-4-((5aS,9aR)-6,7,9,9a-tetrahydro-1H-pyrano[3,4-b]pyrrolo[3′,2′:5,6]pyrido[3,2-e][1,4]oxazin-5(5aH)-yl)nicotinamide,
6-(2-((R)-4-((5-methoxy-6-morpholinopyridin-3-yl)methyl)-2-(o-tolyl)piperazin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)nicotinamide,
2-(2-((R)-4-((5-methoxy-6-morpholinopyridin-3-yl)methyl)-2-(o-tolyl)piperazin-1-yl)-7-azaspiro[3.5]nonan-7-yl)-N-((3-nitro-4-(((tetrahydro-2H-pyran-4-yl)methyl)amino)phenyl)sulfonyl)-4-((5aS,9aR)-6,7,9,9a-tetrahydro-1H-pyrano[3,4-b]pyrrolo[3′,2′:5,6]pyrido[3,2-e][1,4]oxazin-5(5aH)-yl)pyrimidine-5-carboxamide,
5-(2-((R)-4-((5-methoxy-6-morpholinopyridin-3-yl)methyl)-2-(o-tolyl)piperazin-1-yl)-7-azaspiro[3.5]nonan-7-yl)-N-((3-nitro-4-(((tetrahydro-2H-pyran-4-yl)methyl)amino)phenyl)sulfonyl)-3-((5aS,9aR)-6,7,9,9a-tetrahydro-1H-pyrano[3,4-b]pyrrolo[3′,2′:5,6]pyrido[3,2-e][1,4]oxazin-5(5aH)-yl)picolinamide,
5-fluoro-4-(2-((R)-4-((5-methoxy-6-morpholinopyridin-3-yl)methyl)-2-(o-tolyl)piperazin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
6-(2-((R)-4-(3,4-difluorobenzyl)-2-(o-tolyl)piperazin-1-yl)-7-azaspiro[3.5]nonan-7-yl)-N-((3-nitro-4-(((tetrahydro-2H-pyran-4-yl)methyl)amino)phenyl)sulfonyl)-4-((5aS,9aR)-6,7,9,9a-tetrahydro-1H-pyrano[3,4-b]pyrrolo[3′,2′:5,6]pyrido[3,2-e][1,4]oxazin-5(5aH)-yl)nicotinamide,
6-(2-((R)-4-(3,4-difluorobenzyl)-2-(o-tolyl)piperazin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)nicotinamide,
2-(2-((R)-4-(3,4-difluorobenzyl)-2-(o-tolyl)piperazin-1-yl)-7-azaspiro[3.5]nonan-7-yl)-N-((3-nitro-4-(((tetrahydro-2H-pyran-4-yl)methyl)amino)phenyl)sulfonyl)-4-((5aS,9aR)-6,7,9,9a-tetrahydro-1H-pyrano[3,4-b]pyrrolo[3′,2′:5,6]pyrido[3,2-e][1,4]oxazin-5(5aH)-yl)pyrimidine-5-carboxamide,
5-(2-((R)-4-((6-fluoro-5-methoxypyridin-3-yl)methyl)-2-(o-tolyl)piperazin-1-yl)-7-azaspiro[3.5]nonan-7-yl)-N-((3-nitro-4-(((tetrahydro-2H-pyran-4-yl)methyl)amino)phenyl)sulfonyl)-3-((5aS,9aR)-6,7,9,9a-tetrahydro-1H-pyrano[3,4-b]pyrrolo[3′,2′:5,6]pyrido[3,2-e][1,4]oxazin-5(5aH)-yl)picolinamide,
5-fluoro-4-(2-((R)-4-((6-fluoro-5-methoxypyridin-3-yl)methyl)-2-(o-tolyl)piperazin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide.

Compounds of the invention may contain one or more asymmetric carbon atoms. Accordingly, the compounds may exist as diastereomers, enantiomers or mixtures thereof. The syntheses of the compounds may employ racemates, diastereomers or enantiomers as starting materials or as intermediates. Diastereomeric compounds may be separated by chromatographic or crystallization methods. Similarly, enantiomeric mixtures may be separated using the same techniques or others known in the art. Each of the asymmetric carbon atoms may be in the R or S configuration, and both of these configurations are within the scope of the invention.

Compounds having one or more chiral centers can exist in various stereoisomeric forms. Stereoisomers are compounds that differ only in their spatial arrangement. Stereoisomers include all diastereomeric, enantiomeric, and epimeric forms as well as racemates and mixtures thereof.

The term “geometric isomer” refers to cyclic compounds having at least two substituents, wherein the two substituents are both on the same side of the ring (cis) or wherein the substituents are each on opposite sides of the ring (trans). When a disclosed compound is named or depicted by structure without indicating stereochemistry, it is understood that the name or the structure encompasses one or more of the possible stereoisomers, or geometric isomers, or a mixture of the encompassed stereoisomers or geometric isomers.

When a geometric isomer is depicted by name or structure, it is to be understood that the named or depicted isomer exists to a greater degree than another isomer, that is that the geometric isomeric purity of the named or depicted geometric isomer is greater than 50%, such as at least 60%, 70%, 80%, 90%, 99%, or 99.9% pure by weight. Geometric isomeric purity is determined by dividing the weight of the named or depicted geometric isomer in the mixture by the total weight of all of the geometric isomers in the mixture.

Racemic mixture means 50% of one enantiomer and 50% of is corresponding enantiomer. When a compound with one chiral center is named or depicted without indicating the stereochemistry of the chiral center, it is understood that the name or structure encompasses both possible enantiomeric forms (e.g., both enantiomerically-pure, enantiomerically-enriched or racemic) of the compound. When a compound with two or more chiral centers is named or depicted without indicating the stereochemistry of the chiral centers, it is understood that the name or structure encompasses all possible diasteriomeric forms (e.g., diastereomerically pure, diastereomerically enriched and equimolar mixtures of one or more diastereomers (e.g., racemic mixtures) of the compound.

Enantiomeric and diastereomeric mixtures can be resolved into their component enantiomers or stereoisomers by well-known methods, such as chiral-phase gas chromatography, chiral-phase high performance liquid chromatography, crystallizing the compound as a chiral salt complex, or crystallizing the compound in a chiral solvent. Enantiomers and diastereomers also can be obtained from diastereomerically- or enantiomerically-pure intermediates, reagents, and catalysts by well-known asymmetric synthetic methods.

When a compound is designated by a name or structure that indicates a single enantiomer, unless indicated otherwise, the compound is at least 60%, 70%, 80%, 90%, 99% or 99.9% optically pure (also referred to as “enantiomerically pure”). Optical purity is the weight in the mixture of the named or depicted enantiomer divided by the total weight in the mixture of both enantiomers.

When the stereochemistry of a disclosed compound is named or depicted by structure, and the named or depicted structure encompasses more than one stereoisomer (e.g., as in a diastereomeric pair), it is to be understood that one of the encompassed stereoisomers or any mixture of the encompassed stereoisomers is included. It is to be further understood that the stereoisomeric purity of the named or depicted stereoisomers at least 60%, 70%, 80%, 90%, 99% or 99.9% by weight. The stereoisomeric purity in this case is determined by dividing the total weight in the mixture of the stereoisomers encompassed by the name or structure by the total weight in the mixture of all of the stereoisomers.

A modified compound of any one of such compounds including a modification having an improved (e.g., enhanced, greater) pharmaceutical solubility, stability, bioavailability and/or therapeutic index as compared to the unmodified compound is also contemplated. The examples of modifications include but not limited to the prodrug derivatives, and the deuterium-enriched compounds. For example:
Prodrug derivatives: prodrugs, upon administration to a subject, will converted in vivo into active compounds of the present invention [Nature Reviews of Drug Discovery, 2008, Volume 7, p255]. It is noted that in many instances, the prodrugs themselves also fall within the scope of the range of compounds according to the present invention. The prodrugs of the compounds of the present invention can be prepared by standard organic reaction, for example, by reacting with a carbamoylating agent (e.g., 1,1-acyloxyalkylcarbonochloridate, para-nitrophenyl carbonate, or the like) or an acylating agent. Further examples of methods and strategies of making prodrugs are described in Bioorganic and Medicinal Chemistry Letters, 1994, Vol. 4, p. 1985.
Deuterium-enriched compounds: deuterium (D or ²H) is a stable, non-radioactive isotope of hydrogen and has an atomic weight of 2.0144. Hydrogen naturally occurs as a mixture of the isotopes ^{X}H (hydrogen or protium), D (²H or deuterium), and T (³H or tritium). The natural abundance of deuterium is 0.015%. One of ordinary skill in the art recognizes that in all chemical compounds with a H atom, the H atom actually represents a mixture of H and D, with about 0.015% being D. Thus, compounds with a level of deuterium that has been enriched to be greater than its natural abundance of 0.015%, should be considered unnatural and, as a result, novel over their nonenriched counterparts.

It should be recognized that the compounds of the present invention may be present and optionally administered in the form of salts, and solvates. The invention encompasses any pharmaceutically acceptable salts and solvates of any one of the above-described compounds and modifications thereof. For example, it is within the scope of the present invention to convert the compounds of the present invention into and use them in the form of their pharmaceutically acceptable salts derived from various organic and inorganic acids and bases in accordance with procedures well known in the art.

When the compounds of the present invention possess a free base form, the compounds can be prepared as a pharmaceutically acceptable acid addition salt by reacting the free base form of the compound with a pharmaceutically acceptable inorganic or organic acid, e.g., hydrohalides such as hydrochloride, hydrobromide, hydroiodide; other mineral acids such as sulfate, nitrate, phosphate, etc.; and alkyl and monoarylsulfonates such as ethanesulfonate, toluenesulfonate and benzenesulfonate; and other organic acids and their corresponding salts such as acetate, tartrate, maleate, succinate, citrate, benzoate, salicylate and ascorbate. Further acid addition salts of the present invention include, but are not limited to: adipate, alginate, arginate, aspartate, bisulfate, bisulfite, bromide, butyrate, camphorate, camphorsulfonate, caprylate, chloride, chlorobenzoate, cyclopentanepropionate, digluconate, dihydrogenphosphate, dinitrobenzoate, dodecylsulfate, fumarate, galacterate (from mucic acid), galacturonate, glucoheptanoate, gluconate, glutamate, glycerophosphate, hemisuccinate, hemisulfate, heptanoate, hexanoate, hippurate, 2-hydroxyethanesulfonate, iodide, isethionate, iso-butyrate, lactate, lactobionate, malonate, mandelate, metaphosphate, methanesulfonate, methylbenzoate, monohydrogenphosphate, 2-naphthalenesulfonate, nicotinate, oxalate, oleate, pamoate, pectinate, persulfate, phenylacetate, 3-phenylpropionate, phosphonate and phthalate. It should be recognized that the free base forms will typically differ from their respective salt forms somewhat in physical properties such as solubility in polar solvents, but otherwise the salts are equivalent to their respective free base forms for the purposes of the present invention.

When the compounds of the present invention possess a free acid form, a pharmaceutically acceptable base addition salt can be prepared by reacting the free acid form of the compound with a pharmaceutically acceptable inorganic or organic base. Examples of such bases are alkali metal hydroxides including potassium, sodium and lithium hydroxides; alkaline earth metal hydroxides such as barium and calcium hydroxides; alkali metal alkoxides, e.g., potassium ethanolate and sodium propanolate; and various organic bases such as ammonium hydroxide, piperidine, diethanolamine and N-methylglutamine. Also included are the aluminum salts of the compounds of the present invention. Further base salts of the present invention include, but are not limited to: copper, ferric, ferrous, lithium, magnesium, manganic, manganous, potassium, sodium and zinc salts. Organic base salts include, but are not limited to, salts of primary, secondary and tertiary amines, substituted amines including naturally occurring substituted amines, cyclic amines and basic ion exchange resins, e.g., arginine, betaine, caffeine, chloroprocaine, choline, N,N′-dibenzylethylenediamine (benzathine), dicyclohexylamine, diethanolamine, 2-diethylaminoethanol, 2-dimethylaminoethanol, ethanolamine, ethylenediamine, N-ethylmorpholine, N-ethylpiperidine, glucamine, glucosamine, histidine, hydrabamine, iso-propylamine, lidocaine, lysine, meglumine, N-methyl-D-glucamine, morpholine, piperazine, piperidine, polyamine resins, procaine, purines, theobromine, triethanolamine, triethylamine, trimethylamine, tripropylamine and tris-(hydroxymethyl)-methylamine (tromethamine). It should be recognized that the free acid forms will typically differ from their respective salt forms somewhat in physical properties such as solubility in polar solvents, but otherwise the salts are equivalent to their respective free acid forms for the purposes of the present invention.

In one aspect, a pharmaceutically acceptable salt is a hydrochloride salt, hydrobromide salt, methanesulfonate, toluenesulfonate, acetate, fumarate, sulfate, bisulfate, succinate, citrate, phosphate, maleate, nitrate, tartrate, benzoate, biocarbonate, carbonate, sodium hydroxide salt, calcium hydroxide salt, potassium hydroxide salt, tromethamine salt, or mixtures thereof.

Compounds of the present invention that comprise tertiary nitrogen-containing groups may be quaternized with such agents as (C₁₋₄) alkyl halides, e.g., methyl, ethyl, iso-propyl and tert-butyl chlorides, bromides and iodides; di-(C₁₋₄) alkyl sulfates, e.g., dimethyl, diethyl and diamyl sulfates; alkyl halides, e.g., decyl, dodecyl, lauryl, myristyl and stearyl chlorides, bromides and iodides; and aryl (C₁₋₄) alkyl halides, e.g., benzyl chloride and phenethyl bromide. Such salts permit the preparation of both water- and oil-soluble compounds of the invention.

Amine oxides, also known as amine-N-oxide and N-oxide, of anti-cancer agents with tertiary nitrogen atoms have been developed as prodrugs [Mol Cancer Therapy. 2004 March; 3(3):233-44]. Compounds of the present invention that comprise tertiary nitrogen atoms may be oxidized by such agents as hydrogen peroxide (H₂O₂), Caro's acid or peracids like meta-Chloroperoxybenzoic acid (mCPBA) to from amine oxide.

The compounds disclosed therein are bcl-2 inhibitors. The pharmaceutical composition of the present invention comprises one or more bcl-2 inhibitors, or a pharmaceutically acceptable salt thereof, and a pharmaceutically acceptable carrier or diluent.

“Pharmaceutically acceptable carrier” and “pharmaceutically acceptable diluent” refer to a substance that aids the formulation and/or administration of an active agent to and/or absorption by a subject and can be included in the compositions of the present disclosure without causing a significant adverse toxicological effect on the subject. Non-limiting examples of pharmaceutically acceptable carriers and/or diluents include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with or interfere with the activity of the compounds provided herein. One of ordinary skill in the art will recognize that other pharmaceutical excipients are suitable for use with disclosed compounds.

The pharmaceutical compositions of the present invention optionally include one or more pharmaceutically acceptable carriers and/or diluents therefor, such as lactose, starch, cellulose and dextrose. Other excipients, such as flavoring agents; sweeteners; and preservatives, such as methyl, ethyl, propyl and butyl parabens, can also be included. More complete listings of suitable excipients can be found in the Handbook of Pharmaceutical Excipients (5^{th} Ed., Pharmaceutical Press (2005)). A person skilled in the art would know how to prepare formulations suitable for various types of administration routes. Conventional procedures and ingredients for the selection and preparation of suitable formulations are described, for example, in Remington's Pharmaceutical Sciences (2003-20th edition) and in The United States Pharmacopeia: The National Formulary (USP 24 NF19) published in 1999. The carriers, diluents and/or excipients are “acceptable” in the sense of being compatible with the other ingredients of the pharmaceutical composition and not deleterious to the recipient thereof.

The pharmaceutical compositions of the present invention may further comprise other conventional pharmaceutically inactive agents. Any inert excipient that is commonly used as a carrier or diluent may be used in compositions of the present invention, such as sugars, polyalcohols, soluble polymers, salts and lipids. Sugars and polyalcohols which may be employed include, without limitation, lactose, sucrose, mannitol, and sorbitol. Illustrative of the soluble polymers which may be employed are polyoxyethylene, poloxamers, polyvinylpyrrolidone, and dextran. Useful salts include, without limitation, sodium chloride, magnesium chloride, and calcium chloride. Lipids which may be employed include, without limitation, fatty acids, glycerol fatty acid esters, glycolipids, and phospholipids.

In addition, the pharmaceutical compositions of the present invention may further comprise binders (e.g., acacia, cornstarch, gelatin, carbomer, ethyl cellulose, guar gum, hydroxypropyl cellulose, hydroxypropyl methyl cellulose, povidone), disintegrating agents (e.g., cornstarch, potato starch, alginic acid, silicon dioxide, croscarmellose sodium, crospovidone, guar gum, sodium starch glycolate, Primogel), buffers (e.g., tris-HCL, acetate, phosphate) of various pH and ionic strength, additives such as albumin or gelatin to prevent absorption to surfaces, detergents (e.g., Tween 20, Tween 80, Pluronic F68, bile acid salts), protease inhibitors, surfactants (e.g., sodium lauryl sulfate), permeation enhancers, solubilizing agents (e.g., glycerol, polyethylene glycerol, cyclodextrins), a glidant (e.g., colloidal silicon dioxide), anti-oxidants (e.g., ascorbic acid, sodium metabisulfite, butylated hydroxyanisole), stabilizers (e.g., hydroxypropyl cellulose, hydroxypropylmethyl cellulose), viscosity increasing agents (e.g., carbomer, colloidal silicon dioxide, ethyl cellulose, guar gum), sweeteners (e.g., sucrose, aspartame, citric acid), flavoring agents (e.g., peppermint, methyl salicylate, or orange flavoring), preservatives (e.g., Thimerosal, benzyl alcohol, parabens), lubricants (e.g., stearic acid, magnesium stearate, polyethylene glycol, sodium lauryl sulfate), flow-aids (e.g., colloidal silicon dioxide), plasticizers (e.g., diethyl phthalate, triethyl citrate), emulsifiers (e.g., carbomer, hydroxypropyl cellulose, sodium lauryl sulfate, methyl cellulose, hydroxyethyl cellulose, carboxymethylcellulose sodium), polymer coatings (e.g., poloxamers or poloxamines), coating and film forming agents (e.g., ethyl cellulose, acrylates, polymethacrylates) and/or adjuvants.

In one embodiment, the pharmaceutical compositions are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

Additionally, the invention encompasses pharmaceutical compositions comprising any solid or liquid physical form of the compound of the invention. For example, the compounds can be in a crystalline form, in amorphous form, and have any particle size. The particles may be micronized, or may be agglomerated, particulate granules, powders, oils, oily suspensions or any other form of solid or liquid physical form.

When compounds according to the present invention exhibit insufficient solubility, methods for solubilizing the compounds may be used. Such methods are known to those of skill in this art, and include, but are not limited to, pH adjustment and salt formation, using co-solvents, such as ethanol, propylene glycol, polyethylene glycol (PEG) 300, PEG 400, DMA (10-30%), DMSO (10-20%), NMP (10-20%), using surfactants, such as polysorbate 80, polysorbate 20 (1-10%), cremophor EL, Cremophor RH40, Cremophor RH60 (5-10%), Pluronic F68/Poloxamer 188 (20-50%), Solutol HS15 (20-50%), Vitamin E TPGS, and d-α-tocopheryl PEG 1000 succinate (20-50%), using complexation such as HPβCD and SBEβCD (10-40%), and using advanced approaches such as micelle, addition of a polymer, nanoparticle suspensions, and liposome formation.

A wide variety of administration methods may be used in conjunction with the compounds of the present invention. Compounds of the present invention may be administered or coadministered orally, parenterally, intraperitoneally, intravenously, intraarterially, transdermally, sublingually, intramuscularly, rectally, transbuccally, intranasally, liposomally, via inhalation, vaginally, intraocularly, via local delivery (for example by catheter or stent), subcutaneously, intraadiposally, intraarticularly, or intrathecally. The compounds according to the invention may also be administered or coadministered in slow release dosage forms. Compounds may be in gaseous, liquid, semi-liquid or solid form, formulated in a manner suitable for the route of administration to be used. For oral administration, suitable solid oral formulations include tablets, capsules, pills, granules, pellets, sachets and effervescent, powders, and the like. Suitable liquid oral formulations include solutions, suspensions, dispersions, emulsions, oils and the like. For parenteral administration, reconstitution of a lyophilized powder is typically used.

As used herein, “acyl” means a carbonyl containing substituent represented by the formula —C(O)—R in which R is H, alkyl, a carbocycle, a heterocycle, carbocycle-substituted alkyl or heterocycle-substituted alkyl wherein the alkyl, alkoxy, carbocycle and heterocycle are as defined herein. Acyl groups include alkanoyl (e.g. acetyl), aroyl (e.g. benzoyl), and heteroaroyl.

“Aliphatic” means a moiety characterized by a straight or branched chain arrangement of constituent carbon atoms and may be saturated or partially unsaturated with one or more double or triple bonds.

The term “alkyl” refers to a straight or branched hydrocarbon containing 1-20 carbon atoms (e.g., C₁-C₁₀, C₁-C₆). Examples of alkyl include, but are not limited to, methyl, methylene, ethyl, ethylene, n-propyl, i-propyl, n-butyl, i-butyl, and t-butyl. Preferably, the alkyl group has one to ten carbon atoms. More preferably, the alkyl group has one to four carbon atoms.

The term “alkenyl” refers to a straight or branched hydrocarbon containing 2-20 carbon atoms (e.g., C₂-C₁₀, C₂-C₆) and one or more double bonds. Examples of alkenyl include, but are not limited to, ethenyl, propenyl, and allyl. Preferably, the alkylene group has two to ten carbon atoms. More preferably, the alkylene group has two to four carbon atoms.

The term “alkynyl” refers to a straight or branched hydrocarbon containing 2-20 carbon atoms (e.g., C₂-C₁₀, C₂-C₆) and one or more triple bonds. Examples of alkynyl include, but are not limited to, ethynyl, 1-propynyl, 1- and 2-butynyl, and 1-methyl-2-butynyl. Preferably, the alkynyl group has two to ten carbon atoms. More preferably, the alkynyl group has two to four carbon atoms.

The term “alkylamino” refers to an —N(R)-alkyl in which R can be H, alkyl, alkenyl, alkynyl, cycloalkyl, cycloalkenyl, heterocycloalkyl, heterocycloalkenyl, aryl, or heteroaryl.

“Alkoxy” means an oxygen moiety having a further alkyl substituent.

“Alkoxycarbonyl” means an alkoxy group attached to a carbonyl group.

“Oxoalkyl” means an alkyl, further substituted with a carbonyl group. The carbonyl group may be an aldehyde, ketone, ester, amide, acid or acid chloride.

The term “cycloalkyl” refers to a saturated hydrocarbon ring system having 3 to 30 carbon atoms (e.g., C₃-C₁₂, C₃-C₈, C₃-C₆). Examples of cycloalkyl include, but are not limited to, cyclopropyl, cyclobutyl, cyclopentyl, cyclohexyl, cycloheptyl, and cyclooctyl. The term “cycloalkenyl” refers to a non-aromatic hydrocarbon ring system having 3 to 30 carbons (e.g., C₃-C₁₂) and one or more double bonds. Examples include cyclopentenyl, cyclohexenyl, and cycloheptenyl.

The term “heterocycloalkyl” refers to a saturated or unsaturated nonaromatic monocyclic, bicyclic, tricyclic, or tetracyclic system having one or more heteroatoms (such as O, N, S, B, P, Si, or Se), which may be the same or different. Examples of heterocycloalkyl groups include, but are not limited to, piperazinyl, pyrrolidinyl, dioxanyl, morpholinyl, and tetrahydrofuranyl.

The term “heterocycloalkenyl” refers to a nonaromatic monocyclic, bicyclic, tricyclic, or tetracyclic ring system having one or more heteroatoms (such as O, N, S, P, B, Si, or Se) and one or more double bonds.

Spiroalkyl refers to a compound comprising two saturated cyclic alkyl rings sharing only one common atom (also known as a spiro atom), with no heteroatom and no unsaturated bonds on any of the rings. In one embodiment, the spiroalkyl is bicyclic. In another embodiment, the spiroalkyl has more than two cycles. In certain embodiments, the spiroalkyl compound is a polyspiro compound connected by two or more spiroatoms making up three or more rings. In certain embodiments, one of the rings of the bicyclic spiroalkyl has 3, 4, 5, 6, 7, or 8 atoms, including the common spito atom. In one embodiment, the spiroalkyl is a 5 to 20 membered, 5 to 14 membered, or 5 to 10 membered polycyclic spiroalkyl group. Representative examples of spiroalkyl include, but are not limited to the following groups:

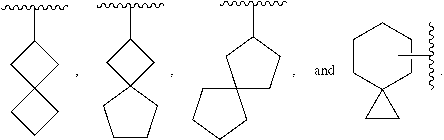

Spiroheterocyclyl refers to a compound comprising two non-saturated rings sharing only one common atom (also known as a spiro atom), with at least one heteroatom on one of the two rings, such as a polycyclic heterocyclyl group with rings connected through one common carbon atom. The common atom can be carbon (C), silicon, or nitrogen (such as a positively charged quaternary nitrogen atom). The heteroatoms can comprise nitrogen, quaternary nitrogen, oxidized nitrogen (e.g., NO), oxygen, silicon, and sulfur, including sulfoxide and sulfone, and the remaining ring atoms are C. In addition, one or more of the rings may contain one or more double bonds. In one embodiment, the spiro heterocyclyl is bicyclic, with heteroatom(s) on either one or both cycles. In certain embodiments, one of the rings of the bicyclic spiro heterocyclyl has 3, 4, 5, 6, 7, or 8 atoms, including the common spito atom. In certain embodiments, the spiro heterocyclic compound is a polyspiro compound connected by two or more spiroatoms making up three or more rings. In one embodiment, the spiro heterocyclyl is a 5 to 20 membered, 5 to 14 membered, or 5 to 10 membered polycyclic heterocyclyl group. Representative examples of spiro heterocyclyl include, but are not limited to the following groups:

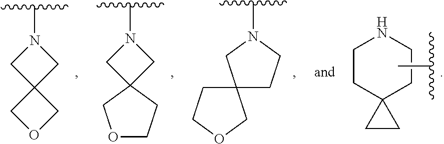

Fused heterocyclyl refers to a polycyclic heterocyclyl group, wherein each ring in the group shares an adjacent pair of atoms (such as carbon atoms) with another ring in the group, wherein one or more rings can contain one or more double bonds, and wherein said rings have one or more heteroatoms, which can be nitrogen, quaternary nitrogen, oxidized nitrogen (e.g., NO), oxygen, and sulfur, including sulfoxide and sulfone, and the remaining ring atoms are C. In certain embodiments, the fused heterocyclyl is bicyclic. In certain embodiments, the fused heterocyclyl contains more than two rings, at least two of which share an adjacent pair of atoms. In one embodiment, the fused heterocyclyl is a 5 to 20 membered, 5 to 12 membered, or 5 to 10 membered polycyclic heterocyclyl group. Representative examples of fused heterocyclyl include, but are not limited to the following groups:

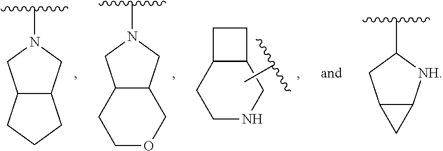

Bridged heterocyclyl refers to a compound having at least two rings sharing three or more common ring atoms, separating the two bridgehead atoms by a bridge containing at least one atom, wherein at least one ring atom is a heteroatom. The bridgehead atoms are the atoms from which three bonds radiate and where the rings meet. The rings of the bridged heterocyclyl can have one or more double bonds, and the ring heteroatom(s) can be nitrogen, quaternary nitrogen, oxidized nitrogen (e.g., NO), oxygen, and sulfur, including sulfoxide and sulfone as ring atoms, while the remaining ring atoms are C. In one embodiment, the bridged heterocyclyl is bicyclic. In one embodiment, the bridged heterocyclyl is a 5 to 20 membered, 5 to 12 membered, or 5 to 10 membered polycyclic heterocyclyl group. Representative examples of bridged heterocyclyl include, but are not limited to the following groups:

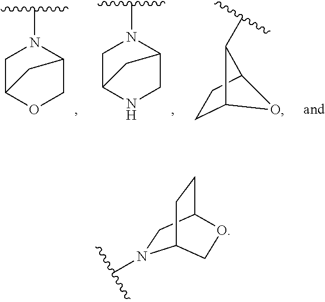

The term “aryl” refers to a 6-carbon monocyclic, 10-carbon bicyclic, 14-carbon tricyclic aromatic ring system. Examples of aryl groups include, but are not limited to, phenyl, naphthyl, and anthracenyl.

The term “heteroaryl” refers to an aromatic monocyclic, bicyclic, tricyclic, or tetracyclic ring system having one or more heteroatoms (such as O, N, S, P, or Se). Examples of heteroaryl groups include pyridyl, furyl, imidazolyl, benzimidazolyl, pyrimidinyl, thienyl, quinolinyl, indolyl, and thiazolyl.

Alkyl, alkenyl, alkynyl, cycloalkyl, heterocycloalkyl, cycloalkenyl, heterocycloalkenyl, alkylamino, spiroheterocyclyl, fused heterocyclyl, bridged heterocyclyl, aryl, and heteroaryl mentioned above include both substituted and unsubstituted moieties. Possible substituents on alkylamino, cycloalkyl, heterocycloalkyl, cycloalkenyl, heterocycloalkenyl, aryl, and heteroaryl include, but are not limited to, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, C₁-C₁₀ alkoxy, aryl, aryloxy, heteroaryl, heteroaryloxy, amino, C₁-C₁₀ alkylamino, arylamino, hydroxy, halo, oxo (O═), thioxo (S═), thio, silyl, C₁-C₁₀ alkylthio, arylthio, C₁-C₁₀ alkylsulfonyl, arylsulfonyl, acylamino, aminoacyl, aminothioacyl, amidino, mercapto, amido, thioureido, thiocyanato, sulfonamido, guanidine, ureido, cyano, nitro, acyl, thioacyl, acyloxy, carbamido, carbamyl, carboxyl, and carboxylic ester. On the other hand, possible substituents on alkyl, alkenyl, or alkynyl include all of the above-recited substituents except C₁-C₁₀ alkyl. Cycloalkyl, cycloalkenyl, heterocycloalkyl, heterocycloalkenyl, aryl, and heteroaryl can also be fused with each other.

“Amino” means a nitrogen moiety having two further substituents where each substituent has a hydrogen or carbon atom alpha bonded to the nitrogen. Unless indicated otherwise, the compounds of the invention containing amino moieties may include protected derivatives thereof. Suitable protecting groups for amino moieties include acetyl, tert-butoxycarbonyl, benzyloxycarbonyl, and the like.

“Aromatic” means a moiety wherein the constituent atoms make up an unsaturated ring system, all atoms in the ring system are sp2 hybridized and the total number of pi electrons is equal to 4n+2. An aromatic ring may be such that the ring atoms are only carbon atoms or may include carbon and non-carbon atoms (see Heteroaryl).

“Carbamoyl” means the radical —OC(O)NRₐR_{b} where Rₐ and R_{b} are each independently two further substituents where a hydrogen or carbon atom is alpha to the nitrogen. It is noted that carbamoyl moieties may include protected derivatives thereof. Examples of suitable protecting groups for carbamoyl moieties include acetyl, tert-butoxycarbonyl, benzyloxycarbonyl, and the like. It is noted that both the unprotected and protected derivatives fall within the scope of the invention.

“Carbonyl” means the radical —C(O)—. It is noted that the carbonyl radical may be further substituted with a variety of substituents to form different carbonyl groups including acids, acid halides, amides, esters, and ketones.

“Carboxy” means the radical —C(O)O—. It is noted that compounds of the invention containing carboxy moieties may include protected derivatives thereof, i.e., where the oxygen is substituted with a protecting group.

Suitable protecting groups for carboxy moieties include benzyl, tert-butyl, and the like.

“Cyano” means the radical —CN.

“Formyl” means the radical —CH═O.

“Formimino” means the radical —HC═NH.

“Halo” means fluoro, chloro, bromo or iodo.

“Halo-substituted alkyl”, as an isolated group or part of a larger group, means “alkyl” substituted by one or more “halo” atoms, as such terms are defined in this Application. Halo-substituted alkyl includes haloalkyl, dihaloalkyl, trihaloalkyl, perhaloalkyl and the like.

“Hydroxy” means the radical —OH.

“Imine derivative” means a derivative comprising the moiety —C(═NR)—, wherein R comprises a hydrogen or carbon atom alpha to the nitrogen.

“Isomers” mean any compound having identical molecular formulae but differing in the nature or sequence of bonding of their atoms or in the arrangement of their atoms in space. Isomers that differ in the arrangement of their atoms in space are termed “stereoisomers.” Stereoisomers that are not mirror images of one another are termed “diastereomers” and stereoisomers that are nonsuperimposable mirror images are termed “enantiomers” or sometimes “optical isomers.” A carbon atom bonded to four nonidentical substituents is termed a “chiral center.” A compound with one chiral center has two enantiomeric forms of opposite chirality. A mixture of the two enantiomeric forms is termed a “racemic mixture.”

“Nitro” means the radical —NO₂.

“Protected derivatives” means derivatives of compounds in which a reactive site are blocked with protecting groups. Protected derivatives are useful in the preparation of pharmaceuticals or in themselves may be active as inhibitors. A comprehensive list of suitable protecting groups can be found in T. W. Greene, Protecting Groups in Organic Synthesis, 3rd edition, Wiley & Sons, 1999.

The term “substituted” means that an atom or group of atoms has replaced hydrogen as the substituent attached to another group. For aryl and heteroaryl groups, the term “substituted” refers to any level of substitution, namely mono-, di-, tri-, tetra-, or penta-substitution, where such substitution is permitted. The substituents are independently selected, and substitution may be at any chemically accessible position. The term “unsubstituted” means that a given moiety may consist of only hydrogen substituents through available valencies (unsubstituted).

If a functional group is described as being “optionally substituted,” the function group may be either (1) not substituted, or (2) substituted. If a carbon of a functional group is described as being optionally substituted with one or more of a list of substituents, one or more of the hydrogen atoms on the carbon (to the extent there are any) may separately and/or together be replaced with an independently selected optional substituent.

“Sulfide” means —S—R wherein R is H, alkyl, carbocycle, heterocycle, carbocycloalkyl or heterocycloalkyl. Particular sulfide groups are mercapto, alkylsulfide, for example methylsulfide (—S-Me); arylsulfide, e.g., phenylsulfide; aralkylsulfide, e.g., benzylsulfide.

“Sulfinyl” means the radical —S(O)—. It is noted that the sulfinyl radical may be further substituted with a variety of substituents to form different sulfinyl groups including sulfinic acids, sulfinamides, sulfinyl esters, and sulfoxides.

“Sulfonyl” means the radical —S(O)(O)—. It is noted that the sulfonyl radical may be further substituted with a variety of substituents to form different sulfonyl groups including sulfonic acids, sulfonamides, sulfonate esters, and sulfones.

“Thiocarbonyl” means the radical —C(S)—. It is noted that the thiocarbonyl radical may be further substituted with a variety of substituents to form different thiocarbonyl groups including thioacids, thioamides, thioesters, and thioketones.

“Animal” includes humans, non-human mammals (e.g., non-human primates, rodents, mice, rats, hamsters, dogs, cats, rabbits, cattle, horses, sheep, goats, swine, deer, and the like) and non-mammals (e.g., birds, and the like).

“Bioavailability” as used herein is the fraction or percentage of an administered dose of a drug or pharmaceutical composition that reaches the systemic circulation intact. In general, when a medication is administered intravenously, its bioavailability is 100%. However, when a medication is administered via other routes (e.g., orally), its bioavailability decreases (e.g., due to incomplete absorption and first-pass metabolism). Methods to improve the bioavailability include prodrug approach, salt synthesis, particle size reduction, complexation, change in physical form, solid dispersions, spray drying, and hot-melt extrusion.

“Disease” specifically includes any unhealthy condition of an animal or part thereof and includes an unhealthy condition that may be caused by, or incident to, medical or veterinary therapy applied to that animal, i.e., the “side effects” of such therapy.

“Pharmaceutically acceptable” means that which is useful in preparing a pharmaceutical composition that is generally safe, non-toxic and neither biologically nor otherwise undesirable and includes that which is acceptable for veterinary use as well as human pharmaceutical use.

“Pharmaceutically acceptable salts” means organic or inorganic salts of compounds of the present invention which are pharmaceutically acceptable, as defined above, and which possess the desired pharmacological activity. Such salts include acid addition salts formed with inorganic acids, or with organic acids. Pharmaceutically acceptable salts also include base addition salts which may be formed when acidic protons present are capable of reacting with inorganic or organic bases. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate,” ethanesulfonate, benzenesulfonate, p-toluenesulfonate, pamoate (i.e., 1,1′-methylene-bis-(2-hydroxy-3-naphthoate)) salts, alkali metal (e.g., sodium and potassium) salts, alkaline earth metal (e.g., magnesium) salts, and ammonium salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

“Pharmacophore,” as defined by The International Union of Pure and Applied Chemistry, is an ensemble of steric and electronic features that is necessary to ensure the optimal supramolecular interactions with a specific biological target and to trigger (or block) its biological response. For example, Camptothecin is the pharmacophore of the well known drug topotecan and irinotecan. Mechlorethamine is the pharmacophore of a list of widely used nitrogen mustard drugs like Melphalan, Cyclophosphamide, Bendamustine, and so on.

“Prodrug” means a compound that is convertible in vivo metabolically into an active pharmaceutical according to the present invention. For example, an inhibitor comprising a hydroxyl group may be administered as an ester that is converted by hydrolysis in vivo to the hydroxyl compound.

“Stability” in general refers to the length of time a drug retains its properties without loss of potency. Sometimes this is referred to as shelf life. Factors affecting drug stability include, among other things, the chemical structure of the drug, impurity in the formulation, pH, moisture content, as well as environmental factors such as temperature, oxidization, light, and relative humidity. Stability can be improved by providing suitable chemical and/or crystal modifications (e.g., surface modifications that can change hydration kinetics; different crystals that can have different properties), excipients (e.g., anything other than the active substance in the dosage form), packaging conditions, storage conditions, etc.

“Therapeutically effective amount” of a composition described herein is meant an amount of the composition which confers a therapeutic effect on the treated subject, at a reasonable benefit/risk ratio applicable to any medical treatment. The therapeutic effect may be objective (i.e., measurable by some test or marker) or subjective (i.e., subject gives an indication of or feels an effect). An effective amount of the composition described above may range from about 0.1 mg/kg to about 500 mg/kg, preferably from about 0.2 to about 50 mg/kg. Effective doses will also vary depending on route of administration, as well as the possibility of co-usage with other agents. It will be understood, however, that the total daily usage of the compositions of the present invention will be decided by the attending physician within the scope of sound medical judgment. The specific therapeutically effective dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or contemporaneously with the specific compound employed; and like factors well known in the medical arts.

As used herein, the term “treating” refers to administering a compound to a subject that has a neoplastic or immune disorder, or has a symptom of or a predisposition toward it, with the purpose to cure, heal, alleviate, relieve, alter, remedy, ameliorate, improve, or affect the disorder, the symptoms of or the predisposition toward the disorder. The term “an effective amount” refers to the amount of the active agent that is required to confer the intended therapeutic effect in the subject. Effective amounts may vary, as recognized by those skilled in the art, depending on route of administration, excipient usage, and the possibility of co-usage with other agents.

A “subject” refers to a human and a non-human animal. Examples of a non-human animal include all vertebrates, e.g., mammals, such as non-human primates (particularly higher primates), dog, rodent (e.g., mouse or rat), guinea pig, cat, and non-mammals, such as birds, amphibians, reptiles, etc. In a preferred embodiment, the subject is a human. In another embodiment, the subject is an experimental animal or animal suitable as a disease model.

“Combination therapy” includes the administration of the subject compounds of the present invention in further combination with other biologically active ingredients (such as, but not limited to, a second and different antineoplastic agent) and non-drug therapies (such as, but not limited to, surgery or radiation treatment). For instance, the compounds of the invention can be used in combination with other pharmaceutically active compounds, or non-drug therapies, preferably compounds that are able to enhance the effect of the compounds of the invention. The compounds of the invention can be administered simultaneously (as a single preparation or separate preparation) or sequentially to the other therapies. In general, a combination therapy envisions administration of two or more drugs/treatments during a single cycle or course of therapy.

In one embodiment, the compounds of the invention are administered in combination with one or more of traditional chemotherapeutic agents. The traditional chemotherapeutic agents encompass a wide range of therapeutic treatments in the field of oncology. These agents are administered at various stages of the disease for the purposes of shrinking tumors, destroying remaining cancer cells left over after surgery, inducing remission, maintaining remission and/or alleviating symptoms relating to the cancer or its treatment. Examples of such agents include, but are not limited to, alkylating agents such as Nitrogen Mustards (e.g., Bendamustine, Cyclophosphamide, Melphalan, Chlorambucil, Isofosfamide), Nitrosureas (e.g., Carmustine, Lomustine and Streptozocin), ethylenimines (e.g., thiotepa, hexamethylmelamine), Alkylsulfonates (e.g., Busulfan), Hydrazines and Triazines (e.g., Altretamine, Procarbazine, Dacarbazine and Temozolomide), and platinum based agents (e.g., Carboplatin, Cisplatin, and Oxaliplatin); plant alkaloids such as Podophyllotoxins (e.g., Etoposide and Teniposide), Taxanes (e.g., Paclitaxel and Docetaxel), Vinca alkaloids (e.g., Vincristine, Vinblastine and Vinorelbine); anti-tumor antibiotics such as Chromomycins (e.g., Dactinomycin and Plicamycin), Anthracyclines (e.g., Doxorubicin, Daunorubicin, Epirubicin, Mitoxantrone, and Idarubicin), and miscellaneous antibiotics such as Mitomycin and Bleomycin; anti-metabolites such as folic acid antagonists (e.g., Methotrexate), pyrimidine antagonists (e.g., 5-Fluorouracil, Floxuridine, Cytarabine, Capecitabine, and Gemcitabine), purine antagonists (e.g., 6-Mercaptopurine and 6-Thioguanine) and adenosine deaminase inhibitors (e.g., Cladribine, Fludarabine, Nelarabine and Pentostatin); topoisomerase inhibitors such as topoisomerase I inhibitors (Topotecan, Irinotecan), topoisomerase II inhibitors (e.g., Amsacrine, Etoposide, Etoposide phosphate, Teniposide), and miscellaneous anti-neoplastics such as ribonucleotide reductase inhibitors (Hydroxyurea), adrenocortical steroid inhibitor (Mitotane), anti-microtubule agents (Estramustine), and retinoids (Bexarotene, Isotretinoin, Tretinoin (ATRA).

In one aspect of the invention, the compounds may be administered in combination with one or more targeted anti-cancer agents that modulate protein kinases involved in various disease states. Examples of such kinases may include, but are not limited ABL1, ABL2/ARG, ACK1, AKT1, AKT2, AKT3, ALK, ALK1/ACVRL1, ALK2/ACVR1, ALK4/ACVR1B, ALK5/TGFBR1, ALK6/BMPR1B, AMPK(A1/B1/G1), AMPK(A1/B1/G2), AMPK(A1/B1/G3), AMPK(A1/B2/G1), AMPK(A2/B1/G1), AMPK(A2/B2/G1), AMPK(A2/B2/G2), ARAF, ARK5/NUAK1, ASK1/MAP3K5, ATM, Aurora A, Aurora B, Aurora C, AXL, BLK, BMPR2, BMX/ETK, BRAF, BRK, BRSK1, BRSK2, BTK, CAMK1a, CAMK1b, CAMK1d, CAMK1g, CAMKIla, CAMKIlb, CAMKIld, CAMKIIg, CAMK4, CAMKK1, CAMKK2, CDC7-DBF4, CDK1-cyclin A, CDK1-cyclin B, CDK1-cyclin E, CDK2-cyclin A, CDK2-cyclin A1, CDK2-cyclin E, CDK3-cyclin E, CDK4-cyclin D1, CDK4-cyclin D3, CDK5-p25, CDK5-p35, CDK6-cyclin D1, CDK6-cyclin D3, CDK7-cyclin H, CDK9-cyclin K, CDK9-cyclin T1, CHK1, CHK2, CK1a1, CK1d, CK1epsilon, CK1g1, CK1g2, CK1g3, CK2a, CK2a2, c-KIT, CLK1, CLK2, CLK3, CLK4, c-MER, c-MET, COT1/MAP3K8, CSK, c-SRC, CSF1R, CTK/MATK, DAPK1, DAPK2, DCAMKL1, DCAMKL2, DDR1, DDR2, DLK/MAP3K12, DMPK, DMPK2/CDC42BPG, DNA-PK, DRAK1/STK17A, DYRK1/DYRK1A, DYRK1B, DYRK2, DYRK3, DYRK4, EEF2K, EGFR, EIF2AK1, EIF2AK2, EIF2AK3, EIF2AK4/GCN2, EPHA1, EPHA2, EPHA3, EPHA4, EPHA5, EPHA6, EPHA7, EPHA8, EPHB1, EPHB2, EPHB3, EPHB4, ERBB2/HER2, ERBB4/HER4, ERK1/MAPK3, ERK2/MAPK1, ERK5/MAPK7, FAK/PTK2, FER, FES/FPS, FGFR1, FGFR2, FGFR3, FGFR4, FGR, FLT1NEGFR1, FLT3, FLT4NEGFR3, FMS, FRK/PTK5, FYN, GCK/MAP4K2, GRK1, GRK2, GRK3, GRK4, GRK5, GRK6, GRK7, GSK3a, GSK3b, Haspin, HCK, HGK/MAP4K4, HIPK1, HIPK2, HIPK3, HIPK4, HPK1/MAP4K1, IGF1R, IKKa/CHUK, IKKb/IKBKB, IKKe/IKBKE, IR, IRAK1, IRAK4, IRR/INSRR, ITK, JAK1, JAK2, JAK3, JNK1, JNK2, JNK3, KDRNEGFR2, KHS/MAP4K5, LATS1, LATS2, LCK, LCK2/ICK, LKB1, LIMK1, LOK/STK10, LRRK2, LYN, LYNB, MAPKAPK2, MAPKAPK3, MAPKAPK5/PRAK, MARK1, MARK2/PAR-1Ba, MARK3, MARK4, MEK1, MEK2, MEKK1, MEKK2, MEKK3, MELK, MINK/MINK1, MKK4, MKK6, MLCK/MYLK, MLCK2/MYLK2, MLK1/MAP3K9, MLK2/MAP3K10, MLK3/MAP3K11, MNK1, MNK2, MRCKa/, CDC42BPA, MRCKb/, CDC42BPB, MSK1/RPS6KA5, MSK2/RPS6KA4, MSSK1/STK23, MST1/STK4, MST2/STK3, MST3/STK24, MST4, mTOR/FRAP1, MUSK, MYLK3, MYO3b, NEK1, NEK2, NEK3, NEK4, NEK6, NEK7, NEK9, NEK11, NIK/MAP3K14, NLK, OSR1/OXSR1, P38a/MAPK14, P38b/MAPK11, P38d/MAPK13, P38g/MAPK12, P70S6K/RPS6KB1, p70S6Kb/, RPS6KB2, PAK1, PAK2, PAK3, PAK4, PAK5, PAK6, PASK, PBK/TOPK, PDGFRa, PDGFRb, PDK1/PDPK1, PDK1/PDHK1, PDK2/PDHK2, PDK3/PDHK3, PDK4/PDHK4, PHKg1, PHKg2, PI3Ka, (p110a/p85a), PI3Kb, (p110b/p85a), PI3Kd, (p110d/p85a), PI3Kg(p120g), PIM1, PIM2, PIM3, PKA, PKAcb, PKAcg, PKCa, PKCb1, PKCb2, PKCd, PKCepsilon, PKCeta, PKCg, PKCiota, PKCmu/PRKD1, PKCnu/PRKD3, PKCtheta, PKCzeta, PKD2/PRKD2, PKG1a, PKG1b, PKG2/PRKG2, PKN1/PRK1, PKN2/PRK2, PKN3/PRK3, PLK1, PLK2, PLK3, PLK4/SAK, PRKX, PYK2, RAF1, RET, RIPK2, RIPK3, RIPK5, ROCK1, ROCK2, RON/MST1R, ROS/ROS1, RSK1, RSK2, RSK3, RSK4, SGK1, SGK2, SGK3/SGKL, SIK1, SIK2, SLK/STK2, SNARK/NUAK2, SRMS, SSTK/TSSK6, STK16, STK22D/TSSK1, STK25/YSK1, STK32b/YANK2, STK32c/YANK3, STK33, STK38/NDR1, STK38L/NDR2, STK39/STLK3, SRPK1, SRPK2, SYK, TAK1, TAOK1, TAOK2/TA01, TAOK3/JIK, TBK1, TEC, TESK1, TGFBR2, TIE2/TEK, TLK1, TLK2, TNIK, TNK1, TRKA, TRKB, TRKC, TRPM7/CHAK1, TSSK2, TSSK3/STK22C, TTBK1, TTBK2, TTK, TXK, TYK1/LTK, TYK2, TYRO3/SKY, ULK1, ULK2, ULK3, VRK1, VRK2, WEE1, WNK1, WNK2, WNK3, YES/YES1, ZAK/MLTK, ZAP70, ZIPK/DAPK3, KINASE, MUTANTS, ABL1(E255K), ABL1(F3171), ABL1(G250E), ABL1(H396P), ABL1(M351T), ABL1(Q₂₅₂H), ABL1(T3151), ABL1(Y253F), ALK (C1156Y), ALK(L1196M), ALK (F1174L), ALK (R1275Q), BRAF(V599E), BTK(E41K), CHK2(1157T), c-Kit(A829P), c-KIT(D816H), c-KIT(D816V), c-Kit(D820E), c-Kit(N822K), C-Kit (T6701), c-Kit(V559D), c-Kit(V559DN654A), c-Kit(V559D/T6701), C-Kit (V560G), c-KIT(V654A), C-MET(D1228H), C-MET(D1228N), C-MET(F12001), c-MET(M1250T), C-MET(Y1230A), C-MET(Y1230C), C-MET(Y1230D), C-MET(Y1230H), c-Src(T341M), EGFR(G719C), EGFR(G719S), EGFR(L858R), EGFR(L861Q), EGFR(T790M), EGFR, (L858R,T790M), EGFR(d746-750/T790M), EGFR(d746-750), EGFR(d747-749/A750P), EGFR(d747-752/P753S), EGFR(d752-759), FGFR1(V561M), FGFR2(N549H), FGFR3(G697C), FGFR3(K650E), FGFR3(K650M), FGFR4(N535K), FGFR4(V550E), FGFR4(V550L), FLT3(D835Y), FLT3(ITD), JAK2 (V617F), LRRK2 (G2019S), LRRK2 (12020T), LRRK2 (R1441C), p38a(T106M), PDGFRa(D842V), PDGFRa(T6741), PDGFRa(V561D), RET(E762Q), RET(G691S), RET(M918T), RET(R749T), RET(R813Q), RET(V804L), RET(V804M), RET(Y791F), TIF2(R849W), TIF2(Y897S), and TIF2(Y1108F).

In another aspect of the invention, the subject compounds may be administered in combination with one or more targeted anti-cancer agents that modulate non-kinase biological targets, pathway, or processes. Such targets pathways, or processes include but not limited to heat shock proteins (e.g. HSP90), poly-ADP (adenosine diphosphate)-ribose polymerase (PARP), hypoxia-inducible factors(HIF), proteasome, Wnt/Hedgehog/Notch signaling proteins, TNF-alpha, matrix metalloproteinase, farnesyl transferase, apoptosis pathway (e.g Bcl-xL, Bcl-2, Bcl-w), histone deacetylases (HDAC), histone acetyltransferases (HAT), and methyltransferase (e.g histone lysine methyltransferases, histone arginine methyltransferase, DNA methyltransferase, etc), and other immunotherapies (e.g anti-PD1, anti-PDL1, anti-CTLA4, CAR-T, IDO, A2A antagonist etc).

In another aspect of the invention, the compounds of the invention are administered in combination with one or more of other anti-cancer agents that include, but are not limited to, gene therapy, RNAi cancer therapy, chemoprotective agents (e.g., amifostine, mesna, and dexrazoxane), antibody conjugate (e.g brentuximab vedotin, ibritumomab tioxetan), cancer immunotherapy such as Interleukin-2, cancer vaccines (e.g., sipuleucel-T) or monoclonal antibodies (e.g., Bevacizumab, Alemtuzumab, Rituximab, Trastuzumab, etc).

In another aspect of the invention, the subject compounds are administered in combination with radiation therapy or surgeries. Radiation is commonly delivered internally (implantation of radioactive material near cancer site) or externally from a machine that employs photon (x-ray or gamma-ray) or particle radiation. Where the combination therapy further comprises radiation treatment, the radiation treatment may be conducted at any suitable time so long as a beneficial effect from the co-action of the combination of the therapeutic agents and radiation treatment is achieved. For example, in appropriate cases, the beneficial effect is still achieved when the radiation treatment is temporally removed from the administration of the therapeutic agents, perhaps by days or even weeks.

In certain embodiments, the compounds of the invention are administered in combination with one or more of radiation therapy, surgery, or anti-cancer agents that include, but are not limited to, DNA damaging agents, anti-metabolites, topoisomerase inhibitors, anti-microtubule agents, kinase inhibitors, epigenetic agents, HSP90 inhibitors, PARP inhibitors, and antibodies targeting VEGF, HER2, EGFR, CD50, CD20, CD30, CD33, etc.

In certain embodiments, the compounds of the invention are administered in combination with one or more of abarelix, abiraterone acetate, aldesleukin, alemtuzumab, altretamine, anastrozole, asparaginase, bendamustine, bevacizumab, bexarotene, bicalutamide, bleomycin, bortezombi, brentuximab vedotin, busulfan, capecitabine, carboplatin, carmustine, cetuximab, chlorambucil, cisplatin, cladribine, clofarabine, clomiphene, crizotinib, cyclophosphamide, dasatinib, daunorubicin liposomal, decitabine, degarelix, denileukin diftitox, denileukin diftitox, denosumab, docetaxel, doxorubicin, doxorubicin liposomal, epirubicin, eribulin mesylate, erlotinib, estramustine, etoposide phosphate, everolimus, exemestane, fludarabine, fluorouracil, fotemustine, fulvestrant, gefitinib, gemcitabine, gemtuzumab ozogamicin, goserelin acetate, histrelin acetate, hydroxyurea, ibritumomab tiuxetan, idarubicin, ifosfamide, imatinib mesylate, interferon alfa 2a, ipilimumab, ixabepilone, lapatinib ditosylate, lenalidomide, letrozole, leucovorin, leuprolide acetate, levamisole, lomustine, mechlorethamine, melphalan, methotrexate, mitomycin C, mitoxantrone, nelarabine, nilotinib, oxaliplatin, paclitaxel, paclitaxel protein-bound particle, pamidronate, panitumumab, pegaspargase, peginterferon alfa-2b, pemetrexed disodium, pentostatin, raloxifene, rituximab, sorafenib, streptozocin, sunitinib maleate, tamoxifen, temsirolimus, teniposide, thalidomide, toremifene, tositumomab, trastuzumab, tretinoin, uramustine, vandetanib, vemurafenib, vinorelbine, zoledronate, pembrolizumab, nivolumab, atezolizumab, durvalumab, avelumab, as tisagenlecleucel, axicabtagene ciloleucel, radiation therapy, or surgery.

The invention further provides methods for the prevention or treatment of a neoplastic disease or autoimmune disease. In one embodiment, the invention relates to a method of treating a neoplastic disease or autoimmune disease, in a subject in need of treatment comprising administering to said subject a therapeutically effective amount of a compound of the invention. In one embodiment, the invention further provides for the use of a compound of the invention in the manufacture of a medicament for halting or decreasing a neoplastic disease or autoimmune disease.

In certain embodiments, the neoplastic disease is a lung cancer, head and neck cancer, central nervous system cancer, prostate cancer, testicular cancer, colorectal cancer, pancreatic cancer, liver cancer, stomach cancer, biliary tract cancer, esophageal cancer, gastrointestinal stromal tumor, breast cancer, cervical cancer, ovarian cancer, uterine cancer, leukemia, lymphomas, multiple myeloma, melanoma, basal cell carcinoma, squamous cell carcinoma, bladder cancer, renal cancer, sarcoma, mesothelioma, thymoma, myelodysplastic syndrome, or myeloproliferative disease.

The autoimmune diseases that can be affected using compounds and compositions according to the invention include, but are not limited to allergy, Alzheimer's disease, acute disseminated encephalomyelitis, Addison's disease, ankylosing spondylitis, antiphospholipid antibody syndrome, asthma, atherosclerosis, autoimmune hemolytic anemia, autoimmune hemolytic and thrombocytopenic states, autoimmune hepatitis, autoimmune inner ear disease, bullous pemphigoid, coeliac disease, chagas disease, chronic obstructive pulmonary disease, chronic Idiopathic thrombocytopenic purpura (ITP), churg-strauss syndrome, Crohn's disease, dermatomyositis, diabetes mellitus type 1, endometriosis, Goodpasture's syndrome (and associated glomerulonephritis and pulmonary hemorrhage), graves' disease, guillain-barré syndrome, hashimoto's disease, hidradenitis suppurativa, idiopathic thrombocytopenic purpura, interstitial cystitis, irritable bowel syndrome, lupus erythematosus, morphea, multiple sclerosis, myasthenia gravis, narcolepsy, neuromyotonia, Parkinson's disease, pemphigus vulgaris, pernicious anaemia, polymyositis, primary biliary cirrhosis, psoriasis, psoriatic arthritis, rheumatoid arthritis, schizophrenia, septic shock, scleroderma, Sjogren's disease, systemic lupus erythematosus (and associated glomerulonephritis), temporal arteritis, tissue graft rejection and hyperacute rejection of transplanted organs, vasculitis (ANCA-associated and other vasculitides), vitiligo, and wegener's granulomatosis.

It should be understood that the invention is not limited to the particular embodiments shown and described herein, but that various changes and modifications may be made without departing from the spirit and scope of the invention as defined by the claims.

The compounds according to the present invention may be synthesized according to a variety of schemes. Necessary starting materials may be obtained by standard procedures of organic chemistry. The compounds and processes of the present invention will be better understood in connection with the following representative synthetic schemes and examples, which are intended as an illustration only and not limiting of the scope of the invention. Various changes and modifications to the disclosed embodiments will be apparent to those skilled in the art and such changes and modifications including, without limitation, those relating to the chemical structures, substituents, derivatives, and/or methods of the invention may be made without departing from the spirit of the invention and the scope of the appended claims.

A typical approach to synthesize the intermediate

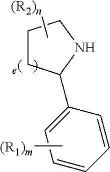

is described in Scheme 1-1 below.

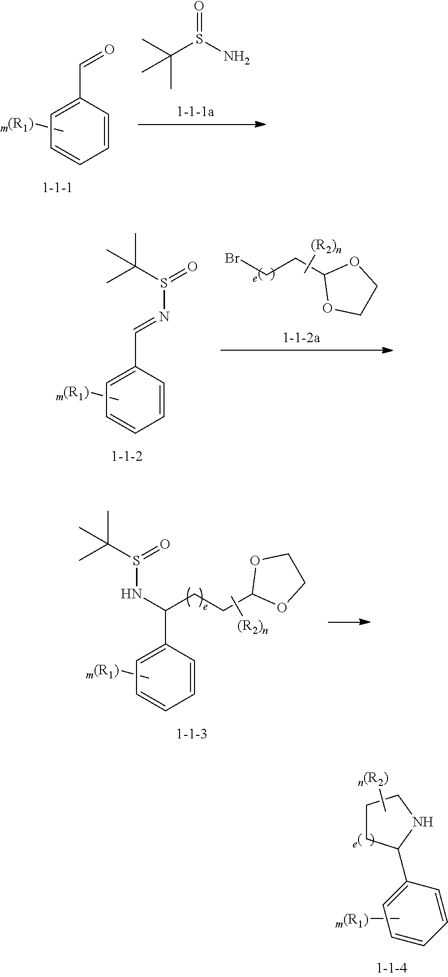

In Scheme 1-1, the condensation of 1-1-1 with the commercially available 1-1-1a results in 1-1-2, and then the reaction of 1-1-2 with 1-1-2a provides 1-1-3. Intramolecular cycliation of 1-1-3 under a suitable condition can give 1-1-4.

Also, the target compounds can be synthesized by alternative methods but not limited to the above procedures.

A typical approach to synthesize the intermediate

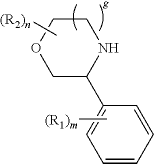

is described in Scheme 1-2 below.

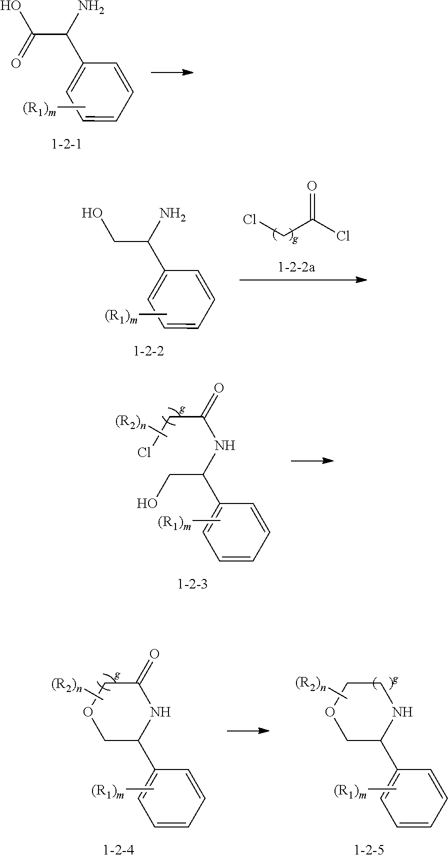

In Scheme 1-2, the starting material 1-2-1 can be prepared by conventional procedures using appropriate compounds and reagents. The starting material 1-2-1 can be reduced to give 1-2-2. After that, the reaction of 1-2-2 with 1-2-2a can afford 1-2-3 readily, which can be converted to 1-2-4 through an intramolecular cyclization reaction. Finally, the intermediate 1-2-4 is reduced to yield the target compounds 1-2-5.

Also, the target compounds can be synthesized by alternative methods but not limited to the above procedures.

A typical approach to synthesize the intermediate

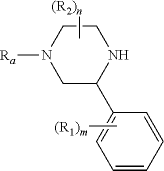

is described in Scheme 1-3 below.

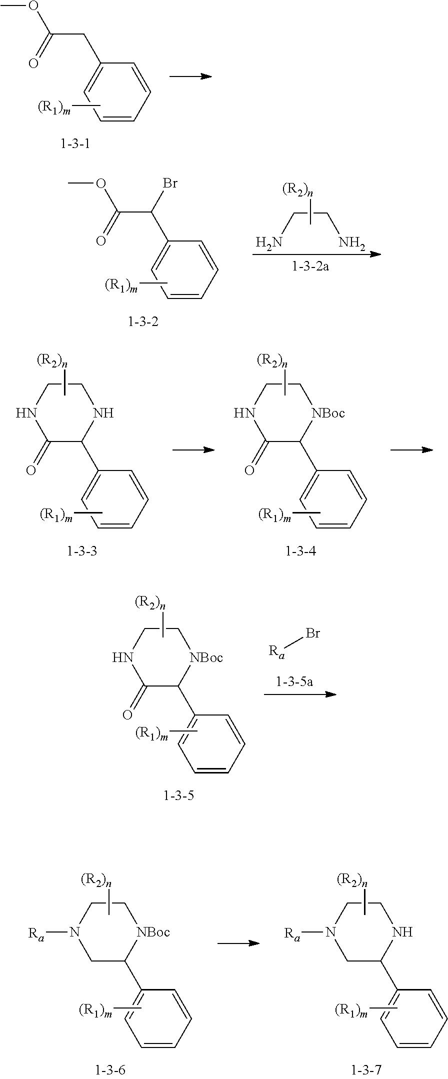

In Scheme 1-3, the starting material 1-3-1 can be prepared by conventional procedures using appropriate compounds and reagents. The bromination of 1-3-1 can generate 1-3-2, which further reacts with 1-3-2a to give 1-3-3. After that, 1-3-3 can be converted to 1-3-4 readily, and then the reduction of 1-3-4 can generate 1-3-5. The intermediate 1-3-5 undergoes an N-substitution reaction with 1-3-5a to give 1-3-6, which is deprotected to afford the intermediate 1-3-7.

Also, the target compounds can be synthesized by alternative methods but not limited to the above procedures.

A typical approach to synthesize the intermediate

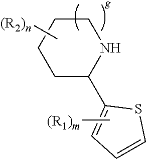

is described in Scheme 1-4 below.

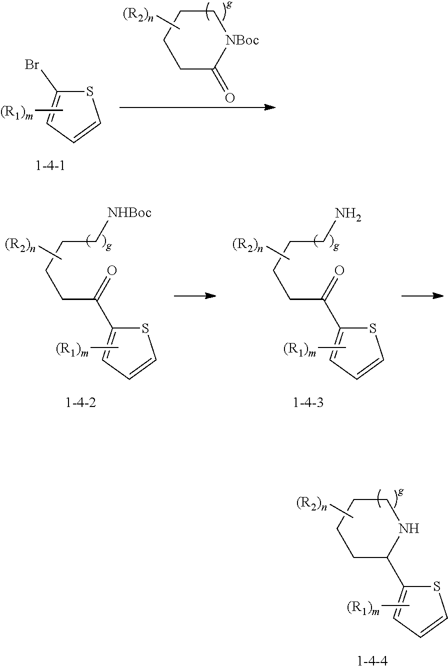

In Scheme 1-4, the starting material 1-4-1 can be prepared by conventional procedures using appropriate compounds and reagents. The starting material 1-4-1 can be converted to 1-4-2 under a literate known condition. Finally, the intermediate 1-4-2 go through a two-step sequence of deprotection reaction and intramolecular reductive amination reaction to generate the target compounds 1-4-4.

Also, the target compounds can be synthesized by alternative methods but not limited to the above procedures.

A typical approach to synthesize the intermediate

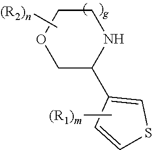

is described in Scheme 1-5 below.

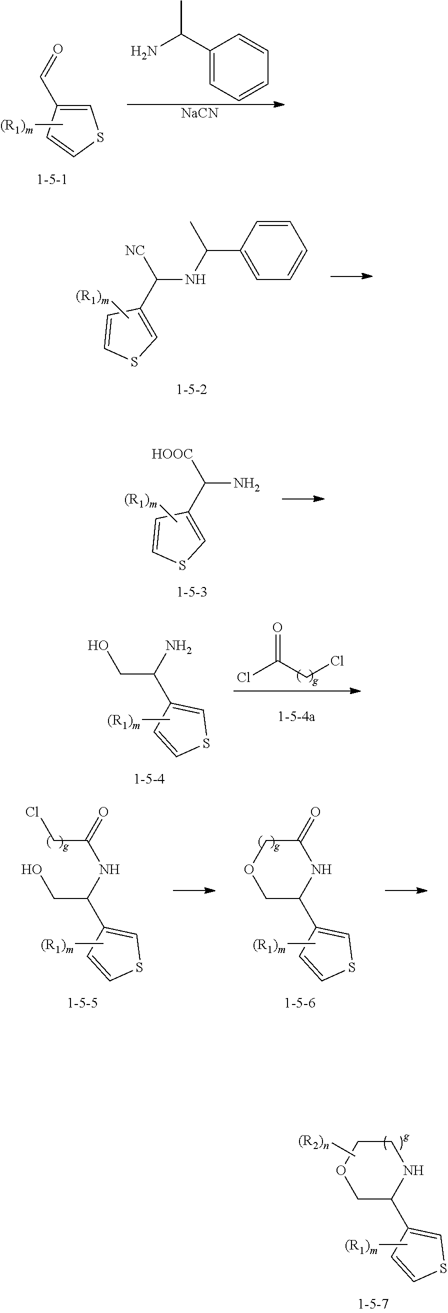

In Scheme 1-5, the starting material 1-5-1 can be prepared by conventional procedures using appropriate compounds and reagents. The starting material 1-5-1 can be converted to 1-5-3 through a two-step sequence of conventional reactions. After that, the carboxylic acid 1-5-3 can be reduced to 1-5-4 readily, which can react with 1-5-4a to give 1-5-5. Finally, 1-5-5 can undergo an intramolecular cyclization to afford 1-5-6, which is further treated with a suitable reducing reagent to afford the target compounds 1-5-7.

Also, the target compounds can be synthesized by alternative methods but not limited to the above procedures.

A typical approach to synthesize the intermediate

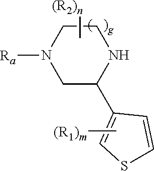

is described in Scheme 1-6 below.

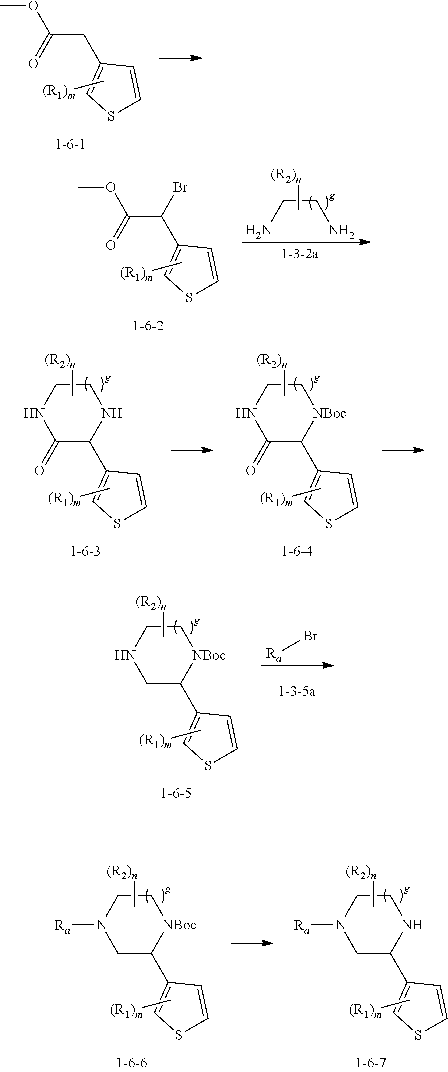

In Scheme 1-6, the starting material 1-6-1 can be prepared by conventional procedures using appropriate compounds and reagents. The bromination of 1-6-1 can generate 1-6-2, which further reacts with 1-3-2a to give 1-6-3. After that, 1-6-3 can be converted to 1-6-4 readily, and then the reduction of 1-6-4 can generate 1-6-5. The intermediate 1-6-5 undergoes an N-substitution reaction with 1-3-5a to give 1-6-6, which can be deprotected to afford the target compounds 1-6-7.

Also, the target compounds can be synthesized by alternative methods but not limited to the above procedures.

A typical approach to synthesize the intermediate

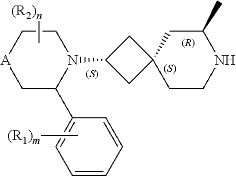

is described in Scheme 1-7 below.

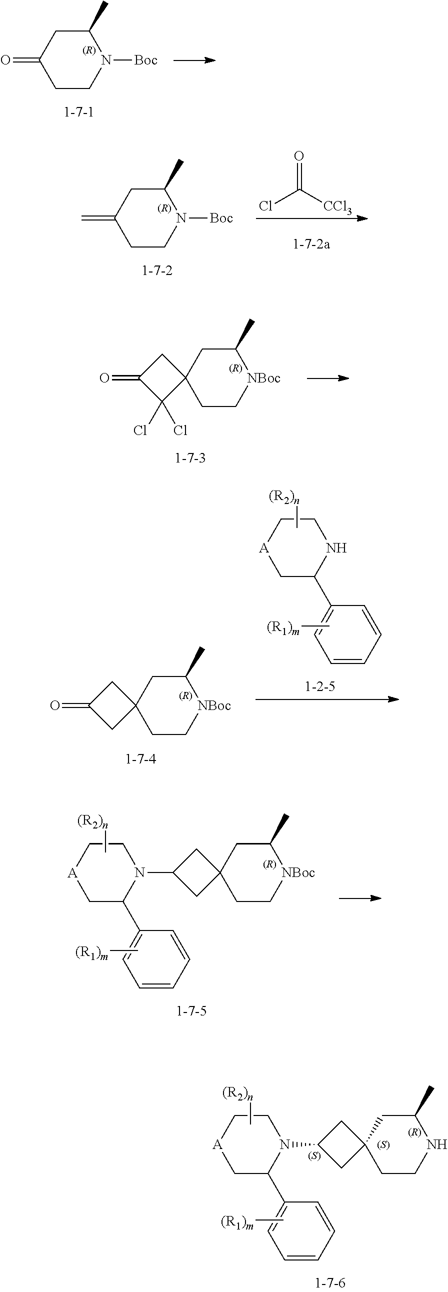

In Scheme 1-7, the starting material 1-7-1 can be prepared by conventional procedures using appropriate compounds and reagents. The starting material 1-7-1 can be converted to 1-7-2 via a general Wittig reaction, which further undergoes Staudinger ketene cycloaddition with 1-7-2a to give 1-7-3. After that, the dichloride 1-7-3 can be reduced to 1-7-4 by a suitable reagent, and then the reaction of 1-7-4 with 1-2-5 can generate 1-7-5. Finally, the deprotection of 1-7-5 can give the target compounds 1-7-6.

Also, the target compounds can be synthesized by alternative methods but not limited to the above procedures.

A typical approach to synthesize the intermediate

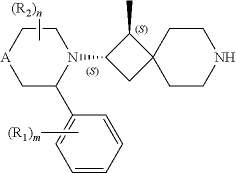

is described in Scheme 1-8 below.

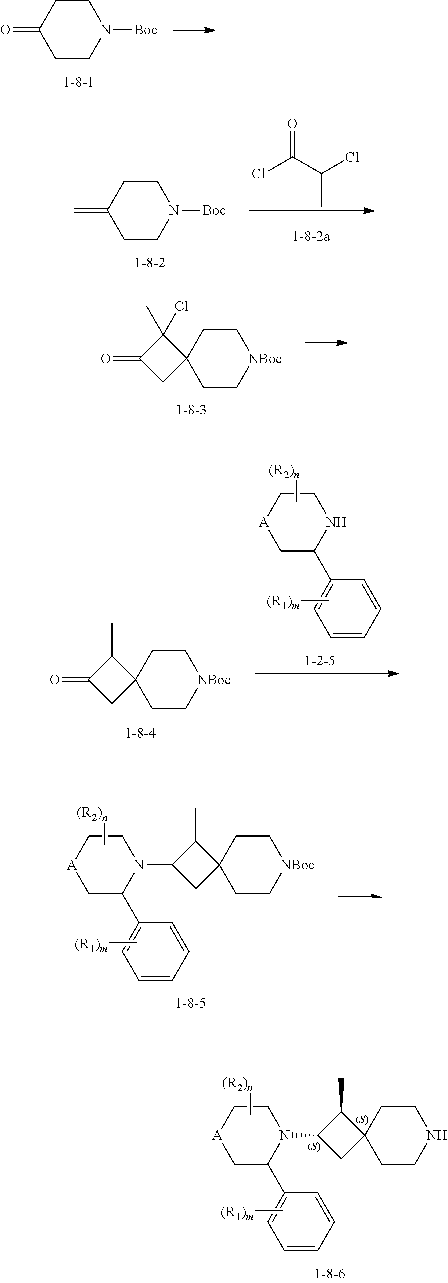

In Scheme 1-8, the starting material 1-8-1 can be prepared by conventional procedures using appropriate compounds and reagents. The starting material 1-8-1 can be converted to 1-8-2 via a general Wittig reaction, which further undergoes Staudinger ketene cycloaddition with 1-8-2a to give 1-8-3. After that, the chloride 1-8-3 can be reduced to 1-8-4 by a suitable reagent, and then the reaction of 1-8-4 with 1-2-5 can generate 1-8-5. Finally, the deprotection of 1-8-5 can give the target compounds 1-8-6.

Also, the target compounds can be synthesized by alternative methods but not limited to the above procedures.

A typical approach to synthesize the intermediate

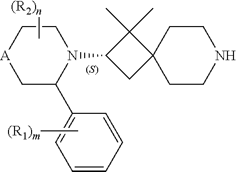

is described in Scheme 1-9 below.

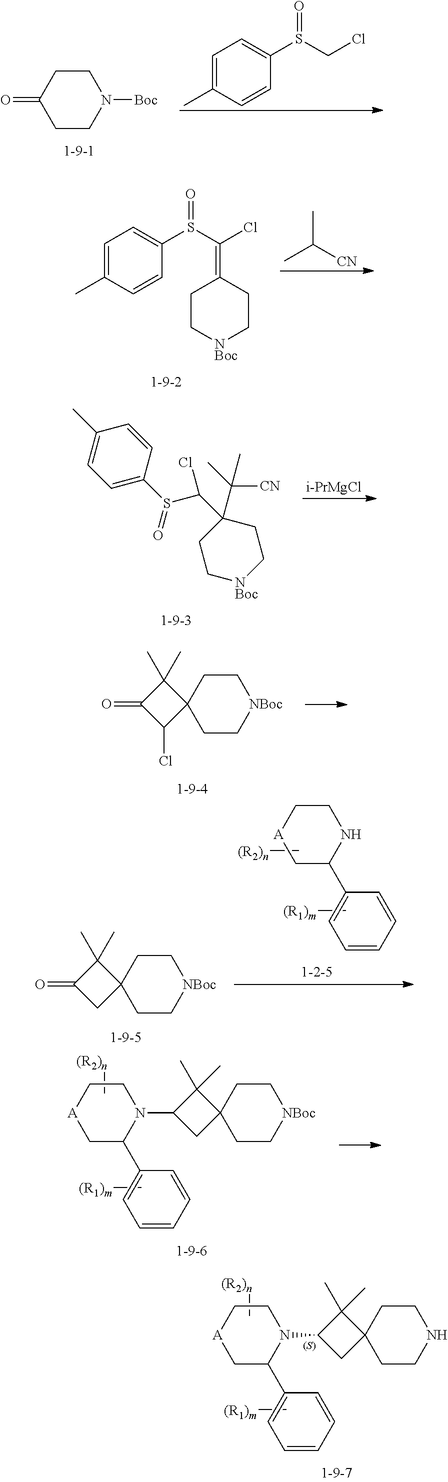

In Scheme 1-9, the starting material 1-9-1 can be prepared by conventional procedures using appropriate compounds and reagents. The starting material 1-9-1 can be converted to 1-9-4 via a sequence of literate known reactions, and then 1-9-4 can be reduced to give 1-9-5. After that, the reductive amination of 1-9-5 with 1-2-5 can generate 1-9-6. Finally, the deprotection of 1-9-6 can give the target compounds 1-9-7.

Also, the target compounds can be synthesized by alternative methods but not limited to the above procedures.

A typical approach to synthesize the intermediate

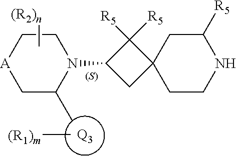

is described in Scheme 1-10 below.

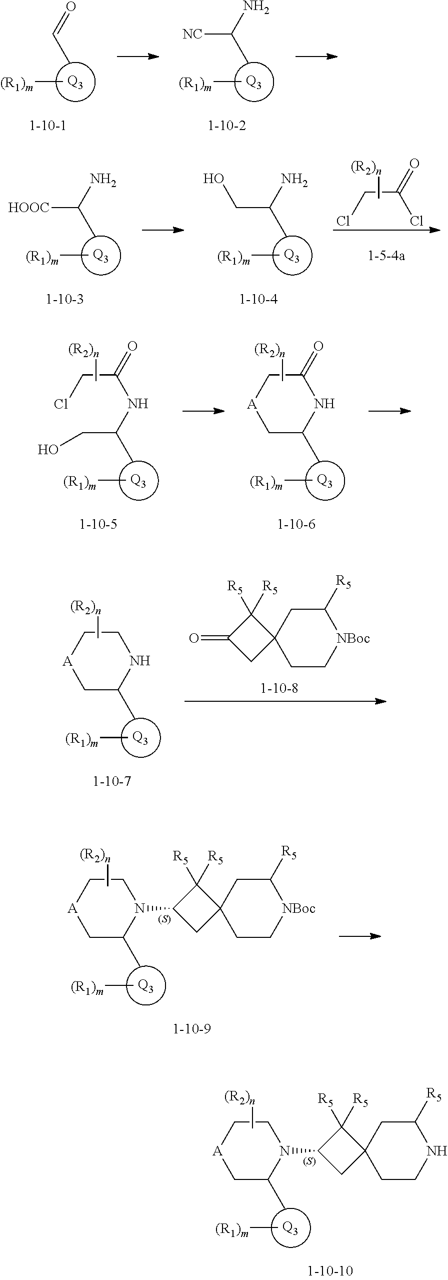

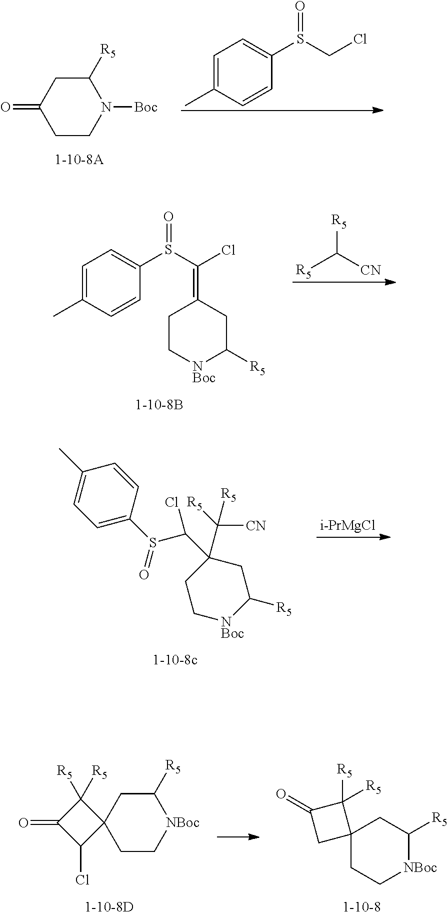

In Scheme 1-10, the starting material 1-10-1 can be prepared by conventional procedures using appropriate compounds and reagents. The starting material 1-10-1 can be converted to 1-10-3 through a two-step sequence of conventional reactions. After that, the carboxylic acid 1-10-3 can be reduced to 1-10-4 readily, which can react with 1-5-4a to give 1-10-5. Next, 1-10-5 can undergo an intramolecular cyclization to give 1-10-6, which is treated with a suitable reducing reagent to afford 1-10-7. And then 1-10-8 can be prepared by the method similar to Scheme 1-9 by using appropriate staring materials, and intermediates. Finally, the reductive amination of 1-10-7 with 1-10-8 can generate 1-10-9, which is deprotected to give the target compounds 1-10-10.

Also, the target compounds can be synthesized by alternative methods but not limited to the above procedures.

A typical approach to synthesize the intermediate

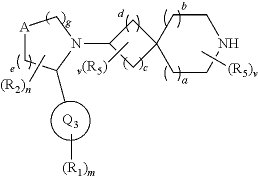

is described in Scheme 1-11 below.

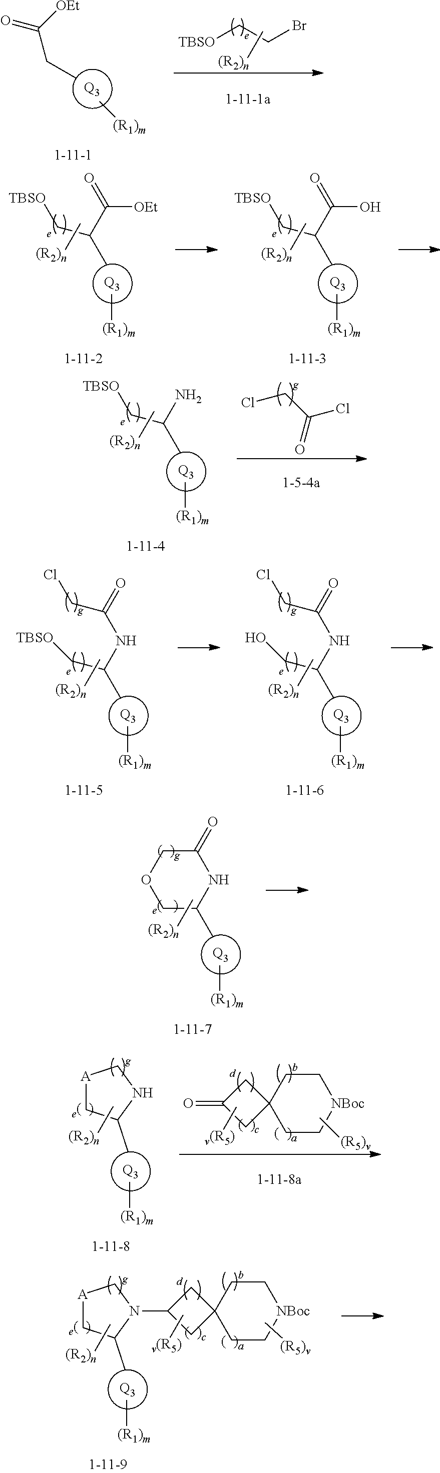

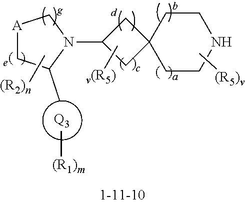

In Scheme 1-11, the starting material 1-11-1 can be prepared by conventional procedures using appropriate compounds and reagents. The starting material 1-11-1 can be converted to 1-11-3 through a two-step sequence of conventional reactions. After that, the carboxylic acid 1-11-3 can be converted to 1-11-4 readily, which can react with 1-5-4a to give 1-11-5. The deprotection of 1-11-5 can generate 1-11-6, which can be converted to 1-11-7 via an intramolecular cyclization reaction. The intermediate 1-11-7 can be reduced to afford 1-11-8, and then the reaction of 1-11-8 with 1-11-8a can give 1-11-9. Finally, the deprotection of 1-11-9 can generate the target compounds 1-11-10.

Also, the target compounds can be synthesized by alternative methods but not limited to the above procedures.

Similarly, the compounds of

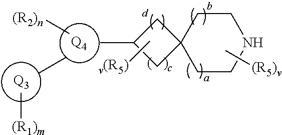

can be prepared by schemes similar to the Scheme 1-Scheme 11 by using appropriate staring materials and intermediates.

Similarly, the compounds of can be prepared by schemes similar to the Scheme A-Scheme E by using appropriate staring materials and intermediates.

Similarly, the compounds of

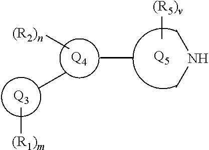

can be prepared by schemes similar to the Scheme 1-Scheme 11 by using appropriate staring materials and intermediates.

A typical approach to synthesize the intermediate

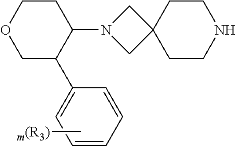

is described in Scheme 1-12 below.

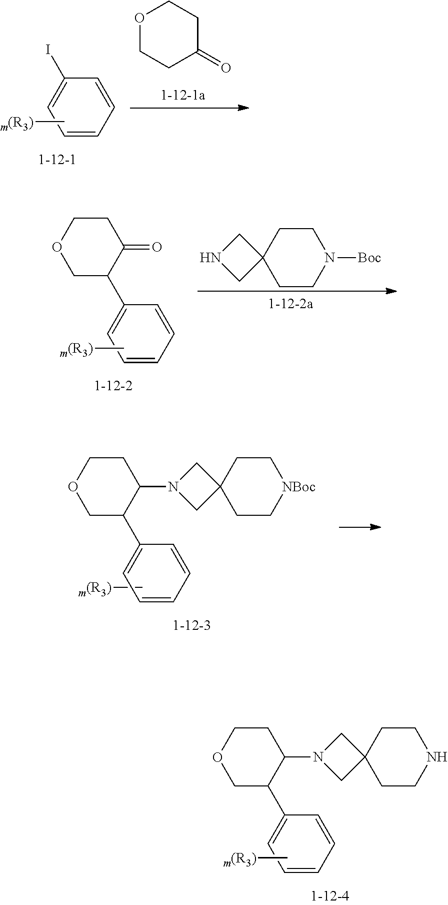

In Scheme 1-12, the starting material 1-12-1 can be prepared by conventional procedures using appropriate compounds and reagents. The starting material 1-12-1 can couple with 1-12-1a to give 1-12-2, which goes through a reductive amination reaction with 1-12-2a to yield the intermediate 1-12-3. Finally, the intermediate 1-12-3 is deprotected to generate the target compounds 1-12-4.

Also, the target compounds can be synthesized by alternative methods but not limited to the above procedures.

A typical approach to synthesize the intermediate

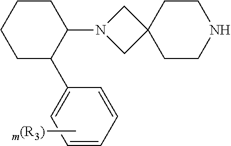

is described in Scheme 1-13 below.

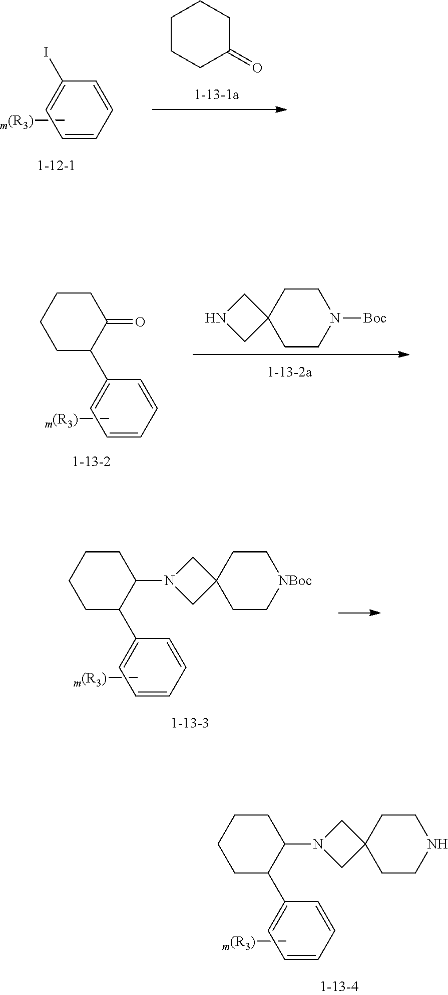

In Scheme 1-13, the starting material 1-12-1 can be prepared by conventional procedures using appropriate compounds and reagents. The starting material 1-12-1 can couple with 1-13-1a to give 1-13-2, which goes through a reductive amination reaction with 1-13-2a to yield the intermediate 1-13-3. Finally, the intermediate 1-13-3 is deprotected to generate the target compounds 1-13-4.

Also, the target compounds can be synthesized by alternative methods but not limited to the above procedures.

A typical approach to synthesize the intermediate

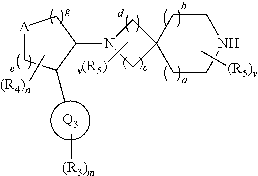

is described in Scheme 1-14 below.

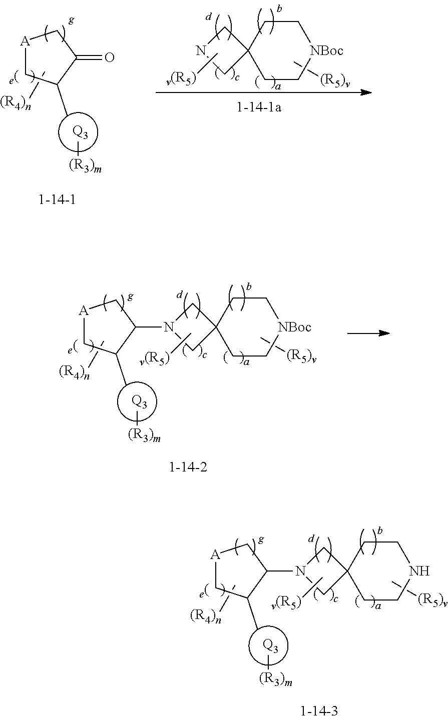

In Scheme 1-14, the starting material 1-14-1 can be prepared by conventional procedures using appropriate compounds and reagents. The starting material 1-14-1 goes through a reductive amination reaction with 1-14-1a to yield the intermediate 1-14-2, which is deprotected to generate the target compounds 1-14-3.

Also, the target compounds can be synthesized by alternative methods but not limited to the above procedures.

A typical approach to synthesize the intermediate

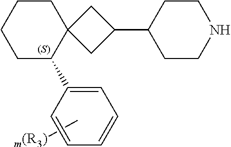

is described in Scheme 1-15 below.

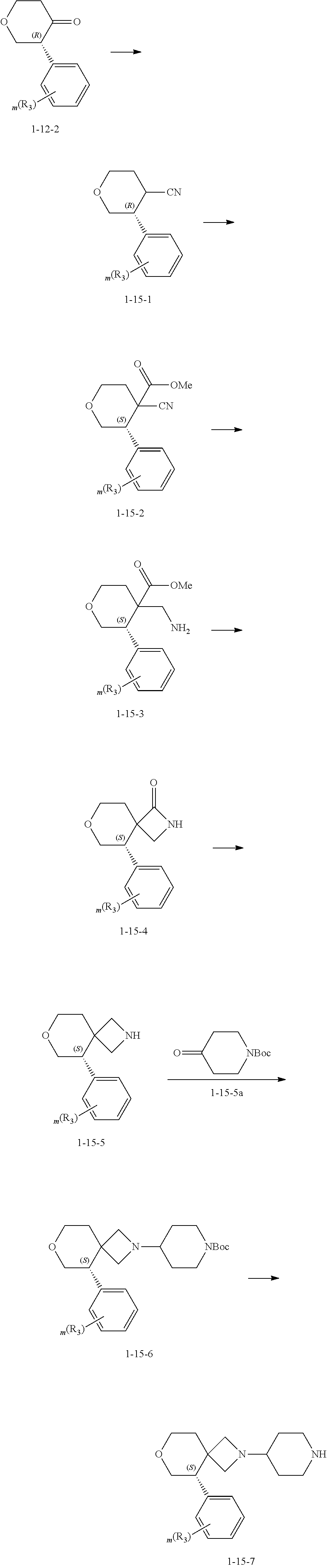

In Scheme 1-15, the starting material 1-12-2 can be prepared by conventional procedures using appropriate compounds and reagents. The starting material 1-12-2 can be converted to 1-15-1 readily, which is converted to 1-15-2 through a conventional organic reaction. After that, the intermediate 1-15-2 is reduced to give 1-15-3, which can be converted to 1-15-4 though an intramolecular cyclization reaction. Next, 1-15-4 is reduced to yield 1-15-5, which can react with 1-15-5a to afford the intermediate 1-15-6. Finally, the intermediate 1-15-6 is deprotected to generate the target compounds 1-15-7.

Also, the target compounds can be synthesized by alternative methods but not limited to the above procedures.

A typical approach to synthesize the intermediate

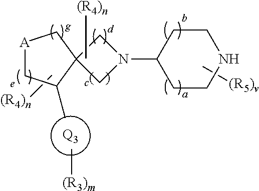

is described in Scheme 1-16 below.

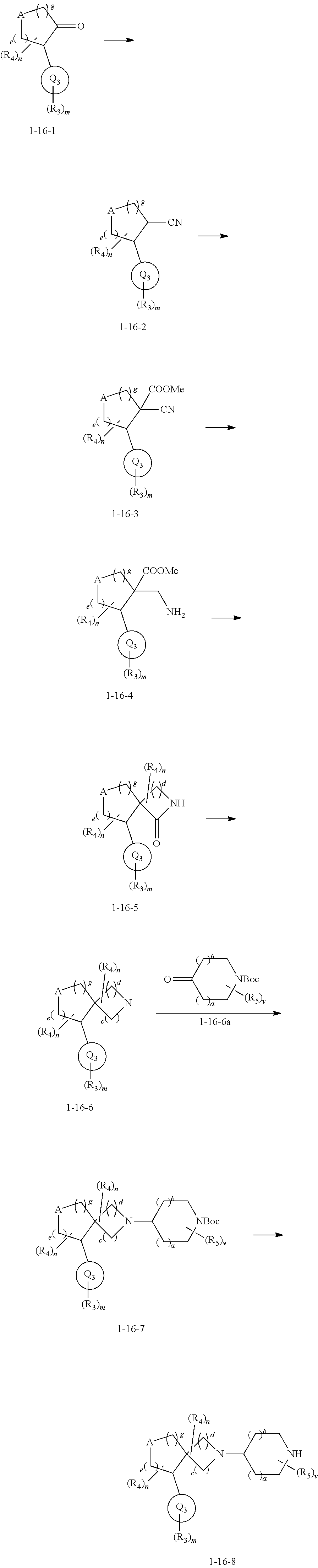

In Scheme 1-16, the starting material 1-16-1 can be prepared by conventional procedures using appropriate compounds and reagents. The starting material 1-16-1 can be converted to 1-16-2 readily, which is converted to 1-16-3 through a conventional organic reaction. After that, the intermediate 1-16-3 is reduced to give 1-16-4, which can be converted to 1-16-5 though an intramolecular cyclization. Next, 1-16-5 is reduced to yield 1-16-6, which can react with 1-16-6a to afford the intermediate 1-16-7. Finally, the intermediate 1-16-7 is deprotected to generate the target compounds 1-16-8.

Also, the target compounds can be synthesized by alternative methods but not limited to the above procedures.

A typical approach to synthesize the intermediate

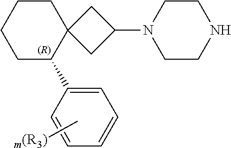

is described in Scheme 1-17 below.

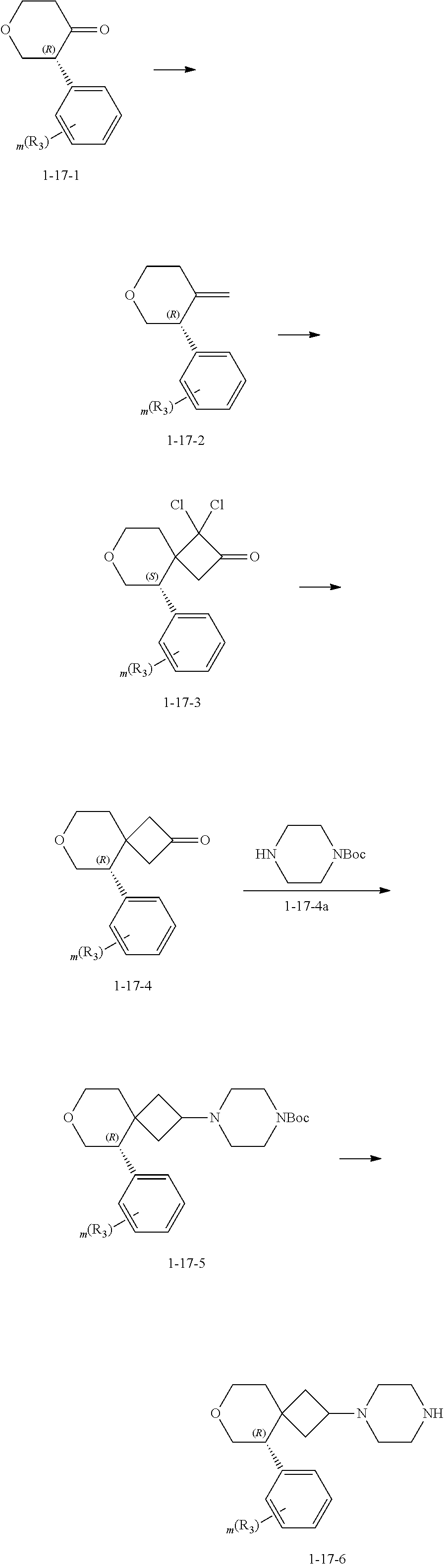

In Scheme 1-17, the starting material 1-17-1 can be prepared by conventional procedures using appropriate compounds and reagents. The starting material 1-17-1 can be converted to 1-17-2 via a general Wittig reaction, which further undergoes Staudinger ketene cycloaddition to give 1-17-3. After that, the dichloride 1-17-3 can be reduced to 1-17-4 by a suitable reagent, and then the reaction of 1-17-4 with 1-17-4a can generate 1-17-5. Finally, the deprotection of 1-17-5 can give the target compounds 1-17-6.

Also, the target compounds can be synthesized by alternative methods but not limited to the above procedures.

A typical approach to synthesize the intermediate

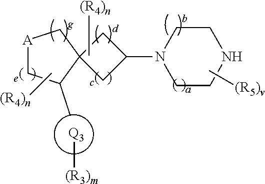

is described in Scheme 1-18 below.

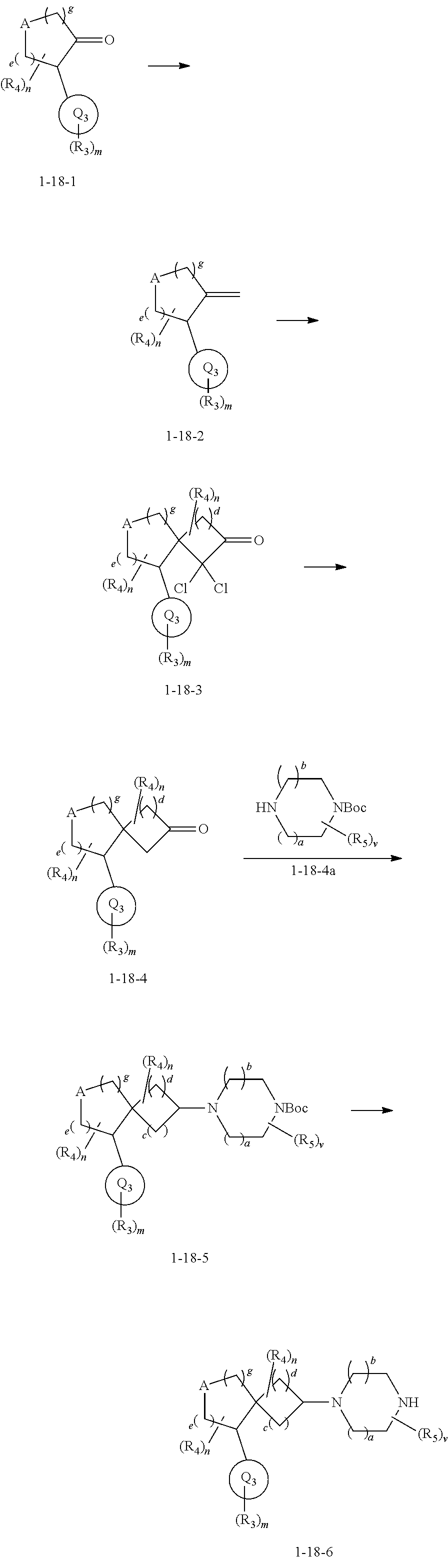

In Scheme 1-18, the starting material 1-18-1 can be prepared by conventional procedures using appropriate compounds and reagents. The starting material 1-18-1 can be converted to 1-18-2 via a general Wittig reaction, which further undergoes Staudinger ketene cycloaddition to give 1-18-3. After that, the dichloride 1-18-3 can be reduced to 1-18-4 by a suitable reagent, and then the reaction of 1-18-4 with 1-18-4a can generate 1-18-5. Finally, the deprotection of 1-18-5 can give the target compounds 1-18-6.

Also, the target compounds can be synthesized by alternative methods but not limited to the above procedures.

A typical approach to synthesize the intermediate

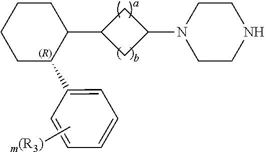

is described in Scheme 1-19 below.

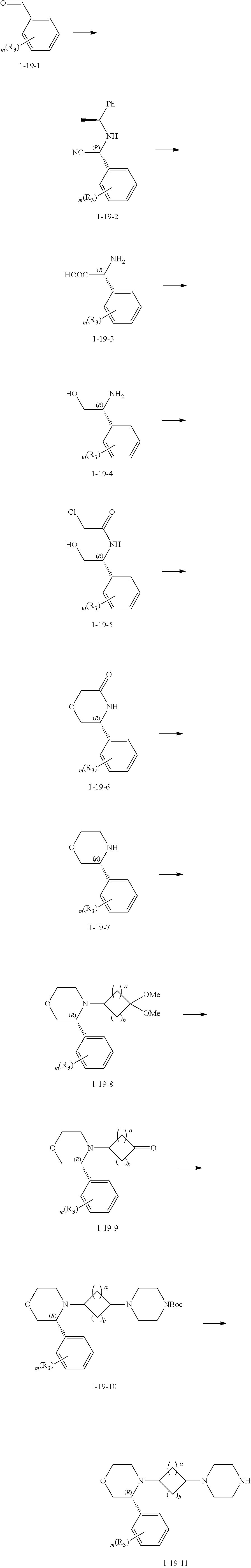

In Scheme 1-19, the starting material 1-19-1 can be prepared by conventional procedures using appropriate compounds and reagents. The starting material 1-19-1 can be converted to 1-19-2 via a Strecker reaction, which can be converted to 1-19-3 readily. After that, the intermediate 1-19-3 is reduced to 1-19-4 by a suitable reagent. The intermediate 1-19-4 can be converted to 1-19-5, which undergoes an intramolecular cyclization to give 1-19-6. Next, the intermediate 1-19-6 is reduced to give 1-19-7, which can be converted to 1-19-9 through a two-step sequence conventional reaction. Finally, the intermediate 1-19-9 can be converted to 1-19-10, which is deprotected to yield the target compounds 1-19-11.

Also, the target compounds can be synthesized by alternative methods but not limited to the above procedures.

A typical approach to synthesize the intermediate

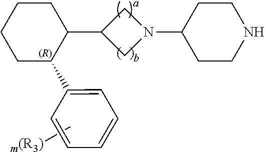

is described in Scheme 1-20 below.

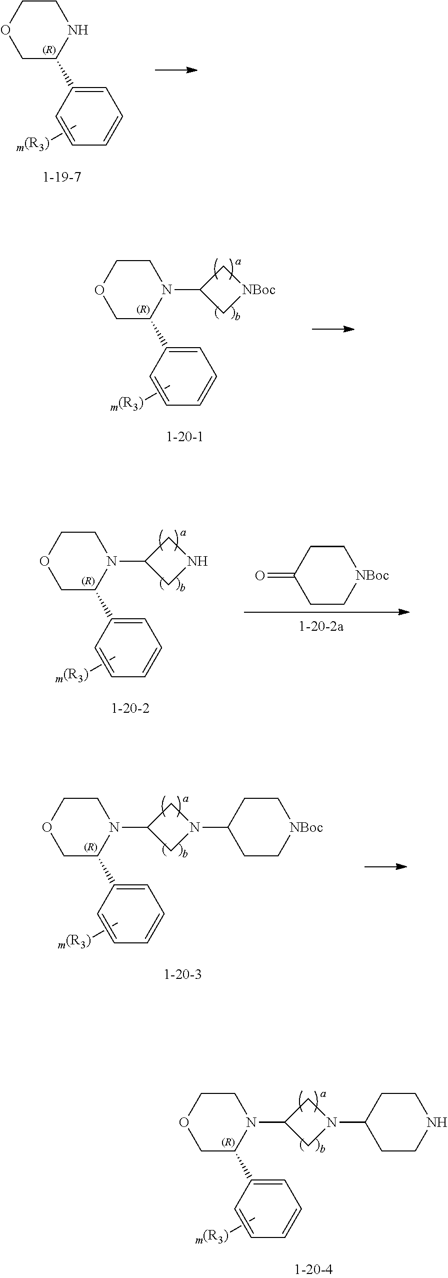

In Scheme 1-20, the starting material 1-19-7 can be prepared by conventional procedures using appropriate compounds and reagents. The starting material 1-19-7 can be converted to 1-20-1 via a reductive amination reaction, which is deprotected to give 1-20-2. Finally, the intermediate 1-20-2 can react with 1-20-2a to afford 1-20-3, which is deprotected to yield the target compounds 1-20-4.

Also, the target compounds can be synthesized by alternative methods but not limited to the above procedures.

Similarly, the compounds of

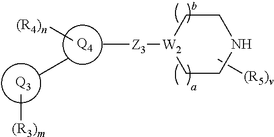

in which W₂ is N or C(Rₐ) can be prepared by schemes similar to the Scheme 1-19 to Scheme 1-20 by using appropriate staring materials and intermediates.

A typical approach to synthesize the intermediate

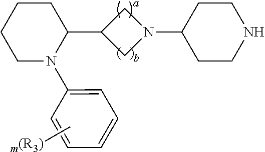

is described in Scheme 1-21 below.

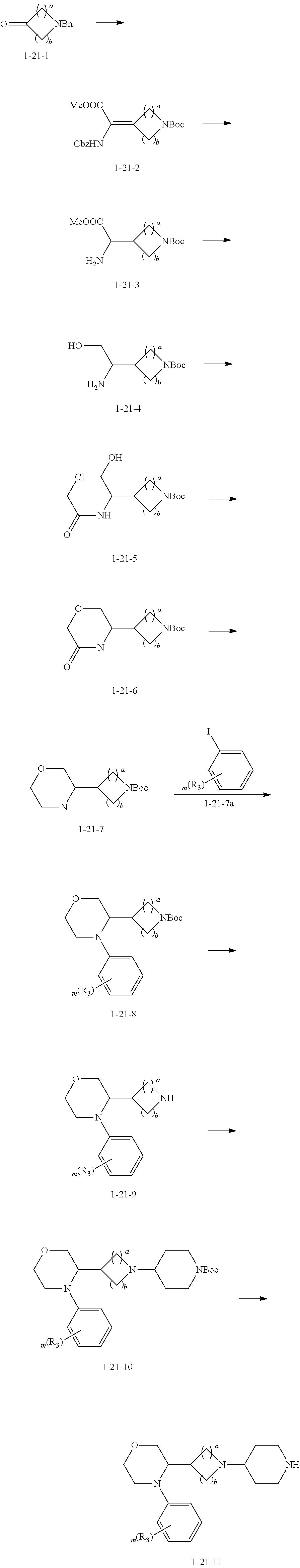

In Scheme 1-21, the starting material 1-21-1 can be prepared by conventional procedures using appropriate compounds and reagents. The starting material 1-21-1 can be converted to 1-21-2 readily, which can be reduced to 1-21-3. After that, the intermediate 1-21-3 is further reduced to 1-21-4 by a suitable reagent. The intermediate 1-21-4 can be converted to 1-21-5, which undergoes an intramolecular cyclization to give 1-21-6. Next, the intermediate 1-21-6 is reduced to give 1-21-7, which can couple with 1-21-a to give 1-21-8. The intermediate 1-21-8 is deprotected to give 1-21-9. Finally, the intermediate 1-21-9 can be converted to 1-21-10, which is deprotected to yield the target compounds 1-21-11.

Also, the target compounds can be synthesized by alternative methods but not limited to the above procedures.

A typical approach to synthesize the intermediate

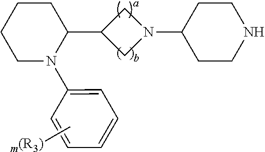

is described in Scheme 1-22 below.

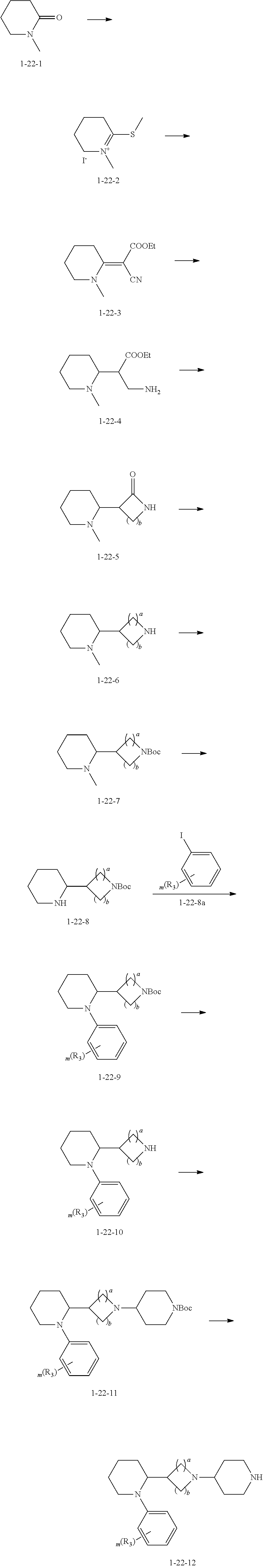

In Scheme 1-22, the starting material 1-22-1 can be prepared by conventional procedures using appropriate compounds and reagents. The starting material 1-22-1 can be converted to 1-22-2 readily, which is converted to 1-22-3 by a literate known condition. After that, the intermediate 1-22-3 is reduced to give 1-22-4 by a suitable reagent. The intermediate 1-22-4 can be converted to 1-22-5 through an intramolecular cyclization, which is reduced to give 1-22-6. Next, the intermediate 1-22-6 is protected to give 1-22-7, and then the de-methylation of 1-22-7 can give the intermediate 1-22-8. The intermediate 1-22-8 can couple with 1-22-8a to afford 1-22-9, which is deprotected to give 1-22-10. Finally, the intermediate 1-22-10 can be converted to 1-22-11, which is deprotected to yield the target compounds 1-22-12.

Also, the target compounds can be synthesized by alternative methods but not limited to the above procedures.

A typical approach to synthesize the intermediate

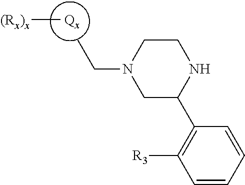

is described in Scheme 1-23 below.

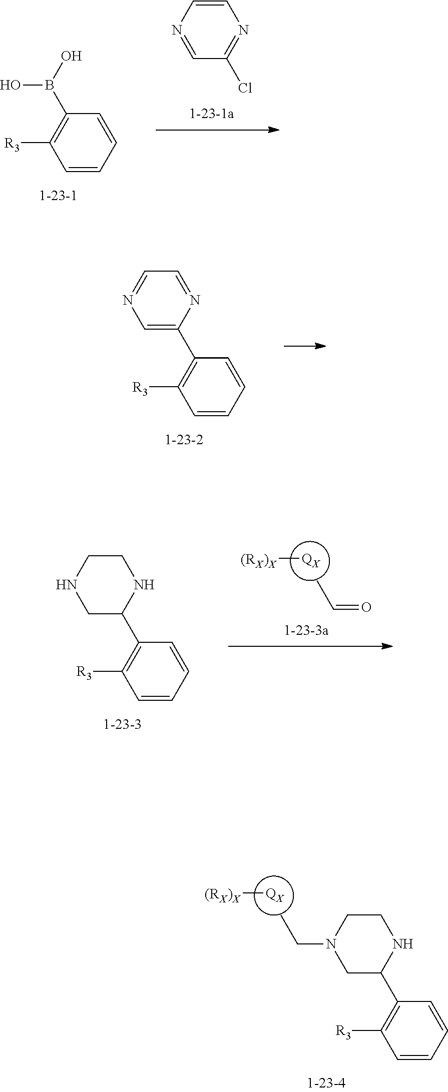

In Scheme 1-23, the starting material 1-23-1 can be prepared by conventional procedures using appropriate compounds and reagents. The starting material 1-23-1 is converted to 1-23-2 via a Suzuki coupling reaction. After that, the intermediate 1-23-2 can be reduced to give 1-23-3 with a suitable condition. Finally, the intermediate 1-23-3 goes through a reductive amination reaction with 1-23-3a to yield the target compounds 1-23-4.

Also, the target compounds can be synthesized by alternative methods but not limited to the above procedures.

A typical approach to synthesize the intermediate

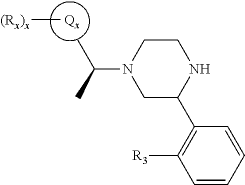

is described in Scheme 1-24 below.

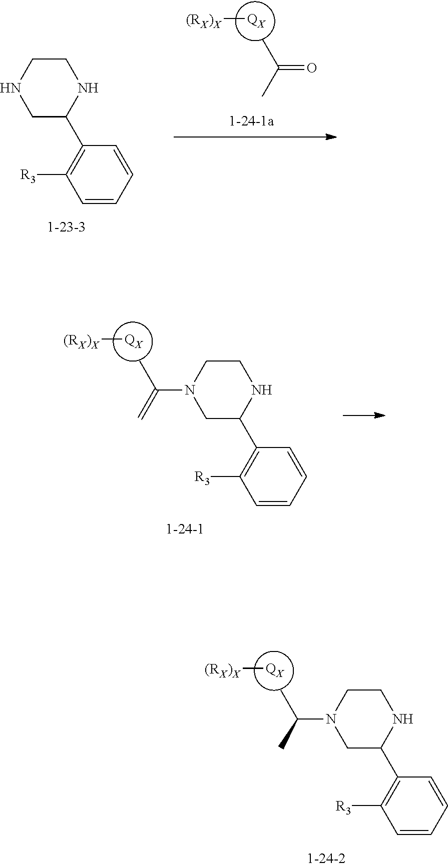

In Scheme 1-24, the starting material 1-23-3 can be prepared by conventional procedures using appropriate compounds and reagents. The starting material 1-23-3 can react with 1-24-1a to give 1-24-1, which is hydrogenated to yield the target compounds 1-24-2.

Also, the target compounds can be synthesized by alternative methods but not limited to the above procedures.

Similarly, the compounds of

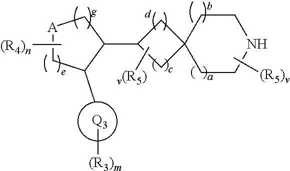

can be prepared by schemes similar to the Scheme 1-21 to Scheme 1-24 by using appropriate staring materials and intermediates.

An approach to synthesize of target compounds of

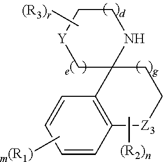

is described in Scheme 1-25 below.

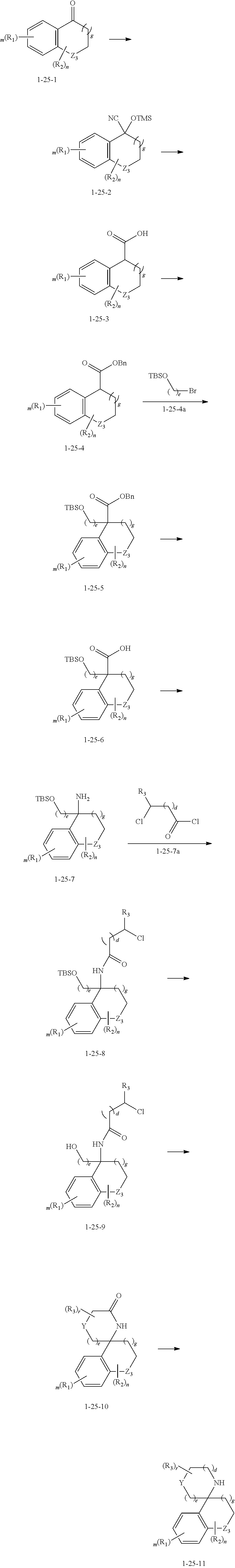

In Scheme 1-25, the starting material 1-25-1 can be prepared by conventional procedures using appropriate compounds and reagents. The starting material 1-25-1 can be converted to 1-25-3 through a two-step sequence of conventional organic reactions. After that, the carboxylic acid 1-25-3 can be converted to 1-25-4 readily, which can react with 1-25-4a to give intermediate 1-25-5. The deprotection of 1-25-5 can generate 1-25-6, which can be converted to 1-25-7 via a Curtius rearrangement reaction. The intermediate 1-25-7 can react with 1-25-7a to afford 1-25-8, and then deprotected of 1-25-8 can give the intermediate 1-25-9. Finally, 1-25-9 can undergo an intramolecular cyclization to afford the intermediate 1-25-10, which is treated with a suitable reductive reagent to afford the target compounds 1-25-11.

Also, the target compounds can be synthesized by alternative methods but not limited to the above procedures.

A typical approach to synthesize the intermediate

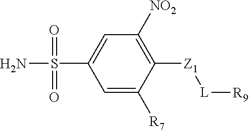

is described in Scheme 2-1 below.

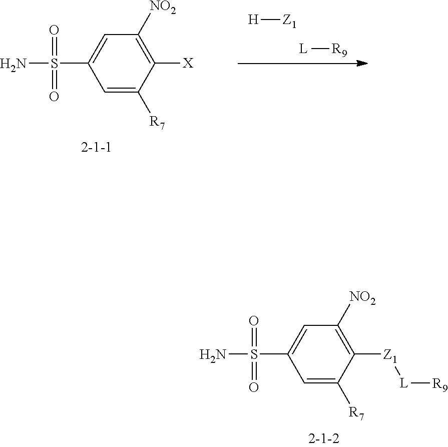

In Scheme 2-1, the starting material 2-1-1 reacts with appropriate alcohol or amine will yield 2-1-2.

Also, the target compounds can be synthesized by alternative methods but not limited to the above procedures.

Similarly, the intermediate of

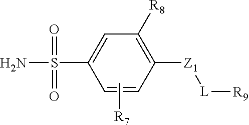

can be prepared by the method similar to Scheme 2-1 by using appropriate staring materials, and intermediates.

A typical approach to synthesize the intermediate in which is described in Scheme 2-2 below.

A typical approach to synthesize the intermediate

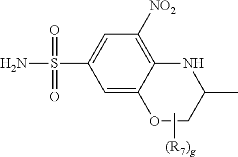

is described in Scheme 2-2 below.

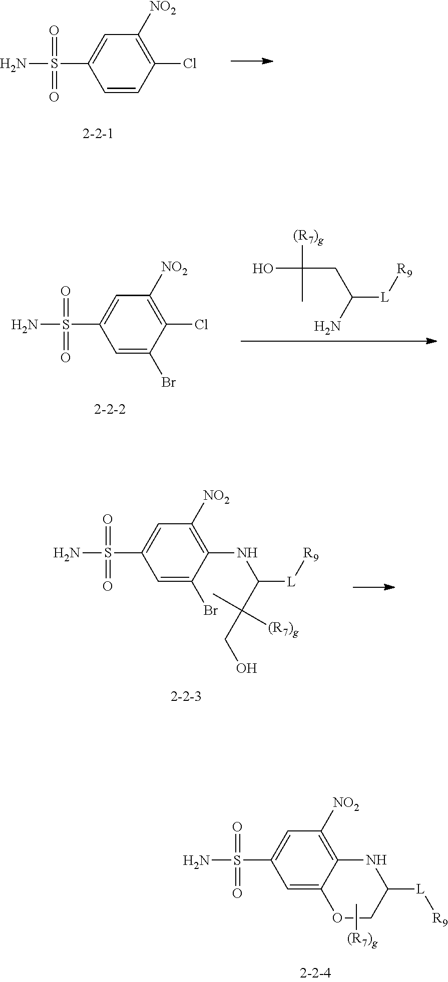

In Scheme 2-2, the bromination of the commercially available 2-2-1 results in 2-2-2, and then the reaction of 2-2-2 with appropriate amine provides 2-2-3. Intramolecular cyclization of 2-2-3 using metal-catalyzed coupling condition such as Buchwald reaction or other coupling reaction known in the literatures give 2-2-4. Alternatively, 2-2-4 can be obtained via a three-step sequence of mesylation of the hydroxyl group of 2-2-3, SN₂ reaction and intramolecular cyclization.

Also, the target compounds can be synthesized by alternative methods but not limited to the above procedures.

A typical approach to synthesize the intermediate

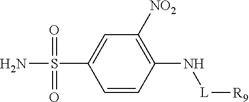

is described in Scheme 2-3 below.

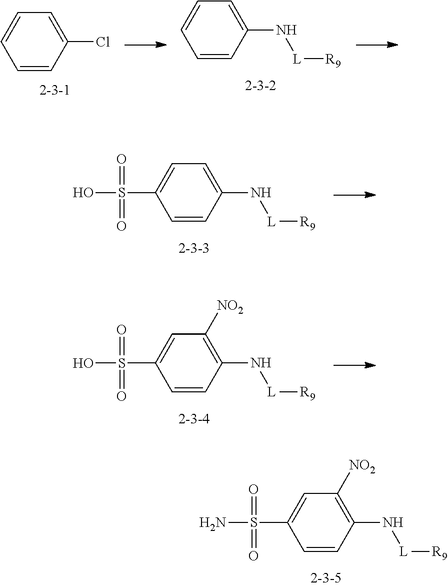

In Scheme 2-3, the starting material 2-3-1 can be prepared by conventional procedures using appropriate compounds and reagents. The starting material 2-3-1 can be converted to 2-3-2 via a SNAr reaction, and then the sulfonylation of 2-3-2 can afford the intermediate 2-3-3. Finally, the intermediate 2-3-3 goes through a two-step sequence of conventional organic reaction to yield the target compounds 2-3-5.

Also, the target compounds can be synthesized by alternative methods but not limited to the above procedures.

A typical approach to synthesize the intermediate

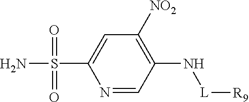

is described in Scheme 2-4 below.

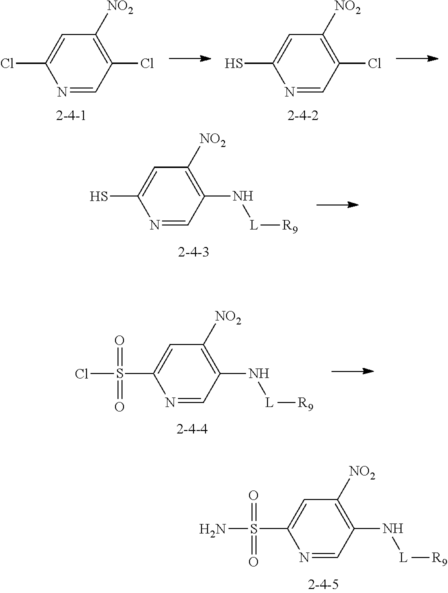

In Scheme 2-4, the starting material 2-4-1 can be prepared by conventional procedures using appropriate compounds and reagents. The starting material 2-4-1 can be converted to 2-4-3 via a two-step sequence of conventional organic reaction. Finally, the intermediate 2-4-3 can be converted to the corresponding sulfonyl chlorides, which can react with ammonia to yield the target compounds 2-4-5.

Also, the target compounds can be synthesized by alternative methods but not limited to the above procedures.

A typical approach to synthesize the intermediate

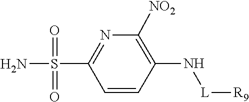

is described in Scheme 2-5 below.

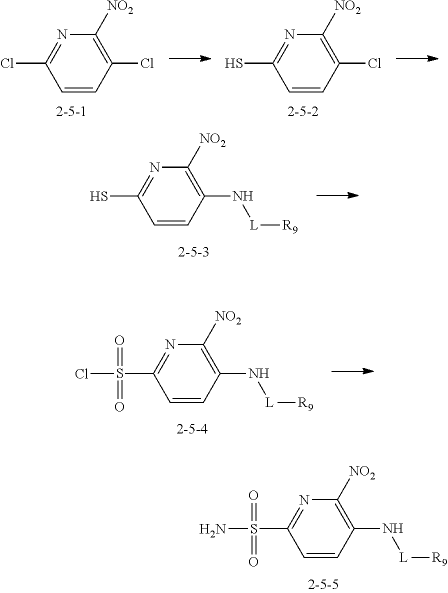

In Scheme 2-5, the starting material 2-5-1 can be prepared by conventional procedures using appropriate compounds and reagents. The starting material 2-5-1 can be converted to 2-5-3 via a two-step sequence conventional organic reaction. Finally, the intermediate 2-5-3 can be converted to the corresponding sulfonyl chlorides 2-5-4, which can react with ammonia to yield the target compounds 2-5-5.

Also, the target compounds can be synthesized by alternative methods but not limited to the above procedures.

A typical approach to synthesize the intermediate

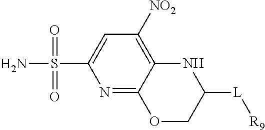

is described in Scheme 2-6 below.

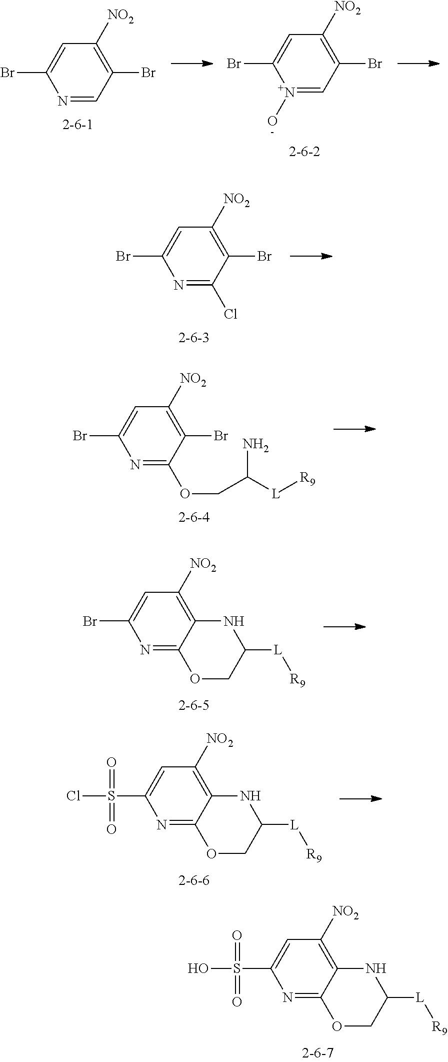

In Scheme 2-6, the starting material 2-6-1 can be prepared by conventional procedures using appropriate compounds and reagents. The starting material 2-6-1 can be oxidized to yield 2-6-2, which can be converted to the intermediate 2-6-3 readily. After that, the intermediate 2-6-3 undergoes a SNAr reaction to give 2-6-4, which is converted to 2-6-5 through an intramolecular cyclization reaction. Finally, the intermediate 2-6-5 can be converted to the corresponding sulfonyl chlorides 2-6-6, which can react with ammonia to yield the target compounds 2-6-7.

Also, the target compounds can be synthesized by alternative methods but not limited to the above procedures.

A typical approach to synthesize the intermediate

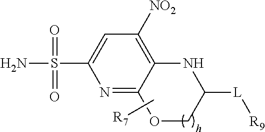

in which h is 0, 1, or 2 is described in Scheme 2-7 below.

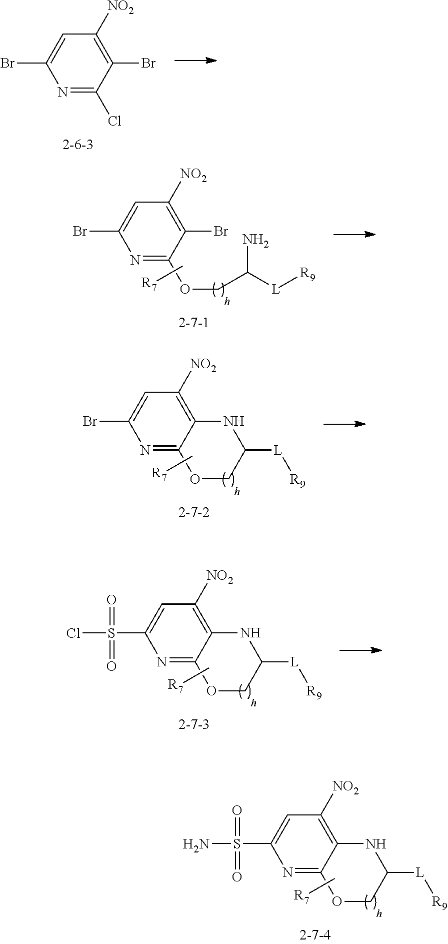

In Scheme 2-7, the starting material 2-6-3 can be prepared by conventional procedures using appropriate compounds and reagents. The starting material 2-6-3 undergoes a SNAr reaction to give 2-7-1, which is converted to 2-7-2 through an intramolecular cyclization reaction. Finally, the intermediate 2-7-2 can be converted to the corresponding sulfonyl chlorides 2-7-3, which can further react with ammonia to yield the target compounds 2-7-4.

Also, the target compounds can be synthesized by alternative methods but not limited to the above procedures.

A typical approach to synthesize the intermediate

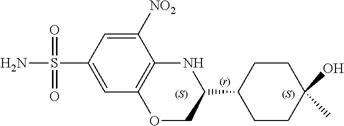

is described in Scheme 2-8 below.

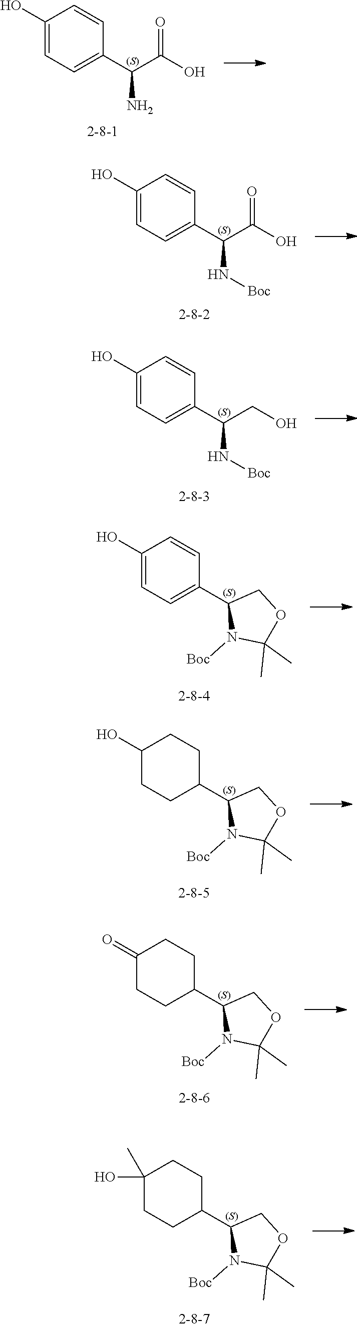

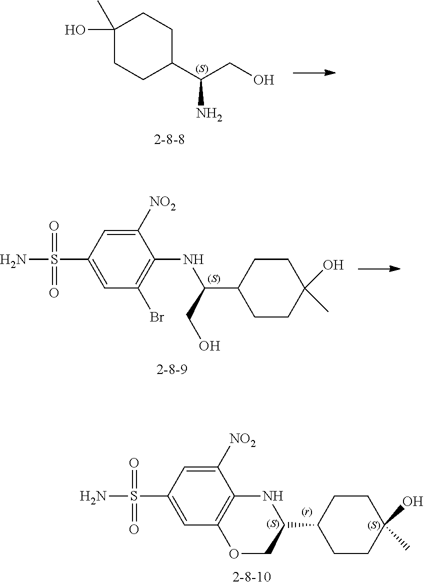

In Scheme 2-8, the starting material 2-8-1 can be prepared by conventional procedures using appropriate compounds and reagents. The starting material 2-8-1 can be converted to 2-8-3 through a two-step sequence of conventional organic reactions. After that, the intermediate 2-8-3 is protected to give 2-8-4, which can be further reduced to yield the intermediate 2-8-5. The intermediate 2-8-5 is oxidized to generate the ketone 2-8-6, which can be converted to 2-8-7 readily. The intermediate 2-8-7 is deprotected to afford 2-8-8, which can be further converted to the intermediate 2-8-9. Finally, 2-8-9 undergoes an intramolecular cyclization to afford the target compounds 2-8-10.

Also, the target compounds can be synthesized by alternative methods but not limited to the above procedures.

A typical approach to synthesize the intermediate

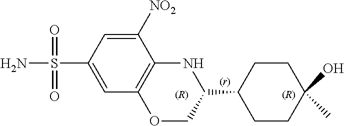

is described in Scheme 2-9 below.

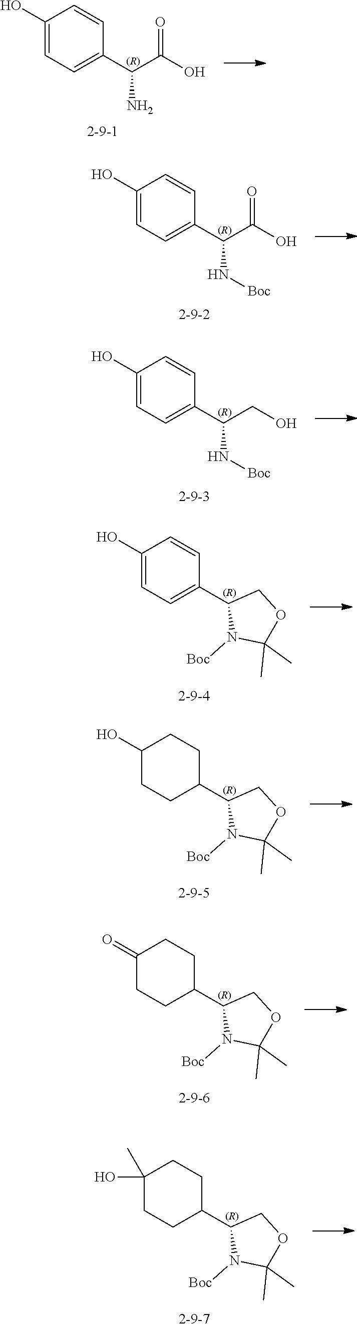

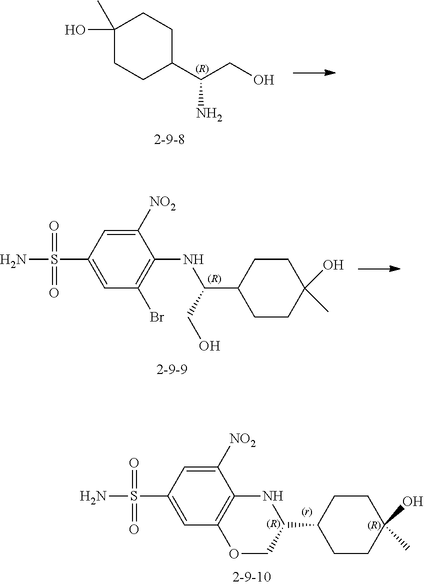

In Scheme 2-9, the starting material 2-9-1 can be prepared by conventional procedures using appropriate compounds and reagents. The starting material 2-9-1 can be converted to 2-9-3 through a two-step sequence of conventional organic reactions. After that, the intermediate 2-9-3 is protected to give 2-9-4, which can be further reduced to yield the intermediate 2-9-5. The intermediate 2-9-5 is oxidized to generate the ketone 2-9-6, which can be converted to 2-9-7 readily. The intermediate 2-9-7 is deprotected to afford 2-9-8, which can be further converted to the intermediate 2-9-9. Finally, 2-9-9 undergoes an intramolecular cyclization to afford the target compounds 2-9-10.

Also, the target compounds can be synthesized by alternative methods but not limited to the above procedures.

A typical approach to synthesize the intermediate

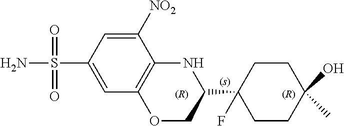

is described in Scheme 2-10 below.

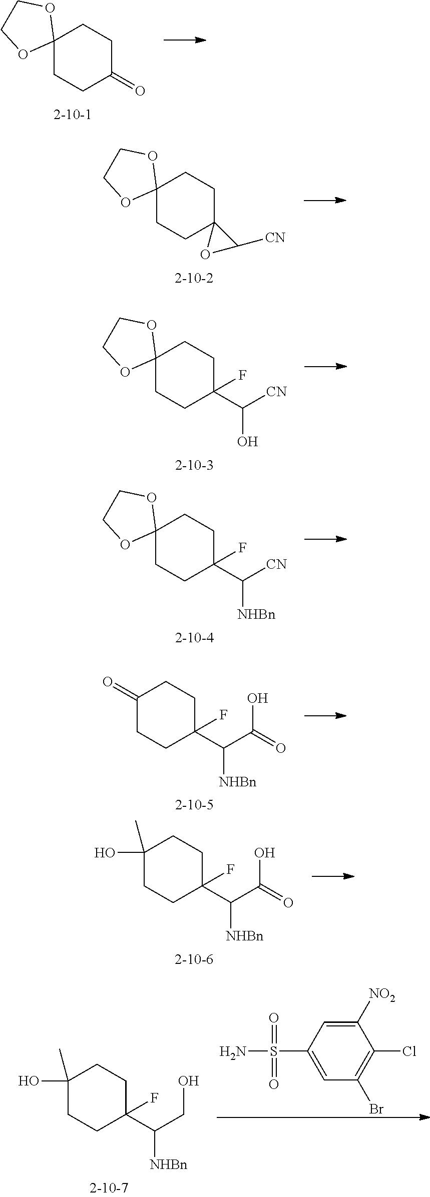

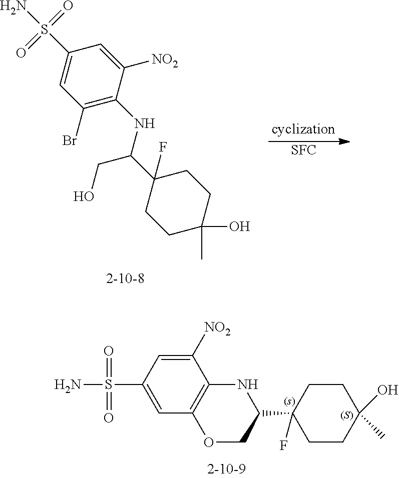

In Scheme 2-10, the starting material 2-10-1 can be prepared by conventional procedures using appropriate compounds and reagents. The starting material 2-10-1 can be converted to 2-10-3 through a two-step sequence of literate reported reactions. After that, the intermediate 2-10-3 can be converted to 2-10-4, which can be further hydrolyzed to yield the intermediate 2-10-5. The intermediate 2-10-5 can be converted to 2-10-6 readily, which is reduced to give 2-10-7. Finally, the intermediate 2-10-7 can be converted to the intermediate 2-10-8, which undergoes an intramolecular cyclization and through a further chiral separation to afford the target compounds 2-10-9.

Also, the target compounds can be synthesized by alternative methods but not limited to the above procedures.

A typical approach to synthesize the intermediate

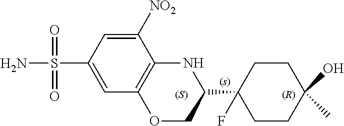

is described in Scheme 2-11 below.

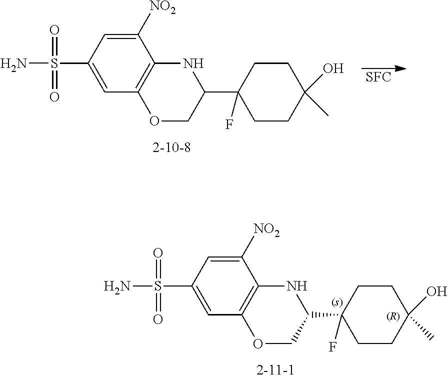

In Scheme 2-11, the starting material 2-10-8 can be prepared by conventional procedures using appropriate compounds and reagents. The starting material 2-10-8 can goes through a chiral separation to afford the target compound 2-11-1.

Also, the target compounds can be synthesized by alternative methods but not limited to the above procedures.

Similarly, the intermediate of

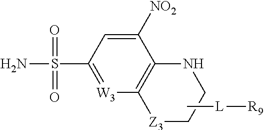

in which W₃ ith is C(Rₐ) or N; and different Z₃ and can be prepared by the method similar to the Scheme 2-2 to Scheme 2-6 by using appropriate staring materials, intermediates, and intramolecular cyclization.

Similarly, the intermediate of

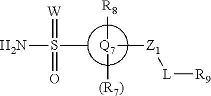

with different R₇ and Q₇ can be prepared by the method similar to the Scheme 2-1 and 2-2 by using appropriate staring materials, and intermediates.

A typical approach to synthesize the intermediate

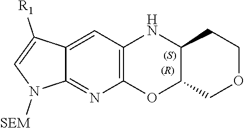

is described in Scheme 3-0-A below.

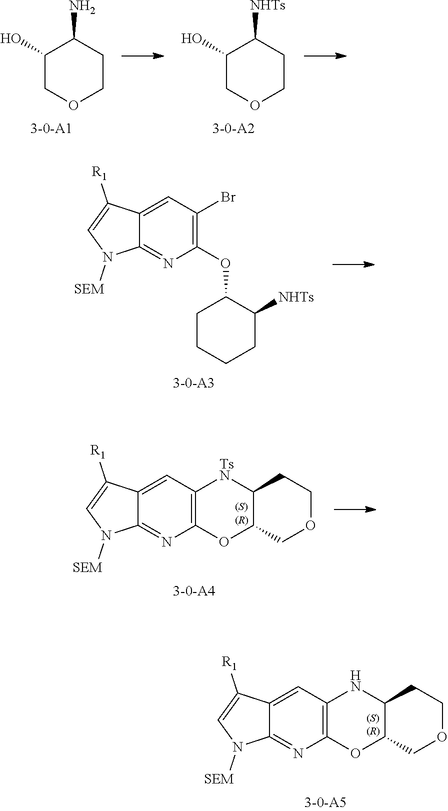

In Scheme 3-0-A, the starting material 3-0-A1 can be prepared by conventional procedures using appropriate compounds and reagents. The starting material 3-0-A1 can be converted to 3-0-A2 readily, which further undergoes a SNAr reaction to give 3-0-A3. Finally, 3-0-A3 can be converted to 3-0-A4 through an intramolecular cyclization reaction, which is deprotected to afford the target compounds 3-0-A5.

Also, the target compounds can be synthesized by alternative methods but not limited to the above procedures.

A typical approach to synthesize the intermediate

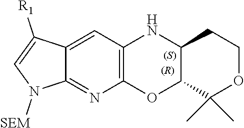

is described in Scheme 3-0-B below.

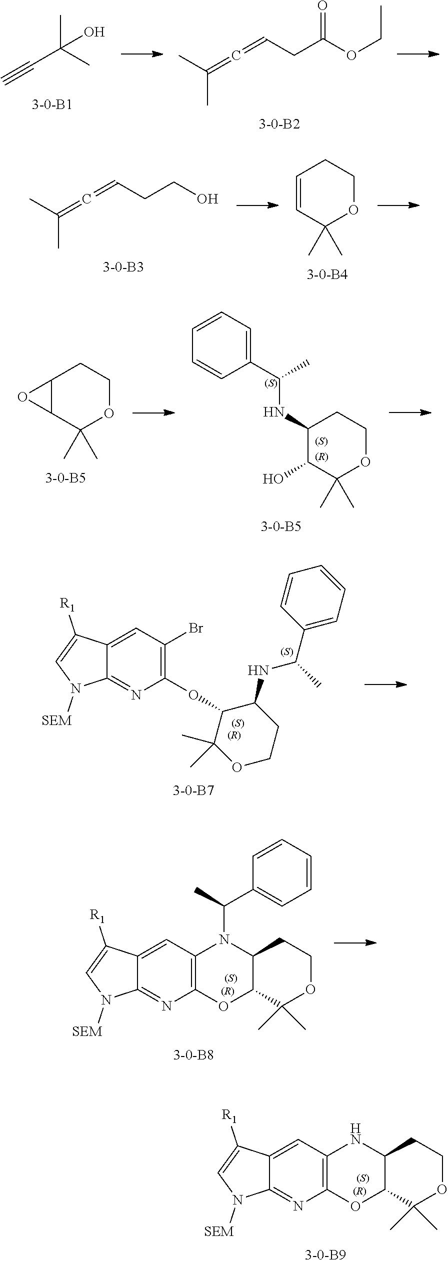

In Scheme 3-0-B, the starting material 3-0-B1 can be prepared by conventional procedures using appropriate compounds and reagents. The starting material 3-0-B1 can be converted to 3-0-B3 via a literature known reactions, which further undergoes a ring closing reaction to give 3-0-B4. After that, 3-0-B4 can be oxidized to 3-0-B5 readily, which is further converted to the intermediate 3-0-B6. Next, the intermediate 3-0-B6 undergoes a SNAr reaction to yield 3-0-B7, which can further go through an intramolecular coupling reaction to give 3-0-B8. Finally, the intermediate 3-0-B8 can be deprotected to afford the target compounds 3-0-B9.

Also, the target compounds can be synthesized by alternative methods but not limited to the above procedures.

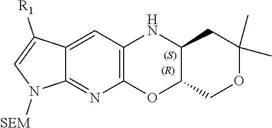

A typical approach to synthesize the intermediate SEM is described in Scheme 3-0-C below.

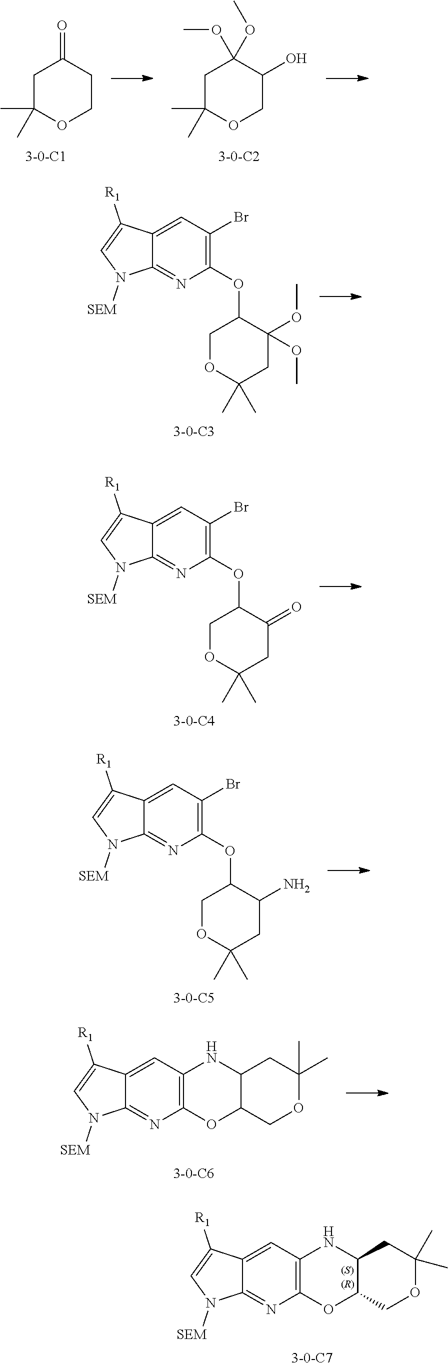

In Scheme 3-0-C, the starting material 3-0-C1 can be prepared by conventional procedures using appropriate compounds and reagents. The starting material 3-0-C1 can be converted to 3-0-C2 via a literature known reactions, which undergoes a SNAr reaction to yield 3-0-C3. After that, the intermediate 3-0-C3 is deprotected to give 3-0-C4, which can be converted to the intermediate 3-0-C5 via a reductive amination reaction. Finally, the intermediate 3-0-C5 goes through an intramolecular coupling reaction to afford the trans-racemate compounds 3-0-C6, which further go through a chiral separation to yield the target compounds 3-0-C7.

Also, the target compounds can be synthesized by alternative methods but not limited to the above procedures.

A typical approach to synthesize the intermediate

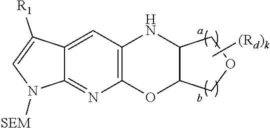

is described in Scheme 3-1 below.

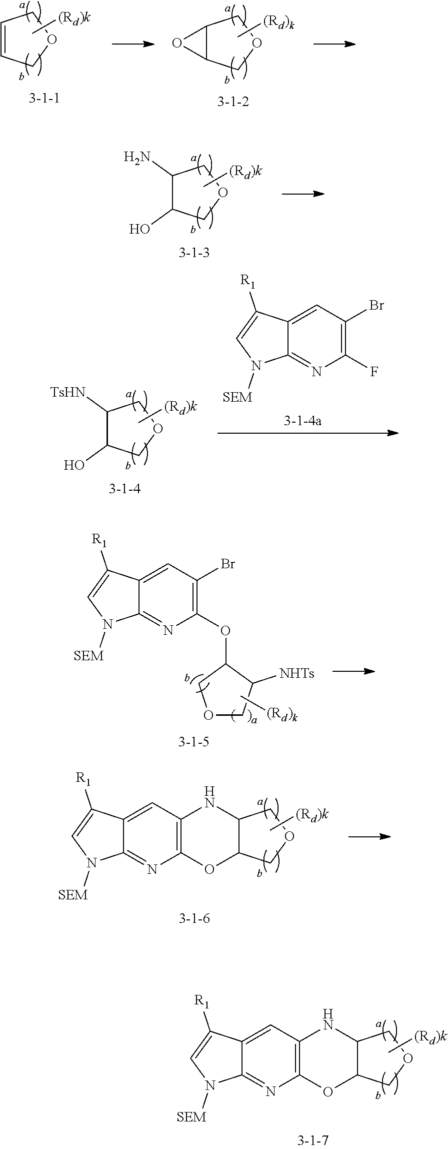

In Scheme 3-1, the starting material 3-1-1 can be prepared by conventional procedures using appropriate compounds and reagents. The starting material 3-1-1 can be converted to 3-1-2 readily, which further undergoes a ring opening reaction to give 3-1-3. After that, 3-1-3 can be converted to 3-1-4 via a general condition, and the reaction of 3-1-3 with 3-1-4a can generate intermediate 3-1-5. The intermediate 3-1-5 undergoes an intramolecular coupling reaction to give 3-1-6, which is further deprotected to afford 3-1-7.

Also, the target compounds can be synthesized by alternative methods but not limited to the above procedures.

A typical approach to synthesize the intermediate

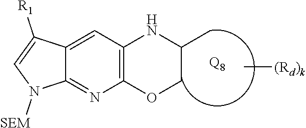

is described in Scheme 3-2 below.

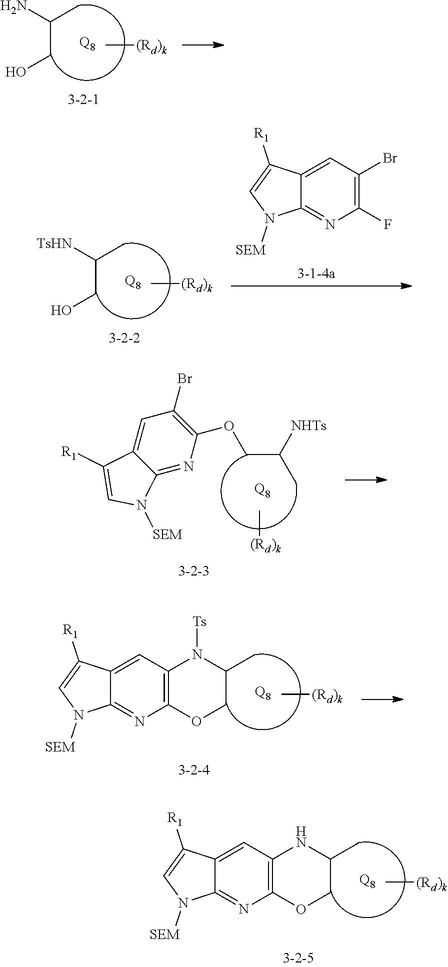

In Scheme 3-2, the starting material 3-2-1 can be prepared by conventional procedures using appropriate compounds and reagents. The starting material 3-2-1 can be converted to 3-2-2 readily, which further undergoes a SNAr reaction with 3-1-4a to give 3-2-3. After that, 3-2-3 undergoes an intramolecular coupling reaction to generate 3-2-4, which can be deprotected to afford 3-2-5.

Also, the target compounds can be synthesized by alternative methods but not limited to the above procedures.

A typical approach to synthesize the intermediate

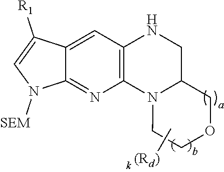

is described in Scheme 3-3 below.

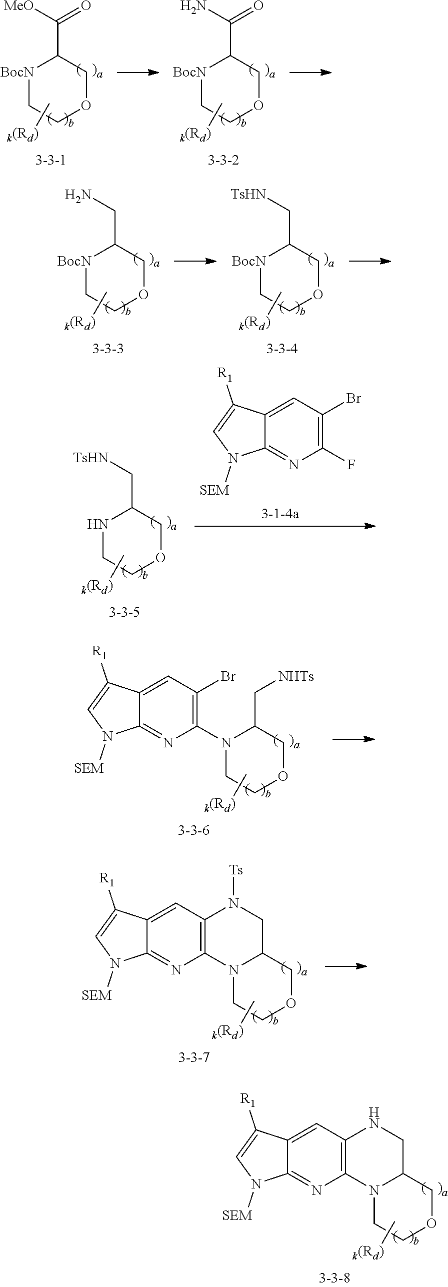

In Scheme 3-3, the starting material 3-3-1 can be converted to 3-3-3 through a two-step sequence of conventional reactions. After that, 3-3-3 can be converted to 3-3-4 readily, which can be deprotected to give 3-3-5. The reaction of 3-3-5 with 3-1-4a can generate 3-3-6, which can be converted to 3-3-7 via an intramolecular cyclization reaction. Finally, the deprotection of 3-3-7 can generate the target compounds 3-3-8.

Also, the target compounds can be synthesized by alternative methods but not limited to the above procedures.

A typical approach to synthesize the intermediate

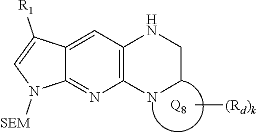

is described in Scheme 3-4 below.

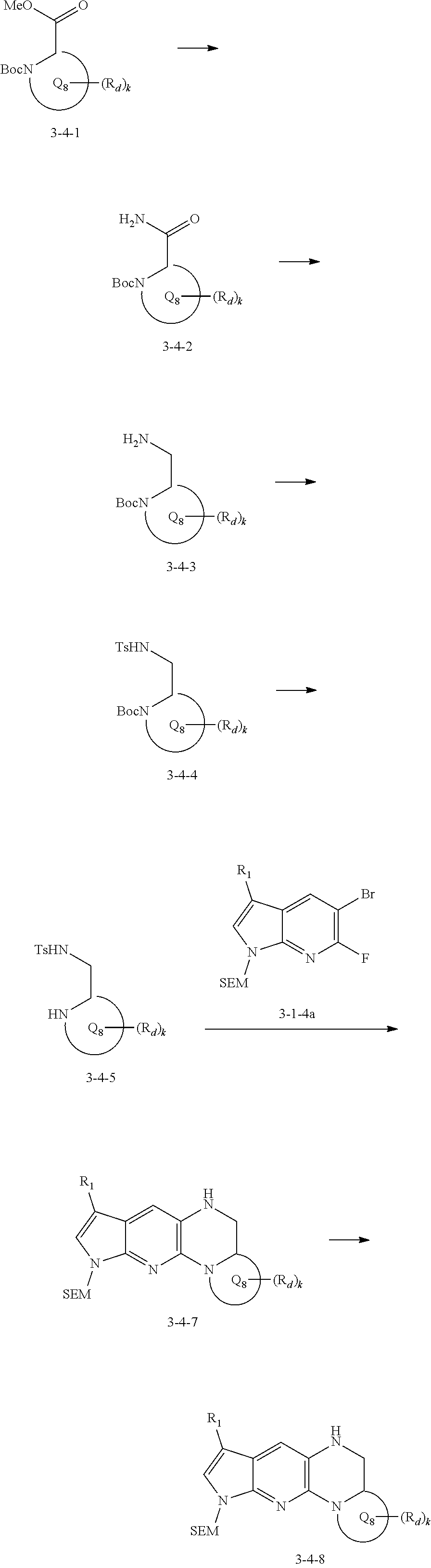

In Scheme 3-4, the starting material 3-4-1 can be converted to 3-4-3 through a two-step sequence of conventional reactions. After that, 3-4-3 can be converted to 3-4-4 readily, which can be deprotected to give 3-4-5. The reaction of 3-4-5 with 3-1-4a can generate 3-4-6, which can be converted to 3-4-7 via an intramolecular cyclization reaction. Finally, the deprotection of 3-4-7 can generated the target compounds 3-4-8.

Also, the target compounds can be synthesized by alternative methods but not limited to the above procedures.

A typical approach to synthesize the intermediate

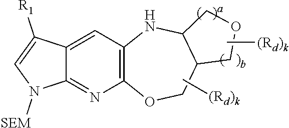

is described in Scheme 3-5 below.

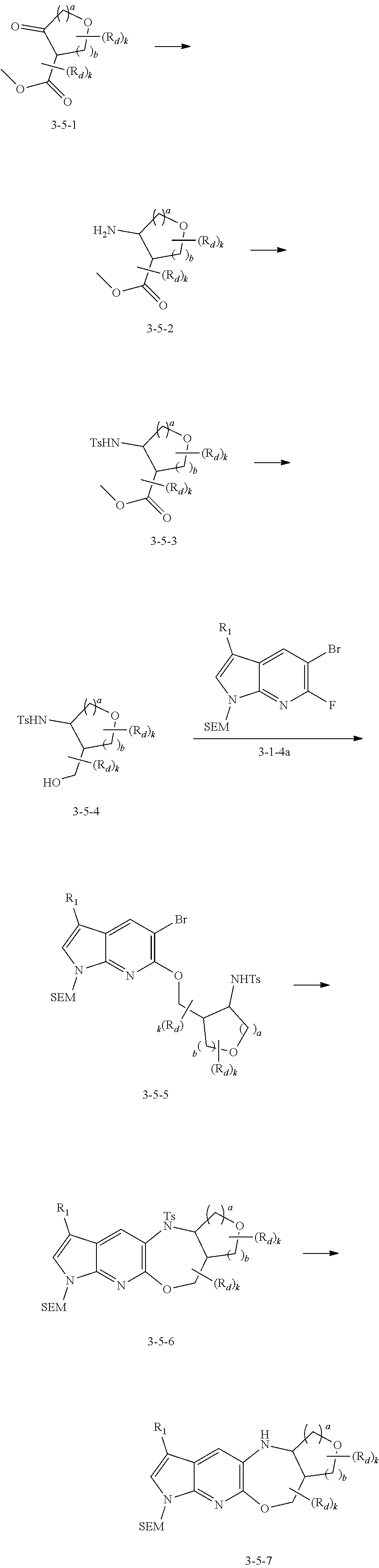

In Scheme 3-5, the starting material 3-5-1 can be converted to 3-5-2 via reductive amination reaction, and then 3-5-2 can be converted to 3-5-3 readily. After that, 3-5-3 can be reduced to 3-5-4. The reaction of 3-5-4 with 3-1-4a can generate 3-5-5, which can be converted to 3-5-6 via an intramolecular cyclization reaction. Finally, the deprotection of 3-5-6 can generate the target compounds 3-5-7.

Also, the target compounds can be synthesized by alternative methods but not limited to the above procedures.

A typical approach to synthesize the intermediate

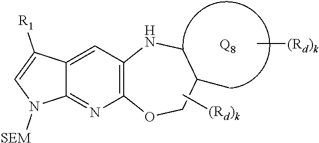

is described in Scheme 3-6 below.

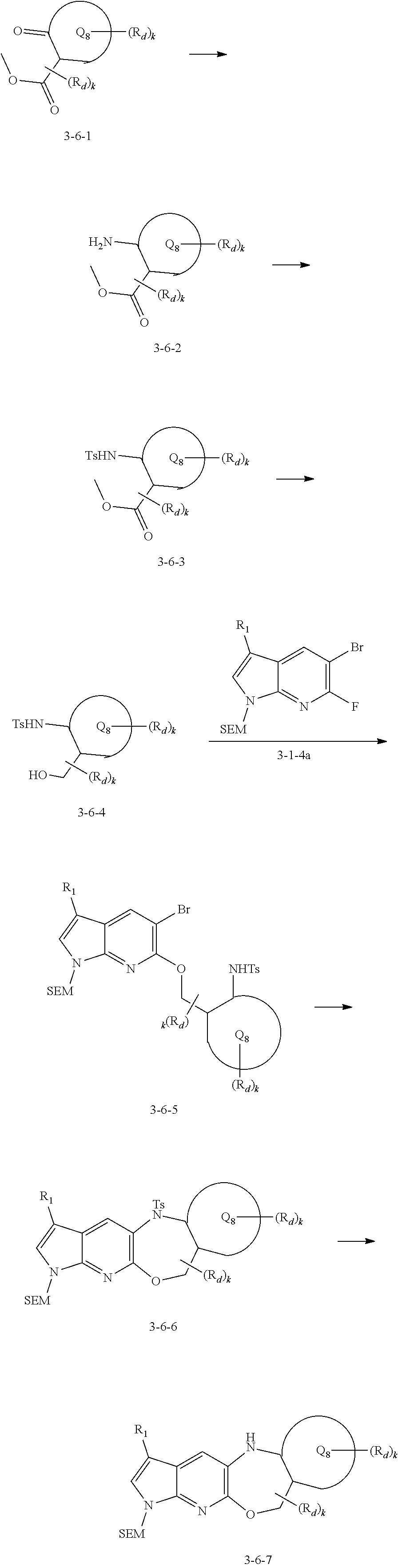

In Scheme 3-6, the starting material 3-6-1 can be converted to 3-6-2 via reductive amination reaction, and then 3-6-2 can be converted to 3-6-3 readily. After that, 3-6-3 can be reduced to 3-6-4. The reaction of 3-6-4 with 3-1-4a can generate 3-6-5, which can be converted to 3-6-6 via an intramolecular cyclization reaction. Finally, the deprotection of 3-6-6 can generate the target compounds 3-6-7.

Also, the target compounds can be synthesized by alternative methods but not limited to the above procedures.

A typical approach to synthesize the intermediate

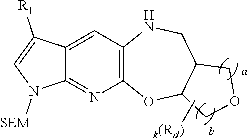

is described in Scheme 3-7 below.

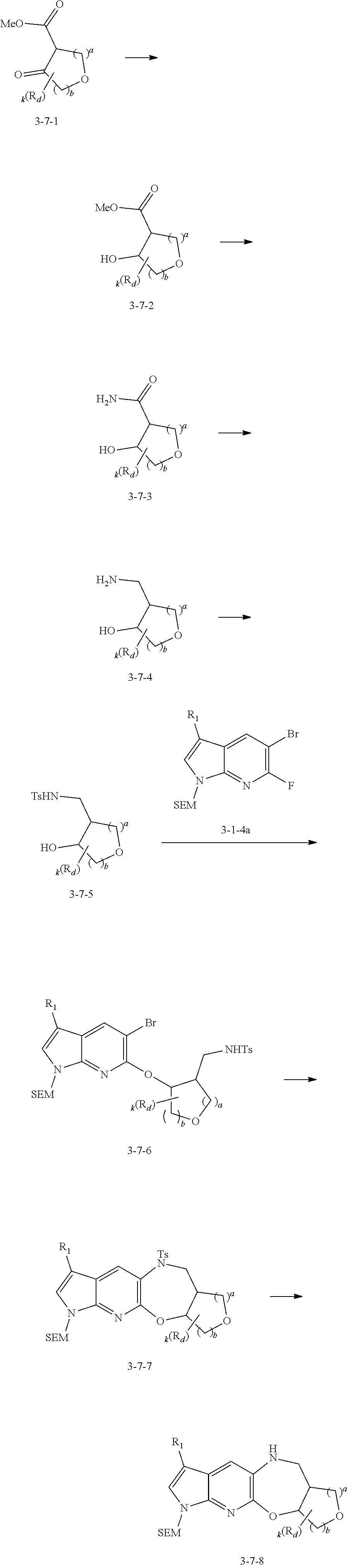

In Scheme 3-7, the starting material 3-7-1 can be reduced to give 3-7-2, and then 3-7-2 can be converted to 3-7-3 readily. After that, 3-7-3 can be converted to 3-7-5 through a two-step sequence of conventional reactions. The reaction of 3-7-5 with 3-1-4a can generate 3-7-6, which can be converted to 3-7-7 via an intramolecular cyclization reaction. Finally, the deprotection of 3-7-7 can generate the target compounds 3-7-8.

Also, the target compounds can be synthesized by alternative methods but not limited to the above procedures.

A typical approach to synthesize the intermediate

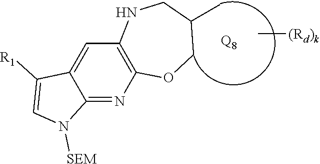

is described in Scheme 3-8 below.

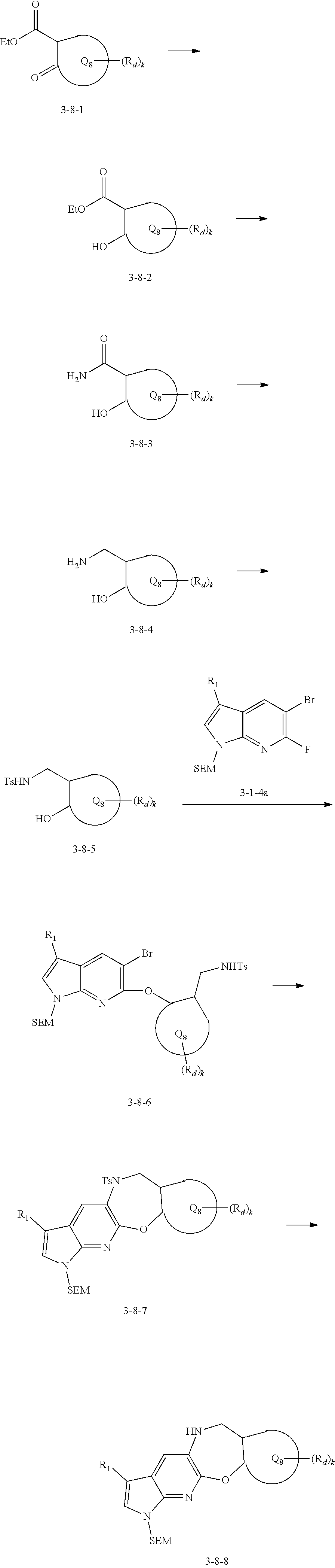

In Scheme 3-8, the starting material 3-8-1 can be reduced to give 3-7-2, and then 3-8-2 can be converted to 3-8-3 readily. After that, 3-8-3 is further converted to 3-8-5 through a two-step sequence of conventional reactions. The reaction of 3-8-5 with 3-1-4a can generate 3-8-6, which can be converted to 3-8-7 via an intramolecular cyclization reaction. Finally, the deprotection of 3-7-7 can generate the target compounds 3-8-8.

Also, the target compounds can be synthesized by alternative methods but not limited to the above procedures.

A typical approach to synthesize the intermediate

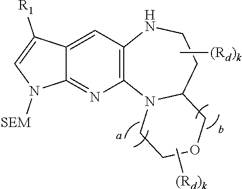

is described in Scheme 3-9

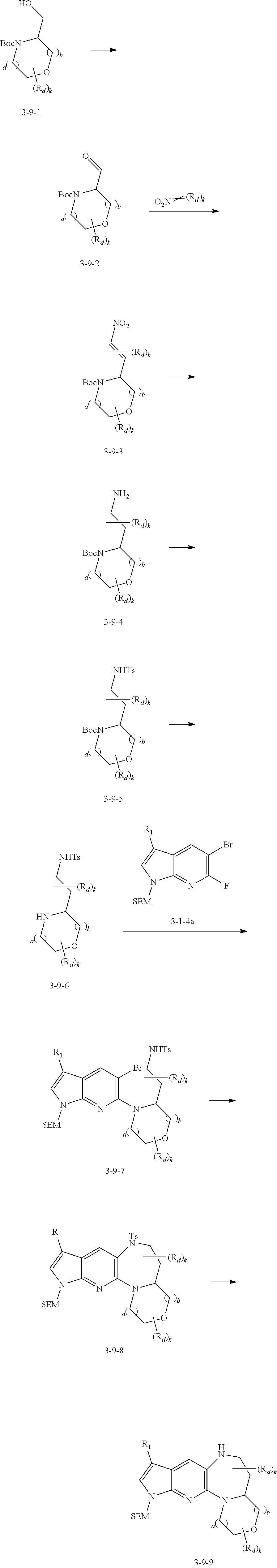

In Scheme 3-9, the starting material 3-9-1 can be oxidized to give 3-9-2, and then 3-9-2 can be converted to 3-9-3 readily. After that, the hydrogenation of 3-9-3 can give 3-9-4, which is further converted to 3-9-6 through a two-step sequence of conventional reactions. The reaction of 3-9-6 with 3-1-4a can generate 3-9-7, which can be converted to 3-9-8 via an intramolecular cyclization reaction. Finally, the deprotection of 3-9-8 can generate the target compounds 3-9-9.

Also, the target compounds can be synthesized by alternative methods but not limited to the above procedures.

A typical approach to synthesize the intermediate

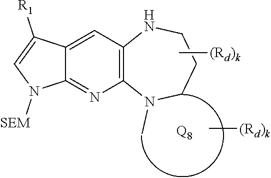

is described in Scheme 3-10 below.

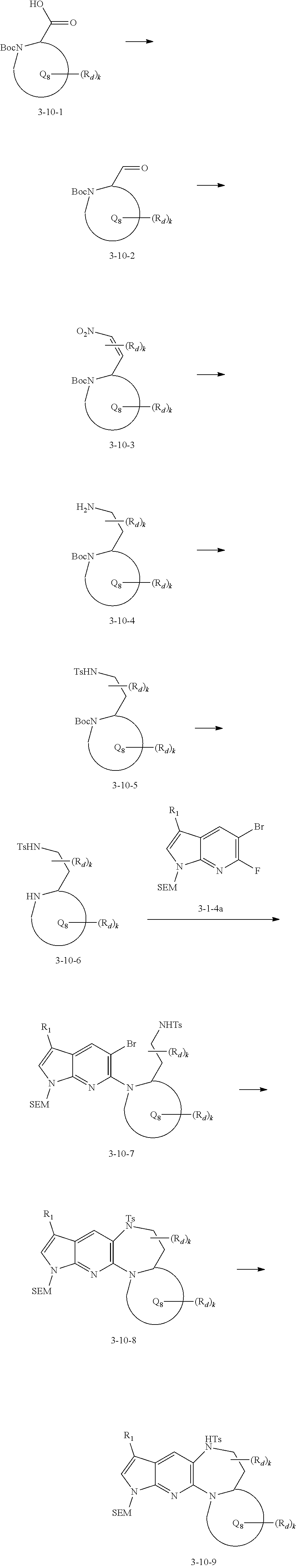

In Scheme 3-10, the starting material 3-10-1 can be reduced to give 3-10-2, and then 3-10-2 can be converted to 3-10-3 readily. After that, the hydrogenation of 3-10-3 can give 3-10-4, which can be converted to 3-10-6 through a two-step sequence of conventional reactions. The reaction of 3-10-6 with 3-1-4a can generate 3-10-7, which can be converted to 3-10-8 via an intramolecular cyclization reaction. Finally, the deprotection of 3-10-8 can generate the target compounds 3-10-9.

Also, the target compounds can be synthesized by alternative methods but not limited to the above procedures.

Similarly, the intermediate of

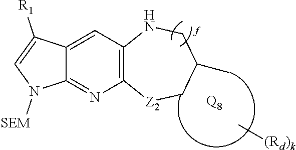

with different Z₂, f, k, R_{d} and Q₈ can be prepared by the method similar to the Scheme 3-1 to 3-10 by using appropriate staring materials, and intermediates.

Similarly, the compounds of

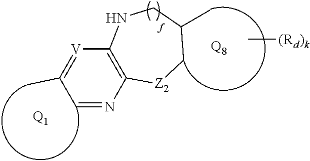

can be prepared by schemes similar to the Scheme 3-1 to 3-10 by using appropriate staring materials and intermediates.

An approach to synthesize compounds of

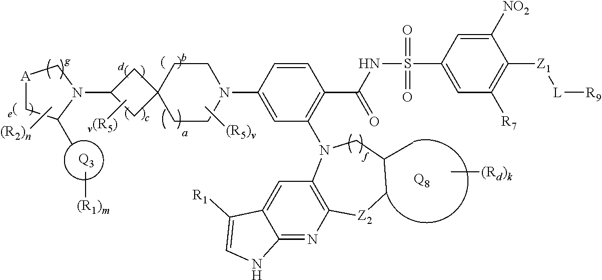

is described in Scheme A:

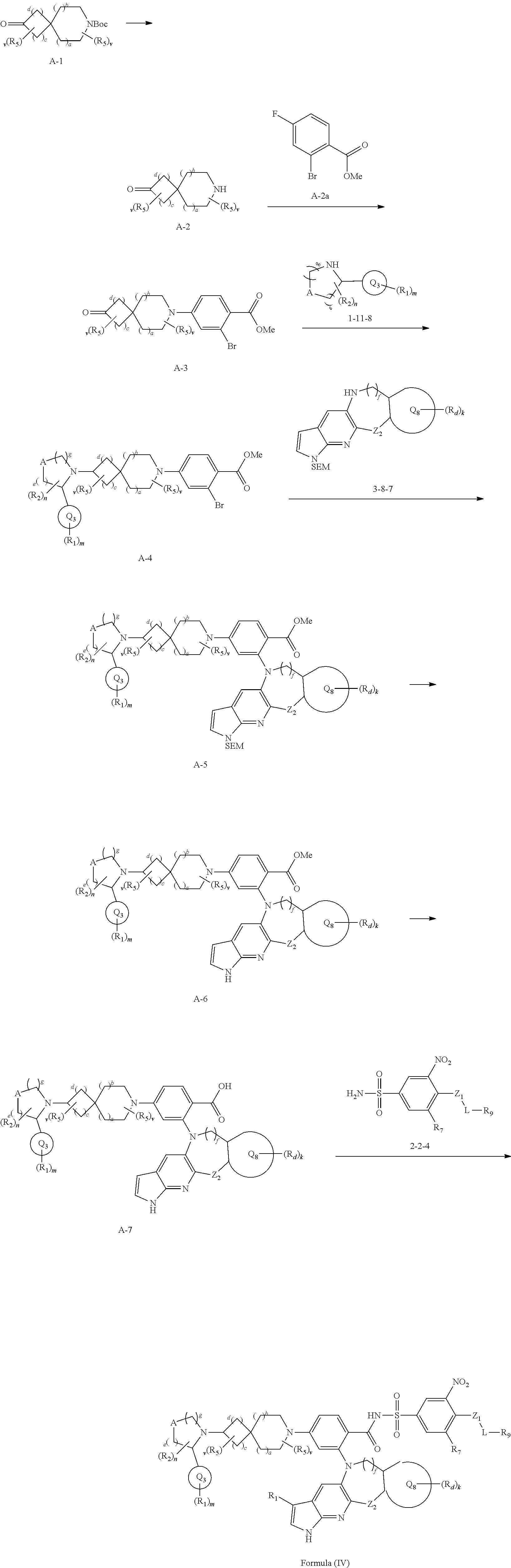

In Scheme A, the starting material A-1 can be prepared by conventional procedures using appropriate compounds and reagents. The starting material A-1 can be converted to A-2 readily, which further undergoes a SNAr reaction with A-2a to give A-3. After that, the intermediate A-3 can be converted to A-4 via a reductive amination reaction with 1-11-8, and A-4 undergoes a coupling reaction with 3-8-7 to give A-5. Next, the deprotection of A-5 can afford A-6, which can be hydrolyzed to generate A-7. Finally, A-7 undergoes a condensation reaction with 2-2-4 to afford the target compounds.

Also, the target compounds can be synthesized by alternative methods but not limited to the above procedures.

An approach to synthesize compounds of

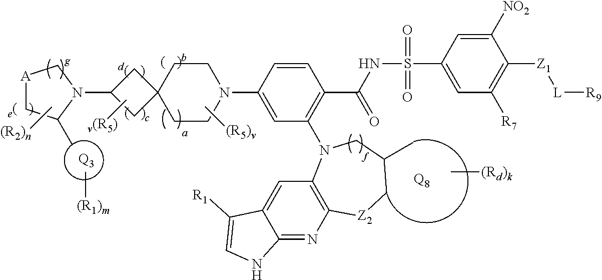

is described in Scheme B:

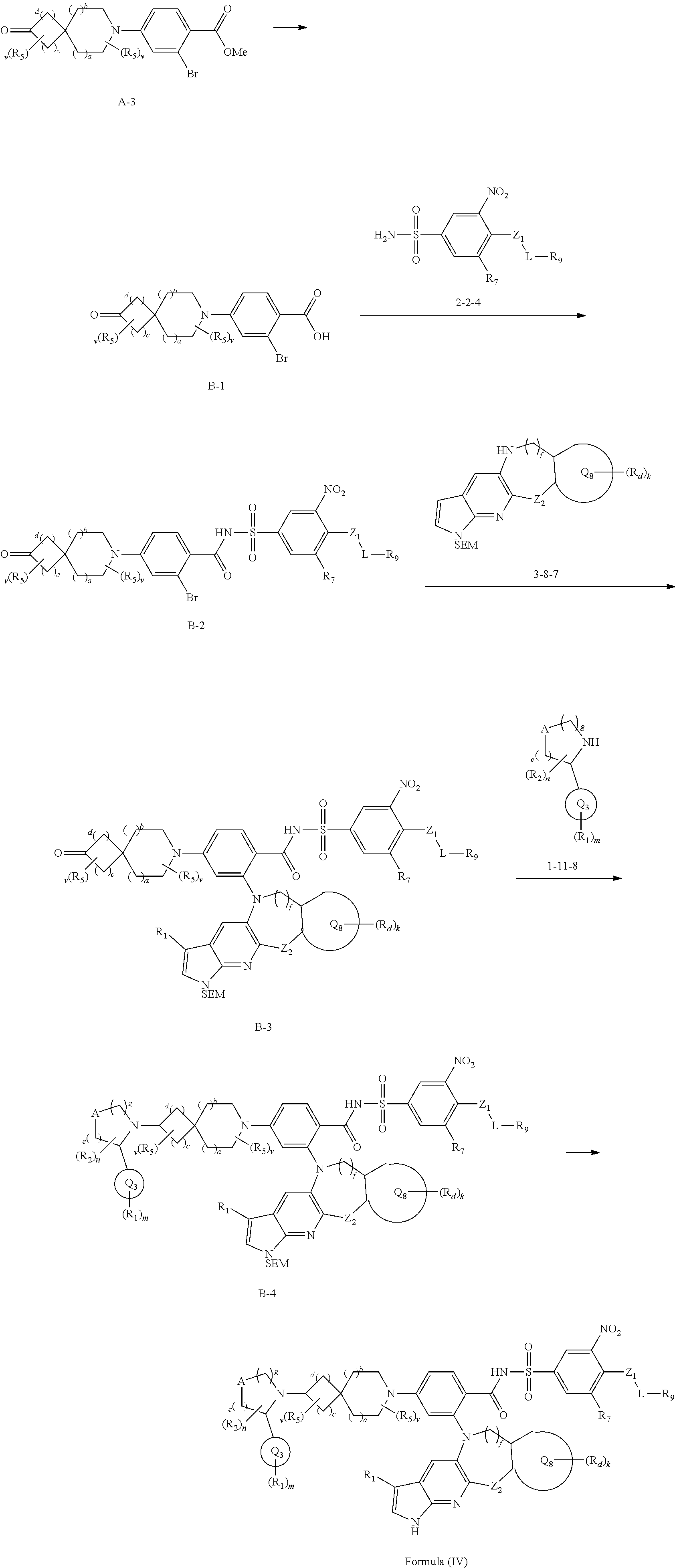

In Scheme B, the starting material A-3 can be prepared by conventional procedures using appropriate compounds and reagents. The starting material A-3 can be hydrolyzed to generate B-1 readily. After that, the intermediate B-1 undergoes a condensation reaction with 2-2-4 to give B-2, which further couples with 3-8-7 to give B-3. The intermediate B-3 can be converted to B-4 via a reductive amination reaction with 1-11-8. Finally, the deprotection of B-4 can generate the target compounds.

Also, the target compounds can be synthesized by alternative methods but not limited to the above procedures.

An approach to synthesize compounds of

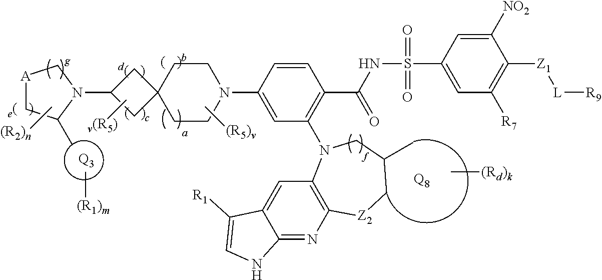

is described in Scheme C:

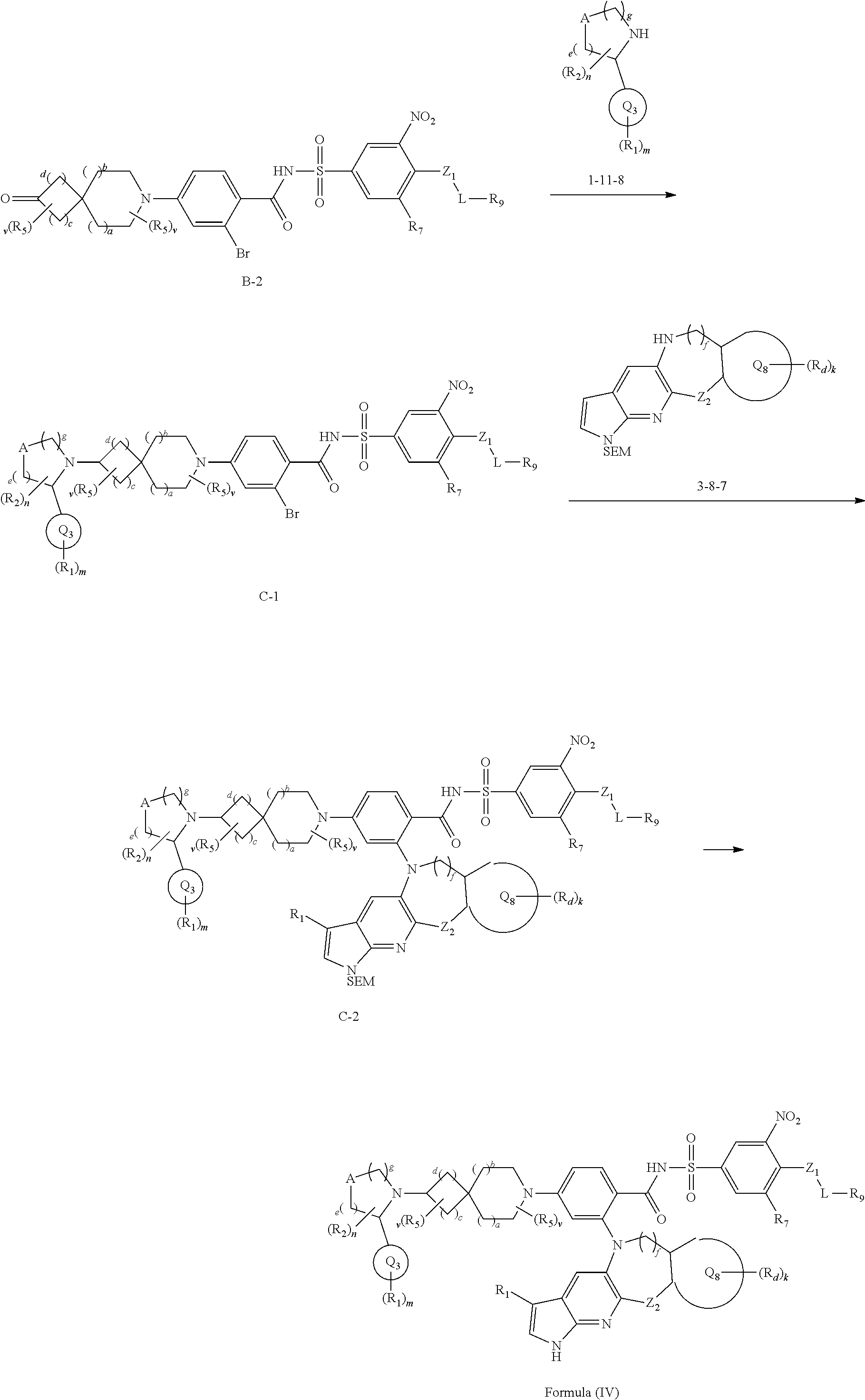

In Scheme C, the starting material B-2 can be prepared by conventional procedures using appropriate compounds and reagents. The starting material B-2 can be converted to C-1 via a reductive amination reaction with 1-11-8, and then C-1 can undergoes a coupling reaction with 3-8-7 to give C-2. Finally, the deprotection of C-2 can generate the target compounds.

Also, the target compounds can be synthesized by alternative methods but not limited to the above procedures.

An approach to synthesize compounds of

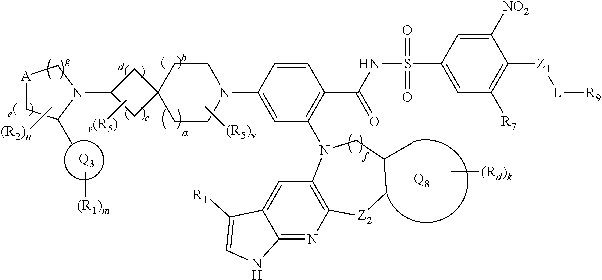

is described in Scheme D:

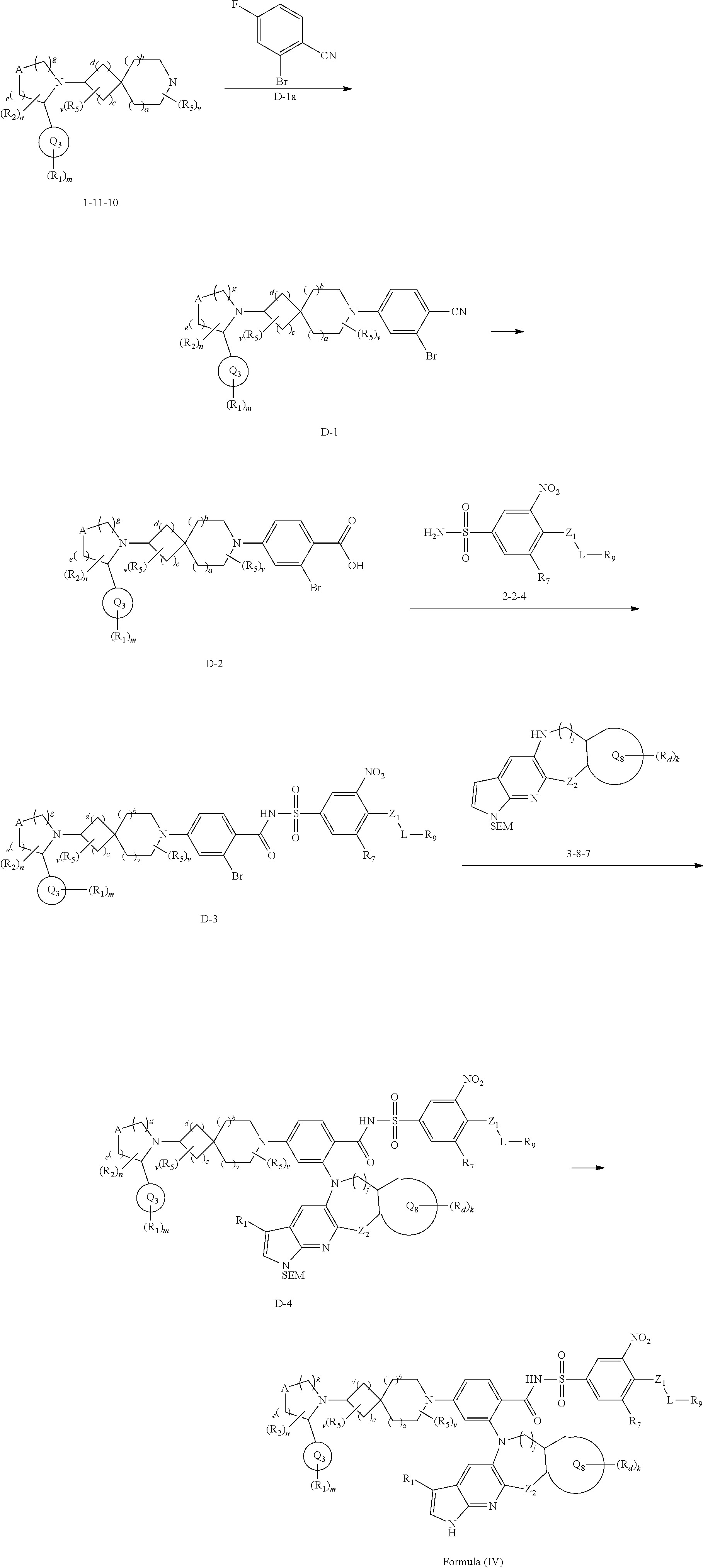

In Scheme D, the starting material 1-11-10 can be prepared by conventional procedures using appropriate compounds and reagents. The starting material 1-11-10 can be converted to D-1 readily, which can be hydrolyzed to generate D-2. After that, the intermediate D-2 undergoes a condensation reaction with 2-2-4 to give D-3, which further couples with 3-8-7 to give D-4. Finally, the deprotection of D-4 can give the target compounds.

Also, the target compounds can be synthesized by alternative methods but not limited to the above procedures.

An approach to synthesize compounds of

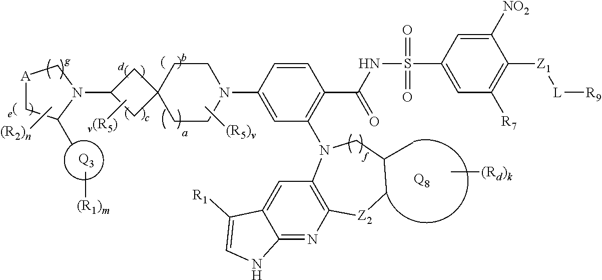

is described in Scheme E:

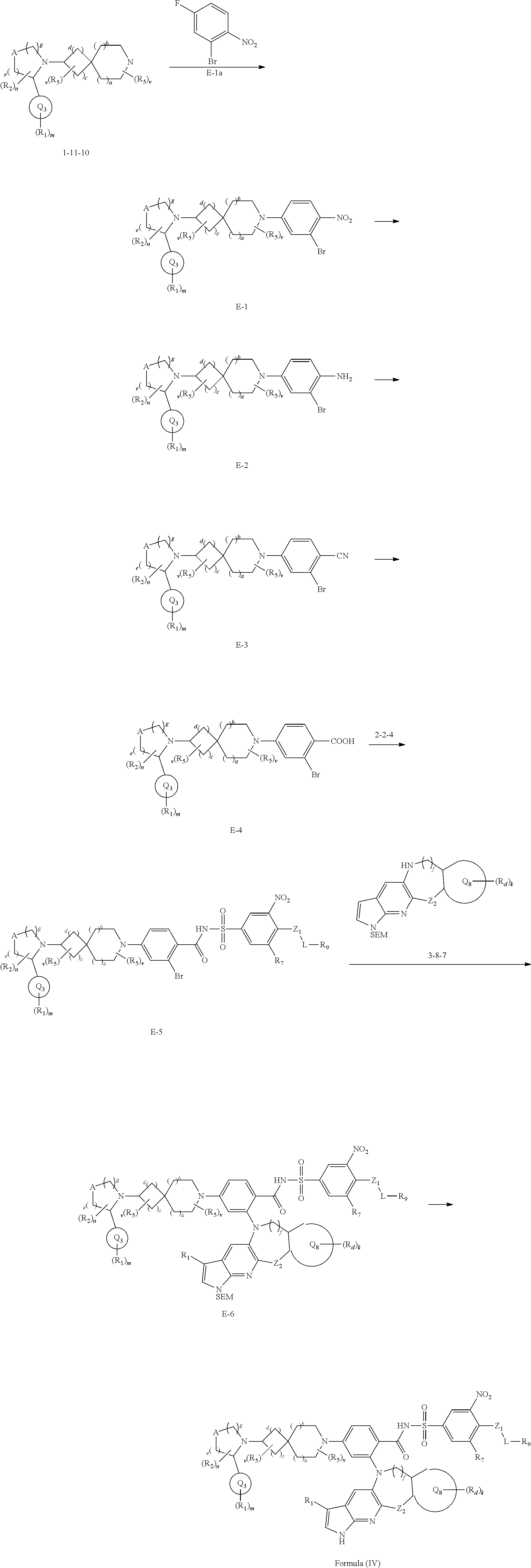

In Scheme E, the starting material 1-11-10 can be prepared by conventional procedures using appropriate compounds and reagents. The starting material 1-11-10 can be converted to E-1 readily, which can be reduced to generate E-2. After that, the intermediate E-2 undergoes a Sandmeyer reaction to give E-3, which can be hydrolyzed to generate E-4. The condensation of intermediate E-4 with 2-2-4 can give E-5. Next, E-5 couples with 3-8-7 to give E-6. Finally, the deprotection of E-6 can give the target compounds.

Also, the target compounds can be synthesized by alternative methods but not limited to the above procedures.

An approach to synthesize compounds of

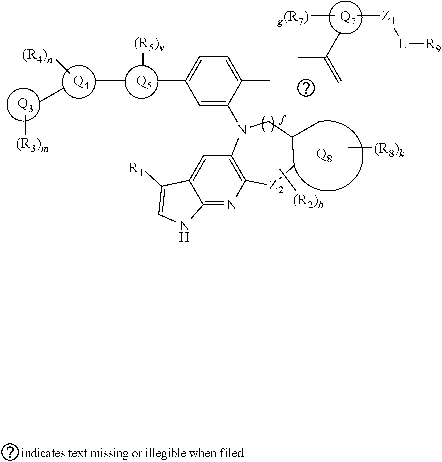

is described in Scheme F:

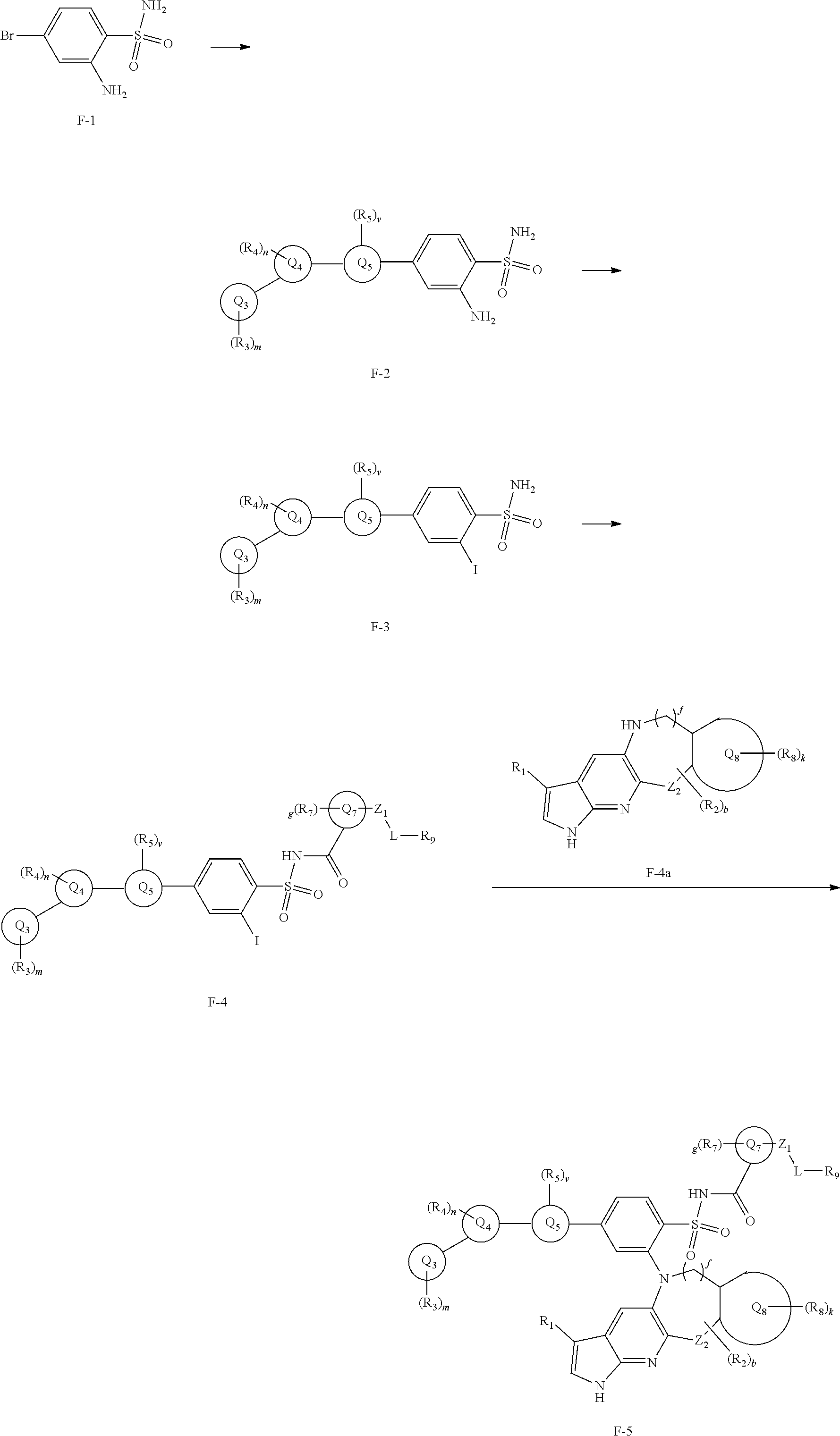

In Scheme F, the starting material F-1 can be prepared by conventional procedures using appropriate compounds and reagents. The starting material F-1 can be converted to F-2 via a coupling reaction, which is further converted to F-3 through Sandmeyer reaction. After that, the condensation of intermediate F-3 with the corresponding carboxylic acid can give F-4. Finally, the intermediate F-4 can couple with F-4a to generate the target compounds F-5.

Also, the target compounds can be synthesized by alternative methods but not limited to the above procedures.

An approach to synthesize of target compounds of

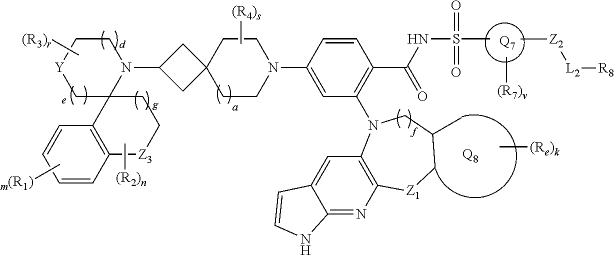

is described in Scheme G:

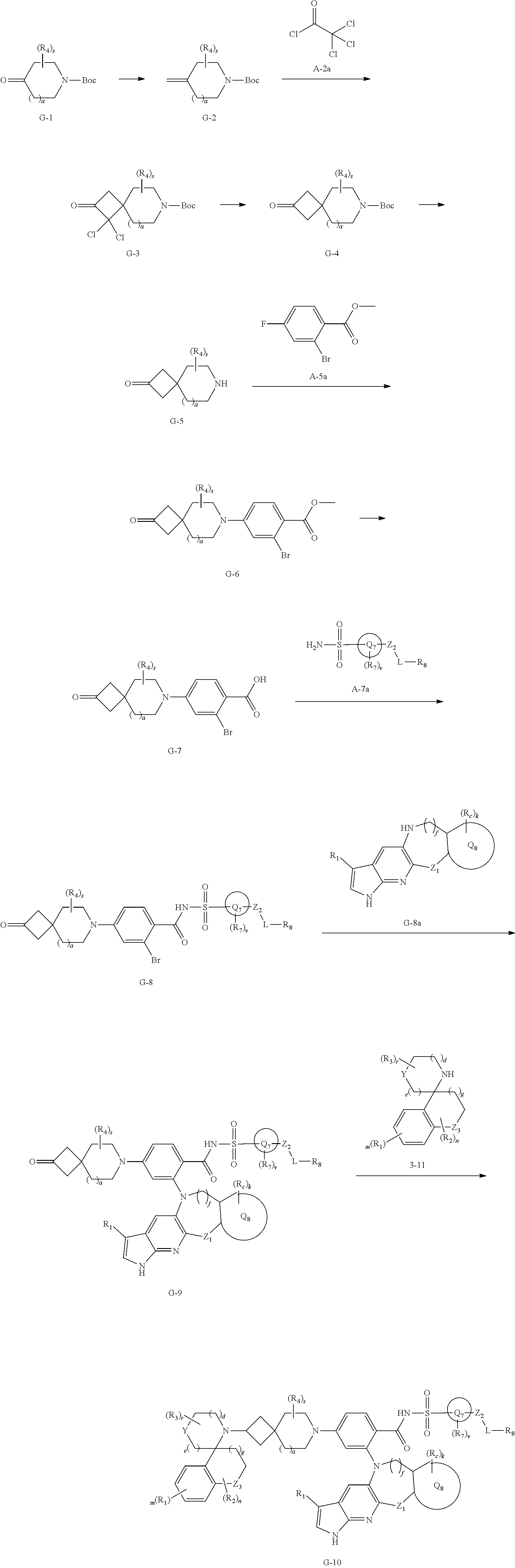

In Scheme G, the starting material G-1 can be prepared by conventional procedures using appropriate compounds and reagents. The starting material G-1 can be converted to G-2 via a general Wittig reaction, which further undergoes Staudinger ketene cycloaddition with G-2a to give G-3. After that, the dichloride G-3 can be reducted to G-4 by a suitable reagent, and then deprotection of G-4 can generate intermediate G-5. The intermediate G-5 undergoes a coupling reaction with G-5a to give G-6, which can be hydrolyzed to afford G-7. Next, the intermediate G-7 can react with G-7a to give G-8, which can couple with G-8a to give G-9. Finally, G-9 can undergo a reductive amination with 3-11 to afford the target compounds G-10.

Also, the target compounds can be synthesized by alternative methods but not limited to the above procedures.

An approach to synthesize of target compounds of

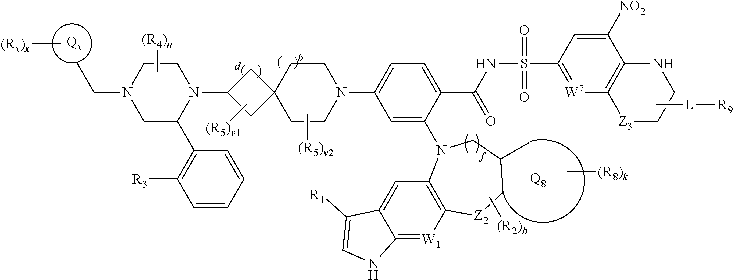

is described in Scheme H:

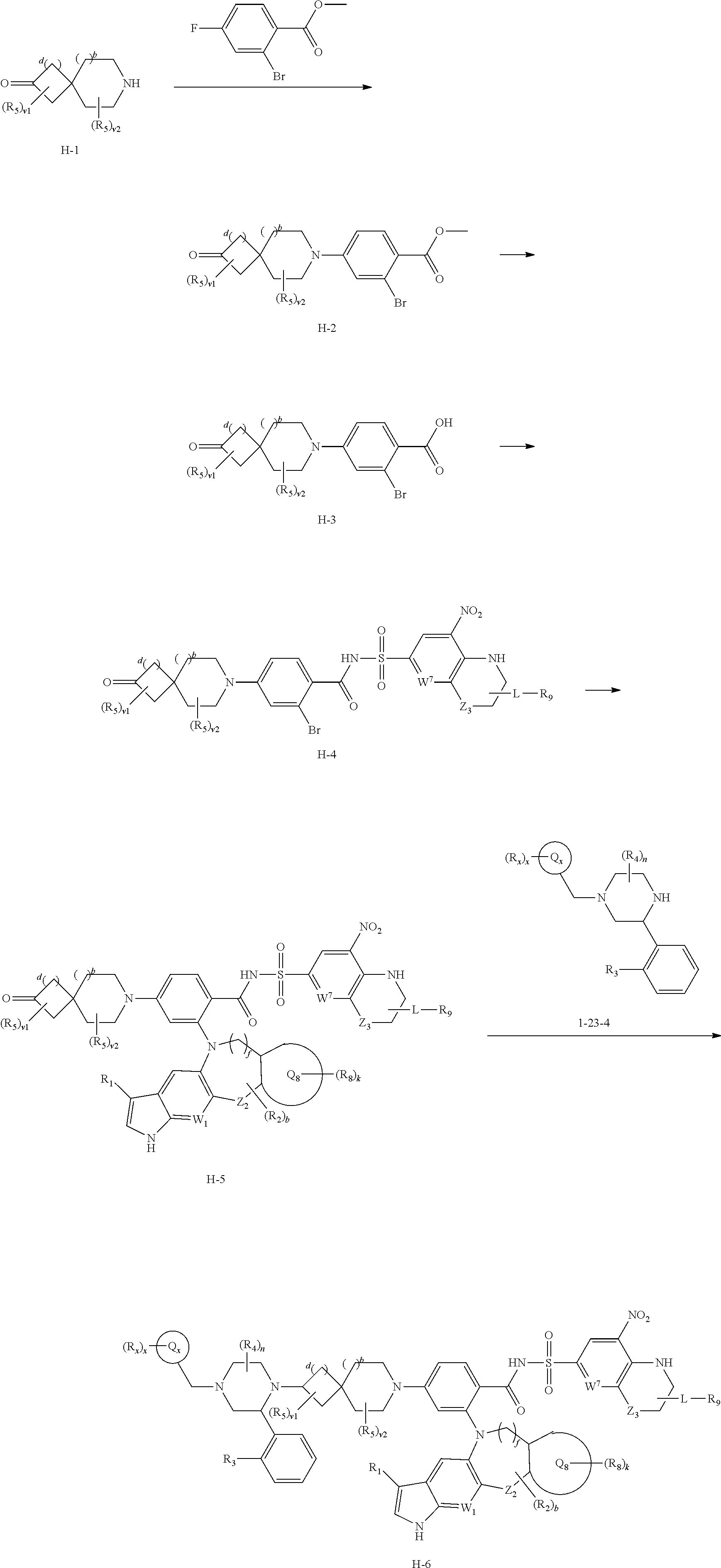

In Scheme H, the starting material H-1 can be prepared by conventional procedures using appropriate compounds and reagents. The starting material H-1 can be converted to H-2 via a SNAr reaction, which is hydrolyzed to afford H-3. After that, the intermediate H-3 can be converted to H-4 readily, which goes through a Buchwald coupling reaction to give H-5. Finally, H-5 react with 1-23-4 via a reductive amination reaction to afford the target compounds H-6.

Also, the target compounds can be synthesized by alternative methods but not limited to the above procedures.

Similarly, the compounds of

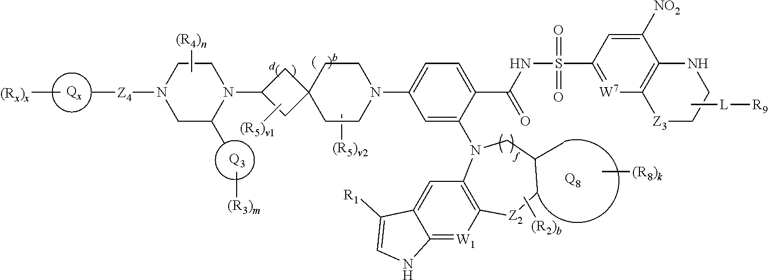

can be prepared by schemes similar to the Scheme A-Scheme H by using appropriate staring materials and intermediates.

Similarly, the compounds of

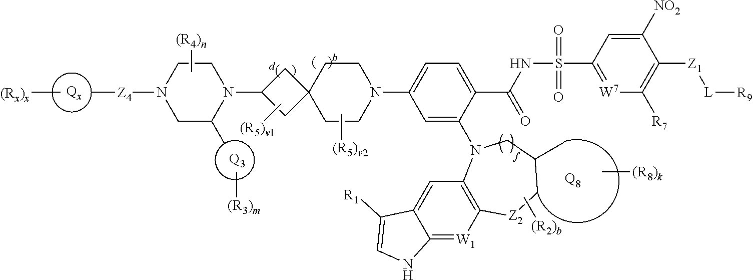

can be prepared by schemes similar to the Scheme A-Scheme H by using appropriate staring materials and intermediates.

Similarly, the compounds of

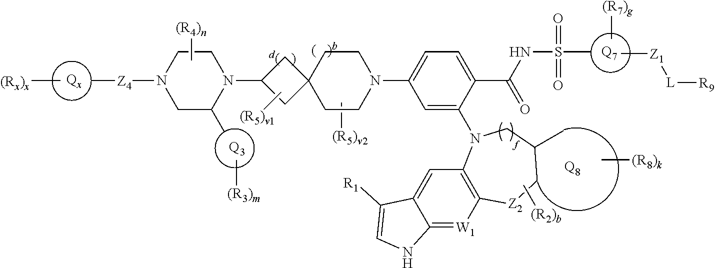

can be prepared by schemes similar to the Scheme A-Scheme H by using appropriate staring materials and intermediates.

Similarly, the compounds of

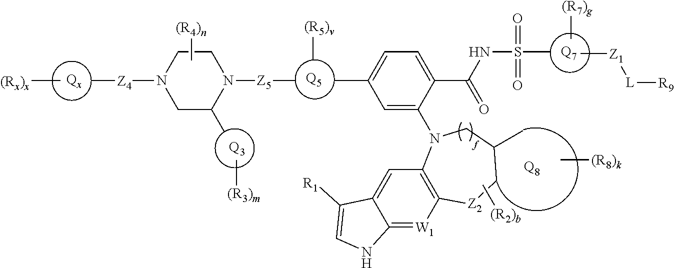

can be prepared by schemes similar to the Scheme A-Scheme H by using appropriate staring materials and intermediates.

Similarly, the compounds of

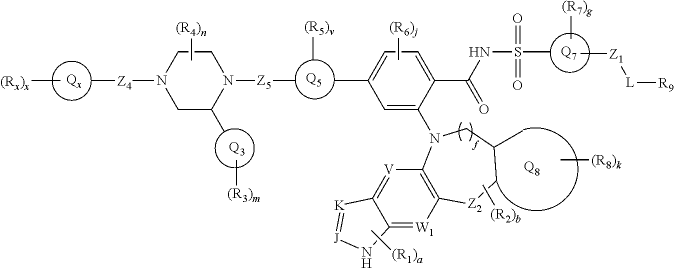

can be prepared by schemes similar to the Scheme A-Scheme H by using appropriate staring materials and intermediates.

Similarly, the compounds of

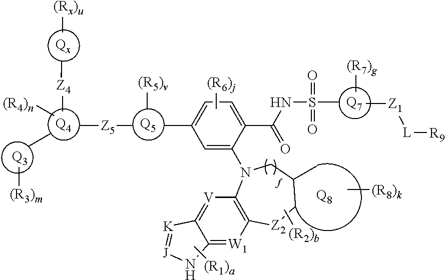

can be prepared by schemes similar to the Scheme A-Scheme H by using appropriate staring materials and intermediates.

Similarly, the compounds of

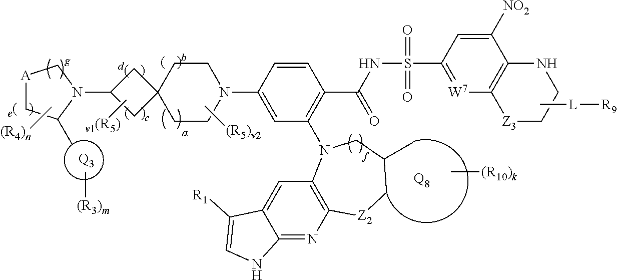

can be prepared by schemes similar to the Scheme A-Scheme H by using appropriate staring materials and intermediates.

Similarly, the compounds of

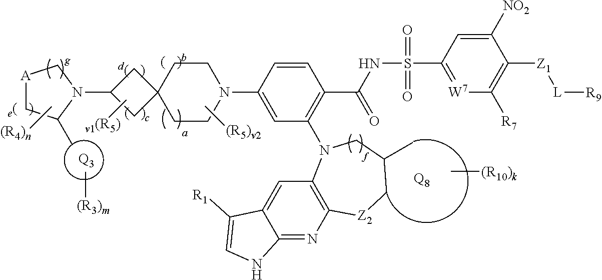

can be prepared by schemes similar to the Scheme A-Scheme H by using appropriate staring materials and intermediates.

Similarly, the compounds of

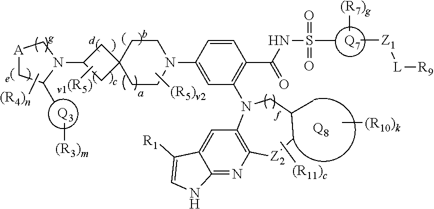

can be prepared by schemes similar to the Scheme A-Scheme H by using appropriate staring materials and intermediates.

Similarly, the compounds of

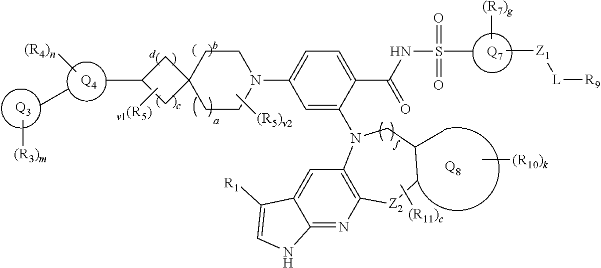

can be prepared by schemes similar to the Scheme A-Scheme H by using appropriate staring materials and intermediates.

Similarly, the compounds of

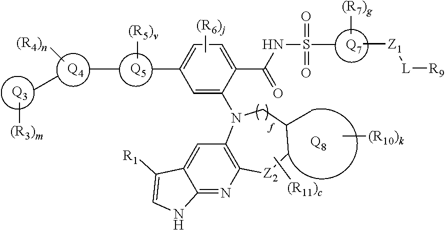

can be prepared by schemes similar to the Scheme A-Scheme H by using appropriate staring materials and intermediates.

Similarly, the compounds of

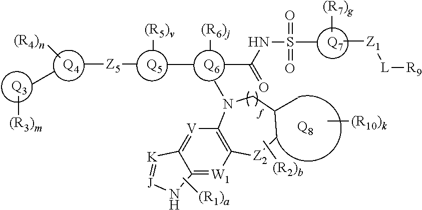

a can be prepared by schemes similar to the Scheme A-Scheme H by using appropriate staring materials and intermediates.

Similarly, the compounds of

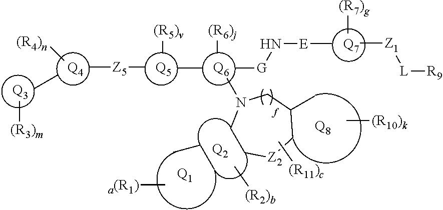

can be prepared by schemes similar to the Scheme A-Scheme H by using appropriate staring materials and intermediates.

The compounds and processes of the present invention will be better understood in connection with the following examples, which are intended as an illustration only and not limiting of the scope of the invention. Various changes and modifications to the disclosed embodiments will be apparent to those skilled in the art and such changes and modifications including, without limitation, those relating to the chemical structures, substituents, derivatives, formulations and/or methods of the invention may be made without departing from the spirit of the invention and the scope of the appended claims.

Where NMR data are presented, ¹H spectra were obtained on XL400 (400 MHz) and are reported as ppm down field from Me₄Si with number of protons, multiplicities, and coupling constants in Hertz indicated parenthetically. Where HPLC data are presented, analyses were performed using an Agilent 1100 system. Where LC/MS data are presented, analyses were performed using an Applied Biosystems API-100 mass spectrometer and Shimadzu SCL-10A LC column:

### Example INT_33 Preparation of (3R)-3-(2-isopropoxyphenyl)morpholine and (3S)-3-(2-isopropoxyphenyl)morpholine

Synthesis of 2-isopropoxybenzaldehyde: Into a 2 L round-bottom flask, were placed 2-hydroxybenzaldehyde (100.0 g, 819.7 mmol, 1.0 eq), 2-iodopropane (181.1 g, 1.1 mol, 1.3 eq), K₂CO₃ (226.0 g, 1.6 mol, 2.0 eq), N, N-dimethylformamide (1.0 L). The reaction mixture was stirred overnight at 60° C. The resulting mixture was quenched by the addition of water (2 L) and extracted with EtOAc (2×1.0 L). The combined organic layer was washed with brine (500 mL), dried over anhydrous Na₂SO₄. After filtration, the filtrate was concentrated under vacuum. The crude residue was applied onto a silica gel column and eluted with ethyl acetate/petroleum ether=1:5 to give 2-isopropoxybenzaldehyde as a brown oil (120.0 g, 89.4%). ¹HNMR (300 MHz, DMSO-d₆) δ 10.39 (s, 1H), 7.69 (dd, J=7.8, 1.8 Hz, 1H), 7.60 (ddd, J=8.4, 7.2, 1.8 Hz, 1H), 7.19 (dd, J=8.4, 0.9 Hz, 1H), 7.02 (ddd, J=7.8, 6.9, 0.9 Hz, 1H), 4.75 (p, J=6.0 Hz, 1H), 1.31 (d, J=6.0 Hz, 6H).

Synthesis of 2-(2-isopropoxyphenyl)-2-(((R)-1-(4-methoxyphenyl)ethyl)amino)acetonitrile: Into a 1 L 3-necked round-bottom, flask were added 2-isopropoxybenzaldehyde (20.0 g, 121.9 mmol 1.0 eq), (R)-1-(4-methoxyphenyl)ethan-1-amine (18.4 g, 121.9 mmol, 1.0 eq), MeOH (200 mL), water (200 mL) at 25° C. After that, NaCN (6.6 g, 134.1 mmol, 1.1 eq) was added in portions at 25° C. The resulting mixture was stirred overnight at 25° C. The resulting mixture was filtered, the filter cake was washed with water (3×50 mL). The residue was dissolved in dichloromethane (300 mL) and then washed with water (3×50 mL) and brine (50 mL), dried over anhydrous Na₂SO₄. After filtration, 2-(2-isopropoxyphenyl)-2-(((R)-1-(4-methoxyphenyl)ethyl)amino)acetonitril (20.0 g, 50.6%) was obtained as a white solid. LC-MS (ESI, m/z) M+1: 325.

Synthesis of amino(2-isopropoxyphenyl)acetic acid: Into a 500 mL round-bottom flask, were placed 2-(2-isopropoxyphenyl)-2-{[(1R)-1-(4-methoxyphenyl)ethyl]amino}acetonitrile (20.0 g, 61.6 mmol, 1.0 eq), HCl (6 M, 200 mL). The resulting mixture was stirred for 4 hours at 100° C. The resulting mixture was extracted with ethyl acetate (2×500 mL). The aqueous layer was concentrated under vacuum to give amino(2-isopropoxyphenyl)acetic acid as a yellow oil (12.0 g, 51.2%). LC-MS (ESI, m/z) M+1: 210.

Synthesis of 2-amino-2-(2-isopropoxyphenyl)ethanol: Into a 500 mL 3-necked round-bottom flask, were placed amino(2-isopropoxyphenyl)acetic acid (12.0 g, 31.5 mmol, 1.0 eq, 55%), tetrahydrofuran (100 mL). After that, BH₃-THF (126 mL, 126.0 mmol, 4.0 eq) was added dropwise at 25° C. The reaction mixture was stirred overnight at 25° C. The resulting mixture was then quenched by the addition of HCl (1 M) and concentrated under vacuum. The resulting mixture was basified to pH=12 with NaOH (4 M) and then extracted with CH₂Cl₂ (3×100 mL), dried over anhydrous Na₂SO₄. After filtration, the filtrate was concentrated under vacuum. The crude residue was applied onto a silica gel column and eluted with dichloromethane/methanol=10:1 to give 2-amino-2-(2-isopropoxyphenyl)ethanol as a yellow oil (5.0 g, 81.2%). ¹HNMR (300 MHz, DMSO-d₆) δ 7.40 (dd, J=7.5, 1.8 Hz, 1H), 7.15 (ddd, J=8.2, 7.3, 1.8 Hz, 1H), 6.93 (dd, J=8.5, 1.1 Hz, 1H), 6.87 (td, J=7.4, 1.1 Hz, 1H), 4.66-4.54 (m, 1H), 4.18 (dd, J=8.2, 3.9 Hz, 1H), 3.52 (dd, J=10.2, 3.9 Hz, 1H), 3.22-3.07 (m, 1H), 1.95 (s, 2H), 1.27 (dd, J=6.0, 1.9 Hz, 6H).

Synthesis of 2-chloro-N-[2-hydroxy-1-(2-isopropoxyphenyl)ethyl]acetamide: Into a 250 mL round-bottom flask, were placed 2-amino-2-(2-isopropoxyphenyl)ethanol (3.1 g, 15.9 mmol, 1.0 eq), dichloromethane (50 mL), triethylamine (2.4 g, 23.8 mmol, 1.5 eq). After that, chloroacetyl chloride (2.0 g, 17.4 mmol, 1.1 eq) was added dropwise at 0° C. The reaction mixture was stirred for additional 2 hours at 25° C. The resulting mixture was quenched by the addition of water (200 mL) and extracted with CH₂Cl₂ (2×50 mL). The combined organic layer was washed with brine (200 mL), dried over anhydrous Na₂SO₄. After filtration, the filtrate was concentrated under vacuum to give 2-chloro-N-[2-hydroxy-1-(2-isopropoxyphenyl)ethyl]acetamide as red oil (4.3 g, 99.7%). LC-MS (ESI, m/z) M+1: 272/274.

Synthesis of 5-(2-isopropoxyphenyl)morpholin-3-one: Into a 500 mL round-bottom flask, were placed 2-chloro-N-[2-hydroxy-1-(2-isopropoxyphenyl)ethyl]acetamide (4.3 g, 15.8 mmol, 1.0 eq), tetrahydrofuran (300 mL). After that, NaH (1.9 g, 47.5 mmol, 3.0 eq., 60%) was added in portions at 0° C. The reaction mixture was stirred for overnight at 25° C. The resulting mixture was quenched by the addition of aqueous NH₄Cl (200 mL) and extracted with CH₂Cl₂ (2×100 mL). The combined organic layer was washed with brine (200 mL), dried over anhydrous Na₂SO₄. After filtration, the filtrate was concentrated under vacuum. The crude residue was applied onto a silica gel column and eluted with ethyl acetate/petroleum ether=3:2 to give 5-(2-isopropoxyphenyl)morpholin-3-one as a light yellow oil (2.9 g, 79.2%). ¹HNMR (400 MHz, Chloroform-d) δ 7.37-7.29 (m, 2H), 6.99 (td, J=7.5, 1.1 Hz, 1H), 6.91 (d, J=8.3 Hz, 1H), 6.21 (s, 1H), 5.14 (ddd, J=6.4, 4.2, 1.6 Hz, 1H), 4.64 (p, J=6.0 Hz, 1H), 4.36-4.20 (m, 2H), 4.12 (dd, J=11.7, 4.2 Hz, 1H), 3.69 (dd, J=11.7, 6.9 Hz, 1H), 1.38 (d, J=6.0 Hz, 6H).

Synthesis of (3R)-3-(2-isopropoxyphenyl)morpholine and (3S)-3-(2-isopropoxyphenyl)morpholine: Into a 100 mL round-bottom flask, were placed 5-(2-isopropoxyphenyl)morpholin-3-one (1.3 g, 5.5 mmol, 1.0 eq), tetrahydrofuran (20 mL). After that, LiAlH₄ (630 mg, 16.6 mmol, 3.0 eq) was added in portions at 0° C. The resulting mixture was stirred overnight at 25° C. The reaction was quenched with Na₂SO₄·10 H₂O at 0° C. After filtration, the filtrate was concentrated under vacuum. The residue product was purified by reverse flash chromatography using the following conditions: column, C18 reverse phase column; mobile phase, acetonitrile in water (0.1% NH₃·H₂O), 30% up to 90% gradient in 10 min; detector, UV 254 nm. The crude product was purified by Prep-CHIRAL-HPLC using the following conditions to give (3R)-3-(2-isopropoxyphenyl)morpholine as a light yellow oil (170 mg, 13.9%) and (3S)-3-(2-isopropoxyphenyl)morpholine as a light yellow oil (170 mg, 13.9%). OA, T_{R}=7 min in CHIRAL-HPLC, Column: CHIRAL ART Cellulose-SB, 3*25 cm, 5 um; Mobile Phase A: Hexane, Mobile Phase B: EtOH; Flow rate: 35 mL/min; Gradient: 5% B to 5% B in 15 min, Wave Length: 220/254 nm. OB, T_{R}=11 min in CHIRAL-HPLC, Column: CHIRAL ART Cellulose-SB, 3*25 cm, 5 um; Mobile Phase A: Hexane, Mobile Phase B: EtOH; Flow rate: 35 mL/min; Gradient: 5% B to 5% B in 15 min, Wave Length: 220/254 nm. ¹HNMR-OA (400 MHz, DMSO-d₆) δ 7.48 (dd, J=7.5, 1.8 Hz, 1H), 7.19 (ddd, J=8.4, 7.3, 1.8 Hz, 1H), 7.00-6.93 (m, 1H), 6.89 (td, J=7.4, 1.1 Hz, 1H), 4.61 (h, J=6.0 Hz, 1H), 4.11 (dd, J=9.7, 3.1 Hz, 1H), 3.74 (td, J=9.5, 8.8, 2.7 Hz, 2H), 3.48-3.38 (m, 1H), 3.00 (dd, J=10.5, 9.7 Hz, 1H), 2.96-2.82 (m, 2H), 2.65 (s, 1H), 1.29 (d, J=6.0 Hz, 6H). ¹HNMR-OB (400 MHz, DMSO-d₆) δ 7.47 (dd, J=7.6, 1.8 Hz, 1H), 7.24-7.15 (m, 1H), 6.99-6.93 (m, 1H), 6.89 (td, J=7.5, 1.1 Hz, 1H), 4.58-4.68 (m, 1H), 4.11 (dd, J=9.7, 3.1 Hz, 1H), 3.74 (tt, J=8.1, 2.8 Hz, 2H), 3.48-3.38 (m, 1H), 3.00 (t, J=10.1 Hz, 1H), 2.96-2.83 (m, 2H), 1.29 (d, J=6.1 Hz, 6H).

### Example INT_34 Preparation of tert-butyl 2-(2-bromophenyl)piperidine-1-carboxylate

Synthesis of tert-butyl N-[5-(2-bromophenyl)-5-oxopentyl]carbamate: Into a 1000 mL 3-necked round-bottom flask purged and maintained with an inert atmosphere of nitrogen, were placed 1-bromo-2-iodobenzene (51.1 g, 180.7 mmol, 1.2 eq), i-PrMgCl-THF (90 mL, 180.0 mmol, 1.2 eq), THE (500 mL) at 0° C. for 3 hours. Another 3000 mL 3-necked round-bottom flask purged and maintained with an inert atmosphere of nitrogen, was placed a solution of tert-butyl 2-oxopiperidine-1-carboxylate (30.0 g, 150.6 mmol, 1.0 eq) in THE (500 mL). The above solution was added dropwise to the previous mixture at −78° C. The resulting solution was stirred for 1 hour at 25° C. The reaction was then quenched by the addition of aqueous NH₄Cl (500 mL) and then extracted CH₂Cl₂ (3×500 mL). The combined organic layers were washed with brine (1000 mL), dried over anhydrous Na₂SO₄. After filtration, the filtrate was concentrated under reduced pressure. The crude residue was applied onto a silica gel column and eluted with ethyl acetate/petroleum ether=4:1 to give tert-butyl N-[5-(2-bromophenyl)-5-oxopentyl]carbamate (25.0 g, 46.6%) as red oil. ¹HNMR (300 MHz, Chloroform-d) δ 7.67-7.58 (m, 1H), 7.38 (dd, J=4.1, 0.9 Hz, 2H), 7.28 (s, 1H), 4.58 (s, 1H), 3.18 (t, J=6.9 Hz, 2H), 2.96 (t, J=7.1 Hz, 2H), 1.78 (p, J=7.1 Hz, 2H), 1.64-1.51 (m, 2H), 1.46 (s, 9H).

Synthesis of 2-(2-bromophenyl)-3,4,5,6-tetrahydropyridine: A mixture of tert-butyl N-[5-(2-bromophenyl)-5-oxopentyl]carbamate (25.0 g, 42.1 mmol, 1.0 eq) in formic acid (300 mL) was stirred for 4 hours at 40° C. The resulting mixture was concentrated under vacuum. The resulting mixture was diluted with aqueous NaOH and extracted with EtOAc (2×500 mL). The combined organic layer was washed with brine (500 mL), dried over anhydrous Na₂SO₄. After filtration, the filtrate was concentrated under vacuum to give 2-(2-bromophenyl)-3,4,5,6-tetrahydropyridine (15.0 g, 89.8%). ¹HNMR (300 MHz, Chloroform-d) δ 7.56 (dd, J=8.0, 1.2 Hz, 1H), 7.38-7.13 (m, 3H), 3.82 (tt, J=5.7, 2.2 Hz, 2H), 2.50 (tt, J=6.5, 2.1 Hz, 2H), 1.93-1.79 (m, 2H), 1.78-1.69 (m, 2H).

Synthesis of 2-(2-bromophenyl)piperidine: Into a 500 mL 3-necked round-bottom flask purged and maintained with an inert atmosphere of nitrogen, were placed 2-(2-bromophenyl)-3,4,5,6-tetrahydropyridine (15.0 g, 62.9 mmol, 1.0 eq), tetrahydrofuran (200 mL). After that, NaBH₄ (7.1 g, 188.9 mmol, 3.0 eq) was added in portions at 0° C. The reaction mixture was stirred for 5 hours at 50° C. The reaction was then quenched by the addition of aqueous NH₄Cl and then concentrated under vacuum. The residue was dissolved in dichloromethane (500 mL) and washed with water (3×500 mL). The organic layer was dried over Na₂SO₄. After filtration, the filtrate was concentrated under vacuum. The crude residue was applied onto a silica gel column and eluted with ethyl acetate/petroleum ether=2:1 to give 2-(2-bromophenyl)piperidine (12.5 g, 82.6%). ¹HNMR (300 MHz, Chloroform-d) δ 7.60 (dd, J=7.8, 1.8 Hz, 1H), 7.53 (dd, J=8.0, 1.3 Hz, 1H), 7.41-7.18 (m, 1H), 7.10 (td, J=7.6, 1.8 Hz, 1H), 4.00 (dd, J=10.8, 2.2 Hz, 1H), 3.21 (ddt, J=11.4, 3.8, 1.7 Hz, 1H), 2.86 (td, J=11.5, 3.1 Hz, 1H), 1.99-1.85 (m, 3H), 1.79-1.31 (m, 4H).

Synthesis of tert-butyl 2-(2-bromophenyl)piperidine-1-carboxylate: Into a 100 mL round-bottom flask, were placed 2-(2-bromophenyl)piperidine (12.0 g, 49.9 mmol, 1.0 eq), dichloromethane (200 mL), TEA (10.1 g, 99.9 mmol, 2.0 eq), DMAP (0.6 g, 4.9 mmol, 0.1 eq), (Boc)₂O (13.1 g, 59.9 mmol, 1.2 eq). The reaction mixture was stirred overnight at 25° C. The resulting mixture was concentrated under vacuum. The crude residue was applied onto a silica gel column and eluted with ethyl acetate/petroleum ether=1:5 to give tert-butyl 2-(2-bromophenyl)piperidine-1-carboxylate as a light yellow oil (9.0 g, 52.9%). ¹HNMR (300 MHz, Chloroform-d) δ 7.54 (d, J=7.9 Hz, 1H), 7.31-7.20 (m, 2H), 7.09 (ddd, J=8.0, 5.1, 3.7 Hz, 1H), 5.30 (dd, J=6.4, 5.1 Hz, 1H), 4.13 (ddt, J=10.5, 5.9, 2.9 Hz, 1H), 3.39 (ddd, J=13.5, 11.6, 4.8 Hz, 1H), 2.08-1.25 (m, 5H), 1.26 (s, 9H), 0.95-0.82 (m, 1H).

### Example INT_35 Preparation of (3R)-1-methyl-3-(2-methylphenyl)piperazine and (3S)-1-methyl-3-(2-methylphenyl)piperazine

Synthesis of (R)-1-(4-methoxyphenyl)ethan-1-amine hydrochloride: Into a 5 L 3-necked round-bottom flask were added (R)-1-(4-methoxyphenyl)ethan-1-amine (100.0 g, 661.3 mmol, 1.0 eq) and HCl in 1,4-dioxane (2.0 M, 1 L) at 25° C. The resulting mixture was stirred for 14 hours at 25° C. The resulting mixture was filtered, the filter cake was washed with Et₂O (3×200 mL). Finally, (R)-1-(4-methoxyphenyl)ethan-1-amine hydrochloride (110.0 g, 88.6%) was obtained as a white solid.

Synthesis of (R)-2-(((R)-1-(4-methoxyphenyl)ethyl)amino)-2-(o-tolyl)acetonitrile hydrochloride: Into a 1 L 3-necked round-bottom flask were added 2-methylbenzaldehyde (20.0 g, 166.5 mmol, 1.0 eq), (R)-1-(4-methoxyphenyl)ethan-1-amine hydrochloride (31.2 g, 166.5 mmol, 1.0 eq), MeOH (200 mL), water (200 mL) at 25° C. After that, NaCN (9.0 g, 183.1 mmol, 1.1 eq) was added in portions at 25° C. The resulting mixture was stirred for 14 hours at 25° C. The resulting mixture was filtered, the filter cake was washed with water (3×50 mL). The filter cake was dissolved in dichloromethane (300 mL). The organic layers were washed with water (3×50 mL) and brine (50 mL), dried over anhydrous Na₂SO₄. After filtration, the filtrate was concentrated under reduced pressure. (2R)-2-{[(1R)-1-(4-methoxyphenyl)ethyl]amino}-2-(2-methylphenyl)acetonitrile hydrochloride (30.0 g, crude) was dissolved in Et₂O (3 L). To the above mixture was added HCl in MeOH (1 M, 200 mL) dropwise at 25° C. The resulting mixture was stirred for additional 14 hours at 25° C. The precipitated solids were collected by filtration and then washed with Et₂O (3×200 mL). Finally, (R)-2-(((R)-1-(4-methoxyphenyl)ethyl)amino)-2-(o-tolyl)acetonitrile hydrochloride (30.2 g, 56.9%) was obtained as a white solid. ¹HNMR (300 MHz, DMSO-d₆) δ 7.59-7.50 (m, 1H), 7.38-7.12 (m, 6H), 6.99-6.84 (m, 1H), 4.36 (d, J=11.2 Hz, 1H), 3.96 (tt, J=6.5, 3.3 Hz, 1H), 3.76 (s, 3H), 3.25 (dd, J=11.2, 2.7 Hz, 1H), 2.07 (s, 3H), 1.35-1.26 (m, 3H).

Synthesis of (R)-2-amino-2-(o-tolyl)acetic acid: Into a 1 L round-bottom flask were added (R)-2-(((R)-1-(4-methoxyphenyl)ethyl)amino)-2-(o-tolyl)acetonitrile hydrochloride (30.0 g, 94.7 mmol, 1.0 eq) and HCl (6 M, 300 mL) at 25° C. The resulting mixture was stirred for 4 hours at 90° C. The resulting mixture was washed with Et₂O (3×50 mL) and then the water phase was concentrated under reduced pressure. Finally, (R)-2-amino-2-(o-tolyl)acetic acid (13.0 g, 83.1%) was obtained as a white solid. ¹HNMR (400 MHz, DMSO-d₆) δ 13.54 (bs, 1H), 8.88 (bs, 2H), 7.40-7.30 (m, 1H), 7.33-7.23 (m, 3H), 5.13 (s, 1H), 2.45 (s, 3H).

(R)-[(tert-butoxycarbonyl)amino](2-methylphenyl)acetic acid: Into a 100 mL round-bottom flask, were placed (R)-amino(2-methylphenyl)acetic acid (6.0 g, 36.3 mmol, 1.0 eq), Dioxane (50 mL), water (50 mL), Na₂CO₃ (7.7 g, 72.7 mmol, 2.0 eq), (Boc)₂O (8.7 g, 40.0 mmol, 1.1 eq). The reaction mixture was stirred overnight at 25° C. The resulting mixture was quenched by the addition of HCl (1.0 M) and then extracted with ethyl acetate (3×100 mL). The combined organic layer was washed with brine (200 mL), dried over anhydrous Na₂SO₄. After filtration, the filtrate was concentrated under vacuum to give (R)-[(tert-butoxycarbonyl)amino](2-methylphenyl)acetic acid as a white solid (5.8 g, 60.2%). LC-MS (ESI, m/z) M+1: 266.

Synthesis of ethyl 2-[(2R)-2-[(tert-butoxycarbonyl)amino]-N-methyl-2-(2-methylphenyl)acetamido]acetate: Into a 100 mL round-bottom flask were placed (R)-[(tert-butoxycarbonyl)amino](2-methylphenyl)acetic acid (2.0 g, 7.5 mmol, 1.0 eq), ethyl 2-(methylamino)acetate (1.8 g, 15.1 mmol, 2.0 eq), EDCl (1.7 g, 9.0 mmol, 1.2 eq), HOBT (1.2 g, 9.0 mmol, 1.2 eq), dichloromethane (40 mL). The reaction mixture was stirred for overnight at 25° C. The resulting mixture was concentrated under vacuum. The crude residue was applied onto a silica gel column and eluted with ethyl acetate/petroleum ether=1:2 to give ethyl 2-[(2R)-2-[(tert-butoxycarbonyl)amino]-N-methyl-2-(2-methylphenyl)acetamido]acetate as a colorless solid (2.1 g, 79.5%). ¹H NMR (300 MHz, Chloroform-d) δ 7.33-7.13 (m, 4H), 5.80-5.71 (m, 1H), 4.53-4.41 (m, 1H), 4.27-4.15 (m, 2H), 3.91-3.79 (m, 1H), 2.85-2.82 (m, 3H), 2.50 (d, J=7.6 Hz, 3H), 1.44 (d, J=3.9 Hz, 9H), 1.28 (t, J=7.1 Hz, 3H).

Synthesis of ethyl 2-[(2R)-2-amino-N-methyl-2-(2-methylphenyl)acetamido]acetate: A solution of ethyl 2-[(2R)-2-[(tert-butoxycarbonyl)amino]-N-methyl-2-(2-methylphenyl)acetamido]acetate (2.0 g, 5.7 mmol, 1.0 eq) in HCl (gas) in 1,4-dioxane (40 mL) was stirred for 1 hour at 25° C. The resulting mixture was concentrated under vacuum. The resulting mixture was diluted with saturated aqueous NaHCO₃ and extracted with CH₂Cl₂ (2×100 mL). The combined organic layer was washed with brine (100 mL), dried over anhydrous Na₂SO₄. After filtration, the filtrate was concentrated under vacuum. The crude product was purified by Prep-HPLC to give ethyl 2-[(2R)-2-amino-N-methyl-2-(2-methylphenyl)acetamido]acetate (1.4 g, 98.0%). ¹H NMR (300 MHz, DMSO-d₆) δ 8.80 (s, 3H), 7.41-7.16 (m, 4H), 5.43 (d, J=15.6 Hz, 1H), 4.33 (d, J=17.1 Hz, 1H), 4.21-3.97 (m, 3H), 2.82 (d, J=81.0 Hz, 3H), 2.50 (d, J=21.8 Hz, 3H), 1.19 (q, J=9.0, 7.1 Hz, 3H).

Synthesis of 1-methyl-3-(2-methylphenyl)piperazine-2,5-dione: Into a 100 mL round-bottom flask, were placed ethyl 2-[(2R)-2-amino-N-methyl-2-(2-methylphenyl)acetamido]acetate (1.4 g, 5.6 mmol, 1.0 eq), K₂CO₃ (3.1 g, 22.3 mmol, 4.0 eq), EtOH (20 mL). The reaction mixture was stirred for overnight at 80° C. The resulting mixture was quenched by the addition of water (200 mL) and extracted with CH₂Cl₂ (2×100 mL). The combined organic layer was washed with brine (200 mL), dried over anhydrous Na₂SO₄. After filtration, the filtrate was concentrated under vacuum to give 1-methyl-3-(2-methylphenyl)piperazine-2,5-dione as a yellow solid (1.0 g, 81.1%). ¹H NMR (300 MHz, DMSO-d₆) δ 8.50 (s, 1H), 7.20 (d, J=1.9 Hz, 4H), 5.24 (d, J=2.2 Hz, 1H), 4.20 (d, J=17.7 Hz, 1H), 4.02 (d, J=17.8 Hz, 1H), 2.84 (s, 3H), 2.39 (s, 3H).

Synthesis of (3R)-1-methyl-3-(2-methylphenyl)piperazine and (3S)-1-methyl-3-(2-methylphenyl)piperazine: Into a 50 mL round-bottom flask, were placed 3-(2-methylphenyl)piperazine-2,5-dione (700 mg, 3.4 mmol, 1.0 eq), tetrahydrofuran (10 mL). After that, LiALH₄ (390 mg, 10.3 mmol, 3.0 eq) was added in portions at 0° C. The reaction mixture was stirred overnight at 25° C. The resulting mixture was then quenched with Na₂SO₄·10 H₂O at 0° C. After filtration, the filtrate was concentrated under vacuum. The crude product was purified by Prep-CHIRAL-HPLC. Finally, (3R)-1-methyl-3-(2-methylphenyl)piperazine was obtained as a light yellow oil (80 mg, 12.3%) and (3S)-1-methyl-3-(2-methylphenyl)piperazine was obtained as a light yellow oil (80 mg, 12.3%). A, T_{R}=0.319 min in CHIRAL-SFC, Column: CHIRAL ART Amylose-C NEO, 3*25 cm, 5 um; Mobile Phase A: CO₂, Mobile Phase B: MeOH (0.1% 2M NH₃-MeOH); Flow rate: 80 mL/min; Gradient: 15% B in 10 min, Wave Length: 220 nm. B, T_{R}=0.437 min in CHIRAL-SFC, Column: CHIRAL ART Amylose-C NEO, 3*25 cm, 5 um; Mobile Phase A: CO₂, Mobile Phase B: MeOH (0.1% 2M NH₃-MeOH); Flow rate: 80 mL/min; Gradient: 15% B in 10 min, Wave Length: 220 nm.

### Example INT_36 Preparation of tert-butyl 2-(2-bromophenyl)-4-methylpiperazine-1-carboxylate

Synthesis of (R)-(2-bromophenyl)[(tert-butoxycarbonyl)amino]acetic acid: Into a 250 mL round-bottom flask, were placed (R)-amino(2-bromophenyl)acetic acid (8.0 g, 34.8 mmol, 1.0 eq), dioxane (100 mL), H₂O (50 mL), Na₂CO₃ (7.4 g, 69.8 mmol, 2.0 eq), Boc₂O (8.4 g, 38.5 mmol, 1.1 eq). The resulting mixture was stirred overnight at 25° C. The resulting mixture was diluted with HCl (1 M) and then extracted with EtOAc (3×50 mL). The combined organic layer was washed with brine (200 mL), dried over anhydrous Na₂SO₄. After filtration, the filtrate was concentrated under vacuum to give (R)-(2-bromophenyl)[(tert-butoxycarbonyl)amino]acetic acid as a yellow oil (11.0 g, 95.8%). ¹HNMR (300 MHz, DMSO-d₆) δ 7.70 (d, J=8.5 Hz, 1H), 7.63 (dd, J=8.0, 1.2 Hz, 1H), 7.45 (dd, J=7.8, 1.9 Hz, 1H), 7.38 (td, J=7.4, 1.3 Hz, 1H), 7.25 (ddd, J=8.0, 7.1, 2.0 Hz, 1H), 6.13 (s, 1H), 5.56 (d, J=8.5 Hz, 1H), 1.39 (s, 9H).

Synthesis of ethyl 2-[(2R)-2-(2-bromophenyl)-2-[(tert-butoxycarbonyl)amino]-N-methylacetamido]acetate: Into a 250 mL round-bottom flask, were placed (R)-(2-bromophenyl)[(tert-butoxycarbonyl)amino]acetic acid (10.6 g, 32.1 mmol, 1.0 eq), dichloromethane (100 mL), ethyl 2-(methylamino)acetate hydrochloride (5.4 g, 35.2 mmol, 1.1 eq), EDCl (7.4 g, 38.6 mmol, 1.2 eq), HOBt (5.2 g, 38.5 mmol, 1.2 eq), TEA (6.5 g, 64.2 mmol, 2.0 eq). The resulting mixture was stirred for overnight at 25° C. The resulting mixture was concentrated under vacuum. The crude residue was applied onto a silica gel column and eluted with petroleum ether/ethyl acetate=7:3 to give ethyl 2-[(2R)-2-(2-bromophenyl)-2-[(tert-butoxycarbonyl)amino]-N-methylacetamido]acetate as a yellow oil (10.4 g, 75.5%). LC-MS (ES, m/z) M+1: 429/431.

Synthesis of ethyl 2-[(2R)-2-amino-2-(2-bromophenyl)-N-methylacetamido]acetate hydrochloride: A solution of ethyl 2-[(2R)-2-(2-bromophenyl)-2-[(tert-butoxycarbonyl)amino]-N-methylacetamido]acetate (10.4 g, 24.2 mmol, 1.0 eq) in HCl/1,4-dioxane (100 mL) was stirred for 1 hour at 25° C. The resulting mixture was concentrated under vacuum to give ethyl 2-[(2R)-2-amino-2-(2-bromophenyl)-N-methylacetamido]acetate hydrochloride as a white solid (8.8 g, 99.3%). LC-MS (ES, m/z) M+1: 329/331.

Synthesis of 3-(2-bromophenyl)-1-methylpiperazine-2,5-dione: Into a 250 mL round-bottom flask, were placed ethyl 2-[(2R)-2-amino-2-(2-bromophenyl)-N-methylacetamido]acetate hydrochloride (8.8 g, 24.1 mmol, 1.0 eq), EtOH (100 mL), K₂CO₃ (13.3 g, 96.2 mmol, 4.0 eq). The resulting mixture was stirred overnight at 80° C. The reaction was quenched by the addition of water (300 mL) and then extracted with CH₂Cl₂ (2×100 mL). The combined organic layer was washed with brine (200 mL), dried over anhydrous Na₂SO₄. After filtration, the filtrate was concentrated under vacuum to give 3-(2-bromophenyl)-1-methylpiperazine-2,5-dione as a white solid (5.1 g, 74.8%). ¹HNMR (300 MHz, DMSO-d₆) δ 8.58 (s, 1H), 7.64 (d, J=7.9 Hz, 1H), 7.40 (d, J=4.3 Hz, 2H), 7.29 (dq, J=8.7, 4.2 Hz, 1H), 5.38 (s, 1H), 4.10 (d, J=7.2 Hz, 2H), 2.84 (s, 3H).

Synthesis of 3-(2-bromophenyl)-1-methylpiperazine: Into a 250 mL round-bottom flask, were placed 3-(2-bromophenyl)-1-methylpiperazine-2,5-dione (4.7 g, 16.6 mmol, 1.0 eq), BH₃-THF (1.0 M, 50 mL). The resulting mixture was stirred for 3 hours at 80° C. under nitrogen atmosphere. The resulting mixture was quenched with HCl (1 M). The resulting mixture was concentrated under vacuum. The residue was basified to pH=12 with NaOH (4 M) and extracted with CH₂Cl₂ (2×100 mL). The combined organic layer was washed with brine (200 mL), dried over anhydrous Na₂SO₄. After filtration, the filtrate was concentrated under vacuum. The crude residue was applied onto a silica gel column and eluted with dichloromethane/methanol=10:1 to give 3-(2-bromophenyl)-1-methylpiperazine as a white solid (2.6 g, 61.4%). ¹HNMR (300 MHz, Methanol-d₄) δ 7.90 (dd, J=7.9, 1.6 Hz, 1H), 7.79 (dd, J=8.1, 1.3 Hz, 1H), 7.58 (td, J=7.7, 1.3 Hz, 1H), 7.44 (td, J=7.7, 1.7 Hz, 1H), 5.20 (dd, J=11.9, 3.1 Hz, 1H), 3.87-3.69 (m, 4H), 3.69-3.43 (m, 2H), 3.01 (s, 3H).

Synthesis of tert-butyl 2-(2-bromophenyl)-4-methylpiperazine-1-carboxylate: Into a 100 mL round-bottom flask, were placed 3-(2-bromophenyl)-1-methylpiperazine (2.4 g, 9.4 mmol, 1.0 eq), dichloromethane (30 mL), triethylamine (1.9 g, 18.8 mmol, 2.0 eq), DMAP (115 mg, 0.9 mmol, 0.1 eq) and Boc₂O (2.5 g, 11.4 mmol, 1.2 eq). The resulting mixture was stirred for 4 hours at 25° C. The reaction was quenched with HCl (0.2 M) and then extracted with CH₂Cl₂ (2×50 mL). The combined organic layer was washed with brine (200 mL), dried over anhydrous Na₂SO₄. After filtration, the filtrate was concentrated under vacuum. The crude residue was applied onto a silica gel column and eluted with dichloromethane/ethyl acetate (including 10% of methanol)=2:1 to give tert-butyl 2-(2-bromophenyl)-4-methylpiperazine-1-carboxylate as a yellow oil (1.9 g, 56.8%). ¹HNMR (400 MHz, Chloroform-d) δ 7.55 (dd, J=7.9, 1.3 Hz, 1H), 7.45 (dd, J=7.8, 1.7 Hz, 1H), 7.30-7.25 (m, 1H), 7.11 (td, J=7.6, 1.7 Hz, 1H), 5.34 (dd, J=5.5, 2.3 Hz, 1H), 4.00 (dt, J=13.7, 3.2 Hz, 1H), 3.58 (td, J=12.6, 3.8 Hz, 1H), 3.50-3.41 (m, 1H), 3.03 (d, J=12.0 Hz, 1H), 2.86 (d, J=11.3 Hz, 1H), 2.49 (s, 1H), 2.27 (s, 3H), 1.31 (s, 9H).

### Example INT_37 Preparation of (3R)-3-(2-isopropylphenyl)-1-methylpiperazine (assumed) and (3S)-3-(2-isopropylphenyl)-1-methylpiperazine (assumed)

Synthesis of tert-butyl 4-methyl-2-[2-(prop-1-en-2-yl)phenyl]piperazine-1-carboxylate: Into a 40 mL vial, were placed tert-butyl 2-(2-bromophenyl)-4-methylpiperazine-1-carboxylate (800 mg, 2.3 mmol, 1.0 eq), dioxane (9 mL), H₂O (1 mL), 4,4,5,5-tetramethyl-2-(prop-1-en-2-yl)-1,3,2-dioxaborolane (949 mg, 5.6 mmol, 2.5 eq), K₂CO₃ (936 mg, 6.8 mmol, 3.0 eq), Pd(dppf)Cl₂·CH₂Cl₂ (185 mg, 0.2 mmol, 0.1 eq). The resulting mixture was stirred for 3 hours at 80° C. under nitrogen atmosphere. The reaction mixture was diluted with water (200 mL) and extracted with EtOAc (2×50 mL). The combined organic layer was washed with brine (200 mL), dried over anhydrous Na₂SO₄. After filtration, the filtrate was concentrated under vacuum. The crude residue was applied onto a silica gel column and eluted with dichloromethane/ethyl acetate (including 10% of methanol)=4:1 to give tert-butyl 4-methyl-2-[2-(prop-1-en-2-yl)phenyl]piperazine-1-carboxylate as a yellow oil (577 mg, 80.9%). ¹HNMR (400 MHz, Chloroform-d) δ 7.45 (dd, J=7.1, 2.1 Hz, 1H), 7.21 (tt, J=7.3, 5.5 Hz, 2H), 7.14-7.07 (m, 1H), 5.41 (dd, J=4.9, 3.0 Hz, 1H), 5.20 (t, J=1.8 Hz, 1H), 4.91 (dd, J=2.2, 1.1 Hz, 1H), 3.91 (dt, J=13.3, 3.5 Hz, 1H), 3.53-3.39 (m, 2H), 3.01 (ddd, J=11.8, 3.0, 1.6 Hz, 1H), 2.72 (d, J=11.1 Hz, 1H), 2.44-2.33 (m, 1H), 2.23 (s, 3H), 2.11 (t, J=1.2 Hz, 3H), 1.38 (s, 9H).

Synthesis of tert-butyl 2-(2-isopropylphenyl)-4-methylpiperazine-1-carboxylate: Into a 50 mL pressure tank reactor, were placed tert-butyl 4-methyl-2-[2-(prop-1-en-2-yl)phenyl]piperazine-1-carboxylate (570 mg, 1.8 mmol, 1.0 eq), methanol (10 mL), Pd/C (100 mg). The resulting mixture was stirred overnight at 50° C. under H₂ (20 atm). The solids were filtered off. The filter cake was washed with methanol and the filtrate was collected and concentrated under vacuum. The crude residue was applied onto a silica gel column and eluted with dichloromethane/ethyl acetate (including 10% of methanol)=6:1 to give tert-butyl 2-(2-isopropylphenyl)-4-methylpiperazine-1-carboxylate as a yellow oil (490 mg, 85.4%). ¹HNMR (400 MHz, Chloroform-d) δ 7.84 (d, J=7.8 Hz, 1H), 7.32 (dd, J=7.8, 1.6 Hz, 1H), 7.25 (td, J=7.5, 1.5 Hz, 1H), 7.15 (td, J=7.5, 1.6 Hz, 1H), 5.41 (dd, J=5.1, 2.2 Hz, 1H), 3.88 (dt, J=13.6, 3.1 Hz, 1H), 3.51-3.40 (m, 1H), 3.37-3.31 (m, 1H), 3.28-3.17 (m, 1H), 3.02 (dt, J=11.8, 1.8 Hz, 1H), 2.77 (dt, J=12.0, 3.1 Hz, 1H), 2.47 (dd, J=11.8, 5.0 Hz, 1H), 2.28 (s, 3H), 1.42 (s, 9H), 1.26 (d, J=6.8 Hz, 3H), 1.20 (d, J=6.7 Hz, 3H).

Synthesis of (3R)-3-(2-isopropylphenyl)-1-methylpiperazine (assumed) and (3S)-3-(2-isopropylphenyl)-1-methylpiperazine (assumed): A solution of tert-butyl 2-(2-isopropylphenyl)-4-methylpiperazine-1-carboxylate (490 mg, 1.5 mmol, 1.0 eq) in HCl (gas)/1,4-dioxane (4 M, 5 mL) was stirred for 1 hour at 25° C. The resulting mixture was concentrated under vacuum. The resulting mixture was diluted with aq.

NaHCO₃ (50 mL) and extracted with CH₂Cl₂ (2×30 mL). The combined organic layer was washed with brine (100 mL), dried over anhydrous Na₂SO₄. After filtration, the filtrate was concentrated under vacuum. The crude product was purified by Prep-CHIRAL-SFC using the following conditions to give (3R)-3-(2-isopropylphenyl)-1-methylpiperazine (assumed) as a yellow oil (120 mg, 35.7%) and (3S)-3-(2-isopropylphenyl)-1-methylpiperazine (assumed) as a yellow oil (100 mg, 29.8%). A, RT=2.7 min in CHIRAL-SFC, Column: CHIRAL ART Amylose-C NEO, 3*25 cm, 5 um; Mobile Phase A: CO₂, Mobile Phase B: EtOH (0.1% 2 M NH₃-MeOH); Flow rate: 75 mL/min; Gradient: 15% B, Wave Length: 220 nm. B, RT=3.9 min in CHIRAL-SFC, Column: CHIRAL ART Amylose-C NEO, 3*25 cm, 5 um; Mobile Phase A: CO₂, Mobile Phase B: EtOH (0.1% 2 M NH₃-MeOH); Flow rate: 75 mL/min; Gradient: 15% B, Wave Length: 220 nm. A: ¹HNMR (300 MHz, Chloroform-d) δ 7.58 (dd, J=7.6, 1.6 Hz, 1H), 7.31 (dd, J=7.7, 2.0 Hz, 1H), 7.27-7.15 (m, 2H), 4.25 (dd, J=10.1, 2.6 Hz, 1H), 3.36 (q, J=6.8 Hz, 1H), 3.15-3.13 (m, 2H), 2.91-2.82 (m, 2H), 2.36 (s, 3H), 2.26-2.18 (m, 1H), 2.09-2.02 (m, 1H), 1.26 (dd, J=6.9, 4.9 Hz, 6H). B: ¹HNMR (300 MHz, Chloroform-d) δ 7.58 (dd, J=7.6, 1.6 Hz, 1H), 7.31 (dd, J=7.7, 2.0 Hz, 1H), 7.27-7.15 (m, 2H), 4.25 (dd, J=10.1, 2.6 Hz, 1H), 3.36 (q, J=6.8 Hz, 1H), 3.15-3.13 (m, 2H), 2.91-2.82 (m, 2H), 2.36 (s, 3H), 2.26-2.18 (m, 1H), 2.09-2.02 (m, 1H), 1.26 (dd, J=6.9, 4.9 Hz, 6H).

### Example INT_38 Preparation of (3R)-3-(2-isopropoxyphenyl)-1-methylpiperazine (assumed) and (3S)-3-(2-isopropoxyphenyl)-1-methylpiperazine (assumed)

Synthesis of tert-butyl 4-methyl-2-[2-(4,4,5,5-tetramethyl-1,3,2-dioxaborolan-2-yl)phenyl]piperazine-1-carboxylate: Into a 40 mL vial, were placed tert-butyl 2-(2-bromophenyl)-4-methylpiperazine-1-carboxylate (591 mg, 1.7 mmol, 1.0 eq), dioxane (5 mL), bis(pinacolato)diboron (1.3 g, 5.0 mmol, 3.0 eq), KOAc (491 mg, 5.0 mmol, 3.0 eq), Pd(dppf)Cl₂·CH₂Cl₂ (136 mg, 0.17 mmol, 0.1 eq). The resulting mixture was stirred for 3 hours at 80° C. under nitrogen atmosphere. The resulting mixture was diluted with water (100 mL) and then extracted with EtOAc (2×50 mL). The combined organic layer was washed with brine (100 mL), dried over anhydrous Na₂SO₄. After filtration, the filtrate was concentrated under vacuum. The crude residue was applied onto a silica gel column and eluted with dichloromethane/ethyl acetate (including 10% of methanol)=6:1 to give tert-butyl 4-methyl-2-[2-(4,4,5,5-tetramethyl-1,3,2-dioxaborolan-2-yl)phenyl]piperazine-1-carboxylate as a dark yellow oil (600 mg, 89.6%). LC-MS (ES, m/z) M+1: 403.

Synthesis of tert-butyl 2-(2-hydroxyphenyl)-4-methylpiperazine-1-carboxylate: Into a 100 mL round-bottom flask, were placed tert-butyl 4-methyl-2-[2-(4,4,5,5-tetramethyl-1,3,2-dioxaborolan-2-yl)phenyl]piperazine-1-carboxylate (600 mg, 1.5 mmol, 1.0 eq), EtOH (12 mL), water (2 mL), m-CPBA (303 mg, 1.5 mmol, 1.00 eq, 85%). The resulting mixture was stirred for 2 hours at 25° C. The resulting mixture was diluted with aq. NaHCO₃ (100 mL) and then extracted with CH₂Cl₂ (2×50 mL). The combined organic layer was washed with brine (100 mL), dried over anhydrous Na₂SO₄. After filtration, the filtrate was concentrated under vacuum. The crude residue was applied onto a silica gel column and eluted with dichloromethane/ethyl acetate=5:1 to give tert-butyl 2-(2-hydroxyphenyl)-4-methylpiperazine-1-carboxylate as a yellow oil (180 mg, 41.3%). LC-MS (ES, m/z) M+1: 293.

Synthesis of tert-butyl 2-(2-isopropoxyphenyl)-4-methylpiperazine-1-carboxylate: Into a 40 mL vial, were placed tert-butyl 2-(2-hydroxyphenyl)-4-methylpiperazine-1-carboxylate (175 mg, 0.6 mmol, 1.0 eq), N, N-dimethylformamide (5 mL). After that, NaH (48 mg, 1.2 mmol, 2.0 eq, 60%) was added in portions at 0° C. The resulting mixture was stirred for additional 30 min. To the above mixture was added 2-bromopropane (110 mg, 0.9 mmol, 1.5 eq) dropwise at 0° C. The resulting mixture was stirred for 7 hours at 25° C. under nitrogen atmosphere. The reaction mixture was then quenched by the addition of water (100 mL) and extracted with EtOAc (3×30 mL). The combined organic layer was washed with brine (100 mL), dried over anhydrous Na₂SO₄. After filtration, the filtrate was concentrated under vacuum. The crude residue was applied onto a silica gel column and eluted with ethyl acetate/petroleum ether=1:3 to give tert-butyl 2-(2-isopropoxyphenyl)-4-methylpiperazine-1-carboxylate as a colorless oil (144 mg, 71.9%). ¹HNMR (300 MHz, Chloroform-d) δ 7.25-7.12 (m, 2H), 6.95-6.81 (m, 2H), 5.41-5.34 (m, 1H), 4.65-4.57 (m, 1H), 4.06 (d, J=13.5 Hz, 1H), 3.50 (t, J=12.5 Hz, 1H), 3.32 (d, J=11.8 Hz, 1H), 2.83 (d, J=11.1 Hz, 1H), 2.50-2.30 (m, 1H), 2.25 (s, 3H), 2.21-2.05 (m, 1H), 1.39 (d, J=6.1 Hz, 3H), 1.36 (s, 9H), 1.32 (d, J=6.0 Hz, 3H).

Synthesis of (3R)-3-(2-isopropoxyphenyl)-1-methylpiperazine (assumed) and (3S)-3-(2-isopropoxyphenyl)-1-methylpiperazine (assumed): A solution of tert-butyl 2-(2-isopropoxyphenyl)-4-methylpiperazine-1-carboxylate (144 mg, 0.4 mmol, 1.0 eq) in HCl(gas)/1,4-dioxane (4 M, 5 mL) was stirred for 1 hour at 25° C. The resulting mixture was concentrated under vacuum. The resulting mixture was diluted with aq. NaHCO₃ (50 mL) and then extracted with CH₂Cl₂ (2×30 mL). The combined organic layer was washed with brine (100 mL), dried over anhydrous Na₂SO₄. After filtration, the filtrate was concentrated under vacuum. The crude product was purified by Prep-CHIRAL-SFC using the following conditions to give (3R)-3-(2-isopropoxyphenyl)-1-methylpiperazine (assumed) as a yellow oil (30 mg, 29.7%) and (3S)-3-(2-isopropoxyphenyl)-1-methylpiperazine (assumed) as a yellow oil (39 mg, 38.6%). A, RT=3.4 min in CHIRAL-SFC, Column: CHIRALPAK IH, 3*25 cm, 5 um; Mobile Phase A: CO₂, Mobile Phase B: EtOH (0.1% 2 M NH₃-MeOH); Flow rate: 85 mL/min; Gradient: 15% B, Wave Length: 220 nm. B, RT=5.6 min in CHIRAL-SFC, Column: CHIRALPAK IH, 3*25 cm, 5 um; Mobile Phase A: CO₂, Mobile Phase B: EtOH (0.1% 2 M NH₃-MeOH); Flow rate: 85 mL/min; Gradient: 15% B, Wave Length: 220 nm. A: ¹HNMR (300 MHz, Chloroform-d) δ 7.44 (dd, J=7.6, 1.8 Hz, 1H), 7.23-7.17 (m, 1H), 6.98-6.82 (m, 2H), 4.63-4.55 (m, 1H), 4.27 (dd, J=10.1, 2.7 Hz, 1H), 3.71 (q, J=7.0 Hz, 1H), 3.42-3.07 (m, 2H), 2.96 (dt, J=10.9, 2.2 Hz, 1H), 2.86-2.82 (m, 1H), 2.33 (s, 3H), 2.03 (t, J=10.5 Hz, 1H), 1.36 (d, J=6.1 Hz, 6H). B: ¹HNMR (300 MHz, Chloroform-d) δ 7.45 (dd, J=7.6, 1.8 Hz, 1H), 7.21 (td, J=7.8, 1.8 Hz, 1H), 6.98-6.82 (m, 2H), 4.63-4.55 (m, J=6.0 Hz, 1H), 4.30 (dd, J=10.2, 2.8 Hz, 1H), 3.13-3.09 (m, 2H), 2.98 (dt, J=11.0, 2.2 Hz, 1H), 2.92-2.77 (m, 1H), 2.35 (s, 3H), 2.29-2.14 (m, 1H), 2.06 (t, J=10.6 Hz, 1H), 1.37 (d, J=6.0 Hz, 6H).

### Example INT_39 Preparation of 2-[(3R,8S)-13-fluoro-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]-N-[3-nitro-4-({[(1r,4r)-4-hydroxy-4-methylcyclohexyl]methyl}amino)benzenesulfonyl]-4-{2-oxo-7-azaspiro[3.5]nonan-7-yl}benzamide

Synthesis of 5-bromo-6-fluoropyridin-2-amine: Into a 10 L 3-necked round-bottom flask were placed 6-fluoropyridin-2-amine (500.0 g, 4460.0 mmol, 1.0 eq) and MeCN (5 L) at 25° C. After that, NBS (817.6 g, 4593.8 mmol, 1.0 eq) was added in portions over 30 min at 15° C. The resulting mixture was stirred for 2 hours at 25° C. The reaction was quenched by the addition of water (500 mL). The resulting mixture was concentrated under vacuum to remove MeCN and the residue (aqueous solution) was extracted with EtOAc (2×5 L). The combined organic layer was washed with brine (5 L), dried over anhydrous Na₂SO₄. After filtration, the filtrate was concentrated under vacuum. The residue was purified by trituration with petroleum ether (2 L). The crude residue was applied onto a silica gel column and eluted with ethyl acetate/petroleum ether=1:1 to give 5-bromo-6-fluoropyridin-2-amine as an off-white solid (800.0 g, 93.9%). LC-MS (ES, m/z) M+1: 191/193. ¹HNMR (300 MHz, DMSO-d₆) δ 7.64 (dd, J=9.6, 8.4 Hz, 1H), 6.56 (bs, 2H), 6.29 (dd, J=8.5, 1.7 Hz, 1H).

Synthesis of ethyl 2-(2-amino-5-bromo-6-fluoropyridin-3-yl)-2,2-difluoroacetate: Into a 5 L 3-necked round-bottom flask were added 5-bromo-6-fluoropyridin-2-amine (200.0 g, 1047.1 mmol, 1.0 eq), DMSO (2 L), ethyl 2-bromo-2,2-difluoroacetate (637.6 g, 3141.3 mmol, 3.0 eq) and ferrocene (19.5 g, 104.7 mmol, 0.1 eq) at 25° C. After that, H₂02 (30%) (237.4 g, 2094.2 mmol, 2.0 eq) was added dropwise over 1 hour at 20° C. The resulting mixture was stirred for additional 4 hours at 25° C. The reaction was quenched by the addition of ice-water (2 L). The precipitated solids were collected by filtration. The filter cake was dissolved in EtOAc (1 L), and then washed with aqueous Na₂SO₃ (1 L) and brine (1 L). The combined organic layer was dried over anhydrous Na₂SO₄. After filtration, the filtrate was concentrated under vacuum. The crude residue was applied onto a silica gel column and eluted with ethyl acetate/petroleum ether=1:1 to give ethyl 2-(2-amino-5-bromo-6-fluoropyridin-3-yl)-2,2-difluoroacetate as an off-white solid (150.0 g, 45.8%). LC-MS (ES, m/z) M+1: 313/315. ¹HNMR (300 MHz, DMSO-d₆) δ 7.92 (d, J=8.5 Hz, 1H), 6.75 (bs, 2H), 4.35 (q, J=7.1 Hz, 2H), 1.26 (t, J=7.1 Hz, 3H).

Synthesis of 5-bromo-3,3,6-trifluoro-1,3-dihydro-2H-pyrrolo[2,3-b]pyridin-2-one: Into a 10 L 3-necked round-bottom flask were placed ethyl 2-(2-amino-5-bromo-6-fluoropyridin-3-yl)-2,2-difluoroacetate (150.0 g, 479.1 mmol, 1.0 eq), DMSO (4500 mL) and Cs₂CO₃ (312.2 g, 958.2 mmol, 2.0 eq) at 25° C. The resulting mixture was stirred for 2 hours at 50° C. The reaction was quenched by the addition of HCl (0.5 M, 3 L) and extracted with EtOAc (2×3 L). The combined organic layer was washed with brine (5 L), dried over anhydrous Na₂SO₄. After filtration, the filtrate was concentrated under vacuum. The residue was purified by trituration with petroleum ether (1 L). The crude residue was applied onto a silica gel column and eluted with ethyl acetate/petroleum ether=1:1 to give 5-bromo-3,3,6-trifluoro-1,3-dihydro-2H-pyrrolo[2,3-b]pyridin-2-one as a yellow solid (110.0 g, 86.0%). LC-MS (ES, m/z) M-1: 265/267. ¹HNMR (300 MHz, DMSO-d₆) δ 12.25 (bs, 1H), 8.71 (dt, J=8.3, 1.5 Hz, 1H).

Synthesis of 5-bromo-3,6-difluoro-1H-pyrrolo[2,3-b]pyridine: Into a 2 L 3-necked round-bottom flask were placed 5-bromo-3,3,6-trifluoro-1,3-dihydro-2H-pyrrolo[2,3-b]pyridin-2-one (50.0 g, 187.3 mmol, 1.0 eq). After that, BH₃-THF (1 L, 1.0 mol, 5.3 eq) was added dropwise over 1 hour at 0° C. The resulting mixture was stirred for 3 hours at 25° C. The reaction was quenched by the addition of aq. NaOH (500 mL, 1 M) at 0° C., and then extracted with CH₂Cl₂ (2×500 mL). The combined organic layer was washed with brine (1 L), dried over anhydrous Na₂SO₄. After filtration, the filtrate was concentrated under vacuum. The crude residue was applied onto a silica gel column and eluted with petroleum ether/ethyl acetate=10:1 to give 5-bromo-3,6-difluoro-1H-pyrrolo[2,3-b]pyridine as a colorless oil (11.0 g, 25.2%). LC-MS (ES, m/z) M-1: 231/233.

Synthesis of 5-bromo-3,6-difluoro-1-((2-(trimethylsilyl)ethoxy)methyl)-1H-pyrrolo[2,3-b]pyridine: Into a 500 mL 3-necked round-bottom flask were added 5-bromo-3,6-difluoro-1H-pyrrolo[2,3-b]pyridine (10.0 g, 42.9 mmol, 1.0 eq) and THE (200 mL) at 25° C. After that, NaH (3.4 g, 85.8 mmol, 2.0 eq, 60%) was added in portions at 0° C. The resulting mixture was stirred for 30 min at 0° C. To the above mixture was added SEM-Cl (10.7 g, 64.4 mmol, 1.5 eq) dropwise at 0° C. The resulting mixture was stirred for additional 2 hours at 25° C. The reaction was quenched by the addition of sat. NH₄Cl (50 mL) at 0° C., and then extracted with EtOAc (2×200 mL). The combined organic layer was washed with brine (300 mL), dried over anhydrous Na₂SO₄. After filtration, the filtrate was concentrated under vacuum. The crude residue was applied onto a silica gel column and eluted with petroleum ether/ethyl acetate=12:1 to give 5-bromo-3,6-difluoro-1-((2-(trimethylsilyl)ethoxy)methyl)-1H-pyrrolo[2,3-b]pyridine as a colorless oil (10.0 g, 64.1%). LC-MS (ES, m/z) M+1: 363/365. ¹HNMR (300 MHz, Chloroform-d) δ 8.20 (dd, J=8.4, 0.6 Hz, 1H), 7.09 (d, J=2.5 Hz, 1H), 5.53 (s, 2H), 3.59-3.40 (m, 2H), 1.00-0.79 (m, 2H), −0.03 (s, 9H).

Synthesis of N-[(3R,4S)-3-[(5-bromo-3-fluoro-1-{[2-(trimethylsilyl)ethoxy]methyl}pyrrolo[2,3-b]pyridin-6-yl)oxy]oxan-4-yl]-4-methylbenzenesulfonamide: Into a 250 mL 3-necked round-bottom flask, were placed N-[(3R,4S)-3-hydroxyoxan-4-yl]-4-methylbenzenesulfonamide (1.7 g, 6.3 mmol, 1.0 eq), tetrahydrofuran (30 mL). After that, NaH (377 mg, 9.4 mmol, 1.5 eq, 60%) was added in portions at 0° C. The resulting mixture was stirred for additional 30 min. To the above mixture was added 5-bromo-3,6-difluoro-1-((2-(trimethylsilyl)ethoxy)methyl)-1H-pyrrolo[2,3-b]pyridine (2.3 g, 6.3 mmol, 1.0 eq) dropwise at 0° C. The resulting mixture was stirred for additional 4 hours at 60° C. The reaction was quenched by the addition of sat. NH₄Cl and then extracted with EtOAc (2×50 mL). The combined organic layer was washed with brine (200 mL), dried over anhydrous Na₂SO₄. After filtration, the filtrate was concentrated under vacuum. The crude residue was applied onto a silica gel column and eluted with petroleum ether/ethyl acetate=7:3 to give N-[(3R,4S)-3-[(5-bromo-3-fluoro-1-{[2-(trimethylsilyl)ethoxy]methyl}pyrrolo[2,3-b]pyridin-6-yl)oxy]oxan-4-yl]-4-methylbenzenesulfonamide as a light yellow oil (2.9 g, 75.3%). LC-MS (ES, m/z) M+1: 614/616.

Synthesis of (3R,8S)-13-fluoro-9-(4-methylbenzenesulfonyl)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraene: Into a 250 mL round-bottom flask, were placed N-[(3R,4S)-3-[(5-bromo-3-fluoro-1-{[2-(trimethylsilyl)ethoxy]methyl}pyrrolo[2,3-b]pyridin-6-yl)oxy]oxan-4-yl]-4-methylbenzenesulfonamide (2.9 g, 4.7 mmol, 1.0 eq), DMSO (30 mL), CuI (540 mg, 2.8 mmol, 0.6 eq), phen (511 mg, 2.8 mmol, 0.6 eq), K₂CO₃ (2.0 g, 14.2 mmol, 3.0 eq). The resulting mixture was stirred for overnight at 120° C. The resulting mixture was diluted with water (300 mL) and extracted with was extracted with EtOAc (2×50 mL). The combined organic layer was washed with brine (300 mL), dried over anhydrous Na₂SO₄. After filtration, the filtrate was concentrated under vacuum. The crude residue was applied onto a silica gel column and eluted with petroleum ether/ethyl acetate=7:3 to give (3R,8S)-13-fluoro-9-(4-methylbenzenesulfonyl)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraene as a light yellow oil (2.0 g, 79.4%). ¹HNMR (300 MHz, Chloroform-d) δ 8.45 (s, 1H), 7.30 (s, 1H), 7.27 (s, 1H), 7.16 (d, J=8.2 Hz, 2H), 7.02 (d, J=2.5 Hz, 1H), 5.49 (s, 2H), 4.22 (dd, J=10.8, 4.5 Hz, 1H), 4.07 (dd, J=11.9, 4.5 Hz, 1H), 3.73-3.42 (m, 5H), 3.37-3.25 (m, 1H), 2.75-2.63 (m, 1H), 2.37 (s, 3H), 2.11 (ddd, J=24.9, 11.9, 5.8 Hz, 1H), 0.96-0.87 (m, 2H), −0.03 (s, 9H).

Synthesis of (3R,8S)-13-fluoro-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraene: Into a 40-mL vial purged and maintained with an inert atmosphere of nitrogen, was placed Na (518 mg, 22.5 mmol, 6.0 eq), Naphthalene (2.9 g, 22.5 mmol, 6.0 eq), 1,2-dimethoxyethane (20 mL). The mixture was stirred for 40 min at 25° C. until the formation of Na/naphthalene is complete. At the same time, another 100 mL 3-necked round-bottom flask purged and maintained with an inert atmosphere of nitrogen, was placed (3R,8S)-13-fluoro-9-(4-methylbenzenesulfonyl)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraene (2.0 g, 3.7 mmol, 1.0 eq), tetrahydrofuran (20 mL). This was followed by the addition of the above solution at −78° C. The resulting mixture was stirred for 3 hours at 25° C. The reaction was then quenched by the addition of sat. NH₄Cl (300 mL) and extracted with EtOAc (3×100 mL). The combined organic layer was washed with brine (300 mL), dried over anhydrous Na₂SO₄. After filtration, the filtrate was concentrated under vacuum. The crude residue was applied onto a silica gel column and eluted with petroleum ether/ethyl acetate=7:3 to give (3R,8S)-13-fluoro-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraene as a light yellow oil (770 mg, 54.1%). LC-MS (ES, m/z) M+1: 380.

Synthesis of 2-bromo-4-{2-oxo-7-azaspiro[3.5]nonan-7-yl}benzoic acid: Into a 100 mL round-bottom flask, were placed methyl 2-bromo-4-{2-oxo-7-azaspiro[3.5]nonan-7-yl}benzoate (2.0 g, 5.7 mmol, 1.0 eq), methanol (5 mL), water (5 mL), NaOH (911 mg, 22.8 mmol, 4.0 eq), The resulting mixture was stirred for 5 hours at 40° C. The resulting mixture was then diluted with water (30 mL) and acidified to pH=5 with HCl (1 M). The precipitated solids were collected by filtration and dried under infrared light to give 2-bromo-4-{2-oxo-7-azaspiro[3.5]nonan-7-yl}benzoic acid as a yellow solid (1.9 g, crude). LC-MS (ES, m/z) M+1: 338/340.

Synthesis of 2-bromo-N-[3-nitro-4-({[(1r,4r)-4-hydroxy-4-methylcyclohexyl]methyl}amino)benzenesulfonyl]-4-{2-oxo-7-azaspiro[3.5]nonan-7-yl}benzamide: Into a 40 mL vial, were placed 2-bromo-4-{2-oxo-7-azaspiro[3.5]nonan-7-yl}benzoic acid (470 mg, 1.4 mmol, 1.0 eq), 3-nitro-4-({[(1r,4r)-4-hydroxy-4-methylcyclohexyl]methyl}amino)benzenesulfonamide (477 mg, 1.4 mmol, 1.0 eq), dichloromethane (10 mL), EDCl (533 mg, 2.8 mmol, 2.0 eq), N,N-dimethylpyridin-4-amine (679 mg, 5.6 mmol, 4.0 eq). The resulting mixture was stirred overnight at 30° C. The resulting mixture was concentrated under vacuum. The crude residue was applied onto a silica gel column and eluted with dichloromethane/ethyl acetate (included 20% methanol)=1:1 to give 2-bromo-N-[3-nitro-4-({[(1r,4r)-4-hydroxy-4-methylcyclohexyl]methyl}amino)benzenesulfonyl]-4-{2-oxo-7-azaspiro[3.5]nonan-7-yl}benzamide as a yellow solid (510 mg, 55.3%). ¹HNMR (300 MHz, DMSO-d₆) δ 12.27 (s, 1H), 8.70-8.53 (m, 2H), 7.92 (dd, J=9.3, 2.3 Hz, 1H), 7.36 (d, J=8.7 Hz, 1H), 7.27 (d, J=9.3 Hz, 1H), 7.11 (d, J=2.4 Hz, 1H), 6.95 (dd, J=9.0, 2.4 Hz, 1H), 4.24 (s, 1H), 3.33 (s, 7H), 2.84 (s, 4H), 1.81-1.61 (m, 7H), 1.55 (d, J=12.6 Hz, 1H), 1.43-0.98 (m, 7H).

Synthesis of 2-[(3R,8S)-13-fluoro-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]-N-[3-nitro-4-({[(1r,4r)-4-hydroxy-4-methylcyclohexyl]methyl}amino)benzenesulfonyl]-4-{2-oxo-7-azaspiro[3.5]nonan-7-yl}benzamide: Into an 8-mL sealed tube purged and maintained with an inert atmosphere of nitrogen, were placed 2-bromo-N-[3-nitro-4-({[(1r,4r)-4-hydroxy-4-methylcyclohexyl]methyl}amino)benzenesulfonyl]-4-{2-oxo-7-azaspiro[3.5]nonan-7-yl}benzamide (200 mg, 0.3 mmol, 1.0 eq), (3R,8S)-13-fluoro-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraene (114 mg, 0.3 mmol, 1.0 eq), N₁, N₂-diphenyloxalamide (43 mg, 0.2 mmol, 0.6 eq), CuI (34 mg, 0.2 mmol, 0.6 eq), t-BuONa (86 mg, 0.9 mmol, 3.0 eq), DMSO (2 mL). The resulting solution was stirred for 2 hours at 120° C. The reaction was quenched by the addition of water (30 mL). and then extracted with ethyl acetate (2×30 mL). The combined organic layer was washed with brine (2×30 mL), dried over anhydrous Na₂SO₄. After filtration, the filtrate was concentrated under vacuum. The crude residue was applied onto a silica gel column and eluted with dichloromethane/methanol=8:1 to give 2-[(3R,8S)-13-fluoro-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]-N-[3-nitro-4-({[(1r,4r)-4-hydroxy-4-methylcyclohexyl]methyl}amino)benzenesulfonyl]-4-{2-oxo-7-azaspiro[3.5]nonan-7-yl}benzamide as a yellow solid (80 mg, 27.6%). LC-MS (ES, m/z) M+1: 962.

### Example INT_40 Preparation of Synthesis of tert-butyl (3R)-3-(2-isopropoxyphenyl)piperazine-1-carboxylate (assumed)

Synthesis of 2-(2-isopropoxyphenyl)pyrazine: Into a 500 mL round-bottom flask were added 2-isopropoxyphenylboronic acid (20.1 g, 115.2 mmol, 1.2 eq), Pd(dppf)Cl₂·CH₂Cl₂ (3.9 g, 4.8 mmol, 0.05 eq), K₂CO₃ (39.8 g, 288.1 mmol, 3.0 eq) and Dioxane/H₂O (200 mL/20 mL) at 25° C. The resulting mixture was stirred for 2 hours at 80° C. under nitrogen atmosphere. The reaction mixture was filtered, and the filter cake was washed with EtOAc (3×100 mL). The filtrate was concentrated under vacuum. The residue was diluted with water (50 mL) and extracted with EtOAc (3×50 mL). The combined organic layer was washed with brine (50 mL), dried over anhydrous Na₂SO₄. After filtration, the filtrate was concentrated under vacuum. The crude residue was applied onto a silica gel column and eluted with ethyl acetate/petroleum ether=1:2 to give 2-(2-isopropoxyphenyl)pyrazine as a yellow solid (15.0 g, 72.9%). ¹HNMR (400 MHz, Chloroform-d) δ 9.25 (d, J=1.6 Hz, 1H), 8.67 (dd, J=2.6, 1.5 Hz, 1H), 8.46 (d, J=2.5 Hz, 1H), 7.87 (dd, J=7.7, 1.8 Hz, 1H), 7.41 (ddd, J=8.3, 7.3, 1.8 Hz, 1H), 7.21-6.95 (m, 2H), 4.66 (p, J=6.1 Hz, 1H), 1.36 (d, J=6.0 Hz, 6H).

Synthesis of 2-(2-isopropoxyphenyl)piperazine hydrochloride: Into a 1000 mL pressure tank reactor were added 2-(2-isopropoxyphenyl)pyrazine (20.0 g, 93.3 mmol, 1.0 eq), HCl (6 mL, 197.4 mmol, 2.1 eq) and EtOH (500 mL) at 25° C. The resulting mixture was stirred for overnight at 25° C. under H₂ atmosphere (5 atm). The resulting mixture was filtered, the filter cake was washed with EtOH (3×30 mL). The filtrate was collected and concentrated under vacuum to give 2-(2-isopropoxyphenyl)piperazine hydrochloride (22.0 g crude), which was used directly to the next step without further purification. LC-MS (ES, m/z) M+1: 220.

Synthesis of tert-butyl 3-(2-isopropoxyphenyl)piperazine-1-carboxylate: A mixture of 2-(2-isopropoxyphenyl)piperazine hydrochloride (1.2 g, 5.447 mmol, 1.0 eq) in DCM (10 mL) was treated with TEA (1.6 g, 16.3 mmol, 3.0 eq), followed by the addition of Boc₂O (1.2 g, 5.4 mmol, 1.0 eq) dropwise at 0° C. The resulting mixture was stirred for 1 hour at 0° C. The reaction was quenched by the addition of water (5 mL) at 0° C. and then extracted with CH₂Cl₂ (3×3 mL). The combined organic layer was washed with brine (10 mL), dried over anhydrous Na₂SO₄. After filtration, the filtrate was concentrated under vacuum. The crude residue was applied onto a silica gel column and eluted with THE/petroleum ether=1:20 to give tert-butyl 3-(2-isopropoxyphenyl)piperazine-1-carboxylate as a brown oil (0.9 g, 48.7%). LC-MS (ES, m/z) M+1: 321.

Synthesis of tert-butyl (3R)-3-(2-isopropoxyphenyl)piperazine-1-carboxylate (assumed) and (3S)-3-(2-isopropoxyphenyl)piperazine-1-carboxylate (assumed): The crude product (7.0 g, 21.8 mmol, 1.0 eq) was purified by Prep-CHIRAL-SFC using the following conditions to give tert-butyl (3R)-3-(2-isopropoxyphenyl)piperazine-1-carboxylate (assumed) (2.4 g, 34.3%) and tert-butyl (3S)-3-(2-isopropoxyphenyl)piperazine-1-carboxylate (assumed) (3.1 g, 44.3%) as an off-white oil. A: RT=3.4 min in CHIRAL ART Cellulose-SZ, 3*25 cm, 5 μm; Mobile Phase A: CO₂, Mobile Phase B: MeOH (0.1% 2 M NH₃-MeOH); Flow rate: 80 mL/min; Gradient: isocratic 15% B; Column Temperature: 35° C.; Back Pressure: 100 bar; Wave Length: 220 nm; Sample Solvent: MeOH—HPLC; Injection Volume: 0.8 mL; Number of Runs: 80. B: RT=4.1 min in CHIRAL ART Cellulose-SZ, 3*25 cm, 5 μm; Mobile Phase A: CO₂, Mobile Phase B: MeOH (0.1% 2 M NH₃-MeOH); Flow rate: 80 mL/min; Gradient: isocratic 15% B; Column Temperature: 35° C.; Back Pressure: 100 bar; Wave Length: 220 nm; Sample Solvent: MeOH—HPLC; Injection Volume: 0.8 mL; Number of Runs: 80. LC-MS (ES, m/z) M+1: 321. A: ¹HNMR (300 MHz, DMSO-d₆) δ 7.61-7.37 (m, 1H), 7.21 (ddd, J=8.2, 7.3, 1.8 Hz, 1H), 7.04-6.95 (m, 1H), 6.90 (td, J=7.5, 1.1 Hz, 1H), 4.65 (p, J=6.0 Hz, 1H), 4.10 (q, J=5.2 Hz, 1H), 3.87 (dd, J=10.2, 3.1 Hz, 2H), 2.98 (d, J=10.5 Hz, 1H), 2.72-2.63 (m, 1H), 1.41 (s, 9H), 1.30 (d, J=6.0 Hz, 6H), 1.11-1.01 (m, 1H), 0.95-0.68 (m, 1H). B: ¹HNMR (300 MHz, DMSO-d₆) δ 7.55-7.37 (m, 1H), 7.20 (ddd, J=8.8, 7.4, 1.8 Hz, 1H), 7.04-6.95 (m, 1H), 6.90 (td, J=7.5, 1.1 Hz, 1H), 4.64 (q, J=6.0 Hz, 1H), 4.07 (s, 2H), 3.87 (dd, J=10.2, 3.1 Hz, 2H), 3.02-2.87 (m, 1H), 2.80-2.58 (m, 1H), 2.38 (t, J=11.2 Hz, 1H), 1.41 (s, 9H), 1.30 (d, J=6.0 Hz, 6H).

### Example INT_41 Preparation of (2R)-2-(2-isopropoxyphenyl)piperidine (assumed) and (2S)-2-(2-isopropoxyphenyl)piperidine (assumed)

Synthesis of 2-(2-isopropoxyphenyl)pyridine: Into a 250 mL round-bottom flask were added 2-isopropoxyphenylboronic acid (6.8 g, 40.0 mmol, 1.2 eq), 2-bromopyridine (5.0 g, 31.6 mmol, 1.0 eq), Pd(dppf)Cl₂·CH₂Cl₂ (1.3 g, 1.6 mmol, 0.05 eq), K₂CO₃ (13.1 g, 94.9 mmol, 3 eq) and dioxane/water (50 mL/10 mL) at 25° C. The resulting mixture was stirred for overnight at 80° C. under nitrogen atmosphere. The resulting mixture was filtered, the filter cake was washed with EtOAc (3×30 mL). The filtrate was concentrated under reduced pressure. The resulting mixture was diluted with water (30 mL) and extracted with EtOAc (3×30 mL). The combined organic layer was washed with brine (30 mL), dried over anhydrous Na₂SO₄. After filtration, the filtrate was concentrated under reduced pressure. The crude residue was applied onto a silica gel column and eluted with ethyl acetate/petroleum ether=1:2 to give 2-(2-isopropoxyphenyl)pyridine as an off-white liquid (6.0 g, 88.9%). ¹HNMR (300 MHz, Chloroform-d) δ 8.72 (ddd, J=4.9, 1.9, 1.0 Hz, 1H), 7.93 (dt, J=8.0, 1.1 Hz, 1H), 7.84 (dd, J=7.7, 1.8 Hz, 1H), 7.69 (ddd, J=8.0, 7.5, 1.9 Hz, 1H), 7.34 (ddd, J=8.2, 7.3, 1.9 Hz, 1H), 7.20 (ddd, J=7.5, 4.9, 1.2 Hz, 1H), 7.09 (td, J=7.5, 1.1 Hz, 1H), 7.01 (dd, J=8.2, 1.2 Hz, 1H), 4.54 (p, J=6.1 Hz, 1H), 1.30 (d, J=6.0 Hz, 6H).

Synthesis of 2-(2-isopropoxyphenyl)piperidine: Into a 250 mL pressure tank reactor were added 2-(2-isopropoxyphenyl)pyridine (3.0 g, 14.0 mmol, 1.0 eq), HCl (1 mL, 32.9 mmol, 2.3 eq) and EtOH (100 mL) at 25° C. The resulting mixture was stirred for overnight at 25° C. under H₂ atmosphere (5 atm). The resulting mixture was filtered, the filter cake was washed with EtOH (3×20 mL). The filtrate was collected and concentrated under reduced pressure. The residue was purified by reverse flash chromatography using the following conditions: column, C18 silica gel; mobile phase, CH₃CN in water (0.05% TFA), 10% to 50% gradient in 10 min; detector, UV 254 nm. Finally, 2-(2-isopropoxyphenyl)piperidine was obtained as a light yellow oil (2.2 g, 71.3%). LC-MS (ES, m/z) M+1: 220.

Synthesis of (2R)-2-(2-isopropoxyphenyl)piperidine (assumed) and (2S)-2-(2-isopropoxyphenyl)piperidine (assumed): The crude product (150 mg) was purified by Prep-CHIRAL-SFC using the following conditions to give (2R)-2-(2-isopropoxyphenyl)piperidine (assumed) as a white solid (60 mg, 42.9%) and (2S)-2-(2-isopropoxyphenyl)piperidine (assumed) as a white oil (70 mg, 50.0%). A: RT=7.2 min in CHIRAL SFC, Column: CHIRALPAK IG, 3*25 cm, 5 μm; Mobile Phase A: CO₂, Mobile Phase B: MeOH (0.1% 2 M NH₃-MEOH); Flow rate: 80 mL/min; Gradient: isocratic 15% B; Wave Length: 220 nm; B: RT=9.0 min in CHIRAL SFC, Column: CHIRALPAK IG, 3*25 cm, 5 μm; Mobile Phase A: CO₂, Mobile Phase B: MeOH (0.1% 2 M NH₃-MeOH); Flow rate: 80 mL/min; Gradient: isocratic 15% B; Wave Length: 220 nm; LC-MS (ES, m/z) M+1: 220. A: ¹HNMR (300 MHz, DMSO-d₆) δ 7.43 (dd, J=7.6, 1.7 Hz, 1H), 7.34 (ddd, J=8.8, 7.4, 1.6 Hz, 1H), 7.10 (d, J=8.3 Hz, 1H), 7.00 (td, J=7.5, 1.1 Hz, 1H), 4.69 (p, J=6.0 Hz, 1H), 4.45-4.21 (m, 1H), 3.36 (d, J=12.4 Hz, 1H), 3.18-2.94 (m, 1H), 1.90-1.86 (m, 3H), 1.74-1.57 (m, 3H), 1.32 (dd, J=12.5, 6.0 Hz, 6H). B: ¹HNMR (300 MHz, DMSO-d₆) δ 7.42 (dd, J=7.6, 1.8 Hz, 1H), 7.16 (ddd, J=8.8, 7.3, 1.8 Hz, 1H), 6.94 (dd, J=8.4, 1.1 Hz, 1H), 6.88 (td, J=7.4, 1.1 Hz, 1H), 4.59 (p, J=6.0 Hz, 1H), 3.87 (dd, J=10.8, 2.3 Hz, 1H), 3.14-2.99 (m, 1H), 2.67 (td, J=11.6, 2.9 Hz, 1H), 1.84-1.68 (m, 3H), 1.61-1.36 (m, 3H), 1.28 (dd, J=6.0, 3.2 Hz, 6H).

### Example INT_42 Preparation of N-((4-((((1r,4r)-4-hydroxy-4-methylcyclohexyl)methyl)amino)-3-nitrophenyl)sulfonyl)-4-(2-oxo-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide

Synthesis of N-((4-((((1r,4r)-4-hydroxy-4-methylcyclohexyl)methyl)amino)-3-nitrophenyl)sulfonyl)-4-(2-oxo-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide: Into a 40 mL vial, were placed N-((4-((((1r,4r)-4-hydroxy-4-methylcyclohexyl)methyl)amino)-3-nitrophenyl)sulfonyl)-4-(2-oxo-7-azaspiro[3.5]nonan-7-yl)-2-((5aS,9aR)-1-((2-(trimethylsilyl)ethoxy)methyl)-6,7,9,9a-tetrahydro-1H-pyrano[3,4-b]pyrrolo[3′,2′:5,6]pyrido[3,2-e][1,4]oxazin-5(5aH)-yl)benzamide (1.5 g, 1.6 mmol, 1.0 eq), ethylenediamine (100 mg, 1.6 mmol, 1.0 eq) and TBAF in THE (1 M, 20 mL). The resulting mixture was stirred for 6 hours at 60° C. under N₂ atmosphere. The reaction mixture was diluted with EtOAc (500 mL) and then washed with water (10×200 mL). The organic layer was dried over anhydrous Na₂SO₄. After filtration, the filtrate was concentrated under vacuum. The crude residue was applied onto a silica gel column and eluted with dichloromethane/methanol=10:1 to give N-((4-((((1r,4r)-4-hydroxy-4-methylcyclohexyl)methyl)amino)-3-nitrophenyl)sulfonyl)-4-(2-oxo-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide as a yellow solid (1.0 g, 77.3%). LC-MS (ES, m/z) M+1: 814.

### Example INT_43 Preparation of 5-(methoxy-d3)-6-morpholinonicotinaldehyde

Synthesis of 2-bromo-3-(methoxy-d3)pyridine: To a stirred solution of 2-bromopyridin-3-ol (20.0 g, 57.5 mmol, 1 eq) in DMF (300 mL) was added iodomethane-d₃ (33.7 g, 222.9 mmol, 2 eq) and K₂CO₃ (46.8 g, 342.4 mmol, 3.0 eq) in portions at 0° C. The resulting mixture was stirred for 16 hours at 25° C. The reaction mixture was diluted with ethyl acetate (400 mL) and washed with water (3×100 mL). The aqueous layer was extracted with ethyl acetate (2×100 mL). The combined organic layer was washed with brine (3×100 mL), dried over anhydrous Na₂SO₄. After filtration, the filtrate was concentrated under reduced pressure. The residue was purified by silica gel column chromatography and eluted with ethyl acetate/petroleum ether=1:1 to afford 2-bromo-3-(methoxy-d₃)pyridine as a yellow oil (15.7 g, 72.8%). LC-MS (ES, m/z) M+1: 191.

Synthesis of 4-(3-(methoxy-d3)pyridin-2-yl)morpholine: Into a 250 mL round-bottom flask were added 2-bromo-3-(methoxy-d3)pyridine (15.0 g, 79.8 mmol, 1.0 eq) and morpholine (100 mL) at 25° C. The resulting mixture was stirred for 48 hours at 125° C. The reaction mixture was diluted with water (300 mL) and then extracted with ethyl acetate (3×100 mL). The mixture was dried over anhydrous Na₂SO₄. After filtration, the filtrate was concentrated under vacuum. The crude residue was purified onto a silica gel column and eluted with ethyl acetate/petroleum ether=1:1 to give 4-(3-(methoxy-d₃) pyridin-2-yl) morpholine as a yellow oil (10.0 g, 64.5%). LC-MS (ES, m/z) M+1: 198.

Synthesis of 4-(5-bromo-3-(methoxy-d₃)pyridin-2-yl)morpholine: Into a 250 mL round-bottom flask were added 4-(3-(methoxy-d₃)pyridin-2-yl)morpholine (10.0 g, 51.5 mmol, 1.0 eq), NBS (11.9 g, 66.9 mmol, 1.3 eq) and DMF (100 mL) at 25° C. The resulting mixture was stirred for 20 hours at 80° C. The reaction mixture was diluted with ethyl acetate (300 mL) and then washed with water (3×100 mL). The organic phase was dried over anhydrous Na₂SO₄. After filtration, the filtrate was concentrated under vacuum. The crude residue was purified onto a silica gel column and eluted with ethyl acetate/petroleum ether=1:1 to give 4-(5-bromo-3-(methoxy-d3)pyridin-2-yl)morpholine as a yellow oil (5.0 g, 35.6%). LC-MS (ES, m/z) M+1: 276.

Synthesis of 5-(methoxy-d3)-6-morpholinonicotinaldehyde: To a solution of 4-(5-bromo-3-(methoxy-d3)pyridin-2-yl)morpholine (5.0 g, 18.3 mmol, 1.0 eq) in THE (100 mL) was treated with t-BuLi (1.3 g, 20.1 mmol, 1.1 eq) for 1 hour at −78° C. under nitrogen atmosphere, which was followed by the addition of DMF (1.6 g, 21.9 mmol, 1.2 eq) in portions at −78° C. The resulting mixture was stirred for 2 hours at −78° C. under N₂ atmosphere. The reaction was quenched by the addition of aq. NH₄Cl (30 mL) at 0° C. and then extracted with ethyl acetate (3×50 mL). The combined organic layer was washed with water (3×15 mL). The mixture was dried over anhydrous Na₂SO₄. After filtration, the filtrate was concentrated under vacuum. The crude residue was purified onto a silica gel column and eluted with ethyl acetate/petroleum ether=1:1 to give 5-(methoxy-d3)-6-morpholinonicotinaldehyde as a yellow solid (2.0 g, 49.2%). LC-MS (ES, m/z) M+1: 226.

### Example INT_44 Preparation of 2H,3H-[1,4]dioxino[2,3-b]yridine-7-carbaldehyde

Synthesis of 2H,3H-[1,4]dioxino[2,3-b]yridine-7-carbaldehyde: A solution of 7-bromo-2H,3H-[1,4]dioxino[2,3-b]pyridine (500 mg, 2.3 mmol, 1.0 eq) in THE (5 mL) was treated with n-BuLi (178 mg, 2.8 mmol, 1.2 eq) for 1 hour at −78° C. under nitrogen atmosphere, which was followed by the addition of DMF (1.0 g, 13.9 mmol, 6.0 eq) dropwise at −78° C. The reaction was quenched by the addition of sat. NH₄Cl (5 mL) at 0° C. and then extracted with EtOAc (3×10 mL). The combined organic layer was washed with brine (10 mL), dried over anhydrous Na₂SO₄. After filtration, the filtrate was concentrated under vacuum. The crude residue was applied onto a silica gel column and eluted with ethyl acetate/petroleum ether=1:3 to give 2H,3H-[1,4]dioxino[2,3-b]pyridine-7-carbaldehyde as a white solid (180 mg, 47.1%). ¹HNMR (300 MHz, Chloroform-d) δ 9.97 (s, 1H), 8.36 (d, J=2.0 Hz, 1H), 7.70 (d, J=2.0 Hz, 1H), 4.66-4.51 (m, 2H), 4.41-4.20 (m, 2H).

### Example INT_45 Preparation of 5-methoxy-6-morpholinonicotinaldehyde

Synthesis of 4-(3-methoxypyridin-2-yl)morpholine: Into a 250 mL round-bottom flask were added 2-bromo-3-methoxypyridine (15.0 g, 79.8 mmol, 1.0 eq) and morpholine (100 mL) at 25° C. The resulting mixture was stirred for 48 hours at 125° C. The resulting mixture was diluted with water (300 mL) and extracted with ethyl acetate (3×100 mL). The combined organic layer was dried over anhydrous Na₂SO₄. After filtration, the filtrate was concentrated under vacuum. The crude residue was purified onto a silica gel column and eluted with ethyl acetate/petroleum ether=1:1 to give 4-(3-methoxypyridin-2-yl)morpholine as a yellow oil (10.0 g, 64.5%). LC-MS (ES, m/z) M+1: 195.

Synthesis of 4-(5-bromo-3-methoxypyridin-2-yl)morpholine: Into a 250 mL round-bottom flask were added 4-(3-methoxypyridin-2-yl)morpholine (10.0 g, 51.5 mmol, 1.0 eq), NBS (11.9 g, 66.9 mmol, 1.3 eq) and DMF (100 mL) at 25° C. The resulting mixture was stirred for 20 hours at 80° C. The reaction mixture was diluted with ethyl acetate (3×100 mL) and then washed with water (3×100 mL). The combined organic layer was dried over anhydrous Na₂SO₄. After filtration, the filtrate was concentrated under vacuum. The crude residue was purified onto a silica gel column and eluted with ethyl acetate/petroleum ether=1:1 to give 4-(5-bromo-3-methoxypyridin-2-yl)morpholine as a yellow oil (5.0 g, 35.6%). LC-MS (ES, m/z) M+1: 273.

Synthesis of 5-methoxy-6-morpholinonicotinaldehyde: A solution of 4-(5-bromo-3-methoxypyridin-2-yl)morpholine (5.0 g, 18.3 mmol, 1.0 eq) in THE (100 mL) was treated with t-BuLi (1.3 g, 20.1 mmol, 1.1 eq) for 1 hour at −78° C. under nitrogen atmosphere, which was followed by the addition of DMF (1.6 g, 21.9 mmol, 1.2 eq) in portions at −78° C. The resulting mixture was stirred for 2 hours at −78° C. under N₂ atmosphere. The reaction was quenched by the addition of aq. NH₄Cl (30 mL) at 0° C. and then extracted with ethyl acetate (3×50 mL). The combined organic layer was washed with water (3×15 mL), and dried over anhydrous Na₂SO₄. After filtration, the filtrate was concentrated under vacuum. The crude residue was purified onto a silica gel column and eluted with ethyl acetate/petroleum ether=1:1 to give 5-methoxy-6-morpholinonicotinaldehyde as a yellow solid (2.0 g, 49.2%). LC-MS (ES, m/z) M+1: 223.

### Example 79: Preparation of 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(3R)-3-(2-isopropoxyphenyl)morpholin-4-yl]-7-azaspiro[3.5]nonan-7-yl}-N-[3-nitro-4-({[(1r,4r)-4-hydroxy-4-methylcyclohexyl]methyl}amino)benzenesulfonyl]benzamide hydrochloride

Synthesis of 4-{2-[(3R)-3-(2-isopropoxyphenyl)morpholin-4-yl]-7-azaspiro[3.5]nonan-7-yl}-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide: Into a 40 mL vial, were placed N-[3-nitro-4-({[(1r,4r)-4-hydroxy-4-methylcyclohexyl]methyl}amino)benzenesulfonyl]-4-{2-oxo-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide (100 mg, 0.1 mmol, 1.0 eq), (3R)-3-(2-isopropoxyphenyl)morpholine (23 mg, 0.1 mmol, 1.0 eq), methanol (5 mL), ZnCl₂ (29 mg, 0.2 mmol, 2.0 eq), NaBH₃CN (33 mg, 0.5 mmol, 5.0 eq). The reaction mixture was stirred for 3 hours at 80° C. under nitrogen atmosphere. The resulting mixture was quenched by the addition of water (100 mL) and extracted with EtOAc (2×50 mL). The combined organic layer was washed with brine (200 mL), dried over anhydrous Na₂SO₄. After filtration, the filtrate was concentrated under vacuum. The residue was purified by Prep-TLC (dichloromethane/methanol=11:1) to give 4-{2-[(3R)-3-(2-isopropoxyphenyl)morpholin-4-yl]-7-azaspiro[3.5]nonan-7-yl}-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide as a yellow solid (110 mg, 90.4%). LC-MS (ESI, m/z) M+1: 1149.

Synthesis of 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(3R)-3-(2-isopropoxyphenyl)morpholin-4-yl]-7-azaspiro[3.5]nonan-7-yl}-N-[3-nitro-4-({[(1r,4r)-4-hydroxy-4-methylcyclohexyl]methyl}amino)benzenesulfonyl]benzamide hydrochloride: Into a 40 mL vial, were placed 4-{2-[(3R)-3-(2-isopropoxyphenyl)morpholin-4-yl]-7-azaspiro[3.5]nonan-7-yl}-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide (110 mg, 0.1 mmol, 1.0 eq), TBAF-THF (5 mL, 1 M), ethylenediamine (115 mg, 1.9 mmol, 20.0 eq). The reaction mixture was stirred for 6 hours at 70° C. The resulting mixture was quenched by the addition of water (100 mL) and extracted with EtOAc (3×30 mL). The combined organic layer was washed with brine (100 mL), dried over anhydrous Na₂SO₄. After filtration, the filtrate was concentrated under vacuum. The crude product was purified by Prep-HPLC using the following conditions (Prep-HPLC-003): Column, SunFire Prep C18 OBD Column, 19*150 mm, 5 μm 10 nm; mobile phase, water (0.05% trifluoroacetic acid) and acetonitrile (35% acetonitrile up to 75% in 10 min); Detector, UV (220 nm). The collected solution was concentrated under vacuum to remove acetonitrile and the resulting solution was dried by lyophilization (added with Conc·HCl (1 drop)) to give 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(3R)-3-(2-isopropoxyphenyl)morpholin-4-yl]-7-azaspiro[3.5]nonan-7-yl}-N-[3-nitro-4-({[(1r,4r)-4-hydroxy-4-methylcyclohexyl]methyl}amino)benzenesulfonyl]benzamide hydrochloride as a yellow solid (38 mg, 37.6%). LC-MS (ESI, m/z) M-HCl+1: 1019. ¹HNMR (400 MHz, DMSO-d₆) δ 12.74 (s, 1H), 12.06 (s, 1H), 11.25 (s, 1H), 8.56 (t, J=5.8 Hz, 1H), 8.38 (d, J=2.3 Hz, 1H), 7.92 (s, 1H), 7.78 (d, J=9.1 Hz, 1H), 7.42 (dd, J=9.2, 2.3 Hz, 1H), 7.39-7.33 (m, 1H), 7.16-7.05 (m, 2H), 7.04-6.80 (m, 4H), 6.44 (s, 1H), 6.05 (dd, J=3.4, 1.9 Hz, 1H), 4.78-4.59 (m, 2H), 4.32-3.88 (m, 5H), 3.88-3.62 (m, 4H), 3.47-2.94 (m, 10H), 2.19 (t, J=10.6 Hz, 1H), 2.09 (d, J=7.6 Hz, 1H), 1.79-1.21 (m, 21H), 1.12 (d, J=12.1 Hz, 4H), 0.98 (s, 1H).

### Example 80: Preparation of 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(3S)-3-(2-isopropoxyphenyl)morpholin-4-yl]-7-azaspiro[3.5]nonan-7-yl}-N-[3-nitro-4-({[(1r,4r)-4-hydroxy-4-methylcyclohexyl]methyl}amino)benzenesulfonyl]benzamide hydrochloride

Synthesis of 4-{2-[(3S)-3-(2-isopropoxyphenyl)morpholin-4-yl]-7-azaspiro[3.5]nonan-7-yl}-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide: Into a 40 mL vial, were placed N-[3-nitro-4-({[(1r,4r)-4-hydroxy-4-methylcyclohexyl]methyl}amino)benzenesulfonyl]-4-{2-oxo-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide (100 mg, 0.1 mmol, 1.0 eq), (3S)-3-(2-isopropoxyphenyl)morpholine (23 mg, 0.1 mmol, 1.0 eq), methanol (5 mL), ZnCl₂ (29 mg, 0.2 mmol, 2.0 eq), NaBH₃CN (33 mg, 0.5 mmol, 5.0 eq). The reaction mixture was stirred for 3 hours at 80° C. under nitrogen atmosphere. The resulting mixture was quenched by the addition of water (100 mL) and extracted with EtOAc (2×50 mL). The combined organic layer was washed with brine (200 mL), dried over anhydrous Na₂SO₄. After filtration, the filtrate was concentrated under vacuum. The residue was purified by Prep-TLC (dichloromethane/methanol=11:1) to give 4-{2-[(3S)-3-(2-isopropoxyphenyl)morpholin-4-yl]-7-azaspiro[3.5]nonan-7-yl}-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide as a yellow solid (110 mg, 90.4%). LC-MS (ESI, m/z) M+1: 1149.

Synthesis of 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(3S)-3-(2-isopropoxyphenyl)morpholin-4-yl]-7-azaspiro[3.5]nonan-7-yl}-N-[3-nitro-4-({[(1r,4r)-4-hydroxy-4-methylcyclohexyl]methyl}amino)benzenesulfonyl]benzamide hydrochloride: Into a 40 mL vial, were placed 4-{2-[(3S)-3-(2-isopropoxyphenyl)morpholin-4-yl]-7-azaspiro[3.5]nonan-7-yl}-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide (110 mg, 0.1 mmol, 1.0 eq), TBAF-THF (5 mL, 1 M), ethylenediamine (115 mg, 1.9 mmol, 20.0 eq). The reaction mixture was stirred for 6 hours at 70° C. The resulting mixture was quenched by the addition of water (100 mL) and extracted with EtOAc (3×30 mL). The combined organic layer was washed with brine (100 mL), dried over anhydrous Na₂SO₄. After filtration, the filtrate was concentrated under vacuum. The residue was purified by Prep-TLC (dichloromethane/methanol=11:1). The crude product was purified by Prep-HPLC using the following conditions (Prep-HPLC-003): Column, SunFire Prep C18 OBD Column, 19*150 mm, 5 μm 10 nm; mobile phase, water (0.05% trifluoroacetic acid) and acetonitrile (35% acetonitrile up to 75% in 10 min); Detector, UV (220 nm). The collected solution was concentrated under vacuum to remove acetonitrile and the resulting solution was dried by lyophilization (added with Conc·HCl (1 drop)) to give 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(3S)-3-(2-isopropoxyphenyl)morpholin-4-yl]-7-azaspiro[3.5]nonan-7-yl}-N-[3-nitro-4-({[(1r,4r)-4-hydroxy-4-methylcyclohexyl]methyl}amino)benzenesulfonyl]benzamide hydrochloride as a yellow solid (37 mg, 36.6%). LC-MS (ESI, m/z) M-HCl+1: 1019. ¹HNMR (400 MHz, DMSO-d₆) δ 12.75 (s, 1H), 12.12 (s, 1H), 11.25 (s, 1H), 8.57 (t, J=5.9 Hz, 1H), 8.38 (d, J=2.3 Hz, 1H), 7.93 (d, J=7.8 Hz, 1H), 7.78 (dd, J=9.0, 2.3 Hz, 1H), 7.45-7.32 (m, 2H), 7.15-7.04 (m, 2H), 6.98 (dt, J=14.9, 8.3 Hz, 2H), 6.84 (d, J=9.7 Hz, 2H), 6.44 (s, 1H), 6.05 (dd, J=3.4, 1.9 Hz, 1H), 4.79-4.59 (m, 2H), 4.29-3.92 (m, 5H), 3.89-3.64 (m, 4H), 3.44-2.98 (m, 10H), 2.28-2.02 (m, 2H), 1.75-1.22 (m, 21H), 1.19-1.05 (m, 4H), 0.96 (s, 1H).

### Example 81: Preparation of 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2S)-2-(2-methylphenyl)piperidin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide hydrochloride

Synthesis of tert-butyl 2-(2-methylphenyl)piperidine-1-carboxylate: Into a 100 mL round-bottom flask, were placed tert-butyl 2-(2-bromophenyl)piperidine-1-carboxylate (1.0 g, 2.9 mmol, 1.0 eq) and trifluoro(methyl)-lambda4-borane potassium (860 mg, 7.1 mmol, 2.4 eq), dioxane (27 mL), water (3 mL), K₂CO₃ (1.2 g, 8.8 mmol, 3.0 eq), Pd(dppf)Cl₂·CH₂Cl₂ (240 mg, 0.3 mmol, 0.1 eq). The reaction mixture was stirred overnight at 80° C. under nitrogen atmosphere. The resulting mixture was quenched by the addition of water (200 mL) and extracted with ethyl acetate (3×50 mL). The combined organic layer was washed with brine (200 mL), dried over anhydrous Na₂SO₄. After filtration, the filtrate was concentrated under vacuum. The crude residue was applied onto a silica gel column and eluted with ethyl acetate/petroleum ether=1:10 to give tert-butyl 2-(2-methylphenyl)piperidine-1-carboxylate as a light yellow oil (700 mg, 86.5%). ¹HNMR (300 MHz, Chloroform-d) δ 7.24 (d, J=5.0 Hz, 1H), 7.21-7.10 (m, 3H), 5.28-5.19 (m, 1H), 4.21-4.01 (m, 1H), 3.33-3.18 (m, 1H), 2.35 (s, 3H), 2.02-1.56 (m, 6H), 1.30 (s, 9H).

Synthesis of (2S)-2-(2-methylphenyl)piperidine and (2R)-2-(2-methylphenyl)piperidine: A solution of tert-butyl 2-(2-methylphenyl)piperidine-1-carboxylate (700 mg, 2.5 mmol, 1.0 eq) in HCl (gas) in 1,4-dioxane (10 mL) was stirred for 1 hour at 25° C. The resulting mixture was concentrated under vacuum. The resulting mixture was diluted with saturated aqueous NaHCO₃ and extracted with CH₂Cl₂ (2×50 mL). The combined organic layer was washed with brine (100 mL), dried over anhydrous Na₂SO₄. After filtration, the filtrate was concentrated under vacuum. The crude product was purified by Prep-CHIRAL-HPLC. Finally, (2S)-2-(2-methylphenyl)piperidine was obtained as a light yellow oil (200 mg, 44.9%) and (2R)-2-(2-methylphenyl)piperidine was obtained as a light yellow oil (200 mg, 44.9%). A, T_{R}=1.116 min in CHIRAL-SFC, Column: CHIRAL ART Amylose-C NEO, 3*25 cm, 5 um; Mobile Phase A: CO₂, Mobile Phase B: MeOH (0.1% 2M NH₃-MeOH); Flow rate: 80 mL/min; Gradient: 10% B to 10% B in 10 min, Wave Length: 220 nm. B, T_{R}=1.740 min in CHIRAL-SFC, Column: CHIRAL ART Amylose-C NEO, 3*25 cm, 5 um; Mobile Phase A: CO₂, Mobile Phase B: MeOH; DCM=2:1; Flow rate: 80 mL/min; Gradient: 10% B to 10% B in 10 min, Wave Length: 220 nm. LC-MS (ESI, m/z) M+1: 176 (peak 1).

Synthesis of 4-{2-[(2S)-2-(2-methylphenyl)piperidin-1-yl]-7-azaspiro[3.5]nonan-7-yl}-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide: Into an 8 mL vial, were placed (2S)-2-(2-methylphenyl)piperidine (15 mg, 0.09 mmol, 1.0 eq), N-[3-nitro-4-({[(1r,4r)-4-hydroxy-4-methylcyclohexyl]methyl}amino)benzenesulfonyl]-4-{2-oxo-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide (81 mg, 0.09 mmol, 1.0 eq), methanol (2 mL), ZnCl₂ (23.3 mg, 0.2 mmol, 2.0 eq), NaBH₃CN (27 mg, 0.4 mmol, 5.0 eq). The reaction mixture was stirred overnight at 80° C. The resulting mixture was quenched by the addition of water (100 mL) and extracted with EtOAc (3×30 mL). The combined organic layer was washed with brine (100 mL), dried over anhydrous Na₂SO₄. After filtration, the filtrate was concentrated under vacuum. The residue was purified by Prep-TLC (dichloromethane/methanol=15:1) to give 4-{2-[(2S)-2-(2-methylphenyl)piperidin-1-yl]-7-azaspiro[3.5]nonan-7-yl}-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide as a yellow solid (80 mg, 84.7%). LC-MS (ESI, m/z) M-HCl+1: 1103.

Synthesis of 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2S)-2-(2-methylphenyl)piperidin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide hydrochloride: Into a 40 mL vial, were placed 4-{2-[(2S)-2-(2-methylphenyl)piperidin-1-yl]-7-azaspiro[3.5]nonan-7-yl}-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide (80 mg, 0.07 mmol, 1.0 eq), TBAF-THF (5 mL, 1 M), ethylenediamine (87 mg, 1.4 mmol, 20.0 eq). The reaction mixture was stirred for 6 hours at 70° C. The resulting mixture was quenched by the addition of water (100 mL), and then extracted with EtOAc (3×30 mL). The combined organic layer was washed with brine (100 mL), dried over anhydrous Na₂SO₄. After filtration, the filtrate was concentrated under vacuum. The residue was purified by Prep-TLC (dichloromethane/methanol=12:1). The crude product was purified by Prep-HPLC using the following conditions (Prep-HPLC-003): Column, SunFire Prep C18 OBD Column, 19*150 mm, 5 μm 10 nm; mobile phase, water (0.05% trifluoroacetic acid) and acetonitrile (35% acetonitrile up to 75% in 10 min); Detector, UV. The collected solution was concentrated under vacuum to remove acetonitrile and the resulting solution was dried by lyophilization (added with Conc·HCl (1 drop). Finally, 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2S)-2-(2-methylphenyl)piperidin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide hydrochloride was obtained as a yellow solid (30 mg, 39.1%). LC-MS (ESI, m/z) M-HCl+1: 973. ¹HNMR (400 MHz, DMSO-d₆) δ 12.75 (s, 1H), 11.25 (s, 1H), 11.08 (bs, 1H), 8.95 (s, 1H), 8.56 (d, J=6.3 Hz, 1H), 8.39 (dd, J=7.3, 2.3 Hz, 1H), 7.90 (s, 1H), 7.78 (d, J=9.0 Hz, 1H), 7.43 (dd, J=9.3, 2.3 Hz, 1H), 7.27-7.18 (m, 3H), 7.14 (t, J=2.9 Hz, 1H), 6.98 (dd, J=8.7, 2.3 Hz, 1H), 6.83 (d, J=3.9 Hz, 1H), 6.44 (s, 1H), 6.07-6.00 (m, 1H), 4.36-4.22 (m, 2H), 4.14-4.04 (m, 1H), 3.81 (d, J=10.8 Hz, 2H), 3.72-3.58 (m, 1H), 3.49-3.22 (m, 4H), 3.21-2.85 (m, 6H), 2.40 (d, J=7.3 Hz, 3H), 2.19-0.91 (m, 27H).

### Example 82: Preparation of 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2R)-2-(2-methylphenyl)piperidin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide hydrochloride

Synthesis of 4-{2-[(2R)-2-(2-methylphenyl)piperidin-1-yl]-7-azaspiro[3.5]nonan-7-yl}-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide: Into an 8 mL vial, were placed (2R)-2-(2-methylphenyl)piperidine (15 mg, 0.09 mmol, 1.0 eq), N-[3-nitro-4-({[(1r,4r)-4-hydroxy-4-methylcyclohexyl]methyl}amino)benzenesulfonyl]-4-{2-oxo-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide (81 mg, 0.09 mmol, 1.0 eq), methanol (2 mL), ZnCl₂ (23 mg, 0.2 mmol, 2.0 eq), NaBH₃CN (27 mg, 0.4 mmol, 5.0 eq). The reaction mixture was stirred overnight at 80° C. The resulting mixture was quenched by the addition of water (100 mL) and extracted with EtOAc (3×30 mL). The combined organic layer was washed with brine (100 mL), dried over anhydrous Na₂SO₄. After filtration, the filtrate was concentrated under vacuum. The residue was purified by Prep-TLC (dichloromethane/methanol=15:1) to give 4-{2-[(2R)-2-(2-methylphenyl)piperidin-1-yl]-7-azaspiro[3.5]nonan-7-yl}-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide as a yellow solid (80 mg, 84.7%). LC-MS (ESI, m/z) M-HCl+1: 1103.

Synthesis of 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2R)-2-(2-methylphenyl)piperidin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide hydrochloride: Into a 40 mL vial, were placed 4-{2-[(2R)-2-(2-methylphenyl)piperidin-1-yl]-7-azaspiro[3.5]nonan-7-yl}-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide (80 mg, 0.07 mmol, 1.0 eq), TBAF-THF (5 mL, 1 M), ethylenediamine (87 mg, 1.4 mmol, 20.0 eq). The reaction mixture was stirred for 5 hours at 70° C. The resulting mixture was quenched by the addition of water (100 mL), and extracted with EtOAc (3×30 mL). The combined organic layer was washed with brine (100 mL), dried over anhydrous Na₂SO₄. After filtration, the filtrate was concentrated under vacuum. The crude product was purified by Prep-HPLC using the following conditions (Prep-HPLC-003): Column, SunFire Prep C18 OBD Column, 19*150 mm, 5 μm 10 nm; mobile phase, water (0.05% trifluoroacetic acid) and acetonitrile (35% acetonitrile up to 75% in 10 min); Detector, UV. The collected solution was concentrated under vacuum to remove acetonitrile and the resulting solution was dried by lyophilization (added with Conc·HCl (1 drop)). Finally, 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2R)-2-(2-methylphenyl)piperidin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide hydrochloride was obtained as a yellow solid (35 mg, 47.8%). LC-MS (ESI, m/z) M-HCl+1: 973. ¹HNMR (400 MHz, DMSO-d₆) δ 12.75 (s, 1H), 11.25 (s, 1H), 10.79 (d, J=42.2 Hz, 1H), 8.95 (s, 1H), 8.56 (d, J=6.3 Hz, 1H), 8.39 (dd, J=7.3, 2.3 Hz, 1H), 7.90 (s, 1H), 7.78 (d, J=9.0 Hz, 1H), 7.43 (dd, J=9.3, 2.3 Hz, 1H), 7.27-7.18 (m, 3H), 7.14 (t, J=2.9 Hz, 1H), 6.98 (dd, J=8.7, 2.3 Hz, 1H), 6.83 (d, J=3.9 Hz, 1H), 6.44 (s, 1H), 6.07-6.00 (m, 1H), 4.36-4.22 (m, 2H), 4.14-4.04 (m, 1H), 3.81 (d, J=10.8 Hz, 2H), 3.72-3.58 (m, 1H), 3.49-3.22 (m, 4H), 3.21-2.85 (m, 6H), 2.40 (d, J=7.3 Hz, 3H), 2.19-0.91 (m, 27H).

### Example 83: Preparation of 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2S)-2-(2-isopropylphenyl)piperidin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide hydrochloride

Synthesis of tert-butyl 2-[2-(prop-1-en-2-yl)phenyl]piperidine-1-carboxylate: Into a 100 mL round-bottom flask, were placed tert-butyl 2-(2-bromophenyl)piperidine-1-carboxylate (1.6 g, 4.7 mmol, 1.0 eq), Dioxane (180 mL), water (20 mL), K₂CO₃ (2.0 g, 14.1 mmol, 3.0 eq), Pd(dppf)Cl₂·CH₂Cl₂ (380 mg, 0.5 mmol, 0.1 eq). The reaction mixture was stirred overnight at 80° C. under nitrogen atmosphere. The resulting mixture was quenched by the addition of water (200 mL) and extracted with ethyl acetate (3×100 mL). The combined organic layer was washed with brine (200 mL), dried over anhydrous Na₂SO₄. After filtration, the filtrate was concentrated under vacuum. The crude residue was applied onto a silica gel column and eluted with ethyl acetate/petroleum ether=1:10 to give tert-butyl 2-[2-(prop-1-en-2-yl)phenyl]piperidine-1-carboxylate as a light yellow oil (800 mg, 56.4%). ¹HNMR (300 MHz, Chloroform-d) δ 7.23-7.08 (m, 4H), 5.43 (t, J=5.4 Hz, 1H), 5.18 (q, J=1.7 Hz, 1H), 4.94 (dd, J=2.1, 1.0 Hz, 1H), 4.13-4.04 (m, 1H), 3.21 (ddd, J=13.5, 11.3, 4.7 Hz, 1H), 2.12 (t, J=1.2 Hz, 3H), 1.98-1.43 (m, 6H), 1.36 (s, 9H).

Synthesis of tert-butyl 2-(2-isopropylphenyl)piperidine-1-carboxylate: Into a 50 mL pressure tank reactor, were placed tert-butyl 2-[2-(prop-1-en-2-yl)phenyl]piperidine-1-carboxylate (800 mg, 2.6 mmol, 1.0 eq), methanol (20 mL), Pd/C (10%, 100 mg). The mixture was hydrogenated at 50° C. under 20 atm of hydrogen pressure overnight, After filtration, the filtrate was concentrated under vacuum. The crude residue was applied onto a silica gel column and eluted with ethyl acetate/petroleum ether=1:10 to give tert-butyl 2-(2-isopropylphenyl)piperidine-1-carboxylate as a colorless oil (700 mg, 86.9%). ¹H NMR (300 MHz, Chloroform-d) δ 7.42-7.07 (m, 4H), 5.37 (dd, J=6.3, 4.6 Hz, 1H), 4.10 (dd, J=14.1, 4.6 Hz, 1H), 3.29-3.13 (m, 2H), 2.09-1.57 (m, 6H), 1.33 (s, 9H), 1.26 (dd, J=12.5, 6.9 Hz, 6H).

Synthesis of (2S)-2-(2-isopropylphenyl)piperidine and (2R)-2-(2-isopropylphenyl)piperidine: A solution of tert-butyl 2-(2-isopropylphenyl)piperidine-1-carboxylate (700 mg, 2.3 mmol, 1.0 eq) in HCl (gas) in 1,4-dioxane (10 mL) was stirred for 1 hour at 25° C. The resulting mixture was concentrated under vacuum. The resulting mixture was diluted with saturated aqueous NaHCO₃ and extracted with CH₂Cl₂ (2×50 mL). The combined organic layer was washed with brine (100 mL), dried over anhydrous Na₂SO₄. After filtration, the filtrate was concentrated under vacuum. The crude product was purified by Prep-CHIRAL-HPLC. Finally, (2S)-2-(2-isopropylphenyl)piperidine was obtained as a colorless oil (130 mg, 27.7%) and (2R)-2-(2-isopropylphenyl)piperidine was obtained as a colorless oil (150 mg, 32.0%). A, T_{R}=0.602 min in CHIRAL-SFC, Column: CHIRAL ART Amylose-C NEO, 3*25 cm, 5 um; Mobile Phase A: CO₂, Mobile Phase B: MeOH (0.1% 2M NH₃-MeOH); Flow rate: 75 mL/min; Gradient: 15% B, Wave Length: 220 nm. B, T_{R}=0.696 min in CHIRAL-SFC, Column: CHIRAL ART Amylose-C NEO, 3*25 cm, 5 um; Mobile Phase A: CO₂, Mobile Phase B: MeOH (0.1% 2M NH₃-MeOH); Flow rate: 75 mL/min; Gradient: 15% B Wave Length: 220 nm. LC-MS (ESI, m/z) M+1: 204 (peak 1).

Synthesis of 4-{2-[(2S)-2-(2-isopropylphenyl)piperidin-1-yl]-7-azaspiro[3.5]nonan-7-yl}-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide: Into an 8 mL vial, were placed (2S)-2-(2-isopropylphenyl)piperidine (15 mg, 0.07 mmol, 1.0 eq), N-[3-nitro-4-({[(1r,4r)-4-hydroxy-4-methylcyclohexyl]methyl}amino)benzenesulfonyl]-4-{2-oxo-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide (70 mg, 0.07 mmol, 1.0 eq), methanol (2 mL), ZnCl₂ (20 mg, 0.2 mmol, 2.0 eq), NaBH₃CN (23 mg, 0.4 mmol, 5.0 eq). The reaction mixture was stirred overnight at 80° C. The resulting mixture was quenched by the addition of water (100 mL) and extracted with EtOAc (3×30 mL). The combined organic layer was washed with brine (100 mL), dried over anhydrous Na₂SO₄. After filtration, the filtrate was concentrated under vacuum. The residue was purified by Prep-TLC (dichloromethane/methanol=15:1) to give 4-{2-[(2S)-2-(2-isopropylphenyl)piperidin-1-yl]-7-azaspiro[3.5]nonan-7-yl}-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide as a yellow solid (70 mg, 83.9%). LC-MS (ESI, m/z) M-HCl+1: 1132.

Synthesis of 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2S)-2-(2-isopropylphenyl)piperidin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide hydrochloride: Into a 40 mL vial, were placed 4-{2-[(2S)-2-(2-isopropylphenyl)piperidin-1-yl]-7-azaspiro[3.5]nonan-7-yl}-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide (70 mg, 0.07 mmol, 1.0 eq), TBAF-THF (5 mL, 1 M), ethylenediamine (79.7 mg, 1.3 mmol, 20.0 eq). The reaction mixture was stirred for 6 hours at 70° C. The resulting mixture was quenched by the addition of water (100 mL), and then extracted with EtOAc (3×30 mL). The combined organic layer was washed with brine (100 mL), dried over anhydrous Na₂SO₄. After filtration, the filtrate was concentrated under vacuum. The residue was purified by Prep-TLC (dichloromethane/methanol=12:1). The crude product was purified by Prep-HPLC using the following conditions (Prep-HPLC-003): Column, SunFire Prep C18 OBD Column, 19*150 mm, 5 μm 10 nm; mobile phase, water (0.05% trifluoroacetic acid) and acetonitrile (35% acetonitrile up to 75% in 10 min); Detector, UV. The collected solution was concentrated under vacuum to remove acetonitrile and the resulting solution was dried by lyophilization (added with Conc·HCl (1 drop). Finally, 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2S)-2-(2-isopropylphenyl)piperidin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide hydrochloride as a yellow solid (20 mg, 31.2%). LC-MS (ESI, m/z) M-HCl+1: 1001. ¹H NMR (300 MHz, DMSO-d₆) δ 12.75 (s, 1H), 11.25 (s, 1H), 10.30 (s, 1H), 8.85 (s, 1H), 8.56 (d, J=6.3 Hz, 1H), 8.38 (d, J=2.3 Hz, 1H), 7.86-7.76 (m, 2H), 7.45-7.22 (m, 3H), 7.14 (t, J=2.9 Hz, 1H), 7.00-6.94 (m, 1H), 6.87-6.79 (m, 2H), 6.46 (d, J=12.3 Hz, 1H), 6.04 (dd, J=3.4, 1.9 Hz, 1H), 4.70-4.10 (m, 3H), 3.80-2.95 (m, 13H), 2.13-0.55 (m, 35H).

### Example 84: Preparation of 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2R)-2-(2-isopropylphenyl)piperidin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide hydrochloride

Synthesis of 4-{2-[(2R)-2-(2-isopropylphenyl)piperidin-1-yl]-7-azaspiro[3.5]nonan-7-yl}-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide: Into an 8 mL vial, were placed (2R)-2-(2-methylphenyl)piperidine (15 mg, 0.07 mmol, 1.0 eq), N-[3-nitro-4-({[(1r,4r)-4-hydroxy-4-methylcyclohexyl]methyl}amino)benzenesulfonyl]-4-{2-oxo-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide (70 mg, 0.07 mmol, 1.0 eq), methanol (2 mL), ZnCl₂ (20 mg, 0.2 mmol, 2.0 eq), NaBH₃CN (23 mg, 0.4 mmol, 5.0 eq). The reaction mixture was stirred for overnight at 80° C. The resulting mixture was quenched by the addition of water (100 mL) and extracted with EtOAc (3×30 mL). The combined organic layer was washed with brine (100 mL), dried over anhydrous Na₂SO₄. After filtration, the filtrate was concentrated under vacuum. The residue was purified by Prep-TLC (dichloromethane/methanol=15:1) to give 4-{2-[(2R)-2-(2-isopropylphenyl)piperidin-1-yl]-7-azaspiro[3.5]nonan-7-yl}-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide as a yellow solid (70 mg, 83.9%). LC-MS (ESI, m/z) M-HCl+1: 1132.

Synthesis of 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2R)-2-(2-isopropylphenyl)piperidin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide hydrochloride: Into a 40 mL vial, were placed 4-{2-[(2R)-2-(2-isopropylphenyl)piperidin-1-yl]-7-azaspiro[3.5]nonan-7-yl}-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide (70 mg, 0.06 mmol, 1.0 eq), TBAF-THF (5 mL, 1 M), ethylenediamine (74 mg, 1.2 mmol, 20.0 eq). The reaction mixture was stirred for 6 hours at 70° C. The resulting mixture was quenched by the addition of water (100 mL), and extracted with EtOAc (3×30 mL). The combined organic layer was washed with brine (100 mL), dried over anhydrous Na₂SO₄. After filtration, the filtrate was concentrated under vacuum. The residue was purified by Prep-TLC (dichloromethane/methanol=12:1). The crude product was purified by Prep-HPLC using the following conditions (Prep-HPLC-003): Column, SunFire Prep C18 OBD Column, 19*150 mm, 5 μm 10 nm; mobile phase, water (0.05% trifluoroacetic acid) and acetonitrile (35% acetonitrile up to 75% in 10 min); Detector, UV. The collected solution was concentrated under vacuum to remove acetonitrile and the resulting solution was dried by lyophilization (added with Conc·HCl (1 drop). Finally, 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2R)-2-(2-isopropylphenyl)piperidin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide hydrochloride was obtained as a yellow solid (15 mg, 23.4%). LC-MS (ESI, m/z) M-HCl+1: 1001. ¹HNMR (300 MHz, DMSO-d₆) b 12.75 (s, 1H), 11.25 (s, 1H), 9.59 (s, 1H), 8.58 (s, 1H), 8.39 (dd, J=7.3, 2.4 Hz, 1H), 7.78 (d, J=9.1 Hz, 1H), 7.62 (d, J=7.9 Hz, 1H), 7.46-7.34 (m, 3H), 7.26 (dd, J=8.1, 6.1 Hz, 1H), 7.14 (t, J=2.9 Hz, 1H), 6.97 (d, J=9.7 Hz, 1H), 6.88-6.79 (m, 2H), 6.44 (s, 1H), 6.05 (dd, J=3.4, 1.9 Hz, 1H), 4.70-4.10 (m, 3H), 3.80-2.95 (m, 13H), 2.13-0.55 (m, 35H).

### Example 85: Preparation of 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2R)-4-methyl-2-(2-methylphenyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide

Synthesis of 4-{2-[(2R)-4-methyl-2-(2-methylphenyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide: Into an 8 mL vial, were placed (3R)-1-methyl-3-(2-methylphenyl)piperazine (14 mg, 0.07 mmol, 1.0 eq), N-[3-nitro-4-({[(1r,4r)-4-hydroxy-4-methylcyclohexyl]methyl}amino)benzenesulfonyl]-4-{2-oxo-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide (70 mg, 0.07 mmol, 1.0 eq), methanol (2 mL), ZnCl₂ (20 mg, 0.2 mmol, 2.0 eq), NaBH₃CN (23 mg, 0.4 mmol, 5.0 eq). The reaction mixture was stirred overnight at 80° C. The resulting mixture was quenched by the addition of water (100 mL) and extracted with EtOAc (3×30 mL). The combined organic layer was washed with brine (100 mL), dried over anhydrous Na₂SO₄. After filtration, the filtrate was concentrated under vacuum. The residue was purified by Prep-TLC (dichloromethane/methanol=12:1) to give 4-{2-[(2R)-4-methyl-2-(2-methylphenyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide as a yellow solid (75 mg, 89.4%). LC-MS (ESI, m/z) M+1: 1119.

Synthesis of 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2R)-4-methyl-2-(2-methylphenyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide: Into a 40 mL vial, were placed 4-{2-[(2R)-4-methyl-2-(2-methylphenyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide (75 mg, 0.07 mmol, 1.0 eq), TBAF-THF (5 mL, 1 M), ethylenediamine (81 mg, 1.3 mmol, 20.0 eq). The reaction mixture was stirred for 6 hours at 70° C. The resulting mixture was quenched by the addition of water (100 mL), and extracted with EtOAc (3×30 mL). The combined organic layer was washed with brine (100 mL), dried over anhydrous Na₂SO₄. After filtration, the filtrate was concentrated under vacuum. The residue was purified by Prep-TLC (dichloromethane/methanol=12:1). The crude product was purified by Prep-HPLC using the following conditions (Prep-HPLC-006): Column, YMC-Actus Triart C18 ExRS, 30*150 mm, 5 μm; mobile phase, H₂O (10 mmol/L NH₄HCO₃+0.1% NH₃·H₂O) and CH₃CN (45% CH₃CN up to 85% in 10 min); Detector, UV. Finally, 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2R)-4-methyl-2-(2-methylphenyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide was obtained as a yellow solid (15 mg, 22.6%). LC-MS (ESI, m/z) M+1: 989. ¹HNMR (400 MHz, DMSO-d₆) δ 11.13 (s, 1H), 10.85 (s, 1H), 8.57-8.25 (m, 2H), 7.65 (dd, J=21.5, 8.7 Hz, 1H), 7.53-7.25 (m, 2H), 7.24-6.96 (m, 4H), 6.95-6.53 (m, 3H), 6.39 (s, 1H), 6.00 (d, J=18.3 Hz, 1H), 4.35-3.65 (m, 5H), 3.26-2.81 (m, 11H), 2.43-2.14 (m, 9H), 1.78-1.12 (m, 24H).

### Example 86: Preparation of 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2S)-4-methyl-2-(2-methylphenyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide

Synthesis of 4-{2-[(2S)-4-methyl-2-(2-methylphenyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide: Into an 8 mL vial, were placed (3S)-1-methyl-3-(2-methylphenyl)piperazine (14 mg, 0.07 mmol, 1.0 eq), N-[3-nitro-4-({[(1r,4r)-4-hydroxy-4-methylcyclohexyl]methyl}amino)benzenesulfonyl]-4-{2-oxo-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide (70 mg, 0.07 mmol, 1.0 eq), methanol (2 mL), ZnCl₂ (20 mg, 0.2 mmol, 2.0 eq), NaBH₃CN (23 mg, 0.4 mmol, 5.0 eq). The reaction mixture was stirred for overnight at 80° C. The resulting mixture was quenched by the addition of water (100 mL) and extracted with EtOAc (3×30 mL). The combined organic layer was washed with brine (100 mL), dried over anhydrous Na₂SO₄. After filtration, the filtrate was concentrated under vacuum. The residue was purified by Prep-TLC (dichloromethane/methanol=12:1) to give 4-{2-[(2S)-4-methyl-2-(2-methylphenyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide as a yellow solid (75 mg, 90.5%). LC-MS (ESI, m/z) M+1: 1119.

Synthesis of 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2S)-4-methyl-2-(2-methylphenyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide: Into a 40 mL vial, were placed 4-{2-[(2S)-4-methyl-2-(2-methylphenyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide (75 mg, 0.07 mmol, 1.0 eq), TBAF-THF (5 mL, 1 M), ethylenediamine (80 mg, 1.3 mmol, 20.0 eq). The reaction mixture was stirred for 6 hours at 70° C. The resulting mixture was quenched by the addition of water (100 mL), and extracted with EtOAc (3×30 mL). The combined organic layer was washed with brine (100 mL), dried over anhydrous Na₂SO₄. After filtration, the filtrate was concentrated under vacuum. The residue was purified by Prep-TLC (dichloromethane/methanol=12:1). The crude product was purified by Prep-HPLC using the following conditions (Prep-HPLC-006): Column, YMC-Actus Triart C18 ExRS, 30*150 mm, 5 μm; mobile phase, H₂O (10 mmol/L NH₄HCO₃+0.1% NH₃·H₂O) and CH₃CN (45% CH₃CN up to 85% in 10 min); Detector, UV. Finally, 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2S)-4-methyl-2-(2-methylphenyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide was obtained as a yellow solid (15 mg, 22.6%). LC-MS (ESI, m/z) M+1: 989. ¹HNMR (400 MHz, DMSO-d₆) δ 11.13 (s, 1H), 10.85 (s, 1H), 8.57-8.25 (m, 2H), 7.65 (dd, J=21.5, 8.7 Hz, 1H), 7.53-7.25 (m, 2H), 7.24-6.96 (m, 4H), 6.95-6.53 (m, 3H), 6.39 (s, 1H), 6.00 (d, J=18.3 Hz, 1H), 4.29-3.62 (m, 5H), 3.26-2.60 (m, 11H), 2.43-2.14 (m, 9H), 1.78-1.12 (m, 24H).

### Example 87: Preparation of 4-(4-{[2-(4-chlorophenyl)-4,4-dimethylcyclohex-1-en-1-yl]methyl}piperazin-1-yl)-2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-N-{3-nitro-4-[(oxan-4-ylmethyl)amino]benzenesulfonyl}benzamide

Synthesis of 4-(4-{[2-(4-chlorophenyl)-4,4-dimethylcyclohex-1-en-1-yl]methyl}piperazin-1-yl)-N-{3-nitro-4-[(oxan-4-ylmethyl)amino]benzenesulfonyl}-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide: Into a 40 mL vial, were placed (3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraene (50 mg, 0.14 mmol, 1.0 eq) and 2-bromo-4-(4-{[2-(4-chlorophenyl)-4,4-dimethylcyclohex-1-en-1-yl]methyl}piperazin-1-yl)-N-{3-nitro-4-[(oxan-4-ylmethyl)amino]benzenesulfonyl}benzamide (180 mg, 0.22 mmol, 1.6 eq), DMSO (3 mL), K₂CO₃ (58 mg, 0.42 mmol, 3.0 eq), CuI (13 mg, 0.07 mmol, 0.5 eq), N, N′-diphenyl-ethanediamide (17 mg, 0.07 mmol, 0.5 eq). The resulting mixture was stirred for 3 hours at 100° C. under nitrogen atmosphere. The resulting mixture was quenched by the addition of water (200 mL) and extracted with EtOAc (3×50 mL). The combined organic layer was washed with brine (200 mL), dried over anhydrous Na₂SO₄. After filtration, the filtrate was concentrated under vacuum. The crude residue was applied onto a silica gel column and eluted with dichloromethane/methanol=10:1 to give 4-(4-{[2-(4-chlorophenyl)-4,4-dimethylcyclohex-1-en-1-yl]methyl}piperazin-1-yl)-N-{3-nitro-4-[(oxan-4-ylmethyl)amino]benzenesulfonyl}-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide as a yellow solid (130 mg, 85.8%). LC-MS (ESI, m/z) M+1: 1096.

Synthesis of 4-(4-{[2-(4-chlorophenyl)-4,4-dimethylcyclohex-1-en-1-yl]methyl}piperazin-1-yl)-2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-N-{3-nitro-4-[(oxan-4-ylmethyl)amino]benzenesulfonyl}benzamide: Into a 40 mL vial, were placed 4-(4-{[2-(4-chlorophenyl)-4,4-dimethylcyclohex-1-en-1-yl]methyl}piperazin-1-yl)-N-{3-nitro-4-[(oxan-4-ylmethyl)amino]benzenesulfonyl}-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide (130 mg, 0.12 mmol, 1.0 eq), TBAF-THF (5 mL, 1 M), ethylenediamine (143 mg, 2.4 mmol, 20.0 eq). The reaction mixture was stirred for 6 hours at 70° C. The resulting mixture was quenched by the addition of water (100 mL) and extracted with EtOAc (3×30 mL). The combined organic layer was washed with brine (100 mL), dried over anhydrous Na₂SO₄. After filtration, the filtrate was concentrated under vacuum. The residue was purified by Prep-TLC (dichloromethane/methanol=11:1). The crude product was purified by Prep-HPLC using the following conditions (Prep-HPLC-006): Column, YMC-Actus Triart C18 ExRS, 30*150 mm, 5 μm; mobile phase, water (10 mmol/L NH₄HCO₃+0.1% NH₃·H₂O) and acetonitrile (45% acetonitrile up to 85% in 10 min); Detector, UV (220 nm). Finally, 4-(4-{[2-(4-chlorophenyl)-4,4-dimethylcyclohex-1-en-1-yl]methyl}piperazin-1-yl)-2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-N-{3-nitro-4-[(oxan-4-ylmethyl)amino]benzenesulfonyl}benzamide was obtained as a yellow solid (15 mg, 13.1%). LC-MS (ESI, m/z) M+1: 965. ¹HNMR (400 MHz, DMSO-d₆) δ 11.17 (s, 1H), 8.67-8.22 (m, 2H), 7.74 (s, 1H), 7.36 (t, J=8.5 Hz, 3H), 7.19-6.88 (m, 4H), 6.88-6.58 (m, 2H), 6.43 (s, 1H), 6.07-5.97 (m, 1H), 4.19 (s, 1H), 4.11-3.98 (m, 1H), 3.91-3.68 (m, 4H), 3.29-3.10 (m, 10H), 2.77 (d, J=13.4 Hz, 2H), 2.37-2.07 (m, 6H), 1.97 (s, 2H), 1.88 (s, 1H), 1.62 (d, J=12.9 Hz, 2H), 1.55-1.10 (m, 7H), 0.94 (d, J=6.2 Hz, 6H).

### Example 88 Preparation of N-((4-((((1r,4r)-4-hydroxy-4-methylcyclohexyl)methyl)amino)-3-nitrophenyl)sulfonyl)-4-(2-((R)-3-(2-isopropylphenyl)morpholino)-6-methyl-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide

Synthesis of tert-butyl 2-methyl-4-methylenepiperidine-1-carboxylate: A solution of methyltriphenylphosphanium bromide (90.5 g, 253.2 mmol, 1.8 eq) in THE (500 mL) was treated with t-BuOK (31.6 g, 281.3 mmol, 2.0 eq) for 1 hour at 0° C. under nitrogen atmosphere. After that, tert-butyl 2-methyl-4-oxopiperidine-1-carboxylate (30.0 g, 140.7 mmol, 1.0 eq) was added in portions at 0° C. The resulting mixture was stirred for 16 hours at 25° C. under nitrogen atmosphere. The reaction was quenched by the addition of water (400 mL) at 0° C. and then extracted with ethyl acetate (3×200 mL). The combined organic layer was dried over anhydrous Na₂SO₄. After filtration, the filtrate was concentrated under vacuum. The crude residue was applied onto a silica gel column and eluted with ethyl acetate/petroleum ether=1:10 to give tert-butyl 2-methyl-4-methylidenepiperidine-1-carboxylate as a yellow solid (25.0 g, 84.1%). ¹HNMR (300 MHz, DMSO-d₆) δ 4.85 (q, J=1.8 Hz, 1H), 4.75 (q, J=1.8 Hz, 1H), 4.46-4.30 (m, 1H), 3.97-3.83 (m, 1H), 2.79 (td, J=12.9, 3.6 Hz, 1H), 2.36-2.13 (m, 2H), 2.09-2.03 (m, 2H), 1.41 (s, 9H), 0.99 (d, J=6.9 Hz, 3H).

Synthesis of tert-butyl 1,1-dichloro-6-methyl-2-oxo-7-azaspiro[3.5]nonane-7-carboxylate: To a mixture of tert-butyl 2-methyl-4-methylidenepiperidine-1-carboxylate (10.0 g, 47.4 mmol, 1.0 eq) in Et₂O (100 mL), was added a solution of trichloroacetyl chloride (47.4 g, 260.3 mmol, 5.5 eq) in DME (10 mL). After that, cupriozinc (67.2 g, 520.6 mmol, 11.0 eq) was added in portions at 0° C. under N₂ atmosphere. The resulting mixture was stirred for 16 hours at 25° C. under N₂ atmosphere. The reaction was quenched with sat. NaHCO₃ at 0° C. The resulting mixture was filtered, the filter cake was washed with ethyl acetate (3×100 mL). The filtrate was washed with brine (100 mL), and then concentrated under vacuum. Finally, tert-butyl 1,1-dichloro-6-methyl-2-oxo-7-azaspiro[3.5]nonane-7-carboxylate was obtained as a yellow solid (15.0 g, 59.0%), which was used in the next step directly without further purification. LC-MS (ES, m/z) M+1: 322.

Synthesis of tert-butyl 6-methyl-2-oxo-7-azaspiro[3.5]nonane-7-carboxylate: Into a 500 mL 3-necked round-bottom flask, were placed tert-butyl 1,1-dichloro-6-methyl-2-oxo-7-azaspiro[3.5]nonane-7-carboxylate (15.0 g, 46.5 mmol, 1.0 eq), sat. NH₄Cl (250 mL), Zn (21.3 g, 325.8 mmol, 7.0 eq) and MeOH (250 mL). The resulting mixture was stirred for 16 hours at 25° C. The resulting mixture was filtered, the filter cake was washed with ethyl acetate (3×100 mL). The filtrate was dried over anhydrous Na₂SO₄, filtered and the filtrate was concentrated under vacuum. The crude residue was applied onto a silica gel column and eluted with ethyl acetate/petroleum ether=1:2 to give tert-butyl 6-methyl-2-oxo-7-azaspiro[3.5]nonane-7-carboxylate as a yellow soild (8.0 g, 67.8%). ¹HNMR (300 MHz, DMSO-d₆) δ 4.30 (dd, J=10.2, 4.8 Hz, 1H), 3.83 (dt, J=14.4, 3.6 Hz, 1H), 3.04-2.78 (m, 4H), 2.74-2.60 (m, 1H), 1.84 (dd, J=13.5, 5.7 Hz, 1H), 1.77 (dt, J=13.5, 2.1 Hz, 1H), 1.68-1.45 (m, 2H), 1.40 (s, 9H), 1.07 (d, J=7.2 Hz, 3H).

Synthesis of 6-methyl-7-azaspiro[3.5]nonan-2-one hydrochloride: Into a 500 mL 3-necked round-bottom flask, were placed tert-butyl 2-oxo-7-azaspiro[3.5]nonane-7-carboxylate (4.0 g, 15.8 mmol, 1.0 eq), ethyl acetate (10 mL), HCl in ethyl acetate (2 M, 40 mL). The resulting mixture was stirred for 4 hours at 25° C. The reaction mixture was concentrated under vacuum to give 6-methyl-7-azaspiro[3.5]nonan-2-one hydrochloride as a white solid (2.5 g, 99.4%). LC-MS (ES, m/z) M+1: 154.

Synthesis of 2-bromo-4-{6-methyl-2-oxo-7-azaspiro[3.5]nonan-7-yl}benzonitrile: Into a 50 mL round-bottom flask, were added 2-bromo-4-fluorobenzonitrile (1.5 g, 7.5 mmol, 1.0 eq), 6-methyl-7-azaspiro[3.5]nonan-2-one hydrochloride (1.4 g, 9.0 mmol, 1.2 eq), Na₂CO₃ (2.4 g, 22.5 mmol, 3.0 eq) and DMSO (10 mL). The resulting mixture was stirred for 16 hours at 80° C. The reaction mixture was diluted with water (10 mL) and then extracted with EtOAc (3×10 mL). The combined organic layer was washed with brine (10 mL), dried over anhydrous Na₂SO₄. After filtration, the filtrate was concentrated under vacuum. The crude residue was applied onto a silica gel column and eluted with ethyl acetate/petroleum ether=1:2 to give 2-bromo-4-{6-methyl-2-oxo-7-azaspiro[3.5]nonan-7-yl}benzonitrile as an orange solid (1.1 g, 44.0%).

Synthesis of 2-bromo-4-{2-[(3R)-3-(2-isopropylphenyl)morpholin-4-yl]-6-methyl-7-azaspiro[3.5]nonan-7-yl}benzonitrile: Into a 50 mL round-bottom flask were added 2-bromo-4-{6-methyl-2-oxo-7-azaspiro[3.5]nonan-7-yl}benzonitrile (1.0 g, 3.0 mmol, 1.2 eq), (3R)-3-(2-isopropylphenyl)morpholine (0.5 g, 2.5 mmol, 1.0 eq), ZnCl₂ (1.7 g, 12.5 mmol, 5.0 eq), NaBH₃CN (0.79 g, 12.5 mmol, 5.0 eq) and MeOH (10 mL). The resulting mixture was stirred for 2 hours at 80° C. The reaction was quenched by the addition of water (5 mL) and then extracted with CH₂Cl₂ (3×10 mL). The combined organic layer was washed with brine (10 mL), dried over anhydrous Na₂SO₄. After filtration, the filtrate was concentrated under vacuum. The crude residue was applied onto a silica gel column and eluted with ethyl acetate/petroleum ether=1:1 to give 2-bromo-4-{2-[(3R)-3-(2-isopropylphenyl)morpholin-4-yl]-6-methyl-7-azaspiro[3.5]nonan-7-yl}benzonitrile as an orange solid (700 mg, 53.6%). LC-MS (ES, m/z) M+1: 522/524.

Synthesis of 2-bromo-4-{2-[(3R)-3-(2-isopropylphenyl)morpholin-4-yl]-6-methyl-7-azaspiro[3.5]nonan-7-yl}benzoic acid: Into a 40 mL vial were added 2-bromo-4-{2-[(3R)-3-(2-isopropylphenyl)morpholin-4-yl]-6-methyl-7-azaspiro[3.5]nonan-7-yl}benzonitrile (600 mg, 1.1 mmol, 1.0 eq) and H₂SO₄/HOAc/H₂O (3 mL/3 mL/3 mL). The resulting mixture was stirred for 6 hours at 110° C. The reaction was quenched by the addition of 15% NaOH (10 mL) and then extracted with EtOAc (3×10 mL). The combined organic layer was washed with brine (10 mL), dried over anhydrous Na₂SO₄. After filtration, the filtrate was concentrated under vacuum to give 2-bromo-4-{2-[(3R)-3-(2-isopropylphenyl)morpholin-4-yl]-6-methyl-7-azaspiro[3.5]nonan-7-yl}benzoic acid (440 mg, 70.8%). LC-MS (ES, m/z) M+1: 541/543.

Synthesis of methyl 2-bromo-4-{2-[(3R)-3-(2-isopropylphenyl)morpholin-4-yl]-6-methyl-7-azaspiro[3.5]nonan-7-yl}benzoate: Into a 40 mL vial were added 2-bromo-4-{2-[(3R)-3-(2-isopropylphenyl)morpholin-4-yl]-6-methyl-7-azaspiro[3.5]nonan-7-yl}benzoic acid (400 mg, 0.7 mmol, 1 eq), TMSCHN₂ (220 mg, 2.2 mmol, 3.0 eq) and EtOAc/MeOH (3 mL/3 mL). The resulting mixture was stirred for 5 hours at 25° C. The reaction was quenched by the addition of water (3 mL) at 0° C. and then extracted with EtOAc (3×5 mL). The combined organic layer was washed with brine (5 mL), dried over anhydrous Na₂SO₄. After filtration, the filtrate was concentrated under vacuum. The residue was purified by reverse flash chromatography using the following conditions: column, C18 silica gel; mobile phase, CH₃CN in water (0.1% TFA), 10% to 50% gradient in 10 min; detector, UV 254 nm. Finally, methyl 2-bromo-4-{2-[(3R)-3-(2-isopropylphenyl)morpholin-4-yl]-6-methyl-7-azaspiro[3.5]nonan-7-yl}benzoate was obtained as a yellow green solid (200 mg, 48.7%). LC-MS (ES, m/z) M+1: 555/557.

Synthesis of 2-bromo-4-(2-((R)-3-(2-isopropylphenyl)morpholino)-6-methyl-7-azaspiro[3.5]nonan-7-yl)benzoic acid: Into an 8 mL vial were added methyl 2-bromo-4-{2-[(3R)-3-(2-isopropylphenyl)morpholin-4-yl]-6-methyl-7-azaspiro[3.5]nonan-7-yl}benzoate (150 mg, 0.270 mmol, 1.0 eq), NaOH (4 M, 0.2 mL) and dioxane/MeOH (1 mL/1 mL). The resulting mixture was stirred for 3 hours at 70° C. The mixture was acidified to pH=5 with conc. HCl and then extracted with EtOAc (3×3 mL). The combined organic layer was washed with brine (3 mL), dried over anhydrous Na₂SO₄. After filtration, the filtrate was concentrated under vacuum to give 2-bromo-4-(2-((R)-3-(2-isopropylphenyl)morpholino)-6-methyl-7-azaspiro[3.5]nonan-7-yl)benzoic acid as a yellow solid (135 mg, 92.3%). LC-MS (ES, m/z) M+1: 541/543.

Synthesis of 2-bromo-N-((4-((((1r,4r)-4-hydroxy-4-methylcyclohexyl)methyl)amino)-3-nitrophenyl)sulfonyl)-4-(2-((R)-3-(2-isopropylphenyl)morpholino)-6-methyl-7-azaspiro[3.5]nonan-7-yl)benzamide: Into an 8 mL vial, were added 2-bromo-4-(2-((R)-3-(2-isopropylphenyl)morpholino)-6-methyl-7-azaspiro[3.5]nonan-7-yl)benzoic acid (130 mg, 0.2 mmol, 1.0 eq), 3-nitro-4-({[(1r,4r)-4-hydroxy-4-methylcyclohexyl]methyl}amino)benzenesulfonamide (82 mg, 0.2 mmol, 1.0 eq), EDCl (184 mg, 1.0 mmol, 4.0 eq), DMAP (59 mg, 0.5 mmol, 2.0 eq) and DCM (3 mL). The resulting mixture was stirred for 4 hours at 25° C. The reaction was quenched by the addition of water (3 mL) and then extracted with EtOAc (3×3 mL). The combined organic layer was washed with brine (3 mL), dried over anhydrous Na₂SO₄. After filtration, the filtrate was concentrated under vacuum. The residue was purified by Prep-TLC (CH₂Cl₂/MeOH=12:1) to give 2-bromo-N-((4-((((1r,4r)-4-hydroxy-4-methylcyclohexyl)methyl)amino)-3-nitrophenyl)sulfonyl)-4-(2-((R)-3-(2-isopropylphenyl)morpholino)-6-methyl-7-azaspiro[3.5]nonan-7-yl)benzamide as a yellow solid (120 mg, 57.7%). LC-MS (ES, m/z) M+1: 866/868.

Synthesis of N-((4-((((1r,4r)-4-hydroxy-4-methylcyclohexyl)methyl)amino)-3-nitrophenyl)sulfonyl)-4-(2-((R)-3-(2-isopropylphenyl)morpholino)-6-methyl-7-azaspiro[3.5]nonan-7-yl)-2-((5aS,9aR)-1-((2-(trimethylsilyl)ethoxy)methyl)-6,7,9,9a-tetrahydro-1H-pyrano[3,4-b]pyrrolo[3′,2′:5,6]pyrido[3,2-e][1,4]oxazin-5(5aH)-yl)benzamide: Into an 8 mL vial were added 2-bromo-N-((4-((((1r,4r)-4-hydroxy-4-methylcyclohexyl)methyl)amino)-3-nitrophenyl)sulfonyl)-4-(2-((R)-3-(2-isopropylphenyl)morpholino)-6-methyl-7-azaspiro[3.5]nonan-7-yl)benzamide (100 mg, 0.1 mmol, 1.0 eq), (3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraene (42 mg, 0.1 mmol, 1.0 eq), CuI (9 mg, 0.05 mmol, 0.4 eq), N₁,N₂-diphenyloxalamide (17 mg, 0.06 mmol, 0.6 eq), K₂CO₃ (48 mg, 0.3 mmol, 3.0 eq) and DMSO (3 mL). The resulting mixture was stirred for 1 hour at 90° C. under nitrogen atmosphere. The reaction was quenched by the addition of water (2 mL) and then extracted with EtOAc (3×3 mL). The combined organic layer was washed with brine (3 mL), dried over anhydrous Na₂SO₄. After filtration, the filtrate was concentrated under vacuum. The residue was purified by Prep-TLC (CH₂Cl₂/MeOH=13:1) to give N-((4-((((1r,4r)-4-hydroxy-4-methylcyclohexyl)methyl)amino)-3-nitrophenyl)sulfonyl)-4-(2-((R)-3-(2-isopropylphenyl)morpholino)-6-methyl-7-azaspiro[3.5]nonan-7-yl)-2-((5aS,9aR)-1-((2-(trimethylsilyl)ethoxy)methyl)-6,7,9,9a-tetrahydro-1H-pyrano[3,4-b]pyrrolo[3′,2′:5,6]pyrido[3,2-e][1,4]oxazin-5(5aH)-yl)benzamide as a yellow solid (80 mg, 60.4%). LC-MS (ES, m/z) M+1: 1148.

Synthesis of N-((4-((((1r,4r)-4-hydroxy-4-methylcyclohexyl)methyl)amino)-3-nitrophenyl)sulfonyl)-4-(2-((R)-3-(2-isopropylphenyl)morpholino)-6-methyl-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide: Into an 8 mL vial, were added N-((4-((((1r,4r)-4-hydroxy-4-methylcyclohexyl)methyl)amino)-3-nitrophenyl)sulfonyl)-4-(2-((R)-3-(2-isopropylphenyl)morpholino)-6-methyl-7-azaspiro[3.5]nonan-7-yl)-2-((5aS,9aR)-1-((2-(trimethylsilyl)ethoxy)methyl)-6,7,9,9a-tetrahydro-1H-pyrano[3,4-b]pyrrolo[3′,2′:5,6]pyrido[3,2-e][1,4]oxazin-5(5aH)-yl)benzamide (50 mg, 0.04 mmol, 1.0 eq), ethylenediamine (52 mg, 0.9 mmol, 20.0 eq) and TBAF in THE (2 mL). The resulting mixture was stirred for overnight at 80° C. The reaction was quenched by the addition of water (3 mL) and extracted with EtOAc (3×3 mL). The combined organic layer was washed with brine (3 mL), and dried over anhydrous Na₂SO₄. After filtration, the filtrate was concentrated in vacuo. The residue was purified by reverse flash chromatography using the following conditions: column, C18 silica gel; mobile phase, CH₃CN in water (0.1% NH₃—H₂O), 10% to 50% gradient in 10 min; detector, UV 254 nm. Finally, N-((4-((((1r,4r)-4-hydroxy-4-methylcyclohexyl)methyl)amino)-3-nitrophenyl)sulfonyl)-4-(2-((R)-3-(2-isopropylphenyl) morpholino)-6-methyl-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide was obtained as a yellow solid (8 mg, 18.0%). LC-MS (ES, m/z) M+1: 1017. ¹HNMR (400 MHz, Methanol-d₄) δ 8.59-8.44 (m, 1H), 8.14 (d, J=24.2 Hz, 1H), 7.95-7.58 (m, 1H), 7.59-7.41 (m, 2H), 7.26 (dt, J=22.9, 8.1 Hz, 1H), 7.11 (dt, J=24.9, 5.1 Hz, 2H), 6.91 (s, 1H), 6.75-6.61 (m, 2H), 6.59-6.45 (m, 1H), 6.23-5.82 (m, 1H), 5.37 (t, J=4.8 Hz, 1H), 4.58 (s, 1H), 4.20 (d, J=51.4 Hz, 2H), 3.93 (t, J=13.3 Hz, 2H), 3.70 (dt, J=43.2, 13.2 Hz, 2H), 3.49 (dd, J=19.1, 7.1 Hz, 4H), 3.28-3.13 (m, 5H), 3.09-2.89 (m, 4H), 2.86-2.58 (m, 8H), 2.51-2.11 (m, 2H), 2.12-1.91 (m, 2H), 1.91-1.67 (m, 4H), 1.67-1.40 (m, 3H), 1.29-1.12 (m, 7H), 1.00 (d, J=7.1 Hz, 2H), 0.92 (t, J=6.5 Hz, 1H), 0.79 (dd, J=29.8, 6.7 Hz, 1H).

### Example 89 Preparation of 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-[(1R,2S)-2-[(3R)-3-(2-isopropylphenyl)morpholin-4-yl]-1-methyl-7-azaspiro[3.5]nonan-7-yl]-N-[3-nitro-4-({[(1r,4r)-4-hydroxy-4-methylcyclohexyl]methyl}amino)benzenesulfonyl]benzamide (assumed)

Synthesis of tert-butyl 4-ethylidenepiperidine-1-carboxylate: Into a 1000 mL round-bottom flask, were placed benzen-2-ylium-1-yl(ethyl)diphenylphosphanium (61.2 g, 210.8 mmol, 1.4 eq), tetrahydrofuran (500 mL). After that, n-BuLi (14.5 g, 225.8 mmol, 1.5 eq) was added in portions at −78° C. The resulting mixture was stirred for additional 1 hour at −78° C. To the above mixture was added tert-butyl 4-oxopiperidine-1-carboxylate (30.0 g, 150.6 mmol, 1.0 eq). The resulting mixture was stirred for overnight at 25° C. The reaction was quenched by the addition of aq. NH₄Cl and extracted with EtOAc (2×500 mL). The combined organic layer was washed with brine (500 mL), dried over anhydrous Na₂SO₄. After filtration, the filtrate was concentrated under vacuum. The crude residue was applied onto a silica gel column and eluted with ethyl acetate/petroleum ether=1:2, to give tert-butyl 4-ethylidenepiperidine-1-carboxylate as a colorless oil (25.0 g, 78.6%). ¹HNMR (300 MHz, Chloroform-d) δ 5.29 (dtd, J=8.1, 6.8, 6.1, 2.0 Hz, 1H), 3.45-3.35 (m, 4H), 2.27-2.12 (m, 4H), 1.61 (d, J=6.7 Hz, 3H), 1.49 (s, 8H).

Synthesis of tert-butyl 1,1-dichloro-3-methyl-2-oxo-7-azaspiro[3.5]nonane-7-carboxylate: Into a 500 mL round-bottom flask purged and maintained with an inert atmosphere of nitrogen, were placed tert-butyl 4-ethylidenepiperidine-1-carboxylate (8.0 g, 37.9 mmol, 1.0 eq) and zinc-copper couple (35.0 g, 275.6 mmol, 7.0 eq) in DME (200 mL). After that, trichloroacetyl chloride (22.7 g, 125.0 mmol, 3.3 eq) was added dropwised with stirring at 0° C. The resulting mixture was stirred for overnight at 25° C. The resulting mixture was filtered through a Celite pad and the filtrate was concentrated under vacuum to give tert-butyl 1,1-dichloro-3-methyl-2-oxo-7-azaspiro[3.5]nonane-7-carboxylate as a colorless oil (12.0 g, 98.4%). LC-MS (ES, m/z) M-t-Bu+CH₃CN: 307.

Synthesis of tert-butyl 1-methyl-2-oxo-7-azaspiro[3.5]nonane-7-carboxylate: Into a 250 mL 3-necked round-bottom flask, were placed tert-butyl 1,1-dichloro-3-methyl-2-oxo-7-azaspiro[3.5]nonane-7-carboxylate (8.0 g, 24.8 mmol, 1.0 eq), methanol (70 mL), sat. NH₄Cl (70 mL), Zn (9.7 g, 149.0 mmol, 6.0 eq). The resulting mixture was stirred for 16 hours at 25° C. under nitrogen atmosphere. The reaction mixture was filtered through a Celite pad and then the filtrate was concentrated under vacuum. The crude residue was applied onto a silica gel column and eluted with ethyl acetate/petroleum ether=1:4, to give tert-butyl 1-methyl-2-oxo-7-azaspiro[3.5]nonane-7-carboxylate as a brown oil (4.0 g, 63.6%). ¹HNMR (300 MHz, Chloroform-d) δ 3.98 (dt, J=13.4, 4.4 Hz, 2H), 3.06-2.84 (m, 3H), 2.82-2.69 (m, 2H), 1.95-1.79 (m, 1H), 1.77-1.57 (m, 3H), 1.48 (s, 10H), 1.07 (d, J=7.5 Hz, 3H).

Synthesis of 1-methyl-7-azaspiro[3.5]nonan-2-one: A solution of tert-butyl 1-methyl-2-oxo-7-azaspiro[3.5]nonane-7-carboxylate (4.0 g, 15.8 mmol, 1.0 eq) in HCl (gas)/1,4-dioxane (50 mL) was stirred for 1 hour at 25° C. The resulting mixture was concentrated under vacuum. The resulting mixture was diluted with sat. NaHCO₃ ((100 mL) and extracted with CH₂Cl₂ (2×200 mL). The combined organic layer was washed with brine (200 mL), dried over anhydrous Na₂SO₄. After filtration, the filtrate was concentrated under vacuum to give 1-methyl-7-azaspiro[3.5]nonan-2-one as a light yellow oil (2.2 g, 90.9%). ¹HNMR (300 MHz, DMSO-d₆) δ 9.14 (s, 1H), 3.36 (s, 2H), 3.26-3.04 (m, 3H), 2.97 (ddd, J=17.1, 3.1, 1.2 Hz, 1H), 2.81 (dd, J=17.0, 2.0 Hz, 1H), 2.03 (ddd, J=14.0, 11.5, 4.1 Hz, 1H), 1.88-1.75 (m, 3H), 1.57 (d, J=14.2 Hz, 1H), 0.97 (d, J=7.5 Hz, 3H).

Synthesis of methyl 2-bromo-4-{1-methyl-2-oxo-7-azaspiro[3.5]nonan-7-yl}benzoate: Into a 250 mL 3-necked round-bottom flask, were placed 1-methyl-7-azaspiro[3.5]nonan-2-one (2.0 g, 13.0 mmol, 1.0 eq), DMSO (100 mL), methyl 2-bromo-4-fluorobenzoate (4.9 g, 14.4 mmol, 1.1 eq), Na₂CO₃ (3.5 g, 32.6 mmol, 2.5 eq). The resulting solution was stirred overnight at 100° C. The reaction was quenched by the addition of water (200 mL) and then extracted with ethyl acetate (3×200 mL). The combined organic layer was washed with brine (200 mL), dried over anhydrous Na₂SO₄. After filtration, the filtrate was concentrated under vacuum. The crude residue was applied onto a silica gel column and eluted with ethyl acetate/petroleum ether=1:10, to give methyl 2-bromo-4-{1-methyl-2-oxo-7-azaspiro[3.5]nonan-7-yl}benzoate as a light yellow oil (3.4 g, 71.1%). ¹HNMR (300 MHz, Chloroform-d) δ 7.84 (d, J=8.9 Hz, 1H), 7.16 (d, J=2.6 Hz, 1H), 6.85 (dd, J=8.9, 2.6 Hz, 1H), 3.89 (s, 3H), 3.81-3.67 (m, 2H), 3.08 (dd, J=5.7, 4.0 Hz, 1H), 3.08-2.96 (m, 2H), 2.90 (ddd, J=16.8, 2.8, 1.3 Hz, 1H), 2.80 (dd, J=16.9, 1.7 Hz, 1H), 2.14-1.99 (m, 1H), 1.91-1.74 (m, 2H), 1.56 (ddd, J=14.1, 8.0, 3.9 Hz, 1H), 1.10 (d, J=7.5 Hz, 3H).

Synthesis of methyl 2-bromo-4-{2-[(3R)-3-(2-isopropylphenyl)morpholin-4-yl]-1-methyl-7-azaspiro[3.5]nonan-7-yl}benzoate (assumed): Into a 40 mL vial, were placed methyl 2-bromo-4-{1-methyl-2-oxo-7-azaspiro[3.5]nonan-7-yl}benzoate (700 mg, 1.9 mmol, 1.0 eq), (3R)-3-(2-isopropylphenyl)morpholine (392 mg, 1.9 mmol, 1.0 eq), methanol (2 mL), NaBH₃CN (600 mg, 9.5 mmol, 5.0 eq), ZnCl₂ (521 mg, 3.8 mmol, 2.0 eq). The resulting mixture was stirred for overnight at 80° C. The reaction was quenched by the addition of water (100 mL) and then extracted with EtOAc (3×100 mL). The combined organic layer was washed with brine (100 mL), dried over anhydrous Na₂SO₄. After filtration, the filtrate was concentrated under vacuum. The crude residue was applied onto a silica gel column and eluted with dichloromethane/ethyl acetate=1:1 to give methyl 2-bromo-4-{2-[(3R)-3-(2-isopropylphenyl)morpholin-4-yl]-1-methyl-7-azaspiro[3.5]nonan-7-yl}benzoate (assumed) as a light yellow oil (100 mg, 9.4%). ¹HNMR (300 MHz, Chloroform-d) δ 7.99 (d, J=7.5 Hz, 1H), 7.80 (d, J=8.9 Hz, 1H), 7.42 (s, 3H), 7.07 (d, J=2.5 Hz, 1H), 6.76 (dd, J=8.9, 2.5 Hz, 1H), 4.34 (dd, J=24.9, 11.6 Hz, 2H), 4.15 (d, J=13.2 Hz, 1H), 3.96 (dt, J=23.1, 11.3 Hz, 2H), 3.87 (s, 3H), 3.69-3.55 (m, 3H), 3.22-3.05 (m, 2H), 3.01-2.74 (m, 3H), 2.37 (t, J=10.4 Hz, 2H), 2.18 (t, J=9.9 Hz, 1H), 1.68-1.35 (m, 10H), 0.12 (d, J=6.8 Hz, 3H).

Synthesis of 2-bromo-4-[(1R,2S)-2-[(3R)-3-(2-isopropylphenyl)morpholin-4-yl]-1-methyl-7-azaspiro[3.5]nonan-7-yl]benzoic acid (assumed): Into a 100 mL round-bottom flask, were placed methyl 2-bromo-4-[(1R)-2-[(3R)-3-(2-isopropylphenyl)morpholin-4-yl]-1-methyl-7-azaspiro[3.5]nonan-7-yl]benzoate (assumed) (80 mg, 0.1 mmol, 1.0 eq), methanol (2 mL), water (2 mL), NaOH (22 mg, 0.6 mmol, 4.0 eq). The resulting mixture was stirred for 5 hours at 70° C. The resulting mixture was then diluted with water (30 mL) and acidified to pH=5 with HCl (1 M). The precipitated solids were collected by filtration and dried under infrared light to give 2-bromo-4-[(1R,2S)-2-[(3R)-3-(2-isopropylphenyl)morpholin-4-yl]-1-methyl-7-azaspiro[3.5]nonan-7-yl]benzoic acid (assumed) as a yellow solid (70 mg, 92.4%). LC-MS (ES, m/z) M+1: 541/543.

Synthesis of 2-bromo-4-[(1R,2S)-2-[(3R)-3-(2-isopropylphenyl)morpholin-4-yl]-1-methyl-7-azaspiro[3.5]nonan-7-yl]-N-[3-nitro-4-({[(1r,4r)-4-hydroxy-4-methylcyclohexyl]methyl}amino)benzenesulfonyl]benzamide (assumed): Into a 40 mL vial, were placed 2-bromo-4-[(1R,2S)-2-[(3R)-3-(2-isopropylphenyl)morpholin-4-yl]-1-methyl-7-azaspiro[3.5]nonan-7-yl]benzoic acid (assumed) (60 mg, 0.1 mmol, 1.0 eq), 3-nitro-4-({[(1r,4r)-4-hydroxy-4-methylcyclohexyl]methyl}amino)benzenesulfonamide (38 mg, 0.1 mmol, 1.0 eq), dichloromethane (2 mL), EDCl (42 mg, 0.2 mmol, 2.0 eq), DMAP (54 mg, 0.4 mmol, 4.0 eq). The resulting mixture was stirred for overnight at 30° C. The resulting mixture was concentrated under vacuum. The crude residue was applied onto a silica gel column and eluted with dichloromethane/ethyl acetate (included 20% of methanol)=1:1, to give 2-bromo-4-[(1R,2S)-2-[(3R)-3-(2-isopropylphenyl)morpholin-4-yl]-1-methyl-7-azaspiro[3.5]nonan-7-yl]-N-[3-nitro-4-({[(1r,4r)-4-hydroxy-4-methylcyclohexyl]methyl}amino)benzenesulfonyl]benzamide (assumed) as a yellow solid (60 mg, 62.5%). LC-MS (ES, m/z) M+1: 866/868.

Synthesis of 4-[(1R,2S)-2-[(3R)-3-(2-isopropylphenyl)morpholin-4-yl]-1-methyl-7-azaspiro[3.5]nonan-7-yl]-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide (assumed): Into an 8 mL vial, were placed 2-bromo-4-[(1R,2S)-2-[(3R)-3-(2-isopropylphenyl)morpholin-4-yl]-1-methyl-7-azaspiro[3.5]nonan-7-yl]-N-[3-nitro-4-({[(1r,4r)-4-hydroxy-4-methylcyclohexyl]methyl}amino)benzenesulfonyl]benzamide (assumed) (65 mg, 0.07 mmol, 1.5 eq), (3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraene (18 mg, 0.05 mmol, 1.0 eq), DMSO (5 mL), K₂CO₃ (14 mg, 0.1 mmol, 2.0 eq), CuI (10 mg, 0.05 mmol, 1.0 eq), N,N′-bis(4-hydroxy-2,6-dimethylphenyl)ethanediamide (16 mg, 0.05 mmol, 1.0 eq). The resulting mixture was stirred for 3 hours at 100° C. under nitrogen atmosphere. The reaction was quenched by the addition of water (200 mL) and extracted with EtOAc (3×50 mL). The combined organic layer was washed with brine (200 mL), dried over anhydrous Na₂SO₄. After filtration, the filtrate was concentrated under vacuum. The crude residue was applied onto a silica gel column and eluted with dichloromethane/methanol=10:1 to give 4-[(1R,2S)-2-[(3R)-3-(2-isopropylphenyl)morpholin-4-yl]-1-methyl-7-azaspiro[3.5]nonan-7-yl]-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide (assumed) as a yellow solid (15 mg, 17.5%). LC-MS (ES, m/z) M+1: 1147.

Synthesis of 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-[(1R,2S)-2-[(3R)-3-(2-isopropylphenyl)morpholin-4-yl]-1-methyl-7-azaspiro[3.5]nonan-7-yl]-N-[3-nitro-4-({[(1r,4r)-4-hydroxy-4-methylcyclohexyl]methyl}amino)benzenesulfonyl]benzamide (assumed): Into a 40 mL vial, were placed 4-[(1R,2S)-2-[(3R)-3-(2-isopropylphenyl)morpholin-4-yl]-1-methyl-7-azaspiro[3.5]nonan-7-yl]-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide (assumed) (15 mg, 0.013 mmol, 1 eq), TBAF-THF (5 mL, 1.0 M), ethylenediamine (16 mg, 0.3 mmol, 20.0 eq). The resulting mixture was stirred for 6 hours at 70° C. The reaction was quenched by the addition of water (100 mL) and then extracted with EtOAc (3×30 mL). The combined organic layer was washed with brine (100 mL), dried over anhydrous Na₂SO₄. After filtration, the filtrate was concentrated under vacuum. The residue was purified by Prep-TLC (dichloromethane/methanol=12:1). The crude product was purified by Prep-HPLC using the following conditions (Prep-HPLC-006): Column, YMC-Actus Triart C18 ExRS, 30*150 mm, 5 μm; mobile phase, water (10 mmol/L NH₄HCO₃+0.1% NH₃·H₂O) and CH₃CN (45% CH₃CN up to 85% in 10 min); Detector, UV. Finally, 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-[(1R,2S)-2-[(3R)-3-(2-isopropylphenyl)morpholin-4-yl]-1-methyl-7-azaspiro[3.5]nonan-7-yl]-N-[3-nitro-4-({[(1r,4r)-4-hydroxy-4-methylcyclohexyl]methyl}amino)benzenesulfonyl]benzamide (assumed) was obtained as a light yellow solid (6 mg, 45.1%). LC-MS (ES, m/z) M+1: 1017. ¹HNMR (400 MHz, DMSO-d₆) δ 10.85 (s, 1H), 8.39-8.29 (m, 1H), 8.30-8.25 (m, 1H), 7.66-7.59 (m, 1H), 7.48 (s, 1H), 7.38-7.11 (m, 5H), 7.00-6.95 (m, 1H), 6.80 (d, J=8.7 Hz, 1H), 6.69-6.51 (m, 2H), 6.05-5.87 (m, 1H), 4.24 (d, J=1.5 Hz, 1H), 4.11-3.98 (m, 2H), 3.97-3.84 (m, 2H), 3.72 (d, J=5.3 Hz, 1H), 3.60 (dd, J=17.0, 7.7 Hz, 4H), 3.54-3.42 (m, 4H), 3.32-2.59 (m, 15H), 2.46-1.27 (m, 11H), 1.21-1.06 (m, 10H), 0.48 (d, J=8.5 Hz, 3H).

### Example 90 Preparation of 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-(4-{[(3R)-3-(2-isopropylphenyl)morpholin-4-yl]methyl}piperidin-1-yl)-N-[3-nitro-4-({[(1r,4r)-4-hydroxy-4-methylcyclohexyl]methyl}amino)benzenesulfonyl]benzamide hydrochloride

Synthesis of piperidine-4-carbaldehyde HCl salt: Into a 250 mL round-bottom flask were added tert-butyl 4-formylpiperidine-1-carboxylate (10.0 g, 46.8 mmol, 1.0 eq), HCl (gas) in ethyl acetate (20 mL) and ethyl acetate (80 mL) at 25° C. The resulting mixture was stirred for 4 hours at 25° C. The reaction mixture was concentrated under reduced pressure to give piperidine-4-carbaldehyde HCl salt as a white solid (4.5 g, 84.8%). LC-MS (ES, m/z) M+1: 113.

Synthesis of methyl 2-bromo-4-(4-formylpiperidin-1-yl)benzoate: Into a 500 mL round-bottom flask were added piperidine-4-carbaldehyde HCl salt (4.5 g, 39.8 mmol, 1.0 eq), DMF (150 mL), methyl 2-bromo-4-fluorobenzoate (10.2 g, 43.7 mmol, 1.1 eq) and K₂CO₃ (13.7 g, 99.4 mmol, 2.5 eq) at 25° C. The resulting mixture was stirred for 16 hours at 80° C. The reaction mixture was diluted with water (100 mL) and then extracted with EtOAc (3×100 mL). The combined organic layer was washed with brine (100 mL), dried over anhydrous Na₂SO₄. After filtration, the filtrate was concentrated under reduced pressure. The crude residue was applied onto a silica gel column and eluted with dichloromethane/ethyl acetate=2:1 to give methyl 2-bromo-4-(4-formylpiperidin-1-yl)benzoate as a white solid (7.0 g, 53.9%). ¹HNMR (400 MHz, DMSO-d₆) δ 9.62 (d, J=0.9 Hz, 1H), 7.73 (d, J=8.9 Hz, 1H), 7.17 (d, J=2.6 Hz, 1H), 6.98 (dd, J=9.0, 2.6 Hz, 1H), 3.77 (s, 3H), 3.04 (ddd, J=13.5, 10.8, 3.0 Hz, 2H), 2.60 (tt, J=10.5, 4.2 Hz, 1H), 2.51 (q, J=1.9 Hz, 2H), 1.91 (dt, J=13.0, 3.9 Hz, 2H), 1.53 (dtd, J=14.4, 3.9 Hz, 2H).

Synthesis of methyl 2-bromo-4-(4-{[(3R)-3-(2-isopropylphenyl)morpholin-4-yl]methyl}piperidin-1-yl)benzoate: Into a 40 mL vial, were placed methyl 2-bromo-4-(4-formylpiperidin-1-yl)benzoate (200 mg, 0.6 mmol, 1.0 eq), (3R)-3-(2-isopropylphenyl)morpholine (120 mg, 0.6 mmol, 1.0 eq), methanol (5 mL), NaBH₃CN (183 mg, 2.9 mmol, 5.0 eq), ZnCl₂ (159 mg, 1.2 mmol, 2.0 eq). The resulting mixture was stirred for overnight at 80° C. The reaction was quenched by the addition of water (100 mL) and extracted with EtOAc (3×100 mL). The combined organic layer was washed with brine (100 mL), dried over anhydrous Na₂SO₄. After filtration, the filtrate was concentrated under vacuum. The crude residue was applied onto a silica gel column and eluted with dichloromethane/ethyl acetate=1:1 to give methyl 2-bromo-4-(4-{[(3R)-3-(2-isopropylphenyl)morpholin-4-yl]methyl}piperidin-1-yl)benzoate as a light yellow oil (230 mg, 76.3%). LC-MS (ES, m/z) M+1: 515/517.

Synthesis of 2-bromo-4-(4-{[(3R)-3-(2-isopropylphenyl)morpholin-4-yl]methyl}piperidin-1-yl)benzoic acid: Into a 100 mL round-bottom flask, were placed methyl 2-bromo-4-(4-{[(3R)-3-(2-isopropylphenyl)morpholin-4-yl]methyl}piperidin-1-yl)benzoate (200 mg, 0.4 mmol, 1.0 eq), methanol (2 mL), water (2 mL), NaOH (62 mg, 1.5 mmol, 4.0 eq). The resulting mixture was stirred for 5 hours at 70° C. The resulting mixture was then diluted with water (30 mL) and acidified to pH=5 with HCl (1 M). The precipitated solids were collected by filtration and dried under infrared light to give 2-bromo-4-(4-{[(3R)-3-(2-isopropylphenyl)morpholin-4-yl]methyl}piperidin-1-yl)benzoic acid as a yellow solid (170 mg, 87.4%). ¹HNMR (300 MHz, Chloroform-d) δ 7.94 (d, J=9.0 Hz, 1H), 7.55 (s, 1H), 7.28-7.16 (m, 3H), 7.07 (d, J=2.5 Hz, 1H), 6.74 (dd, J=9.1, 2.5 Hz, 1H), 3.98 (d, J=11.2 Hz, 1H), 3.83-3.66 (m, 3H), 3.36 (d, J=25.7 Hz, 1H), 3.07 (d, J=11.7 Hz, 1H), 2.84 (q, J=11.4 Hz, 2H), 2.30 (d, J=29.1 Hz, 2H), 2.11 (d, J=8.8 Hz, 2H), 1.81 (d, J=25.3 Hz, 2H), 1.58 (d, J=12.7 Hz, 1H), 1.25 (t, J=6.3 Hz, 6H), 1.14-0.95 (m, 4H).

Synthesis of 2-bromo-4-(4-{[(3R)-3-(2-isopropylphenyl)morpholin-4-yl]methyl}piperidin-1-yl)-N-[3-nitro-4-({[(1r,4r)-4-hydroxy-4-methylcyclohexyl]methyl}amino)benzenesulfonyl]benzamide: Into a 40 mL vial, were placed 2-bromo-4-(4-{[(3R)-3-(2-isopropylphenyl)morpholin-4-yl]methyl}piperidin-1-yl)benzoic acid (170 mg, 0.3 mmol, 1.0 eq), 3-nitro-4-({[(1r,4r)-4-hydroxy-4-methylcyclohexyl]methyl}amino)benzenesulfonamide (116 mg, 0.3 mmol, 1.0 eq), dichloromethane (2 mL), EDCl (130 mg, 0.7 mmol, 2.0 eq), DMAP (166 mg, 1.4 mmol, 4.0 eq). The resulting mixture was stirred for overnight at 30° C. The resulting mixture was concentrated under vacuum. The crude residue was applied onto a silica gel column and eluted with dichloromethane/ethyl acetate (included 20% of methanol)=1:1 to give 2-bromo-4-(4-{[(3R)-3-(2-isopropylphenyl)morpholin-4-yl]methyl}piperidin-1-yl)-N-[3-nitro-4-({[(1r,4r)-4-hydroxy-4-methylcyclohexyl]methyl}amino)benzenesulfonyl]benzamide as a yellow solid (180 mg, 64.2%). ¹HNMR (300 MHz, Chloroform-d) δ 8.92 (d, J=2.3 Hz, 1H), 8.55 (t, J=5.4 Hz, 1H), 8.17 (dd, J=9.2, 2.3 Hz, 1H), 7.64 (dd, J=9.0, 2.0 Hz, 1H), 7.53 (d, J=7.5 Hz, 1H), 7.25-7.12 (m, 2H), 6.99-6.87 (m, 2H), 6.72 (dd, J=9.0, 2.5 Hz, 1H), 3.96 (d, J=11.3 Hz, 1H), 3.83-3.61 (m, 6H), 3.40 (d, J=11.6 Hz, 1H), 3.27 (d, J=6.0 Hz, 2H), 3.05 (d, J=11.8 Hz, 1H), 2.82 (dtd, J=13.7, 10.4, 9.5, 4.9 Hz, 2H), 2.39-2.18 (m, 2H), 2.10 (d, J=13.5 Hz, 1H), 1.94-1.70 (m, 6H), 1.52 (ddt, J=19.2, 10.0, 4.9 Hz, 4H), 1.27 (d, J=4.5 Hz, 9H), 1.23 (d, J=4.2 Hz, 4H), 1.00-0.81 (m, 3H).

Synthesis of 4-(4-{[(3R)-3-(2-isopropylphenyl)morpholin-4-yl]methyl}piperidin-1-yl)-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide: Into an 8 mL vial, were placed 2-bromo-4-(4-{[(3R)-3-(2-isopropylphenyl)morpholin-4-yl]methyl}piperidin-1-yl)-N-[3-nitro-4-({[(1r,4r)-4-hydroxy-4-methylcyclohexyl]methyl}amino)benzenesulfonyl]benzamide (171 mg, 0.2 mmol, 1.5 eq), (3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraene (50 mg, 0.1 mmol, 1.0 eq), DMSO (5 mL), K₂CO₃ (38 mg, 0.3 mmol, 2.0 eq), CuI (26 mg, 0.1 mmol, 1.0 eq), methyl [(2-methylphenyl)carbamoyl]formate (27 mg, 0.1 mmol, 1.0 eq). The resulting mixture was stirred for 3 hours at 100° C. under nitrogen atmosphere. The reaction was quenched by the addition of water (200 mL) and extracted with EtOAc (3×50 mL). The combined organic layer was washed with brine (200 mL), dried over anhydrous Na₂SO₄. After filtration, the filtrate was concentrated under vacuum. The crude residue was applied onto a silica gel column and eluted with dichloromethane/methanol=10:1 to give 4-(4-{[(3R)-3-(2-isopropylphenyl)morpholin-4-yl]methyl}piperidin-1-yl)-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide as a yellow solid (60 mg, 39.2%). LC-MS (ES, m/z) M+1: 1107.

Synthesis of 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-(4-{[(3R)-3-(2-isopropylphenyl)morpholin-4-yl]methyl}piperidin-1-yl)-N-[3-nitro-4-({[(1r,4r)-4-hydroxy-4-methylcyclohexyl]methyl}amino)benzenesulfonyl]benzamide hydrochloride: Into a 40 mL vial, were placed 4-(4-{[(3R)-3-(2-isopropylphenyl)morpholin-4-yl]methyl}piperidin-1-yl)-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide (60 mg, 0.05 mmol, 1.0 eq), TBAF-THF (5 mL, 1.0 M), ethylenediamine (65 mg, 1.1 mmol, 20.0 eq). The resulting mixture was stirred for 6 hours at 70° C. The reaction was quenched by the addition of water (100 mL) and then extracted with EtOAc (3×30 mL). The combined organic layer was washed with brine (100 mL), dried over anhydrous Na₂SO₄. After filtration, the filtrate was concentrated under vacuum. The residue was purified by Prep-TLC (dichloromethane/methanol=12:1). The crude product was purified by Prep-HPLC using the following conditions (Prep-HPLC-003): Column, SunFire Prep C18 OBD Column, 19*150 mm, 5 μm 10 nm; mobile phase, water (0.05% trifluoroacetic acid) and acetonitrile (35% acetonitrile up to 75% in 10 min); Detector, UV (220 nm). The collected solution was concentrated under vacuum to remove acetonitrile and the resulting solution was dried by lyophilization (added with Conc. HCl (1 drop)). Finally, 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-(4-{[(3R)-3-(2-isopropylphenyl)morpholin-4-yl]methyl}piperidin-1-yl)-N-[3-nitro-4-({[(1r,4r)-4-hydroxy-4-methylcyclohexyl]methyl}amino)benzenesulfonyl]benzamide hydrochloride was obtained as a light yellow solid (15 mg, 27.3%). LC-MS (ES, m/z) M+1: 977. ¹HNMR (300 MHz, DMSO-d₆) δ 12.74 (s, 1H), 11.26 (s, 1H), 10.58 (s, 1H), 8.93 (s, 1H), 8.56 (d, J=5.9 Hz, 1H), 8.37 (d, J=0.3 Hz, 1H), 8.05 (d, J=7.8 Hz, 1H), 7.78 (d, J=9.0 Hz, 1H), 7.46- 7.36 (m, 2H), 7.36-7.20 (m, 1H), 7.20-7.11 (m, 1H), 7.03-6.92 (m, 1H), 6.84 (d, J=9.7 Hz, 1H), 6.45 (d, J=13.0 Hz, 1H), 6.05 (d, J=3.0 Hz, 1H), 4.80 (dd, J=12.2, 5.4 Hz, 1H), 4.49-4.34 (m, 1H), 4.31-4.17 (m, 1H), 4.16-3.92 (m, 5H), 3.89-3.75 (m, 4H), 3.44-3.19 (m, 6H), 2.92 (s, 1H), 2.77-2.62 (m, 4H), 2.13-1.88 (m, 2H), 1.79-1.50 (m, 12H), 1.42-1.08 (m, 11H).

### Example 91 Preparation of 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2R)-2-(2-isopropylphenyl)-4-methylpiperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide hydrochloride (assumed)

Synthesis of 4-{2-[(2R)-2-(2-isopropylphenyl)-4-methylpiperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide (assumed): Into a 40 mL vial, were placed N-[3-nitro-4-({[(1r,4r)-4-hydroxy-4-methylcyclohexyl]methyl}amino)benzenesulfonyl]-4-{2-oxo-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide (80 mg, 0.08 mmol, 1.0 eq), (3R)-3-(2-isopropylphenyl)-1-methylpiperazine (assumed) (24 mg, 0.1 mmol, 1.3 eq), methanol (3 mL), ZnCl₂ (23 mg, 0.2 mmol, 2.0 eq), NaBH₃CN (27 mg, 0.4 mmol, 5.0 eq). The resulting mixture was stirred for 4 hours at 80° C. under nitrogen atmosphere. The reaction was quenched by the addition of water (100 mL) and extracted with EtOAc (2×50 mL). The combined organic layer was washed with brine (200 mL), dried over anhydrous Na₂SO₄. After filtration, the filtrate was concentrated under vacuum. The residue was purified by Prep-TLC (dichloromethane/methanol=11:1) to give 4-{2-[(2R)-2-(2-isopropylphenyl)-4-methylpiperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide (assumed) as a yellow solid (86 mg, 88.5%). LC-MS (ES, m/z) M+1: 1147.

Synthesis of 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2R)-2-(2-isopropylphenyl)-4-methylpiperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide hydrochloride (assumed): Into a 40 mL vial, were placed 4-{2-[(2R)-2-(2-isopropylphenyl)-4-methylpiperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide (assumed) (86 mg, 0.07 mmol, 1.0 eq), TBAF-THF (5 mL, 1 M), ethylenediamine (90 mg, 1.5 mmol, 20.0 eq). The resulting mixture was stirred for 5 hours at 70° C. The reaction was quenched by the addition of water (100 mL) and extracted with EtOAc (3×30 mL). The combined organic layer was washed with brine (100 mL), dried over anhydrous Na₂SO₄. After filtration, the filtrate was concentrated under vacuum. The residue was purified by Prep-TLC (dichloromethane/methanol=11:1). The crude product was purified by Prep-HPLC using the following conditions (Prep-HPLC-003): Column, SunFire Prep C18 OBD Column, 19*150 mm, 5 μm 10 nm; mobile phase, water (0.05% trifluoroacetic acid) and acetonitrile (35% acetonitrile up to 75% in 10 min); Detector, UV. The collected solution was concentrated under vacuum to remove acetonitrile and the resulting solution was dried by lyophilization (added with Conc·HCl (1 drop)). Finally, 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2R)-2-(2-isopropylphenyl)-4-methylpiperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide hydrochloride (assumed) was obtained as a yellow solid (25 mg, 31.7%). LC-MS (ES, m/z) M-HCl+1: 1016. ¹HNMR (300 MHz, DMSO-d₆) δ 12.76 (s, 1H), 11.25 (s, 1H), 8.57 (s, 1H), 8.38 (d, J=2.3 Hz, 1H), 8.07 (s, 1H), 7.78 (d, J=9.0 Hz, 1H), 7.42 (d, J=6.7 Hz, 3H), 7.29 (s, 1H), 7.17-7.09 (m, 1H), 7.02-6.92 (m, 1H), 6.89-6.79 (m, 2H), 6.44 (s, 1H), 6.05 (dd, J=3.4, 1.8 Hz, 1H), 4.30-4.16 (m, 1H), 3.72-2.95 (m, 13H), 2.86 (s, 3H), 2.13 (s, 1H), 1.74-1.47 (m, 10H), 1.38-1.26 (m, 11H), 1.22-1.07 (m, 8H), 0.94 (t, J=7.3 Hz, 4H), 0.74 (s, 1H).

### Example 92 Preparation of 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2S)-2-(2-isopropylphenyl)-4-methylpiperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide hydrochloride (assumed)

Synthesis of 4-{2-[(2S)-2-(2-isopropylphenyl)-4-methylpiperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide (assumed): Into a 40 mL vial, were placed N-[3-nitro-4-({[(1r,4r)-4-hydroxy-4-methylcyclohexyl]methyl}amino)benzenesulfonyl]-4-{2-oxo-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide (82 mg, 0.09 mmol, 1.0 eq), (3S)-3-(2-isopropylphenyl)-1-methylpiperazine (25 mg, 0.1 mmol, 1.3 eq), methanol (3 mL), ZnCl₂ (23 mg, 0.2 mmol, 20.0 eq), NaBH₃CN (27 mg, 0.4 mmol, 5.0 eq). The resulting mixture was stirred for 4 hours at 80° C. under nitrogen atmosphere. The reaction was quenched by the addition of water (100 mL) and extracted with EtOAc (2×50 mL). The combined organic layer was washed with brine (200 mL), dried over anhydrous Na₂SO₄. After filtration, the filtrate was concentrated under vacuum. The residue was purified by Prep-TLC (dichloromethane/methanol=11:1) to give 4-{2-[(2S)-2-(2-isopropylphenyl)-4-methylpiperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide (assumed) as a yellow solid (76 mg, 76.3%). LC-MS (ES, m/z) M+1: 1147.

Synthesis of 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2S)-2-(2-isopropylphenyl)-4-methylpiperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide hydrochloride (assumed): Into a 40 mL vial, were placed 4-{2-[(2S)-2-(2-isopropylphenyl)-4-methylpiperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide (assumed) (76 mg, 0.07 mmol, 1.0 eq), TBAF-THF (5 mL, 1 M), ethylenediamine (80 mg, 1.3 mmol, 20.0 eq). The resulting mixture was stirred for 5 hours at 70° C. The reaction was quenched by the addition of water (100 mL) and extracted with EtOAc (3×30 mL). The combined organic layer was washed with brine (100 mL), dried over anhydrous Na₂SO₄. After filtration, the filtrate was concentrated under vacuum. The residue was purified by Prep-TLC (dichloromethane/methanol=11:1). The crude product was purified by Prep-HPLC using the following conditions (Prep-HPLC-003): Column, SunFire Prep C18 OBD Column, 19*150 mm, 5 μm 10 nm; mobile phase, water (0.05% trifluoroacetic acid) and acetonitrile (35% acetonitrile up to 75% in 10 min); Detector, UV. The collected solution was concentrated under vacuum to remove acetonitrile and the resulting solution was dried by lyophilization (added with Conc·HCl (1 drop)). Finally, 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2S)-2-(2-isopropylphenyl)-4-methylpiperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide hydrochloride (assumed) was obtained as a yellow solid (25 mg, 35.8%). LC-MS (ES, m/z) M-HCl+1: 1016. ¹HNMR (300 MHz, DMSO-d₆) δ 12.76 (s, 1H), 11.25 (s, 1H), 8.57 (s, 1H), 8.38 (d, J=2.3 Hz, 1H), 8.07 (s, 1H), 7.78 (d, J=9.0 Hz, 1H), 7.42 (d, J=6.7 Hz, 3H), 7.29 (s, 1H), 7.17-7.09 (m, 1H), 7.02-6.92 (m, 1H), 6.89-6.79 (m, 2H), 6.44 (s, 1H), 6.05 (dd, J=3.4, 1.8 Hz, 1H), 4.30-4.16 (m, 1H), 3.72-2.95 (m, 13H), 2.86 (s, 3H), 2.13 (s, 1H), 1.74-1.47 (m, 10H), 1.38-1.26 (m, 11H), 1.22-1.07 (m, 8H), 0.94 (t, J=7.3 Hz, 4H), 0.74 (s, 1H).

### Example 93 Preparation of 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2R)-2-(2-isopropoxyphenyl)-4-methylpiperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed)

Synthesis of 4-{2-[(2R)-2-(2-isopropoxyphenyl)-4-methylpiperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide (assumed): Into a 40 mL vial, were placed N-[3-nitro-4-({[(1r,4r)-4-hydroxy-4-methylcyclohexyl]methyl}amino)benzenesulfonyl]-4-{2-oxo-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide (80 mg, 0.08 mmol, 1.0 eq), (3R)-3-(2-isopropoxyphenyl)-1-methylpiperazine (assumed) (20 mg, 0.08 mmol, 1.0 eq), methanol (3 mL), ZnCl₂ (23 mg, 0.17 mmol, 2.0 eq), NaBH₃CN (27 mg, 0.4 mmol, 5.0 eq). The resulting mixture was stirred for 6 hours at 80° C. under nitrogen atmosphere. The reaction was quenched by the addition of water (100 mL) and extracted with EtOAc (2×50 mL). The combined organic layer was washed with brine (200 mL), dried over anhydrous Na₂SO₄. After filtration, the filtrate was concentrated under vacuum. The residue was purified by Prep-TLC (dichloromethane/methanol=11:1) to give 4-{2-[(2R)-2-(2-isopropoxyphenyl)-4-methylpiperazin-1-yl]-7-azaspiro[3.5]nonan-7-y}—N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide (assumed) as a yellow solid (80 mg, 81.2%). LC-MS (ES, m/z) M+1: 1163.

Synthesis of 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2R)-2-(2-isopropoxyphenyl)-4-methylpiperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed): Into a 40 mL vial, were placed 4-{2-[(2R)-2-(2-isopropoxyphenyl)-4-methylpiperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide (80 mg, 0.07 mmol, 1.0 eq), TBAF (5 mL, 1.0 M), ethylenediamine (83 mg, 1.4 mmol, 20.0 eq). The resulting mixture was stirred for 5 hours at 70° C. The reaction was quenched by the addition of water (100 mL) and extracted with EtOAc (3×30 mL). The combined organic layer was washed with brine (100 mL), dried over anhydrous Na₂SO₄. After filtration, the filtrate was concentrated under vacuum. The residue was purified by Prep-TLC (dichloromethane/methanol=11:1). The crude product was purified by Prep-HPLC using the following conditions (Prep-HPLC-006): Column, YMC-Actus Triart C18 ExRS, 30*150 mm, 5 μm; mobile phase, water (10 mmol/L NH₄HCO₃+0.1% NH₃—H₂O) and acetonitrile (45% acetonitrile up to 85% in 10 min); Detector, UV. The collected solution was concentrated under vacuum to remove acetonitrile to give 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2R)-2-(2-isopropoxyphenyl)-4-methylpiperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed) as a yellow solid (14 mg, 19.7%). LC-MS (ES, m/z) M+1: 1032. ¹HNMR (300 MHz, DMSO-d₆) δ 10.86 (s, 1H), 8.50 (s, 1H), 8.38-8.27 (m, 1H), 7.80-7.54 (m, 1H), 7.43-7.42 (s, 2H), 7.26-6.51 (m, 8H), 6.40 (s, 1H), 6.07-5.92 (m, 1H), 4.63 (s, 1H), 4.24 (s, 1H), 4.23-3.64 (m, 5H), 3.27-2.69 (m, 10H), 2.40-2.04 (m, 5H), 1.94-0.91 (m, 31H).

### Example 94 Preparation of 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2S)-2-(2-isopropoxyphenyl)-4-methylpiperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed)

Synthesis of 4-{2-[(2S)-2-(2-isopropoxyphenyl)-4-methylpiperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide (assumed): Into a 40 mL vial, were placed N-[3-nitro-4-({[(1r,4r)-4-hydroxy-4-methylcyclohexyl]methyl}amino)benzenesulfonyl]-4-{2-oxo-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide (70 mg, 0.07 mmol, 1.0 eq), (3S)-3-(2-isopropoxyphenyl)-1-methylpiperazine (assumed) (18 mg, 0.07 mmol, 1.0 eq), methanol (3 mL), ZnCl₂ (20 mg, 0.15 mmol, 2.0 eq), NaBH₃CN (23 mg, 0.4 mmol, 5.0 eq). The resulting mixture was stirred for 6 hours at 80° C. under nitrogen atmosphere. The resulting mixture was diluted with water (100 mL) and extracted with EtOAc (2×50 mL). The combined organic layer was washed with brine (200 mL), dried over anhydrous Na₂SO₄. After filtration, the filtrate was concentrated under vacuum. The residue was purified by Prep-TLC (dichloromethane/methanol=11:1) to give 4-{2-[(2S)-2-(2-isopropoxyphenyl)-4-methylpiperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide (assumed) as a yellow solid (70 mg, 81.2%). LC-MS (ES, m/z) M+1: 1163.

Synthesis of 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2S)-2-(2-isopropoxyphenyl)-4-methylpiperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed): Into a 40 mL vial, were placed 4-{2-[(2S)-2-(2-isopropoxyphenyl)-4-methylpiperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide (70 mg, 0.06 mmol, 1.0 eq), TBAF (5 mL, 1.0 M), ethylenediamine (72 mg, 1.2 mmol, 20.0 eq). The resulting mixture was stirred for 5 hours at 70° C. The reaction was quenched by the addition of water (100 mL) and then extracted with EtOAc (3×30 mL). The combined organic layer was washed with brine (100 mL), dried over anhydrous Na₂SO₄. After filtration, the filtrate was concentrated under vacuum. The residue was purified by Prep-TLC (dichloromethane/methanol=11:1). The crude product was re-purified by Prep-HPLC using the following conditions (Prep-HPLC-006): Column, YMC-Actus Triart C18 ExRS, 30*150 mm, 5 μm; mobile phase, water (10 mmol/L NH₄HCO₃+0.1% NH₃—H₂O) and acetonitrile (45% acetonitrile up to 85% in 10 min); Detector, UV. The collected solution was concentrated under vacuum to remove acetonitrile to give 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2S)-2-(2-isopropoxyphenyl)-4-methylpiperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed) as a yellow solid (5 mg, 8.0%). LC-MS (ES, m/z) M+1: 1032. ¹HNMR (300 MHz, Methanol-d₄) δ 8.64-8.35 (m, 2H), 7.84-7.65 (m, 1H), 7.55-7.35 (m, 2H), 7.30-7.17 (m, 1H), 7.06 (d, J=3.4 Hz, 1H), 7.03-6.84 (m, 3H), 6.70-6.48 (m, 3H), 5.99 (dd, J=16.3, 3.4 Hz, 1H), 4.71-4.60 (m, 1H), 4.34-3.75 (m, 4H), 3.74-3.58 (m, 1H), 3.51-3.41 (m, 1H), 3.29-2.96 (m, 9H), 2.74 (d, J=12.7 Hz, 1H), 2.54-2.29 (m, 6H), 2.02-1.14 (m, 29H).

### Example 95 Preparation of N-((4-((((1r,4r)-4-hydroxy-4-methylcyclohexyl)methyl)amino)-3-nitrophenyl)sulfonyl)-4-(2-((S)-2-(2-isopropoxyphenyl)piperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed)

Synthesis of N-((4-((((1r,4r)-4-hydroxy-4-methylcyclohexyl)methyl)amino)-3-nitrophenyl)sulfonyl)-4-(2-((S)-2-(2-isopropoxyphenyl)piperidin-1-yl)-7-azaspiro[3.5]nonan-7-yl)-2-((5aS,9aR)-1-((2-(trimethylsilyl)ethoxy)methyl)-6,7,9,9a-tetrahydro-1H-pyrano[3,4-b]pyrrolo[3′,2′:5,6]pyrido[3,2-e][1,4]oxazin-5(5aH)-yl)benzamide (assumed): Into an 8 mL round-bottom flask were added N-[3-nitro-4-({[(1r,4r)-4-hydroxy-4-methylcyclohexyl]methyl}amino)benzenesulfonyl]-4-{2-oxo-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide (70 mg, 0.074 mmol, 1.0 eq), (2S)-2-(2-isopropoxyphenyl)piperidine (49 mg, 0.2 mmol, 3.0 eq), NaBH₃CN (23 mg, 0.4 mmol, 5.0 eq), ZnCl₂ (51 mg, 0.37 mmol, 5.0 eq), and MeOH (2 mL) at 25° C. The resulting mixture was stirred for 2 hours at 80° C. The reaction was quenched by the addition of water (5 mL) and extracted with EtOAc (5 mL). The combined organic layer was washed with brine (2×5 mL), dried over anhydrous Na₂SO₄. After filtration, the filtrate was concentrated under vacuum. Finally, N-((4-((((1r,4r)-4-hydroxy-4-methylcyclohexyl)methyl)amino)-3-nitrophenyl)sulfonyl)-4-(2-((S)-2-(2-isopropoxyphenyl)piperidin-1-yl)-7-azaspiro[3.5]nonan-7-yl)-2-((5aS,9aR)-1-((2-(trimethylsilyl)ethoxy)methyl)-6,7,9,9a-tetrahydro-1H-pyrano[3,4-b]pyrrolo[3′,2′:5,6]pyrido[3,2-e][1,4]oxazin-5(5aH)-yl)benzamide (assumed) was obtained as a yellow solid. (50 mg, 58.8%). LC-MS (ES, m/z) M+1: 1148.

Synthesis of N-((4-((((1r,4r)-4-hydroxy-4-methylcyclohexyl)methyl)amino)-3-nitrophenyl)sulfonyl)-4-(2-((S)-2-(2-isopropoxyphenyl)piperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed): Into an 8 mL round-bottom flask were added N-((4-((((1r,4r)-4-hydroxy-4-methylcyclohexyl)methyl)amino)-3-nitrophenyl)sulfonyl)-4-(2-((S)-2-(2-isopropoxyphenyl)piperidin-1-yl)-7-azaspiro[3.5]nonan-7-yl)-2-((5aS,9aR)-1-((2-(trimethylsilyl)ethoxy)methyl)-6,7,9,9a-tetrahydro-1H-pyrano[3,4-b]pyrrolo[3′,2′:5,6]pyrido[3,2-e][1,4]oxazin-5(5aH)-yl)benzamide (40 mg, 0.035 mmol, 1.0 eq), TBAF-THF (5 mL, 1 M), ethylenediamine (42 mg, 0.7 mmol, 20.0 eq). The resulting mixture was stirred for 6 hours at 70° C. The reaction was quenched by the addition of water (5 mL) and then extracted with EtOAc (3×5 mL). The combined organic layer was washed with brine (5 mL), dried over anhydrous Na₂SO₄. After filtration, the filtrate was concentrated under vacuum. The residue was purified by Prep-TLC (dichloromethane/methanol=12:1). The crude product was purified by Prep-HPLC using the following conditions (Prep-HPLC-006): Column, YMC-Actus Triart C18 ExRS, 30*150 mm, 5 μm; mobile phase, water (10 mmol/L NH₄HCO₃+0.1% NH₃·H₂O) and CH₃CN (45% CH₃CN up to 85% in 10 min); Detector, UV. Finally, N-((4-((((1r,4r)-4-hydroxy-4-methylcyclohexyl)methyl)amino)-3-nitrophenyl)sulfonyl)-4-(2-((S)-2-(2-isopropoxyphenyl)piperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed) was obtained as a light yellow solid (10 mg, 28.2%). LC-MS (ES, m/z) M+1: 1017. ¹HNMR (300 MHz, Methanol-d₄) δ 8.48 (d, J=2.2 Hz, 1H), 7.90 (d, J=9.0 Hz, 1H), 7.75-7.58 (m, 1H), 7.53 (t, J=8.7 Hz, 2H), 7.39 (t, J=7.7 Hz, 1H), 7.13-6.96 (m, 3H), 6.75 (dd, J=9.4, 5.3 Hz, 2H), 6.54 (s, 1H), 6.05 (d, J=3.4 Hz, 1H), 4.75 (d, J=5.4 Hz, 3H), 4.56 (q, J=7.0 Hz, 3H), 4.30 (d, J=7.6 Hz, 2H), 3.92 (d, J=8.8 Hz, 2H), 3.74 (d, J=8.0 Hz, 2H), 3.62 (d, J=11.6 Hz, 1H), 3.57-3.43 (m, 2H), 3.28 (d, J=6.8 Hz, 3H), 3.22 (s, 2H), 3.16 (s, 2H), 2.93 (d, J=33.5 Hz, 2H), 2.26 (d, J=16.7 Hz, 3H), 2.00 (d, J=17.2 Hz, 4H), 1.75 (s, 3H), 1.68 (d, J=7.0 Hz, 6H), 1.42 (dd, J=11.4, 6.0 Hz, 4H), 1.31 (s, 2H), 1.26 (d, J=4.3 Hz, 4H), 1.09 (s, 1H).

### Example 96 Preparation of N-((4-((((1r,4r)-4-hydroxy-4-methylcyclohexyl)methyl)amino)-3-nitrophenyl)sulfonyl)-4-(2-((R)-2-(2-isopropoxyphenyl)piperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed)

Synthesis of N-((4-((((1r,4r)-4-hydroxy-4-methylcyclohexyl)methyl)amino)-3-nitrophenyl)sulfonyl)-4-(2-((R)-2-(2-isopropoxyphenyl)piperidin-1-yl)-7-azaspiro[3.5]nonan-7-yl)-2-((5aS,9aR)-1-((2-(trimethylsilyl)ethoxy)methyl)-6,7,9,9a-tetrahydro-1H-pyrano[3,4-b]pyrrolo[3′,2′:5,6]pyrido[3,2-e][1,4]oxazin-5(5aH)-yl)benzamide (assumed): Into an 8 mL round-bottom flask, were added N-[3-nitro-4-({[(1r,4r)-4-hydroxy-4-methylcyclohexyl]methyl}amino)benzenesulfonyl]-4-{2-oxo-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide (80 mg, 0.09 mmol, 1.0 eq), (2R)-2-(2-isopropoxyphenyl)piperidine (19 mg, 0.08 mmol, 1.0 eq), NaBH₃CN (27 mg, 0.4 mmol, 5.0 eq), ZnCl₂ (28 mg, 0.4 mmol, 5.0 eq), and MeOH (2 mL) at 25° C. The resulting mixture was stirred for 2 hours at 80° C. The reaction mixture was diluted with EtOAc (5 mL), and washed with brine (2×5 mL). The organic layer was dried over anhydrous Na₂SO₄. After filtration, the filtrate was concentrated under vacuum. Finally, N-((4-((((1r,4r)-4-hydroxy-4-methylcyclohexyl)methyl)amino)-3-nitrophenyl)sulfonyl)-4-(2-((R)-2-(2-isopropoxyphenyl)piperidin-1-yl)-7-azaspiro[3.5]nonan-7-yl)-2-((5aS,9aR)-1-((2-(trimethylsilyl)ethoxy)methyl)-6,7,9,9a-tetrahydro-1H-pyrano[3,4-b]pyrrolo[3′,2′:5,6]pyrido[3,2-e][1,4]oxazin-5(5aH)-yl)benzamide (assumed) was obtained as a yellow solid (80 mg, 82.3%). LC-MS (ES, m/z) M+1: 1148.

Synthesis of N-((4-((((1r,4r)-4-hydroxy-4-methylcyclohexyl)methyl)amino)-3-nitrophenyl)sulfonyl)-4-(2-((R)-2-(2-isopropoxyphenyl)piperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed): Into an 8 mL round-bottom flask were added N-((4-((((1r,4r)-4-hydroxy-4-methylcyclohexyl)methyl)amino)-3-nitrophenyl)sulfonyl)-4-(2-((R)-2-(2-isopropoxyphenyl)piperidin-1-yl)-7-azaspiro[3.5]nonan-7-yl)-2-((5aS,9aR)-1-((2-(trimethylsilyl)ethoxy)methyl)-6,7,9,9a-tetrahydro-1H-pyrano[3,4-b]pyrrolo[3′,2′:5,6]pyrido[3,2-e][1,4]oxazin-5(5aH)-yl)benzamide (70 mg, 0.06 mmol, 1.0 eq), TBAF-THF (5 mL, 1 M), ethylenediamine (73 mg, 1.2 mmol, 20.0 eq). The resulting mixture was stirred for 6 hours. at 70° C. The reaction was quenched by the addition of water (5 mL) and then extracted with EtOAc (3×5 mL). The combined organic layer was washed with brine (5 mL), dried over anhydrous Na₂SO₄. After filtration, the filtrate was concentrated under vacuum. The residue was purified by Prep-TLC (dichloromethane/methanol=12:1). The crude product was purified by Prep-HPLC using the following conditions (Prep-HPLC-006): Column, YMC-Actus Triart C18 ExRS, 30*150 mm, 5 μm; mobile phase, water (10 mmol/L NH₄HCO₃+0.1% NH₃·H₂O) and CH₃CN (45% CH₃CN up to 85% in 10 min); Detector, UV. Finally, N-((4-((((1r,4r)-4-hydroxy-4-methylcyclohexyl)methyl)amino)-3-nitrophenyl)sulfonyl)-4-(2-((R)-2-(2-isopropoxyphenyl)piperidin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed) was obtained as a light yellow solid (20 mg, 32.2%). LC-MS (ES, m/z) M+1: 1017. ¹HNMR (300 MHz, Methanol-d₄) δ 8.48 (d, J=2.3 Hz, 1H), 7.93 (d, J=9.1 Hz, 1H), 7.61-7.30 (m, 3H), 7.17-6.91 (m, 3H), 6.73 (d, J=9.2 Hz, 2H), 6.48 (s, 2H), 6.03 (d, J=3.5 Hz, 1H), 4.75 (s, 1H), 4.56 (q, J=7.0 Hz, 3H), 4.31 (s, 2H), 3.91 (d, J=8.8 Hz, 2H), 3.74 (s, 2H), 3.62 (d, J=11.6 Hz, 1H), 3.49 (s, 2H), 3.27 (d, J=6.9 Hz, 2H), 3.22 (s, 2H), 3.14 (s, 1H), 2.91 (d, J=33.5 Hz, 2H), 2.24 (d, J=16.7 Hz, 3H), 1.96 (d, J=17.2 Hz, 4H), 1.87 (s, 3H), 1.78-1.60 (m, 6H), 1.61-1.44 (m, 4H), 1.40 (dd, J=6.0, 3.8 Hz, 4H), 1.31 (m, 4H), 1.26 (d, J=5.6 Hz, 3H).

### Example 97 Preparation of 2-((5aS,9aR)-3-fluoro-6,7,9,9a-tetrahydro-1H-pyrano[3,4-b]pyrrolo[3′,2′:5,6]pyrido[3,2-e][1,4]oxazin-5(5aH)-yl)-N-((4-((((1r,4r)-4-hydroxy-4-methylcyclohexyl)methyl)amino)-3-nitrophenyl)sulfonyl)-4-(2-((R)-3-(2-isopropoxyphenyl)morpholino)-7-azaspiro[3.5]nonan-7-yl)benzamide

Synthesis of 2-[(3R,8S)-13-fluoro-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]-N-[3-nitro-4-({[(1r,4r)-4-hydroxy-4-methylcyclohexyl]methyl}amino)benzenesulfonyl]-4-{2-oxo-7-azaspiro[3.5]nonan-7-yl}benzamide: Into an 8-mL sealed tube purged and maintained with an inert atmosphere of nitrogen, were placed 2-bromo-N-[3-nitro-4-({[(1r,4r)-4-hydroxy-4-methylcyclohexyl]methyl}amino)benzenesulfonyl]-4-{2-oxo-7-azaspiro[3.5]nonan-7-yl}benzamide (200 mg, 0.3 mmol, 1.0 eq), (3R,8S)-13-fluoro-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraene (114 mg, 0.3 mmol, 1.0 eq), N₁,N₂-diphenyloxalamide (43 mg, 0.2 mmol, 0.6 eq), CuI (34 mg, 0.2 mmol, 0.6 eq), t-BuONa (86 mg, 0.9 mmol, 3.0 eq), DMSO (2 mL). The resulting solution was stirred for 2 hours at 120° C. The reaction was then quenched by the addition of water (30 mL) and extracted with ethyl acetate (2×30 mL). The combined organic layer was washed with brine (2×30 mL) and dried over anhydrous Na₂SO₄. After filtration, the filtrate was concentrated under vacuum. The crude residue was applied onto a silica gel column and eluted with dichloromethane/methanol=8:1 to give 2-[(3R,8S)-13-fluoro-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]-N-[3-nitro-4-({[(1r,4r)-4-hydroxy- 4-methylcyclohexyl]methyl}amino)benzenesulfonyl]-4-{2-oxo-7-azaspiro[3.5]nonan-7-yl}benzamide as a yellow solid (80 mg, 27.6%). LC-MS (ES, m/z) M+1: 962.

Synthesis of 2-[(3R,8S)-13-fluoro-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(3R)-3-(2-isopropoxyphenyl)morpholin-4-yl]-7-azaspiro[3.5]nonan-7-yl}-N-[3-nitro-4-({[(1r,4r)-4-hydroxy-4-methylcyclohexyl]methyl}amino)benzenesulfonyl]benzamide: Into an 8-mL sealed tube purged and maintained with an inert atmosphere of nitrogen, were placed 2-[(3R,8S)-13-fluoro-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]-N-[3-nitro-4-({[(1r,4r)-4-hydroxy-4-methylcyclohexyl]methyl}amino)benzenesulfonyl]-4-{2-oxo-7-azaspiro[3.5]nonan-7-yl}benzamide (75 mg, 0.1 mmol, 1.0 eq), (3R)-3-(2-isopropoxyphenyl)morpholine (17 mg, 0.1 mmol, 1.0 eq), ZnCl₂ (32 mg, 0.2 mmol, 3.0 eq), NaBH₃CN (15 mg, 0.2 mmol, 3.0 eq), MeOH (1 mL). The resulting solution was stirred for 2 hours at 45° C. The reaction was then quenched by the addition of water (30 mL), and extracted with ethyl acetate (2×30 mL). The combined organic layer was washed with brine (2×30 mL) and dried over anhydrous Na₂SO₄. After filtration, the filtrate was concentrated under vacuum. The crude residue was applied onto a silica gel column and eluted with dichloromethane/methanol=8:1 to give 2-[(3R,8S)-13-fluoro-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(3R)-3-(2-isopropoxyphenyl)morpholin-4-yl]-7-azaspiro[3.5]nonan-7-yl}-N-[3-nitro-4-({[(1r,4r)-4-hydroxy-4-methylcyclohexyl]methyl}amino)benzenesulfonyl]benzamide as a yellow solid (50 mg, 54.9%). LC-MS (ES, m/z) M+1: 1168.

Synthesis of 2-((5aS,9aR)-3-fluoro-6,7,9,9a-tetrahydro-1H-pyrano[3,4-b]pyrrolo[3′,2′:5,6]pyrido[3,2-e][1,4]oxazin-5(5aH)-yl)-N-((4-((((1r,4r)-4-hydroxy-4-methylcyclohexyl)methyl)amino)-3-nitrophenyl)sulfonyl)-4-(2-((R)-3-(2-isopropoxyphenyl)morpholino)-7-azaspiro[3.5]nonan-7-yl)benzamide: Into an 8-mL sealed tube, were placed 2-[(3R,8S)-13-fluoro-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(3R)-3-(2-isopropoxyphenyl)morpholin-4-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (50 mg, 0.04 mmol, 1.0 eq), ethylenediamine (51 mg, 0.9 mmol, 20.0 eq) and TBAF in THE (1.0 M, 1 mL). The resulting solution was stirred for 4 hours at 80° C. The resulting mixture was filtered, the filtrate was concentrated under vacuum. The crude residue was applied onto a silica gel column and eluted with dichloromethane/methanol=8:1 to give crude product. The residue was purified by reverse flash chromatography using the following conditions: column, C18 silica gel; mobile phase, CH₃CN in Water (0.1% HCl), 10% to 50% gradient in 10 min; detector, UV 254 nm. Finally, 2-[(3R,8S)-13-fluoro-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(3R)-3-(2-isopropoxyphenyl)morpholin-4-yl]-7-azaspiro[3.5]nonan-7-yl}-N-[3-nitro-4-({[(1r,4r)-4-hydroxy-4-methylcyclohexyl]methyl}amino)benzenesulfonyl]benzamide was obtained as a yellow solid (2.2 mg, 5.0%). LC-MS (ES, m/z) M+1: 1037. ¹HNMR (300 MHz, Methanol-d₄) δ 8.51-8.43 (m, 1H), 7.89 (d, J=9.0 Hz, 1H), 7.61 (dd, J=9.3, 2.3 Hz, 1H), 7.47 (dd, J=18.9, 7.8 Hz, 2H), 7.14 (d, J=8.3 Hz, 1H), 7.09-6.96 (m, 2H), 6.87 (d, J=2.4 Hz, 1H), 6.84-6.73 (m, 2H), 6.34 (s, 1H), 4.55 (q, J=7.0 Hz, 1H), 4.34-4.15 (m, 3H), 4.03 (d, J=11.1 Hz, 1H), 3.98-3.87 (m, 4H), 3.56-3.45 (m, 2H), 3.30-3.20 (m, 6H), 2.32 (s, 1H), 2.09-1.96 (m, 1H), 1.85 (d, J=14.2 Hz, 2H), 1.69 (t, J=7.3 Hz, 7H), 1.54 (s, 13H), 1.50-1.35 (dd, J=11.2, 6.0 Hz, 8H).

### Example 98 Preparation of 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2S)-2-(2-isopropoxyphenyl)-4-(oxan-4-yl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed)

Synthesis of (3S)-3-(2-isopropoxyphenyl)-1-(oxan-4-yl)piperazine: Into an 8 mL vial were added (2S)-2-(2-isopropoxyphenyl)piperazine (100 mg, 0.4 mmol, 1.0 eq), 4H-pyran-4-one, tetrahydro- (55 mg, 0.55 mmol, 1.2 eq), NaBH₃CN (142 mg, 2.3 mmol, 5.0 eq), ZnCl₂ (309 mg, 2.3 mmol, 5.0 eq) and MeOH (3 mL). The resulting mixture was stirred for 4 hours at 25° C. The reaction was quenched by the addition of water (2 mL) and then extracted with CH₂Cl₂ (3×10 mL). The combined organic layer was washed with brine (10 mL), dried over anhydrous Na₂SO₄. After filtration, the filtrate was concentrated under vacuum. The residue was purified by Prep-TLC (CH₂Cl₂/MeOH=10:1) to give (3S)-3-(2-isopropoxyphenyl)-1-(oxan-4-yl)piperazine as a white solid (80 mg, 57.9%). ¹HNMR (300 MHz, Chloroform-d) δ 7.46-7.30 (m, 2H), 7.02-6.90 (m, 2H), 4.66 (hept, J=6.3 Hz, 1H), 4.46 (dd, J=10.6, 3.0 Hz, 1H), 4.08-3.86 (m, 2H), 3.38 (t, J=11.5 Hz, 3H), 3.27-3.03 (m, 3H), 2.91-2.66 (m, 2H), 1.89-1.74 (m, 2H), 1.74-1.54 (m, 2H), 1.42 (t, J=6.1 Hz, 6H).

Synthesis of 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2S)-2-(2-isopropoxyphenyl)-4-(oxan-4-yl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed): Into an 8 mL vial were added (3S)-3-(2-isopropoxyphenyl)-1-(oxan-4-yl)piperazine (50 mg, 0.16 mmol, 1.0 eq), 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-N-[3-nitro-4-({[(1r,4r)-4-hydroxy- 4-methylcyclohexyl]methyl}amino)benzenesulfonyl]-4-{2-oxo-7-azaspiro[3.5]nonan-7-yl}benzamide (100 mg, 0.1 mmol, 0.8 eq), NaBH₃CN (52 mg, 0.8 mmol, 5.0 eq), ZnCl₂ (112 mg, 0.8 mmol, 5.0 eq) and MeOH (2 mL). The resulting mixture was stirred for 4 hours at 70° C. The reaction was quenched by the addition of water (2 mL) and extracted with CH₂Cl₂ (3×5 mL). The combined organic layer was washed with brine (5 mL), dried over anhydrous Na₂SO₄. After filtration, the filtrate was concentrated under vacuum. The residue was purified by Prep-TLC (CH₂Cl₂/MeOH=20:1) to give 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2S)-2-(2-isopropoxyphenyl)-4-(oxan-4-yl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide as a yellow solid (35 mg, 19.33%). LC-MS (ES, m/z) M+1: 1103. ¹HNMR (300 MHz, Methanol-d₄) δ 8.62-8.41 (m, 1H), 7.87 (d, J=8.7 Hz, 1H), 7.70-7.37 (m, 2H), 7.25 (t, J=7.7 Hz, 1H), 7.07 (d, J=3.4 Hz, 3H), 7.02-6.82 (m, 2H), 6.68 (d, J=9.4 Hz, 1H), 6.51 (d, J=14.7 Hz, 1H), 5.99 (dd, J=19.2, 3.5 Hz, 1H), 4.76-4.59 (m, 1H), 4.29 (d, J=7.1 Hz, 2H), 4.12 (s, 1H), 3.94 (d, J=26.0 Hz, 4H), 3.70 (s, 1H), 3.46 (d, J=12.3 Hz, 4H), 3.26 (d, J=6.9 Hz, 4H), 3.14 (d, J=16.9 Hz, 3H), 2.85 (s, 2H), 2.53 (s, 4H), 2.08-1.78 (m, 5H), 1.73 (d, J=11.7 Hz, 6H), 1.53 (s, 5H), 1.45-1.36 (m, 6H), 1.34 (d, J=6.0 Hz, 3H), 1.31 (d, J=2.1 Hz, 3H), 1.26 (d, J=4.2 Hz, 3H), 0.91 (d, J=6.9 Hz, 1H).

### Example 99 Preparation of 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2S)-2-(2-isopropoxyphenyl)-4-[(4-methoxyphenyl)methyl]piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed)

Synthesis of tert-butyl (3S)-3-(2-isopropoxyphenyl)-4-[7-(4-{[3-nitro-4-({[(1r,4r)-4-hydroxy-4-methylcyclohexyl]methyl}amino)benzenesulfonyl]carbamoyl}-3-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]phenyl)-7-azaspiro[3.5]nonan-2-yl]piperazine-1-carboxylate (assumed): Into a 40 mL vial were added N-[3-nitro-4-({[(1r,4r)-4-hydroxy-4-methylcyclohexyl]methyl}amino)benzenesulfonyl]-4-{2-oxo-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide (800 mg, 0.8 mmol, 1.0 eq), tert-butyl (3S)-3-(2-isopropoxyphenyl)piperazine-1-carboxylate (272 mg, 0.8 mmol, 1.0 eq), NaBH₃CN (266 mg, 4.2 mmol, 5.0 eq), ZnCl₂ (577 mg, 4.2 mmol, 5.0 eq) and MeOH (10 mL). The resulting mixture was stirred for 4 hours at 70° C. The reaction was quenched by the addition of water (5 mL) and extracted with CH₂Cl₂ (3×20 mL). The combined organic layer was washed with brine (20 mL), dried over anhydrous Na₂SO₄. After filtration, the filtrate was concentrated under vacuum. The crude residue was purified by silica gel column chromatography and eluted with CH₂Cl₂/MeOH=30:1 to give tert-butyl (3S)-3-(2-isopropoxyphenyl)-4-[7-(4-{[3-nitro-4-({[(1r,4r)-4-hydroxy-4-methylcyclohexyl]methyl}amino)benzenesulfonyl]carbamoyl}-3-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]phenyl)-7-azaspiro[3.5]nonan-2-yl]piperazine-1-carboxylate (assumed) as a yellow solid (800 mg, 75.6%). LC-MS (ES, m/z) M+1: 1249.

Synthesis of 2-[(3R,8S)-15-(hydroxymethyl)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2S)-2-(2-isopropoxyphenyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed): Into a 50 mL round-bottom flask, were added tert-butyl (3S)-3-(2-isopropoxyphenyl)-4-[7-(4-{[3-nitro-4-({[(1r,4r)-4-hydroxy-4-methylcyclohexyl]methyl}amino)benzenesulfonyl]carbamoyl}-3-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]phenyl)-7-azaspiro[3.5]nonan-2-yl]piperazine-1-carboxylate (600 mg, 0.5 mmol, 1.0 eq) and TFA/DCM (3 mL/3 mL) at 0° C. The resulting mixture was stirred for 2 hours at 0° C. The mixture was basified to pH=8 with sat. NaHCO₃ and then extracted with CH₂Cl₂ (3×10 mL). The combined organic layer was washed with brine (10 mL), dried over anhydrous Na₂SO₄. After filtration, the filtrate was concentrated under vacuum. The crude residue was purified by Prep-TLC (CH₂Cl₂/MeOH=8:1) to give 2-[(3R,8S)-15-(hydroxymethyl)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2S)-2-(2-isopropoxyphenyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed) as a yellow solid (280 mg, 55.6%). LC-MS (ES, m/z) M+1: 1048.

Synthesis of 2-[(3R,8S)-15-(hydroxymethyl)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2S)-2-(2-isopropoxyphenyl)-4-[(4-methoxyphenyl)methyl]piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed): Into an 8 mL vial were added 2-[(3R,8S)-15-(hydroxymethyl)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2S)-2-(2-isopropoxyphenyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (100 mg, 0.1 mmol, 1.0 eq), anisaldehyde (20 mg, 0.1 mmol, 1.5 eq), NaBH₃CN (30 mg, 0.5 mmol, 5.0 eq), ZnCl₂ (65 mg, 0.5 mmol, 5.0 eq) and MeOH (3 mL). The resulting mixture was stirred for 4 hours at 25° C. The reaction was quenched by the addition of water (2 mL) and then extracted with CH₂Cl₂ (3×5 mL). The combined organic layer was washed with brine (5 mL), dried over anhydrous Na₂SO₄. After filtration, the filtrate was concentrated under vacuum. The crude residue was purified by Prep-TLC (CH₂Cl₂/MeOH=12:1) to give 2-[(3R,8S)-15-(hydroxymethyl)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2S)-2-(2-isopropoxyphenyl)-4-[(4-methoxyphenyl)methyl]piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide as a yellow solid (assumed) (70 mg, 62.8%). LC-MS (ES, m/z) M+1: 1168.

Synthesis of 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2S)-2-(2-isopropoxyphenyl)-4-[(4-methoxyphenyl)methyl]piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed): Into an 8 mL vial were added 2-[(3R,8S)-15-(hydroxymethyl)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2S)-2-(2-isopropoxyphenyl)-4-[(4-methoxyphenyl)methyl]piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (65 mg, 0.06 mmol, 1.0 eq), ethylenediamine (100 mg, 1.7 mmol, 30.0 eq) and THE (1 mL). The resulting mixture was stirred for 2 hours at 70° C. The reaction was quenched by the addition of water (3 mL) and then extracted with CH₂Cl₂ (3×3 mL). The combined organic layer was washed with brine (3 mL), dried over anhydrous Na₂SO₄. After filtration, the filtrate was concentrated under vacuum. The residue was purified by Prep-TLC (CH₂Cl₂/MeOH=0:1) to give 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2S)-2-(2-isopropoxyphenyl)-4-[(4-methoxyphenyl)methyl]piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed) as a yellow solid (16 mg, 25.3%). LC-MS (ES, m/z) M+1: 1139. ¹HNMR (300 MHz, Methanol-d₄) δ 8.48 (d, J=2.2 Hz, 1H), 8.11 (s, 1H), 7.88 (d, J=9.1 Hz, 2H), 7.62 (d, J=8.7 Hz, 1H), 7.58-7.36 (m, 2H), 7.27 (d, J=8.5 Hz, 2H), 7.08 (d, J=3.4 Hz, 1H), 7.04-6.83 (m, 3H), 6.69 (d, J=9.4 Hz, 2H), 6.62-6.41 (m, 1H), 6.11-5.89 (m, 1H), 4.62 (d, J=12.8 Hz, 2H), 4.30 (d, J=7.2 Hz, 3H), 3.93 (d, J=13.1 Hz, 1H), 3.79 (d, J=2.1 Hz, 2H), 3.66 (s, 2H), 3.60-3.38 (m, 2H), 3.26 (d, J=6.8 Hz, 3H), 3.14 (d, J=16.1 Hz, 4H), 2.93-2.70 (m, 2H), 2.52 (s, 3H), 2.35-2.15 (m, 1H), 2.13-1.92 (m, 2H), 1.84 (d, J=13.5 Hz, 2H), 1.71 (s, 3H), 1.62-1.42 (m, 5H), 1.42-1.29 (m, 8H), 1.29-1.11 (m, 7H), 0.91 (d, J=7.1 Hz, 2H).

### Example 100 Preparation of 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2S)-2-(2-isopropoxyphenyl)-4-{[6-(morpholin-4-yl)pyridin-3-yl]methyl}piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed)

Synthesis of 6-(morpholin-4-yl)pyridine-3-carbaldehyde: Into a 50 mL round-bottom flask, were placed 6-fluoropyridine-3-carbaldehyde (3.0 g, 23.9 mmol, 1.0 eq), morpholine (4.2 g, 47.9 mmol, 2.0 eq), K₂CO₃ (6.6 g, 47.9 mmol, 2.0 eq), DMSO (45 mL). The resulting solution was stirred for 3 hours at 130° C. The reaction was then quenched by the addition of water (50 mL) and extracted with ethyl acetate (2×50 mL). The combined organic layer was washed with water (2×50 mL), brine (2×50 mL) and dried over anhydrous Na₂SO₄. After filtration, the filtrate was concentrated under vacuum. The crude residue was applied onto a silica gel column and eluted with ethyl acetate/petroleum ether=1:1 to give 6-(morpholin-4-yl)pyridine-3-carbaldehyde as a light yellow solid (4.0 g, 86.8%). ¹HNMR (300 MHz, DMSO-d₆) δ 9.76 (s, 1H), 8.61 (d, J=2.4 Hz, 1H), 7.90 (dd, J=9.0, 2.4 Hz, 1H), 6.94 (d, J=9.0 Hz, 1H), 3.69 (s, 8H).

Synthesis of 4-(5-{[(3S)-3-(2-isopropoxyphenyl)piperazin-1-yl]methyl}pyridin-2-yl)morpholine (assumed): Into an 8-mL sealed-tube, were placed (2S)-2-(2-isopropoxyphenyl)piperazine (70 mg, 0.3 mmol, 1.0 eq), 6-(morpholin-4-yl)pyridine-3-carbaldehyde (49 mg, 0.3 mmol, 0.8 eq), DCE (2 mL), NaBH(OAc)₃ (202 mg, 0.9 mmol, 3.0 eq). The resulting solution was stirred for 2 hours at 25° C. The reaction was then quenched by the addition of water (20 mL) and extracted with dichloromethane/methanol=20:1 (2×20 mL). The combined organic layer was washed with brine (2×40 mL) and dried over anhydrous Na₂SO₄. After filtration, the filtrate was concentrated under vacuum. The crude residue was purified onto a silica gel column and eluted with dichloromethane/methanol=10:1 to give 4-(5-{[(3S)-3-(2-isopropoxyphenyl)piperazin-1-yl]methyl}pyridin-2-yl)morpholine (assumed) as an off-white solid (85 mg, 67.5%). LC-MS (ES, m/z) M+1: 397.

Synthesis of 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2S)-2-(2-isopropoxyphenyl)-4-{[6-(morpholin-4-yl)pyridin-3-yl]methyl}piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed): Into an 8-mL sealed-tube, were placed 4-(5-{[(3S)-3-(2-isopropoxyphenyl)piperazin-1-yl]methyl}pyridin-2-yl)morpholine (39 mg, 0.1 mmol, 1.0 eq), 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-N-[3-nitro-4-({[(1r,4r)-4-hydroxy-4-methylcyclohexyl]methyl}amino)benzenesulfonyl]-4-{2-oxo-7-azaspiro[3.5]nonan-7-yl}benzamide (80 mg, 0.1 mmol, 1.0 eq), MeOH (1 mL), ZnCl₂ (40 mg, 0.3 mmol, 3.0 eq), NaBH₃CN (19 mg, 0.3 mmol, 3.0 eq). The resulting solution was stirred for 4 hours at 70° C. The reaction was then quenched by the addition of water (20 mL) and extracted with dichloromethane/methanol=10:1 (2×30 mL). The combined organic layer was washed with brine (2×20 mL) and dried over anhydrous Na₂SO₄. After filtration, the filtrate was concentrated under vacuum. The residue was purified by Prep-TLC (dichloromethane/methanol=15:1) to give 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2S)-2-(2-isopropoxyphenyl)-4-{[6-(morpholin-4-yl)pyridin-3-yl]methyl}piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed) as a light yellow solid (25 mg, 21.3%). LC-MS (ES, m/z) M+1: 1195. ¹HNMR (400 MHz, Methanol-d₄) δ 8.50 (dd, J=9.4, 2.4 Hz, 2H), 8.04 (d, J=2.4 Hz, 1H), 7.87 (d, J=9.2 Hz, 1H), 7.66-7.54 (m, 1H), 7.49 (dd, J=9.2, 2.4 Hz, 1H), 7.46-7.40 (m, 1H), 7.23 (t, J=8.0 Hz, 1H), 7.07 (d, J=3.5 Hz, 1H), 6.96-6.88 (m, 2H), 6.83-6.80 (m, 1H), 6.69-6.65 (m, 2H), 6.49 (s, 1H), 6.02 (d, J=3.4 Hz, 1H), 4.66-4.57 (m, 1H), 4.29 (d, J=7.6 Hz, 1H), 4.12 (br, 1H), 3.91 (d, J=11.3 Hz, 1H), 3.82-3.75 (m, 4H), 3.70 (br, 2H), 3.56-3.44 (m, 9H), 3.29-3.24 (m, 2H), 3.18-3.09, (m, 7H), 3.00-2.97 (m, 1H), 2.72-2.66 (m, 1H), 2.50-2.32 (m, 2H), 1.98-1.82 (m, 2H), 1.75-1.66 (m, 4H), 1.56-1.49 (m, 4H), 1.45-1.36 (m, 6H), 1.32-1.25 (m, 11H).

### Example 101 Preparation of N-((4-((((1r,4r)-4-hydroxy-4-methylcyclohexyl)methyl)amino)-3-nitrophenyl)sulfonyl)-4-(2-((S)-2-(2-isopropoxyphenyl)-4-((5-methoxy-6-morpholinopyridin-3-yl)methyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed)

Synthesis of 4-(5-{[(3S)-4-benzyl-3-(2-isopropoxyphenyl)piperazin-1-yl]methyl}-3-methoxypyridin-2-yl)morpholine (assumed): Into a 40-mL sealed-tube, were placed (2S)-1-benzyl-2-(2-isopropoxyphenyl)piperazine (assumed) (150 mg, 0.5 mmol, 1.0 eq), 5-methoxy-6-morpholinonicotinaldehyde (118 mg, 0.5 mmol, 1.1 eq), DCE (4 mL), NaBH(OAc)₃ (307 mg, 1.4 mmol, 3.0 eq). The resulting solution was stirred for 2 hours at 25° C. The reaction was then quenched by the addition of water (40 mL) and extracted with ethyl acetate (2×40 mL). The combined organic layer was washed with brine (2×40 mL) and dried over anhydrous Na₂SO₄. After filtration, the filtrate was concentrated under vacuum. The crude residue was purified onto a silica gel column and eluted with ethyl acetate/petroleum ether=1:2 to give 4-(5-{[(3S)-4-benzyl-3-(2-isopropoxyphenyl)piperazin-1-yl]methyl}-3-methoxypyridin-2-yl)morpholine (assumed) as a light yellow solid (110 mg, 44.1%). LC-MS (ES, m/z) M+1: 517.

Synthesis of 4-(5-{[(3S)-3-(2-isopropoxyphenyl)piperazin-1-yl]methyl}-3-methoxypyridin-2-yl)morpholine (assumed): Into a 50-mL round-bottom flask, were placed 4-(5-{[(3S)-4-benzyl-3-(2-isopropoxyphenyl)piperazin-1-yl]methyl}-3-methoxypyridin-2-yl)morpholine (assumed) (90 mg, 0.2 mmol, 1.0 eq), THE (3 mL), Pd/C (9 mg, 0.1 mmol, 0.5 eq), Pd(OH)₂/C (12 mg, 0.1 mmol, 0.5 eq). The mixture was hydrogenated at 25° C. under H₂ (2 atm) for 8 hours. The resulting mixture was filtered and the filtrate was concentrated under vacuum to give 4-(5-{[(3S)-3-(2-isopropoxyphenyl)piperazin-1-yl]methyl}-3-methoxypyridin-2-yl)morpholine (assumed) as a light yellow oil (70 mg, crude). LC-MS (ES, m/z) M+1: 427.

Synthesis of N-((4-((((1r,4r)-4-hydroxy-4-methylcyclohexyl)methyl)amino)-3-nitrophenyl)sulfonyl)-4-(2-((S)-2-(2-isopropoxyphenyl)-4-((5-methoxy-6-morpholinopyridin-3-yl)methyl)piperazin-1-yl)-7-azaspiro[3.5]nonan-7-yl)-2-((5aS,9aR)-1-((2-(trimethylsilyl)ethoxy)methyl)-6,7,9,9a-tetrahydro-1H-pyrano[3,4-b]pyrrolo[3′,2′:5,6]pyrido[3,2-e][1,4]oxazin-5(5aH)-yl)benzamide (assumed): Into an 8 mL vial, were added N-[3-nitro-4-({[(1r,4r)-4-hydroxy-4-methylcyclohexyl]methyl}amino)benzenesulfonyl]-4-{2-oxo-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide (87 mg, 0.1 mmol, 0.7 eq), 4-(5-{[(3S)-3-(2-isopropoxyphenyl)piperazin-1-yl]methyl}-3-methoxypyridin-2-yl)morpholine (assumed) (45 mg, 0.1 mmol, 1.0 eq), NaBH₃CN (41 mg, 0.7 mmol, 5.0 eq), ZnCl₂ (90 mg, 0.7 mmol, 5.0 eq) and MeOH (2 mL). The resulting mixture was stirred for 5 hours at 70° C. The reaction was quenched by the addition of water (2 mL) and then extracted with CH₂Cl₂ (3×3 mL). The combined organic layer was washed with brine (3 mL), dried over anhydrous Na₂SO₄. After filtration, the filtrate was concentrated under vacuum. The residue was purified by Prep-TLC (CH₂Cl₂/MeOH=20:1) to give N-((4-((((1r,4r)-4-hydroxy-4-methylcyclohexyl)methyl)amino)-3-nitrophenyl)sulfonyl)-4-(2-((S)-2-(2-isopropoxyphenyl)-4-((5-methoxy-6-morpholinopyridin-3-yl)methyl)piperazin-1-yl)-7-azaspiro[3.5]nonan-7-yl)-2-((5aS,9aR)-1-((2-(trimethylsilyl)ethoxy)methyl)-6,7,9,9a-tetrahydro-1H-pyrano[3,4-b]pyrrolo[3′,2′:5,6]pyrido[3,2-e][1,4]oxazin-5(5aH)-yl)benzamide (assumed) as a yellow solid (35 mg, 20.9%). LC-MS (ES, m/z) M+1: 1355.

Synthesis of N-((4-((((1r,4r)-4-hydroxy-4-methylcyclohexyl)methyl)amino)-3-nitrophenyl)sulfonyl)-4-(2-((S)-2-(2-isopropoxyphenyl)-4-((5-methoxy-6-morpholinopyridin-3-yl)methyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed): Into an 8 mL vial were added 4-{2-[(2S)-2-(2-isopropoxyphenyl)-4-{[5-methoxy-6-(morpholin-4-yl)pyridin-3-yl]methyl}piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide (assumed) (33 mg, 0.03 mmol, 1.0 eq), ethylenediamine (31 mg, 0.5 mmol, 20.0 eq) and TBAF in THE (1 M, 1 mL). The resulting mixture was stirred for 6 hours at 80° C. The reaction was quenched by the addition of water (3 mL) and then extracted with CH₂Cl₂ (3×3 mL). The combined organic layer was washed with brine (5×3 mL), dried over anhydrous Na₂SO₄. After filtration, the filtrate was concentrated under vacuum. After filtration, the filtrate was concentrated under vacuum. The residue was purified by Prep-TLC (CH₂Cl₂/MeOH=20:1) to give N-((4-((((1r,4r)-4-hydroxy-4-methylcyclohexyl)methyl)amino)-3-nitrophenyl)sulfonyl)-4-(2-((S)-2-(2-isopropoxyphenyl)-4-((5-methoxy-6-morpholinopyridin-3-yl)methyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed) as a yellow solid (10.5 mg, 36.3%). LC-MS (ES, m/z) M+1: 1225. ¹HNMR (300 MHz, Chloroform-d) δ 12.58 (s, 1H), 8.65 (d, J=2.1 Hz, 1H), 8.46 (s, 2H), 8.11 (d, J=9.3 Hz, 1H), 7.81 (d, J=8.4 Hz, 2H), 7.10 (d, J=3.0 Hz, 1H), 6.98 (s, 1H), 6.83 (d, J=9.3 Hz, 3H), 6.71 (d, J=9.3 Hz, 1H), 6.63 (s, 1H), 6.43 (s, 1H), 6.11 (s, 1H), 4.54 (s, 4H), 4.52-4.41 (m, 1H), 3.98 (d, J=10.2 Hz, 2H), 3.86 (s, 9H), 3.58 (s, 2H), 3.50 (t, J=10.2 Hz, 4H), 3.41 (d, J=17.5 Hz, 5H), 3.21 (t, J=6.0 Hz, 2H), 2.76 (s, 1H), 2.36 (s, 3H), 2.06 (s, 1H), 1.89 (s, 2H), 1.56-1.46 (m, 1H), 1.44 (d, J=22.2 Hz, 1H), 1.19 (d, J=12.5 Hz, 12H), 0.88 (s, 3H), 0.09 (s, 12H).

### Example 102 Preparation of 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2S)-2-(2-isopropoxyphenyl)-4-[(6-methoxypyridin-3-yl)methyl]piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed)

Synthesis of tert-butyl (3S)-3-(2-isopropoxyphenyl)-4-[7-(4-{[3-nitro-4-({[(1r,4r)-4-hydroxy-4-methylcyclohexyl]methyl}amino)benzenesulfonyl]carbamoyl}-3-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]phenyl)-7-azaspiro[3.5]nonan-2-yl]piperazine-1-carboxylate (assumed): Into a 40 mL vial were added N-[3-nitro-4-({[(1r,4r)-4-hydroxy-4-methylcyclohexyl]methyl}amino)benzenesulfonyl]-4-{2-oxo-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide (800 mg, 0.8 mmol, 1.0 eq), tert-butyl (3S)-3-(2-isopropoxyphenyl)piperazine-1-carboxylate (272 mg, 0.8 mmol, 1.0 eq), NaBH₃CN (266 mg, 4.2 mmol, 5.0 eq), ZnCl₂ (577 mg, 4.2 mmol, 5.0 eq) and MeOH (10 mL). The resulting mixture was stirred for 2 hours at 70° C. The reaction was quenched by the addition of water (5 mL) and then extracted with CH₂Cl₂ (3×20 mL). The combined organic layer was washed with brine (20 mL), dried over anhydrous Na₂SO₄. After filtration, the filtrate was concentrated under vacuum. The crude residue was purified by silica gel column chromatography and eluted with CH₂Cl₂/MeOH=30:1 to give tert-butyl (3S)-3-(2-isopropoxyphenyl)-4-[7-(4-{[3-nitro-4-({[(1r,4r)-4-hydroxy-4-methylcyclohexyl]methyl}amino)benzenesulfonyl]carbamoyl}-3-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]phenyl)-7-azaspiro[3.5]nonan-2-yl]piperazine-1-carboxylate (assumed) as a yellow solid (800 mg, 75.6%). LC-MS (ES, m/z) M+1: 1248.

Synthesis of 2-[(3R,8S)-15-(hydroxymethyl)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2S)-2-(2-isopropoxyphenyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed): Into a 50 mL round-bottom flask were added tert-butyl (3S)-3-(2-isopropoxyphenyl)-4-[7-(4-{[3-nitro-4-({[(1r,4r)-4-hydroxy-4-methylcyclohexyl]methyl}amino)benzenesulfonyl]carbamoyl}-3-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]phenyl)-7-azaspiro[3.5]nonan-2-yl]piperazine-1-carboxylate (600 mg, 0.5 mmol, 1.0 eq) and TFA/DCM (3 mL/3 mL) at 0° C. The resulting mixture was stirred for 2 hours at 0° C. The mixture was basified to pH=8 with sat. NaHCO₃ and then extracted with CH₂Cl₂ (3×10 mL). The combined organic layer was washed with brine (10 mL), dried over anhydrous Na₂SO₄. After filtration, the filtrate was concentrated under vacuum. The crude residue was purified by Prep-TLC (CH₂Cl₂/MeOH=8:1) to give 2-[(3R,8S)-15-(hydroxymethyl)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2S)-2-(2-isopropoxyphenyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed) as a yellow solid (280 mg, 55.6%). LC-MS (ES, m/z) M+1: 1048.

Synthesis of 2-[(3R,8S)-15-(hydroxymethyl)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2S)-2-(2-isopropoxyphenyl)-4-[(6-methoxypyridin-3-yl)methyl]piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed): Into an 8 mL vial were added 2-[(3R,8S)-15-(hydroxymethyl)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2S)-2-(2-isopropoxyphenyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (100 mg, 0.1 mmol, 1.0 eq), NaBH₃CN (30 mg, 0.5 mmol, 5.0 eq), ZnCl₂ (65 mg, 0.5 mmol, 5.0 eq) and MeOH (2 mL). The resulting mixture was stirred for 4 hours. The reaction was quenched by the addition of water (3 mL) and then extracted with CH₂Cl₂ (3×5 mL). The combined organic layer was washed with brine (5 mL), dried over anhydrous Na₂SO₄. After filtration, the filtrate was concentrated under vacuum. The crude residue was purified by Prep-TLC (CH₂Cl₂/MeOH=12:1) to give 2-[(3R,8S)-15-(hydroxymethyl)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2S)-2-(2-isopropoxyphenyl)-4-[(6-methoxypyridin-3-yl)methyl]piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed) as a yellow solid (65 mg, 58.3%). LC-MS (ES, m/z) M+1: 1170.

Synthesis of 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2S)-2-(2-isopropoxyphenyl)-4-[(6-methoxypyridin-3-yl)methyl]piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed): Into an 8 mL vial were added 2-[(3R,8S)-15-(hydroxymethyl)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2S)-2-(2-isopropoxyphenyl)-4-[(6-methoxypyridin-3-yl)methyl]piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (60 mg, 0.05 mmol, 1.0 eq) ethylenediamine (92 mg, 1.5 mmol, 30.0 eq) and THE (1 mL). The resulting mixture was stirred for 2 hours at 70° C. The reaction was quenched by the addition of water (3 mL) and then extracted with CH₂Cl₂ (3×3 mL). The combined organic layer was washed with brine (3 mL), dried over anhydrous Na₂SO₄. After filtration, the filtrate was concentrated under vacuum. The residue was purified by Prep-TLC (CH₂Cl₂/MeOH=25:1) to give 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2S)-2-(2-isopropoxyphenyl)-4-[(6-methoxypyridin-3-yl)methyl]piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed) as a yellow solid (12 mg, 20.53%). LC-MS (ES, m/z) M+1: 1140. ¹HNMR (300 MHz, Methanol-d₄) δ 8.49 (d, J=2.3 Hz, 1H), 8.13-8.00 (m, 1H), 7.86 (d, J=9.3 Hz, 1H), 7.80-7.61 (m, 1H), 7.61-7.38 (m, 2H), 7.23 (t, J=7.6 Hz, 2H), 7.07 (d, J=3.4 Hz, 1H), 7.03-6.89 (m, 2H), 6.89-6.77 (m, 1H), 6.77-6.63 (m, 1H), 6.51 (d, J=13.8 Hz, 1H), 5.99 (dd, J=17.5, 3.4 Hz, 2H), 4.60 (d, J=5.6 Hz, 1H), 4.28 (d, J=7.6 Hz, 2H), 4.08 (d, J=24.3 Hz, 1H), 3.90 (d, J=2.0 Hz, 4H), 3.70 (s, 3H), 3.64-3.41 (m, 3H), 3.25 (d, J=4.8 Hz, 7H), 2.92 (d, J=28.4 Hz, 1H), 2.66 (d, J=12.4 Hz, 3H), 2.39 (s, 2H), 2.20 (d, J=16.0 Hz, 1H), 1.96 (s, 1H), 1.84 (d, J=14.0 Hz, 2H), 1.71 (s, 3H), 1.64-1.45 (m, 4H), 1.45-1.35 (m, 2H), 1.32 (d, J=5.6 Hz, 6H), 1.25 (t, J=3.3 Hz, 6H), 1.00-0.85 (m, 2H).

### Example 103 Preparation of 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2S)-2-(2-isopropoxyphenyl)-4-[(5-methoxypyridin-2-yl)methyl]piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed)

Synthesis of (2S)-1-benzyl-2-(2-isopropoxyphenyl)-4-[(5-methoxypyridin-2-yl)methyl]piperazine (assumed): Into a 40 mL vial were added (2S)-1-benzyl-2-(2-isopropoxyphenyl)piperazine (150 mg, 0.5 mmol, 1.0 eq), 5-methoxypyridine-2-carbaldehyde (80 mg, 0.6 mmol, 1.2 eq), ZnCl₂ (329 mg, 2.4 mmol, 5.0 eq) and MeOH (5 mL). The resulting mixture was stirred for 30 min at 25° C. To the above mixture was added NaBH₃CN (152 mg, 2.4 mmol, 5.0 eq) at 25° C. The resulting mixture was stirred for additional 2 hours at 25° C. The reaction was quenched by the addition of water (5 mL) and then extracted with EtOAc (3×5 mL). The combined organic layer was washed with brine (5 mL), dried over anhydrous Na₂SO₄. After filtration, the filtrate was concentrated under vacuum. The residue was applied onto a silica gel column and eluted with ethyl acetate/petroleum ether=1:20 to give (2S)-1-benzyl-2-(2-isopropoxyphenyl)-4-[(5-methoxypyridin-2-yl)methyl]piperazine (assumed) as a colorless oil (100 mg, 47.9%). LC-MS (ES, m/z) M+1: 432.

Synthesis of (3S)-3-(2-isopropoxyphenyl)-1-[(5-methoxypyridin-2-yl)methyl]piperazine: Into a 50 mL round-bottom flask were added (2S)-1-benzyl-2-(2-isopropoxyphenyl)-4-[(5-methoxypyridin-2-yl)methyl]piperazine (80 mg, 0.19 mmol, 1.0 eq), Pd(OH)₂/C (13 mg, 0.1 mmol, 0.5 eq), Pd/C (10 mg, 0.01 mmol, 0.5 eq) and MeOH (2 mL). The resulting mixture was stirred for 3 hours at 0° C. under H₂ (1 atm). The resulting mixture was filtered, the filter cake was washed with MeOH (3×5 mL). The filtrate was concentrated under vacuum to give (3S)-3-(2-isopropoxyphenyl)-1-[(5-methoxypyridin-2-yl)methyl]piperazine as a colorless oil (50 mg, crude), which was used in the next step directly without further purification to give. ¹HNMR (300 MHz, Chloroform-d) δ 8.26 (d, J=3.0 Hz, 1H), 7.71 (s, 1H), 7.16 (dd, J=8.4, 3.1 Hz, 3H), 6.96 (t, J=7.6 Hz, 1H), 6.87 (d, J=8.2 Hz, 1H), 4.56 (p, J=6.1 Hz, 1H), 4.10 (s, 1H), 3.85 (s, 3H), 3.64 (s, 2H), 2.89 (d, J=12.1 Hz, 2H), 2.30-2.52 (m. 2H), 2.05 (s, 2H), 1.37 (d, J=6.1 Hz, 3H), 1.28 (d, J=4.4 Hz, 3H).

Synthesis of 4-{2-[(2S)-2-(2-isopropoxyphenyl)-4-[(5-methoxypyridin-2-yl)methyl]piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide (assumed): Into an 8 mL vial were added N-[3-nitro-4-({[(1r,4r)-4-hydroxy-4-methylcyclohexyl]methyl}amino)benzenesulfonyl]-4-{2-oxo-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide (87 mg, 0.1 mmol, 0.7 eq), (3S)-3-(2-isopropoxyphenyl)-1-[(5-methoxypyridin-2-yl)methyl]piperazine (45 mg, 0.1 mmol, 1.0 eq), NaBH₃CN (41 mg, 0.7 mmol, 5.0 eq), ZnCl₂ (90 mg, 0.7 mmol, 5 eq) and MeOH (2 mL). The resulting mixture was stirred for 2 hours at 70° C. The reaction was quenched by the addition of water (2 mL) and extracted with CH₂Cl₂ (3×3 mL). The combined organic layer was washed with brine (3 mL), and then dried over anhydrous Na₂SO₄. After filtration, the filtrate was concentrated under vacuum. The residue was purified by Prep-TLC (CH₂Cl₂/MeOH=20:1) to give 4-{2-[(2S)-2-(2-isopropoxyphenyl)-4-[(5-methoxypyridin-2-yl)methyl]piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide (assumed) as a yellow solid (35 mg, 20.9%). LC-MS (ES, m/z) M+1: 1269.

Synthesis of 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2S)-2-(2-isopropoxyphenyl)-4-[(5-methoxypyridin-2-yl)methyl]piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed): Into an 8 mL vial were added 4-{2-[(2S)-2-(2-isopropoxyphenyl)-4-[(5-methoxypyridin-2-yl)methyl]piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide (33 mg, 0.03 mmol, 1.0 eq), ethylenediamine (31 mg, 0.5 mmol, 20.0 eq) and TBAF in THF (1 M, 1 mL). The resulting mixture was stirred for 3 hours at 80° C. The reaction was quenched by the addition of water (3 mL) and then extracted with CH₂Cl₂ (3×3 mL). The combined organic layer was washed with brine (5×3 mL), dried over anhydrous Na₂SO₄. After filtration, the filtrate was concentrated under vacuum. The crude residue was purified by Prep-TLC (CH₂Cl₂/MeOH=12:1) to give crude product. The residue was purified by reverse flash chromatography using the following conditions: column, C18 silica gel; mobile phase, MeCN in water (0.1% FA), 10% to 50% gradient in 10 min; detector, UV 254 nm. Finally, 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2S)-2-(2-isopropoxyphenyl)-4-[(5-methoxypyridin-2-yl)methyl]piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed) (3.6 mg, 12.2%) was obtained as a yellow solid. LC-MS (ES, m/z) M+1: 1140. ¹HNMR (300 MHz, Methanol-d₄) δ 8.48 (d, J=2.4 Hz, 2H), 8.09 (d, J=10.0 Hz, 1H), 7.92 (dd, J=8.9, 4.6 Hz, 2H), 7.68 (s, 1H), 7.54 (dd, J=9.2, 2.3 Hz, 1H), 7.43 (t, J=7.5 Hz, 1H), 7.11 (s, 2H), 7.04 (dd, J=14.8, 7.3 Hz, 3H), 6.81-6.69 (m, 2H), 6.53 (s, 1H), 4.56 (d, J=7.1 Hz, 3H), 4.31 (d, J=7.5 Hz, 3H), 4.13 (s, 3H), 4.04 (d, J=1.6 Hz, 4H), 3.66 (s, 3H), 3.50-3.44 (m, 4H), 3.28 (d, J=6.7 Hz, 6H), 3.16 (s, 4H), 2.31 (s, 1H), 1.85 (d, J=13.9 Hz, 3H), 1.72 (s, 4H), 1.69 (s, 3H), 1.67 (s, 3H), 1.53 (s, 3H), 1.48 (d, J=6.0 Hz, 2H), 1.41 (t, J=7.0 Hz, 1H), 1.31 (s, 2H), 1.26 (d, J=4.4 Hz, 3H).

### Example 104 Preparation of 4-{2-[(2S)-4-[(5,6-dimethoxypyridin-3-yl)methyl]-2-(2-isopropoxyphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-N-[3-nitro-4-({[(1r,4r)-4-hydroxy-4-methylcyclohexyl]methyl}amino)benzenesulfonyl]benzamide (assumed)

Synthesis of 5-bromo-2,3-dimethoxypyridine: Into a 50-mL round-bottom flask, were placed 2,3-dimethoxypyridine (8.0 g, 57.5 mmol, 1.0 eq), Br₂ (10.1 g, 63.2 mmol, 1.1 eq), dichloromethane (80 mL). The resulting mixture was stirred for 14 hours at 25° C. The reaction was then quenched by the addition of water (50 mL), and then extracted with ethyl acetate (50 mL). The organic layer was washed with water (2×50 mL) and brine (2×50 mL), and dried over Na₂SO₄. After filtration, the resulting mixture was concentrated under vacuum. The crude residue was applied onto a silica gel column and eluted with ethyl acetate/petroleum ether=1:2 to give 5-bromo-2,3-dimethoxypyridine as an off-white oil (5.0 g, 40.0%). LC-MS (ES, m/z) M+1: 218. ¹HNMR (300 MHz, Chloroform-d) δ 7.73 (d, J=2.1 Hz, 1H), 7.09 (d, J=2.1 Hz, 1H), 3.95 (s, 3H), 3.83 (s, 3H).

Synthesis of 5,6-dimethoxypyridine-3-carbaldehyde: Into a 250 mL 3-necked round-bottom flask, were placed 5-bromo-2,3-dimethoxypyridine (4.7 g, 21.6 mmol, 1.0 eq), THE (50 mL). After that, n-BuLi (1.1 eq) was added dropwised at −78° C. The resulting mixture was stirred for 30 minutes at −78° C. To the above mixture was added DMF (2.2 g, 30.2 mmol, 1.4 eq) at −78° C. The resulting mixture was stirred for additional 30 minutes at −78° C. The reaction was then quenched by the addition of water (200 mL) and extracted with ethyl acetate (2×100 mL). The combined organic layer was washed with brine (2×100 mL). The mixture was dried over anhydrous Na₂SO₄. After filtration, the filtrate was concentrated under vacuum. The crude residue was purified onto a silica gel column and eluted with ethyl acetate/petroleum ether=1:1 to give 5,6-dimethoxypyridine-3-carbaldehyde as a light pink solid (2.7 g, 74.9%). ¹HNMR (300 MHz, DMSO-d₆) δ 9.95 (s, 1H), 8.35 (d, J=1.8 Hz, 1H), 7.53 (d, J=1.8 Hz, 1H), 3.99 (s, 3H), 3.87 (s, 3H).

Synthesis of (2S)-1-benzyl-4-[(5,6-dimethoxypyridin-3-yl)methyl]-2-(2-isopropoxyphenyl)piperazine (assumed): Into a 40-mL sealed-tube, were placed (2S)-1-benzyl-2-(2-isopropoxyphenyl)piperazine (150 mg, 0.5 mmol, 1.0 eq), 5,6-dimethoxypyridine-3-carbaldehyde (89 mg, 0.5 mmol, 1.1 eq), DCE (4 mL), NaBH(OAc)₃ (307 mg, 1.4 mmol, 3.0 eq). The resulting solution was stirred for 2 hours at 25° C. The reaction was then quenched by the addition of water (40 mL) and then extracted with ethyl acetate (2×40 mL). The combined organic layer was washed with brine (2×40 mL) and dried over anhydrous Na₂SO₄. After filtration, the filtrate was concentrated under vacuum. The crude residue was purified onto a silica gel column and eluted with ethyl acetate/petroleum ether=1:2 to give (2S)-1-benzyl-4-[(5,6-dimethoxypyridin-3-yl)methyl]-2-(2-isopropoxyphenyl)piperazine (assumed) as a light yellow oil (140 mg, 62.8%). LC-MS (ES, m/z) M+1: 462.

Synthesis of (3S)-1-[(5,6-dimethoxypyridin-3-yl)methyl]-3-(2-isopropoxyphenyl)piperazine (assumed): Into a 50-mL round-bottom flask, were placed (2S)-1-benzyl-4-[(5,6-dimethoxypyridin-3-yl)methyl]-2-(2-isopropoxyphenyl)piperazine (120 mg, 0.3 mmol, 1.0 eq), THE (3 mL), Pd/C (14 mg, 0.1 mmol, 0.5 eq), Pd(OH)₂/C (18 mg, 0.1 mmol, 0.5 eq). The mixture was hydrogenated at 25° C. under H₂ (2 atm) for 4 hours. The resulting mixture was filtered. The filtrate was concentrated under vacuum to give (3S)-1-[(5,6-dimethoxypyridin-3-yl)methyl]-3-(2-isopropoxyphenyl)piperazine (assumed) as a light yellow oil (90 mg, crude).

Synthesis of 4-{2-[(2S)-4-[(5,6-dimethoxypyridin-3-yl)methyl]-2-(2-isopropoxyphenyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide (assumed): Into an 8-mL sealed-tube, were placed (3S)-1-[(5,6-dimethoxypyridin-3-yl)methyl]-3-(2-isopropoxyphenyl)piperazine (80 mg, 0.2 mmol, 1.0 eq), N-[3-nitro-4-({[(1r,4r)-4-hydroxy-4-methylcyclohexyl]methyl}amino)benzenesulfonyl]-4-{2-oxo-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide (163 mg, 0.2 mmol, 0.8 eq), MeOH (1 mL), ZnCl₂ (88 mg, 0.6 mmol, 3.0 eq), NaBH₃CN (41 mg, 0.6 mmol, 3.0 eq). The resulting solution was stirred for 3 hours at 70° C. The reaction was then quenched by the addition of water (20 mL) and then extracted with dichloromethane/methanol=10:1 (2×20 mL). The combined organic layer was washed with brine (2×20 mL) and dried over anhydrous Na₂SO₄. After filtration, the filtrate was concentrated under vacuum. The crude residue was purified onto a silica gel column and eluted with dichloromethane/methanol=15:1 to give 4-{2-[(2S)-4-[(5,6-dimethoxypyridin-3-yl)methyl]-2-(2-isopropoxyphenyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide (assumed) as a light yellow solid (55 mg, 19.7%). LC-MS (ES, m/z) M+1: 1300.

Synthesis of 4-{2-[(2S)-4-[(5,6-dimethoxypyridin-3-yl)methyl]-2-(2-isopropoxyphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-N-[3-nitro-4-({[(1r,4r)-4-hydroxy-4-methylcyclohexyl]methyl}amino)benzenesulfonyl]benzamide (assumed): Into an 8 mL sealed-tube, were placed 4-{2-[(2S)-4-[(5,6-dimethoxypyridin-3-yl)methyl]-2-(2-isopropoxyphenyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide (50 mg, 0.04 mmol, 1.0 eq), ethylenediamine (69 mg, 1.1 mmol, 30.0 eq) and TBAF in THE (1 M, 2 mL). The resulting mixture was stirred for 8 hours at 70° C. The reaction was quenched by the addition of water (10 mL) and then extracted with EtOAc (3×30 mL). The combined organic layer was washed with brine (30 mL), dried over anhydrous Na₂SO₄. After filtration, the filtrate was concentrated under vacuum. The residue was purified by Prep-TLC (dichloromethane/methanol=15:1) to give 4-{2-[(2S)-4-[(5,6-dimethoxypyridin-3-yl)methyl]-2-(2-isopropoxyphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-N-[3-nitro-4-({[(1r,4r)-4-hydroxy- 4-methylcyclohexyl]methyl}amino)benzenesulfonyl]benzamide (assumed) as a yellow solid (17 mg, 37.8%). LC-MS (ES, m/z) M+1: 1170. ¹HNMR (400 MHz, Methanol-d₄) δ 8.46 (d, J=2.4 Hz, 1H), 7.93 (d, J=9.2 Hz, 1H), 7.66 (s, 1H), 7.64-7.47 (m, 2H), 7.42 (t, J=8.0 Hz, 1H), 7.31 (s, 1H), 7.16-7.06 (m, 2H), 7.06-6.93 (m, 2H), 6.74-6.71 (m, 2H), 6.47 (s, 1H), 6.02 (d, J=3.6 Hz, 1H), 4.77-4.69 (m, 1H), 4.35-4.29 (m, 1H), 3.94 (s, 3H), 3.92-3.90 (m, 1H), 3.87 (s, 3H), 3.78-3.57 (m, 3H), 3.49 (t, J=11.8 Hz, 2H), 3.27 (d, J=6.8 Hz, 2H), 3.24-3.20 (m, 3H), 3.16-3.10 (m, 1H), 2.26-2.18 (m, 1H), 2.03-1.96 (m, 1H) 1.87-1.81 (m, 2H), 1.76-1.70 (m, 4H), 1.68-1.66 (m, 1H) 1.60-1.42 (m, 10H), 1.40-1.38 (m, 4H), 1.35-1.31 (m, 10H), 1.28-1.25 (m, 4H).

### Example 105 Preparation of 4-{2-[(2S)-4-[(5,6-dimethoxypyridin-2-yl)methyl]-2-(2-isopropoxyphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-N-[3-nitro-4-({[(1r,4r)-4-hydroxy-4-methylcyclohexyl]methyl}amino)benzenesulfonyl]benzamide

Synthesis of 6-iodo-2,3-dimethoxypyridine: Into a 50 mL round-bottom flask, were placed 2-bromo-6-iodo-3-methoxypyridine (7.0 g, 22.3 mmol, 1.0 eq), sodium methoxide (1.8 g, 33.4 mmol, 1.5 eq), DMSO (35 mL). The resulting solution was stirred for 16 hours at 100° C. The reaction was then quenched by the addition of water (50 mL) and extracted with ethyl acetate (2×50 mL). The combined organic layer was washed with water (2×50 mL), brine (2×50 mL) and dried over anhydrous Na₂SO₄. After filtration, the filtrate was concentrated under vacuum to give 6-iodo-2,3-dimethoxypyridine as an off-white solid (5.0 g, 98.1%). LC-MS (ES, m/z) M+1: 266. ¹HNMR (300 MHz, DMSO-d₆) δ 7.33 (d, J=8.1 Hz, 1H), 7.05 (d, J=8.1 Hz, 1H), 3.84 (s, 3H), 3.76 (s, 3H).

Synthesis of 5,6-dimethoxypyridine-2-carbaldehyde: Into a 250 mL 3-necked round-bottom flask, were placed 6-iodo-2,3-dimethoxypyridine (5.0 g, 18.9 mmol, 1.0 eq), THE (50 mL), which was followed by the addition of n-BuLi (1.8 g, 28.3 mmol, 1.5 eq) at −78° C. The resulting mixture was stirred for 30 minutes at −78° C. To the above mixture was added DMF (1.9 g, 26.4 mmol, 1.4 eq) at −78° C. The resulting mixture was stirred for additional 30 minutes at −78° C. The reaction was then quenched by the addition of water (50 mL) and extracted with ethyl acetate (2×50 mL). The combined organic layer was washed with brine (2×50 mL). The mixture was dried over anhydrous Na₂SO₄. After filtration, the filtrate was concentrated under vacuum. The crude residue was purified onto a silica gel column and eluted with ethyl acetate/petroleum ether=1:1 to give 5,6-dimethoxypyridine-2-carbaldehyde as a pink solid (3.0 g, 95.1%). LC-MS (ES, m/z) M+1: 168. ¹HNMR (300 MHz, DMSO-d₆) δ 9.78 (s, 1H), 7.64 (d, J=8.1 Hz, 1H), 7.46 (d, J=8.1 Hz, 1H), 3.96 (s, 3H), 3.90 (s, 3H).

Synthesis of (2S)-1-benzyl-4-[(5,6-dimethoxypyridin-2-yl)methyl]-2-(2-isopropoxyphenyl)piperazine (assumed): Into a 40-mL sealed-tube, were placed (2S)-1-benzyl-2-(2-isopropoxyphenyl)piperazine (assumed) (150 mg, 0.5 mmol, 1.0 eq), 5,6-dimethoxypyridine-2-carbaldehyde (97 mg, 0.6 mmol, 1.2 eq), MeOH (3 mL), ZnCl₂ (198 mg, 1.4 mmol, 3.0 eq), NaBH₃CN (91 mg, 1.4 mmol, 3.0 eq). The resulting solution was stirred for 2 hours at 25° C. The reaction was then quenched by the addition of water (40 mL) and extracted with ethyl acetate (2×40 mL). The combined organic layer was washed with brine (2×40 mL) and dried over anhydrous Na₂SO₄. After filtration, the filtrate was concentrated under vacuum. The crude residue was purified onto a silica gel column and eluted with ethyl acetate/petroleum ether=1:2 to give (2S)-1-benzyl-4-[(5,6-dimethoxypyridin-2-yl)methyl]-2-(2-isopropoxyphenyl)piperazine (assumed) as a light yellow oil (80 mg, 35.9%). LC-MS (ES, m/z) M+1: 462.

Synthesis of (3S)-1-[(5,6-dimethoxypyridin-2-yl)methyl]-3-(2-isopropoxyphenyl)piperazine (assumed): Into a 50-mL round-bottom flask, were placed (2S)-1-benzyl-4-[(5,6-dimethoxypyridin-2-yl)methyl]-2-(2-isopropoxyphenyl)piperazine (assumed) (70 mg, 0.2 mmol, 1.0 eq), THE (3 mL), Pd/C (8 mg, 0.1 mmol, 0.5 eq), Pd(OH)₂/C (11 mg, 0.1 mmol, 0.5 eq). The mixture was hydrogenated at 25° C. under H₂ (2 atm) for 16 hours. The resulting mixture was filtered. The filtrate was concentrated under vacuum to give (3S)-1-[(5,6-dimethoxypyridin-2-yl)methyl]-3-(2-isopropoxyphenyl)piperazine (assumed) as a light yellow oil (55 mg crude).

Synthesis of 4-{2-[(2S)-4-[(5,6-dimethoxypyridin-2-yl)methyl]-2-(2-isopropoxyphenyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide (assumed): Into an 8 mL vial were added N-[3-nitro-4-({[(1r,4r)-4-hydroxy-4-methylcyclohexyl]methyl}amino)benzenesulfonyl]-4-{2-oxo-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide (assumed) (87 mg, 0.1 mmol, 0.7 eq), (3S)-1-[(5,6-dimethoxypyridin-2-yl)methyl]-3-(2-isopropoxyphenyl)piperazine (45 mg, 0.1 mmol, 1.0 eq), NaBH₃CN (41 mg, 0.7 mmol, 5.0 eq), ZnCl₂ (90 mg, 0.7 mmol, 5 eq) and MeOH (2 mL). The resulting mixture was stirred for 2 hours at 70° C. The reaction was quenched by the addition of water (2 mL) and extracted with CH₂Cl₂ (3×3 mL). The combined organic layer was washed with brine (3 mL), dried over anhydrous Na₂SO₄. After filtration, the filtrate was concentrated under vacuum. The residue was purified by Prep-TLC (CH₂Cl₂/MeOH=20:1) to give 4-{2-[(2S)-4-[(5,6-dimethoxypyridin-2-yl)methyl]-2-(2-isopropoxyphenyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide (assumed) as a yellow solid (35 mg, 20.9%). LC-MS (ES, m/z) M+1: 1300.

Synthesis of 4-{2-[(2S)-4-[(5,6-dimethoxypyridin-2-yl)methyl]-2-(2-isopropoxyphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-N-[3-nitro-4-({[(1r,4r)-4-hydroxy-4-methylcyclohexyl]methyl}amino)benzenesulfonyl]benzamide (assumed): Into an 8 mL vial were added 4-{2-[(2S)-4-[(5,6-dimethoxypyridin-2-yl)methyl]-2-(2-isopropoxyphenyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide (33 mg, 0.03 mmol, 1.0 eq), ethylenediamine (31 mg, 0.5 mmol, 20.0 eq) and TBAF in THF (1 M, 1 mL). The resulting mixture was stirred for 3 hours at 60° C. The reaction was quenched by the addition of water (3 mL) and extracted with CH₂Cl₂ (3×3 mL). The combined organic layer was washed with brine (5×3 mL), dried over anhydrous Na₂SO₄. After filtration, the filtrate was concentrated under vacuum. After filtration, the filtrate was concentrated under vacuum. The residue was purified by Prep-TLC (CH₂Cl₂/MeOH=20:1) to give 4-{2-[(2S)-4-[(5,6-dimethoxypyridin-2-yl)methyl]-2-(2-isopropoxyphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-N-[3-nitro-4-({[(1r,4r)-4-hydroxy-4-methylcyclohexyl]methyl}amino)benzenesulfonyl]benzamide as a yellow solid (7.2 mg, 24.3%). LC-MS (ES, m/z) M+1: 1169. ¹H NMR (300 MHz, Chloroform-d) δ 12.56 (s, 1H), 8.62 (d, J=2.1 Hz, 1H), 8.44 (s, 1H), 8.41 (s, 1H), 8.08 (d, J=9.3 Hz, 1H), 7.83-7.74 (m, 1H), 7.43 (s, 1H), 7.15 (s, 1H), 7.12-7.04 (m, 1H), 6.97 (d, J=7.8 Hz, 1H), 6.81 (d, J=9.0 Hz, 3H), 6.68 (d, J=9.3 Hz, 1H), 6.61 (s, 1H), 6.41 (s, 1H), 6.08 (d, J=3.5 Hz, 1H), 4.52 (d, J=5.4 Hz, 2H), 4.51-4.37 (m, 1H), 3.96 (s, 3H), 3.84 (s, 3H), 3.62 (s, 2H), 3.54-3.35 (m, 3H), 3.19 (t, J=6.0 Hz, 2H), 3.03 (s, 1H), 1.84 (d, J=13.5 Hz, 12H), 1.58-1.40 (m, 2H), 1.28 (d, J=13.2 Hz, 14H), 0.91-0.80 (m, 14H).

### Example 106 Preparation of 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2S)-2-(2-isopropoxyphenyl)-4-[(6-methoxy-5-methylpyridin-2-yl)methyl]piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed)

Synthesis of 6-methoxy-5-methylpyridine-2-carbaldehyde: A solution of 1-methyl-piperazine (6.0 g, 59.9 mmol, 1.6 eq) in THE was treated with n-BuLi in hexanes (22 mL, 233.5 mmol, 6.4 eq) for 30 min at −78° C. under nitrogen atmosphere, which was followed by the addition of 6-methoxypyridine-2-carbaldehyde (5.0 g, 36.5 mmol, 1.0 eq) dropwise at −78° C. The resulting mixture was stirred for 30 min at −78° C. under nitrogen atmosphere. To the above mixture was added t-BuLi (50 mL) dropwise over 20 min at −78° C. The resulting mixture was stirred for additional 3 hours at −78° C. The resulting mixture was allowed to warm to −40° C. for 15 min under nitrogen atmosphere. CH₃I (35.5 g, 250.1 mmol, 6.9 eq) was added slowly at −78° C., and the resulting mixture was allowed to 25° C. The resulting mixture was poured into a vigorously stirred cold brine (50 mL) and then extracted with ethyl ether (3×50 mL). The combined organic layer was dried over anhydrous Na₂SO₄, filtered and concentrated under vacuum. The crude residue was applied onto a silica gel column and eluted with ethyl acetate/petroleum ether=1:20 to give 6-methoxy-5-methylpyridine-2-carbaldehyde as a white solid (2.2 g, 39.9%). LC-MS (ES, m/z) M+1: 152.

Synthesis of (2S)-1-benzyl-2-(2-isopropoxyphenyl)-4-[(6-methoxy-5-methylpyridin-2-yl)methyl]piperazine (assumed): A solution of (2S)-1-benzyl-2-(2-isopropoxyphenyl)piperazine (150 mg, 0.5 mmol, 1.0 eq) and 6-methoxy-5-methylpyridine-2-carbaldehyde (88 mg, 0.56 mmol, 1.2 eq) in MeOH (4 mL) was treated with ZnCl₂ (329 mg, 2.4 mmol, 5.0 eq) for 30 min at 25° C., which was followed by the addition of NaBH₃CN (152 mg, 2.4 mmol, 5.0 eq) dropwise at 25° C. The resulting mixture was stirred for 2 hours at 25° C. The reaction was quenched by the addition of water (2 mL) and then extracted with EtOAc (3×10 mL). The combined organic layer was washed with brine (10 mL), dried over anhydrous Na₂SO₄. After filtration, the filtrate was concentrated under vacuum. The crude residue was applied onto a silica gel column and eluted with THE/petroleum ether=1:20 to give (2S)-1-benzyl-2-(2-isopropoxyphenyl)-4-[(6-methoxy-5-methylpyridin-2-yl)methyl]piperazine (assumed) (100 mg, 46.4%) as a colorless oil. LC-MS (ES, m/z) M+1: 446.

Synthesis of (3S)-3-(2-isopropoxyphenyl)-1-[(6-methoxy-5-methylpyridin-2-yl)methyl]piperazine (assumed): Into a 50 mL round-bottom flask were added (2S)-1-benzyl-2-(2-isopropoxyphenyl)-4-[(6-methoxy-5-methylpyridin-2-yl)methyl]piperazine (90 mg, 0.2 mmol, 1.0 eq), Pd(OH)₂/C (14 mg, 0.1 mmol, 0.5 eq), Pd/C (11 mg, 0.1 mmol, 0.5 eq) and THE (2 mL) at 0° C. The resulting mixture was stirred for 3 hours at 0° C. under H₂ (1 atm). The resulting mixture was filtered, the filter cake was washed with THE (3×5 mL). The filtrate was concentrated under vacuum. The crude product (3S)-3-(2-isopropoxyphenyl)-1-[(6-methoxy-5-methylpyridin-2-yl)methyl]piperazine (assumed) was used directly to the next step without further purification (65 mg, 90.5%).

Synthesis of 4-{2-[(2S)-2-(2-isopropoxyphenyl)-4-[(6-methoxy-5-methylpyridin-2-yl)methyl]piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide (assumed): Into an 8 mL vial were added N-[3-nitro-4-({[(1r,4r)-4-hydroxy-4-methylcyclohexyl]methyl}amino)benzenesulfonyl]-4-{2-oxo-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide (112 mg, 0.1 mmol, 0.7 eq), NaBH₃CN (53 mg, 0.8 mmol, 5.0 eq), ZnCl₂ (115 mg, 0.8 mmol, 5.0 eq) and MeOH (1 mL). The resulting mixture was stirred for 2 hours 25° C. The reaction was quenched by the addition of water (2 mL) and then extracted with CH₂Cl₂ (3×3 mL). The combined organic layer was washed with brine (3 mL), dried over anhydrous Na₂SO₄. After filtration, the filtrate was concentrated under vacuum. The residue was purified by Prep-TLC (CH₂Cl₂/MeOH=20:1) to give 4-{2-[(2S)-2-(2-isopropoxyphenyl)-4-[(6-methoxy-5-methylpyridin-2-yl)methyl]piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide (assumed) as a yellow solid (50 mg, 23.1%). LC-MS (ES, m/z) M+1: 1284.

Synthesis of 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2S)-2-(2-isopropoxyphenyl)-4-[(6-methoxy-5-methylpyridin-2-yl)methyl]piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed): Into an 8 mL vial were added 4-{2-[(2S)-2-(2-isopropoxyphenyl)-4-[(6-methoxy-5-methylpyridin-2-yl)methyl]piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide (48 mg, 0.04 mmol, 1.0 eq), ethylenediamine (45 mg, 0.7 mmol, 20.0 eq) and TBAF in THF (1 M, 2 mL). The resulting mixture was stirred for 3 hours at 70° C. The reaction was quenched by the addition of water (2 mL) and then extracted with CH₂Cl₂ (3×5 mL). The combined organic layer was washed with aq. HCl (1 M, 3×3 mL), and dried over anhydrous Na₂SO₄. After filtration, the filtrate was concentrated under vacuum. The residue was purified by Prep-TLC (CH₂Cl₂/MeOH=25:1) to give 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2S)-2-(2-isopropoxyphenyl)-4-[(6-methoxy-5-methylpyridin-2-yl)methyl]piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed) (10 mg, 23.19%) as a yellow solid. LC-MS (ES, m/z) M+1: 1154. ¹HNMR (300 MHz, Methanol-d₄) δ 8.49 (dd, J=6.2, 2.3 Hz, 1H), 7.86 (d, J=9.3 Hz, 1H), 7.66-7.46 (m, 3H), 7.42 (d, J=7.1 Hz, 1H), 7.24 (t, J=7.5 Hz, 1H), 7.12-6.81 (m, 4H), 6.67 (d, J=9.4 Hz, 2H), 6.51 (d, J=13.8 Hz, 1H), 5.99 (dd, J=17.7, 3.4 Hz, 1H), 4.71-4.54 (m, 1H), 4.36-4.09 (m, 3H), 3.93 (d, J=3.4 Hz, 5H), 3.70 (d, J=14.3 Hz, 4H), 3.54 (d, J=34.3 Hz, 3H), 2.94-2.74 (m, 2H), 2.56 (d, J=12.5 Hz, 3H), 2.37-2.18 (m, 2H), 2.15 (s, 3H), 2.06-1.91 (m, 2H), 1.84 (d, J=13.4 Hz, 5H), 1.73 (d, J=12.2 Hz, 6H), 1.53 (t, J=12.6 Hz, 5H), 1.44-1.36 (m, 3H), 1.33 (d, J=2.1 Hz, 3H), 1.31 (d, J=2.6 Hz, 3H), 1.26 (d, J=6.8 Hz, 2H), 0.91 (d, J=6.9 Hz, 2H).

### Example 107 Preparation of 2-[(3R,8S)-13-fluoro-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2S)-2-(2-isopropoxyphenyl)-4-methylpiperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed)

Synthesis of 2-[(3R,8S)-13-fluoro-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]-N-[3-nitro-4-({[(1r,4r)-4-hydroxy-4-methylcyclohexyl]methyl}amino)benzenesulfonyl]-4-{2-oxo-7-azaspiro[3.5]nonan-7-yl}benzamide: Into an 8-mL sealed tube purged and maintained with an inert atmosphere of nitrogen, were placed 2-bromo-N-[3-nitro-4-({[(1r,4r)-4-hydroxy-4-methylcyclohexyl]methyl}amino)benzenesulfonyl]-4-{2-oxo-7-azaspiro[3.5]nonan-7-yl}benzamide (200 mg, 0.3 mmol, 1.0 eq), (3R,8S)-13-fluoro-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraene (114 mg, 0.3 mmol, 1.0 eq), N₁,N₂-diphenyloxalamide (43 mg, 0.2 mmol, 0.6 eq), CuI (34 mg, 0.2 mmol, 0.6 eq), t-BuONa (86 mg, 0.9 mmol, 3.0 eq), DMSO (2 mL). The resulting solution was stirred for 2 hours at 120° C. The reaction was then quenched by the addition of water (30 mL) and then extracted with ethyl acetate (2×30 mL). The combined organic layer was washed with brine (2×30 mL) and dried over anhydrous Na₂SO₄. After filtration, the filtrate was concentrated under vacuum. The crude residue was applied onto a silica gel column and eluted with dichloromethane/methanol=8:1 to give 2-[(3R,8S)-13-fluoro-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]-N-[3-nitro-4-({[(1r,4r)-4-hydroxy- 4-methylcyclohexyl]methyl}amino)benzenesulfonyl]-4-{2-oxo-7-azaspiro[3.5]nonan-7-yl}benzamide as a yellow solid (80 mg, 27.6%). LC-MS (ES, m/z) M+1: 962.

Synthesis of 2-[(3R,8S)-13-fluoro-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2S)-2-(2-isopropoxyphenyl)-4-methylpiperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed): Into an 8-mL sealed tube purged and maintained with an inert atmosphere of nitrogen, were placed 2-[(3R,8S)-13-fluoro-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]-N-[3-nitro-4-({[(1r,4r)-4-hydroxy-4-methylcyclohexyl]methyl}amino)benzenesulfonyl]-4-{2-oxo-7-azaspiro[3.5]nonan-7-yl}benzamide (100 mg, 0.1 mmol, 1.0 eq), (3S)-3-(2-isopropoxyphenyl)-1-methylpiperazine (assumed) (24 mg, 0.1 mmol, 1.0 eq), ZnCl₂ (20 mg, 0.3 mmol, 3.0 eq), NaBH₃CN (20 mg, 0.3 mmol, 3.0 eq), MeOH (2 mL). The resulting solution was stirred for 3 hours at 80° C. The reaction was then quenched by the addition of water (30 mL) and then extracted with ethyl acetate (2×30 mL). The combined organic layer was washed with brine (2×30 mL) and dried over anhydrous Na₂SO₄. After filtration, the filtrate was concentrated under vacuum. The crude residue was applied onto a silica gel column and eluted with dichloromethane/methanol=10:1 to give 2-[(3R,8S)-13-fluoro-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2S)-2-(2-isopropoxyphenyl)-4-methylpiperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed) as a yellow solid (50 mg, 40.8%). LC-MS (ES, m/z) M+1: 1181.

Synthesis of 2-[(3R,8S)-13-fluoro-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2S)-2-(2-isopropoxyphenyl)-4-methylpiperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed): Into an 8-mL sealed tube, were placed 2-[(3R,8S)-13-fluoro-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2S)-2-(2-isopropoxyphenyl)-4-methylpiperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed) (50 mg, 0.04 mmol, 1.0 eq), ethylenediamine (51 mg, 0.8 mmol, 20.0 eq) and TBAF in THF (1 M, 1 mL). The resulting solution was stirred for 4 hours at 50° C. The resulting mixture was filtered, the filtrate was concentrated under vacuum. The crude residue was applied onto a silica gel column and eluted with dichloromethane/methanol=15:1 to give 2-[(3R,8S)-13-fluoro-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2S)-2-(2-isopropoxyphenyl)-4-methylpiperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed) as a yellow solid (1.5 mg, 3.4%). LC-MS (ES, m/z) M+1: 1050. ¹HNMR (300 MHz, Methanol-d₄) δ 8.60-8.45 (m, 1H), 7.84-7.57 (m, 2H), 7.49-7.40 (m, 1H), 7.31-7.21 (m, 1H), 7.08-6.79 (m, 4H), 6.77-6.62 (m, 2H), 6.41 (d, J=16.8 Hz, 1H), 4.77-4.56 (m, 2H), 4.36-4.09 (m, 3H), 3.97-3.43 (m, 8H), 3.24-3.02 (m, 5H), 2.93-2.39 (m, 7H), 2.09-1.66 (m, 10H), 1.61-1.47 (m, 7H), 1.31-1.19 (m, 10H).

### Example 108 Preparation of 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2S)-2-(2-isopropoxyphenyl)-4-[(1r,4r)-4-hydroxy-4-methylcyclohexyl]piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed)

Synthesis of (1r,4r)-4-[(3S)-3-(2-isopropoxyphenyl)piperazin-1-yl]-1-methylcyclohexan-1-ol (assumed) and (1s,4s)-4-[(3S)-3-(2-isopropoxyphenyl)piperazin-1-yl]-1-methylcyclohexan-1-ol (assumed): Into a 40 mL vial were added 4-hydroxy-4-methylcyclohexan-1-one (200 mg, 1.6 mmol, 1.0 eq), (2S)-2-(2-isopropoxyphenyl)piperazine hydrochloride (412 mg, 1.9 mmol, 1.2 eq), NaBH₃CN (490 mg, 7.8 mmol, 5.0 eq), ZnCl₂ (1.1 g, 7.8 mmol, 5.0 eq) and MeOH (5 mL). The resulting mixture was stirred for 6 hours. The crude residue was purified by reverse flash chromatography using the following conditions: column, C18 silica gel; mobile phase, MeCN in water (0.1% NH₃—H₂O), 10% to 50% gradient in 10 min; detector, UV 254 nm. Finally, (1r,4r)-4-[(3S)-3-(2-isopropoxyphenyl)piperazin-1-yl]-1-methylcyclohexan-1-ol (assumed) (65 mg, 12.5%) and (1s,4s)-4-[(3S)-3-(2-isopropoxyphenyl)piperazin-1-yl]-1-methylcyclohexan-1-ol (assumed) was obtained as an off-white solid (60 mg, 11.6%). LC-MS (ES, m/z) M+1: 333.

Synthesis of 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2S)-2-(2-isopropoxyphenyl)-4-[(1r,4r)-4-hydroxy-4-methylcyclohexyl]piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed): Into an 8 mL vial were added (1r,4r)-4-[(3S)-3-(2-isopropoxyphenyl)piperazin-1-yl]-1-methylcyclohexan-1-ol (40 mg, 0.1 mmol, 1.0 eq), 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-N-[3-nitro-4-({[(1r,4r)-4-hydroxy-4-methylcyclohexyl]methyl}amino)benzenesulfonyl]-4-{2-oxo-7-azaspiro[3.5]nonan-7-yl}benzamide (90 mg, 0.1 mmol, 0.9 eq), NaBH₃CN (38 mg, 0.6 mmol, 5 eq), ZnCl₂ (82 mg, 0.6 mmol, 5.0 eq) and MeOH (2 mL). The resulting mixture was stirred for 2 hours at 70° C. The reaction was quenched by the addition of water (2 mL) and then extracted with CH₂Cl₂ (3×5 mL). The combined organic layer was washed with brine (5 mL), dried over anhydrous Na₂SO₄. After filtration, the filtrate was concentrated under vacuum. The crude residue was purified by Prep-TLC (CH₂Cl₂/MeOH=20:1) to give 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2S)-2-(2-isopropoxyphenyl)-4-[(1r,4r)-4-hydroxy-4-methylcyclohexyl]piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed) as a yellow solid (28 mg, 20.6%). LC-MS (ES, m/z) M+1: 1131. ¹HNMR (300 MHz, Methanol-d₄) δ 8.54 (d, J=2.2 Hz, 1H), 7.81 (d, J=9.0 Hz, 1H), 7.53-7.32 (m, 2H), 7.32-7.19 (m, 1H), 7.06 (d, J=3.5 Hz, 1H), 6.99 (t, J=9.7 Hz, 1H), 6.95-6.85 (m, 2H), 6.71-6.60 (m, 2H), 6.54 (s, 1H), 6.01 (d, J=3.4 Hz, 1H), 4.67 (dq, J=12.0, 5.8 Hz, 1H), 4.34-4.07 (m, 3H), 4.07-3.75 (m, 2H), 3.65 (s, 1H), 3.46 (d, J=11.9 Hz, 1H), 3.30-3.21 (m, 3H), 3.09 (d, J=18.2 Hz, 6H), 2.99-2.85 (m, 1H), 2.82-2.32 (m, 5H), 2.19 (d, J=17.9 Hz, 1H), 2.08-1.79 (m, 6H), 1.79-1.62 (m, 7H), 1.62-1.41 (m, 3H), 1.40-1.35 (m, 4H), 1.35-1.28 (m, 8H), 1.25 (d, J=2.2 Hz, 3H), 1.21 (d, J=2.1 Hz, 4H), 0.91 (dt, J=14.4, 7.9 Hz, 2H).

### Example 109 Preparation of 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2S)-2-(2-isopropoxyphenyl)-4-[(1s,4s)-4-hydroxy-4-methylcyclohexyl]piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed)

Synthesis of 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2S)-2-(2-isopropoxyphenyl)-4-[(1s,4s)-4-hydroxy-4-methylcyclohexyl]piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed): Into an 8 mL vial were added (1s,4s)-4-[(3S)-3-(2-isopropoxyphenyl)piperazin-1-yl]-1-methylcyclohexan-1-ol (40 mg, 0.1 mmol, 1.0 eq), 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-N-[3-nitro-4-({[(1r,4r)-4-hydroxy-4-methylcyclohexyl]methyl}amino)benzenesulfonyl]-4-{2-oxo-7-azaspiro[3.5]nonan-7-yl}benzamide (90 mg, 0.1 mmol, 0.9 eq), NaBH₃CN (38 mg, 0.6 mmol, 5.0 eq), ZnCl₂ (82 mg, 0.6 mmol, 5.0 eq) and MeOH (2 mL). The resulting mixture was stirred for 2 hours at 70° C. The reaction was quenched by the addition of water (2 mL) and then extracted with CH₂Cl₂ (3×5 mL). The combined organic layer was washed with brine (5 mL), dried over anhydrous Na₂SO₄. After filtration, the filtrate was concentrated under vacuum. The crude residue was purified by Prep-TLC (CH₂Cl₂/MeOH=10:1) to give 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2S)-2-(2-isopropoxyphenyl)-4-[(1s,4s)-4-hydroxy-4-methylcyclohexyl]piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed) as a yellow solid (32 mg, 23.5%). LC-MS (ES, m/z) M+1: 1131. ¹HNMR (300 MHz, Methanol-d₄) δ 8.51 (dd, J=10.3, 2.2 Hz, 1H), 7.84 (d, J=9.0 Hz, 1H), 7.65 (d, J=8.7 Hz, 1H), 7.55-7.40 (m, 2H), 7.27 (t, J=8.3 Hz, 1H), 7.07 (d, J=3.5 Hz, 1H), 7.01 (s, 1H), 6.97 (d, J=6.0 Hz, 1H), 6.96-6.88 (m, 1H), 6.71-6.62 (m, 1H), 6.53 (d, J=15.2 Hz, 1H), 5.99 (dd, J=20.1, 3.5 Hz, 1H), 4.74-4.62 (m, 1H), 4.60 (s, 1H), 4.34-4.09 (m, 5H), 3.91 (d, J=7.5 Hz, 4H), 3.64 (d, J=12.4 Hz, 3H), 3.47 (d, J=6.5 Hz, 5H), 3.26 (d, J=6.8 Hz, 2H), 3.02 (s, 4H), 2.87 (s, 2H), 2.58-2.29 (m, 2H), 1.74 (d, J=12.3 Hz, 9H), 1.61-1.40 (m, 8H), 1.40-1.29 (m, 7H), 1.26 (d, J=3.6 Hz, 3H), 1.20 (d, J=2.6 Hz, 3H), 0.96 (d, J=6.9 Hz, 2H).

### Example 110 Preparation of 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2S)-2-(2-isopropoxyphenyl)-4-({8-methoxy-4,4-dimethyl-2H,3H-pyrano[2,3-c]pyridin-6-yl}methyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide hydrochloride (assumed)

Synthesis of 2-chloro-6-iodo-3-[(3-methylbut-3-en-1-yl)oxy]pyridine: Into a 500-mL 3-necked round-bottom flask, were placed 2-chloro-6-iodopyridin-3-ol (20.0 g, 78.3 mmol, 1.0 eq), 3-methyl-3-buten-1-ol (6.7 g, 78.3 mmol, 1.0 eq), PPh₃ (22.6 g, 86.1 mmol, 1.1 eq), THE (200 mL). After that, DIAD (17.4 g, 86.1 mmol, 1.1 eq) was added at 0° C. under N₂ atmosphere. The mixture was stirred at 25° C. for 2 hours under N₂ atmosphere. The reaction was then quenched by the addition of water (300 mL) and then extracted with ethyl acetate (2×300 mL). The combined organic layer was washed with brine (2×300 mL) and dried over anhydrous Na₂SO₄. After filtration, the filtrate was concentrated under vacuum. The crude residue was purified onto a silica gel column and eluted with ethyl acetate/petroleum ether=1:2 to give 2-chloro-6-iodo-3-[(3-methylbut-3-en-1-yl)oxy]pyridine as a light yellow oil (17.0 g, 67.1%). ¹HNMR (400 MHz, DMSO-d₆) δ 7.78 (d, J=8.4 Hz, 1H), 7.40 (d, J=8.4 Hz, 1H), 4.82-479 (m, 2H), 4.21 (t, J=6.8 Hz, 2H), 2.50-2.42 (m, 2H), 1.77 (t, J=1.2 Hz, 3H).

Synthesis of 6-iodo-2-methoxy-3-[(3-methylbut-3-en-1-yl)oxy]pyridine: Into a 500-mL round-bottom flask, were placed 2-chloro-6-iodo-3-[(3-methylbut-3-en-1-yl)oxy]pyridine (17.0 g, 52.5 mmol, 1.0 eq), MeONa (4.3 g, 78.8 mmol, 1.5 eq), DMSO (170 mL). The resulting solution was stirred for 2 hours at 100° C. The reaction was then quenched by the addition of water (300 mL) and then extracted with ethyl acetate (2×300 mL). The combined organic layer was washed with brine (3×300 mL) and dried over anhydrous Na₂SO₄. After filtration, the filtrate was concentrated under vacuum. The crude residue was purified onto a silica gel column and eluted with ethyl acetate/petroleum ether=1:8 to give 6-iodo-2-methoxy-3-[(3-methylbut-3-en-1-yl)oxy]pyridine as a light yellow oil (3.8 g, 22.7%). ¹HNMR (400 MHz, DMSO-d₆) δ 7.32 (d, J=8.0 Hz, 1H), 7.11 (d, J=8.2 Hz, 1H), 4.83-4.75 (m, 2H), 4.07 (t, J=7.0 Hz, 2H), 3.83 (s, 3H), 2.43 (t, J=6.8 Hz, 2H), 1.76 (t, J=1.2 Hz, 3H).

Synthesis of 6-iodo-8-methoxy-4,4-dimethyl-2H,3H-pyrano[2,3-c]pyridine: Into a 100-mL round-bottom flask, were placed 6-iodo-2-methoxy-3-[(3-methylbut-3-en-1-yl)oxy]pyridine (3.5 g, 11.0 mmol, 1.0 eq), EtOH (40 mL), TFA (2.5 g, 21.9 mmol, 2.0 eq), Fe(acac)₃ (1.9 g, 5.5 mmol, 0.5 eq), phenylsilane (3.0 g, 27.4 mmol, 2.5 eq), 2-(tert-butylperoxy)-2-methylpropane (4.8 g, 32.9 mmol, 3.0 eq). The resulting solution was stirred for 3 hours at 60° C. The reaction was then quenched by the addition of water (100 mL) and extracted with ethyl acetate (2×150 mL). The combined organic layer was washed with brine (3×100 mL) and dried over anhydrous Na₂SO₄. After filtration, the filtrate was concentrated under vacuum. The crude residue was purified onto a silica gel column and eluted with ethyl acetate/petroleum ether=1:10 to give 6-iodo-8-methoxy-4,4-dimethyl-2H,3H-pyrano[2,3-c]pyridine as a light yellow oil (300 mg, 8.6%). ¹HNMR (400 MHz, DMSO-d₆) δ 7.33 (s, 1H), 4.20-4.13 (m, 2H), 3.80 (s, 3H), 1.86-1.73 (m, 2H), 1.26 (s, 6H).

Synthesis of 8-methoxy-4,4-dimethyl-2H,3H-pyrano[2,3-c]pyridine-6-carbaldehyde: Into a 50-mL 3-necked round-bottom flask, were placed 6-iodo-8-methoxy-4,4-dimethyl-2H,3H-pyrano[2,3-c]pyridine (270 mg, 0.846 mmol, 1.0 eq), THE (4 mL). After that, n-BuLi (2.5 M in hexanes, 0.4 mL) was added at −78° C. Then the mixture was stirred at −78° C. for 30 minutes. After that, DMF (87 mg, 1.2 mmol, 1.4 eq) was added dropwise at −78° C. The mixture was stirred at −78° C. for 2 hours. The reaction was then quenched by the addition of aq. NH₄Cl (10 mL) and extracted with ethyl acetate (2×30 mL). The combined organic layer was washed with brine (2×30 mL). The combined organic phase was dried over anhydrous Na₂SO₄. After filtration, the filtrate was concentrated under vacuum. The crude residue was purified onto a silica gel column and eluted with ethyl acetate/petroleum ether=1:5 to give 8-methoxy-4,4-dimethyl-2H,3H-pyrano[2,3-c]pyridine-6-carbaldehyde as a light yellow oil (110 mg, 58.8%). ¹HNMR (300 MHz, Chloroform-d) δ 9.85 (s, 1H), 7.62 (s, 1H), 4.44-4.35 (m, 2H), 4.11 (s, 3H), 1.94-1.89 (m, 3H), 1.38 (d, J=2.1 Hz, 6H).

Synthesis of (3S)-3-(2-isopropoxyphenyl)-1-({8-methoxy-4,4-dimethyl-2H,3H-pyrano[2,3-c]pyridin-6-yl}methyl)piperazine (assumed): Into a 20-mL sealed-tube, were placed (2S)-2-(2-isopropoxyphenyl)piperazine (assumed) (110 mg, 0.5 mmol, 1.0 eq), 8-methoxy-4,4-dimethyl-2H,3H-pyrano[2,3-c]pyridine-6-carbaldehyde (110 mg, 0.5 mmol, 1.0 eq), DCE (3 mL), NaBH(OAc)₃ (317 mg, 1.5 mmol, 3.0 eq). The resulting solution was stirred for 2 hours at 25° C. The reaction was then quenched by the addition of water (20 mL) and extracted with dichloromethane/methanol=20:1 (2×20 mL). The combined organic layer was washed with brine (2×40 mL) and dried over anhydrous Na₂SO₄. After filtration, the filtrate was concentrated under vacuum. The crude residue was purified onto a silica gel column and eluted with dichloromethane/methanol=15:1 to give (3S)-3-(2-isopropoxyphenyl)-1-({8-methoxy-4,4-dimethyl-2H,3H-pyrano[2,3-c]pyridin-6-yl}methyl)piperazine (assumed) as an off-white solid (90 mg, 42.4%). LC-MS (ES, m/z) M+1: 426.

Synthesis of 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2S)-2-(2-isopropoxyphenyl)-4-({8-methoxy-4,4-dimethyl-2H,3H-pyrano[2,3-c]pyridin-6-yl}methyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide hydrochloride (assumed): Into an 8-mL sealed-tube, were placed (3S)-3-(2-isopropoxyphenyl)-1-({8-methoxy-4,4-dimethyl-2H,3H-pyrano[2,3-c]pyridin-6-yl}methyl)piperazine (assumed) (40 mg, 0.1 mmol, 1.0 eq), 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-N-[3-nitro-4-({[(1r,4r)-4-hydroxy- 4-methylcyclohexyl]methyl}amino)benzenesulfonyl]-4-{2-oxo-7-azaspiro[3.5]nonan-7-yl}benzamide (77 mg, 0.1 mmol, 1.0 eq), MeOH (1 mL), ZnCl₂ (384 mg, 2.8 mmol, 3.0 eq), NaBH₃CN (18 mg, 0.3 mmol, 3.0 eq). The resulting solution was stirred for 3 hours at 70° C. The reaction was then quenched by the addition of water (20 mL) and extracted with dichloromethane/methanol=10:1 (2×20 mL). The combined organic layer was washed with brine (2×20 mL) and the organic layers combined. The mixture was dried over anhydrous Na₂SO₄. After filtration, the filtrate was concentrated under vacuum. The crude product was purified by Prep-HPLC using the following conditions (Prep-HPLC-006): Column, YMC-Actus Triart C18 ExRS, 30*150 mm, 5 μm; mobile phase, water (0.05% HCl) and CH₃CN (30% CH3CN up to 45% in 7 min). Finally, 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2S)-2-(2-isopropoxyphenyl)-4-({8-methoxy-4,4-dimethyl-2H,3H-pyrano[2,3-c]pyridin-6-yl}methyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide hydrochloride (assumed) was obtained as a light yellow solid (15 mg, 12.7%). LC-MS (ES, m/z) M+1: 1224. ¹HNMR (400 MHz, Methanol-d₄) δ 8.52 (dd, J=17.4, 2.4 Hz, 1H), 7.84-7.74 (m, 3H), 7.69-7.60 (m, 1H), 7.49 (ddd, J=8.8, 7.6, 1.6 Hz, 1H), 7.20-7.13 (m, 3H), 7.11-7.06 (m, 2H), 6.96-6.78 (m, 2H), 6.70 (s, 1H), 5.47 (br, 1H), 4.84-4.77 (m, 1H), 4.52-4.41 (m, 2H), 4.32-4.17 (m, 4H), 4.12 (t, J=12.8 Hz, 1H), 3.99 (d, J=3.8 Hz, 3H), 3.97-3.82 (m, 6H), 3.76-3.44 (m, 4H), 3.37-3.15 (m, 7H), 2.33-2.22 (m, 2H), 1.91-1.83 (m, 4H), 1.75-1.70 (m, 3H), 1.66-1.60 (m, 2H), 1.56-1.45 (m, 6H), 1.43 (d, J=6.0 Hz, 6H), 1.36-1.35 (m, 7H), 1.28-1.26 (m, 5H).

### Example 111 Preparation of 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2S)-2-(2-isopropoxyphenyl)-4-({5-methoxy-6-[(3R)-3-methylmorpholin-4-yl]pyridin-3-yl}methyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed)

Synthesis of (3R)-4-(3-methoxypyridin-2-yl)-3-methylmorpholine: Into a 500 mL round-bottom flask were added 2-bromo-3-methoxypyridine (10.0 g, 53.2 mmol, 1.0 eq), (3R)-3-methylmorpholine (8.1 g, 80.0 mmol, 1.5 eq), Cs₂CO₃ (10.1 g, 132.9 mmol, 2.5 eq) and DMSO (120 mL). The resulting mixture was stirred for 36 hours at 140° C. The reaction was quenched by the addition of water (50 mL) and extracted with EtOAc (3×200 mL). The combined organic layer was washed with brine (100 mL), dried over anhydrous Na₂SO₄. After filtration, the filtrate was concentrated under vacuum. The crude residue was applied onto a silica gel column and eluted with ethyl acetate/petroleum ether=1:5 to give (3R)-4-(3-methoxypyridin-2-yl)-3-methylmorpholine as an orange solid (4.1 g, 37.0%). LC-MS (ES, m/z) M+1: 209.

Synthesis of (3R)-4-(5-bromo-3-methoxypyridin-2-yl)-3-methylmorpholine: Into a 50 mL round-bottom flask were added (3R)-4-(3-methoxypyridin-2-yl)-3-methylmorpholine (4.0 g, 19.2 mmol, 1.0 eq), NBS (6.8 g, 38.4 mmol, 2.0 eq) and DMF (10 mL). The resulting mixture was stirred for 4 hours at 80° C. The reaction was quenched by the addition of water (5 mL) and then extracted with EtOAc (3×20 mL). The combined organic layer was washed with brine (20 mL), dried over anhydrous Na₂SO₄. After filtration, the filtrate was concentrated under vacuum. The crude residue was applied onto a silica gel column and eluted with ethyl acetate/petroleum ether=1:5 to give (3R)-4-(5-bromo-3-methoxypyridin-2-yl)-3-methylmorpholine as a red solid (1.8 g, 32.6%). ¹HNMR (300 MHz, Chloroform-d) δ 7.94 (d, J=2.0 Hz, 1H), 7.15 (d, J=2.0 Hz, 1H), 4.15 (qt, J=7.4, 3.6 Hz, 1H), 3.93 (dt, J=10.0, 3.7 Hz, 2H), 3.86 (s, 3H), 3.77 (ddd, J=11.3, 9.9, 3.0 Hz, 1H), 3.64 (dd, J=11.3, 3.3 Hz, 1H), 3.48 (ddd, J=13.2, 9.8, 3.4 Hz, 1H), 3.29 (dt, J=13.0, 3.2 Hz, 1H), 1.15 (d, J=6.6 Hz, 3H).

Synthesis of 5-methoxy-6-[(3R)-3-methylmorpholin-4-yl]pyridine-3-carbaldehyde: A solution of (3R)-4-(5-bromo-3-methoxypyridin-2-yl)-3-methylmorpholine (500 mg, 1.7 mmol, 1.0 eq) in THE (8 mL) was treated with n-BuLi in hexanes (133.9 mg, 2.1 mmol, 1.2 eq) for 1 hour at −78° C., which was followed by the addition of DMF (763 mg, 10.4 mmol, 6.0 eq) dropwise at −78° C. under nitrogen atmosphere. The resulting mixture was allowed to 25° C. The reaction was quenched by the addition of sat. NH₄Cl (10 mL) at 0° C., and extracted with EtOAc (3×10 mL). The combined organic layer was washed with brine (10 mL), dried over anhydrous Na₂SO₄. After filtration, the filtrate was concentrated under vacuum. The crude residue was applied onto a silica gel column and eluted with ethyl acetate/petroleum ether=1:5 to give 5-methoxy-6-[(3R)-3-methylmorpholin-4-yl]pyridine-3-carbaldehyde as an orange solid (180 mg, 43.8%). ¹HNMR (300 MHz, Chloroform-d) δ 9.85 (s, 1H), 8.24 (d, J=1.8 Hz, 1H), 7.42 (d, J=1.8 Hz, 1H), 4.68 (d, J=7.5 Hz, 1H), 4.12 (d, J=13.4 Hz, 1H), 3.97 (d, J=12.1 Hz, 1H), 3.89 (s, 3H), 3.83 (dd, J=11.5, 3.1 Hz, 1H), 3.79-3.65 (m, 2H), 3.53 (ddd, J=13.4, 11.8, 3.4 Hz, 1H), 1.35 (d, J=6.8 Hz, 3H).

Synthesis of (3R)-4-(5-{[(3S)-3-(2-isopropoxyphenyl)piperazin-1-yl]methyl}-3-methoxypyridin-2-yl)-3-methylmorpholine (assumed): Into an 8 mL vial were added 5-methoxy-6-[(3R)-3-methylmorpholin-4-yl]pyridine-3-carbaldehyde (60 mg, 0.3 mmol, 1.0 eq), (2S)-2-(2-isopropoxyphenyl)piperazine (56 mg, 0.2 mmol, 1.0 eq), NaBH₃CN (80 mg, 1.3 mmol, 5.0 eq), ZnCl₂ (173 mg, 1.3 mmol, 5.0 eq) and MeOH (2 mL). The resulting mixture was stirred for 4 hours at 25° C. The reaction was quenched by the addition of water (2 mL) and then extracted with CH₂Cl₂ (3×5 mL). The combined organic layer was washed with brine (5 mL), dried over anhydrous Na₂SO₄. After filtration, the filtrate was concentrated under vacuum. The crude residue was purified by Prep-TLC (CH₂Cl₂/MeOH=10:1) to give (3R)-4-(5-{[(3S)-3-(2-isopropoxyphenyl)piperazin-1-yl]methyl}-3-methoxypyridin-2-yl)-3-methylmorpholine (assumed) as an off-white solid (62 mg, 55.4%). ¹HNMR (300 MHz, Chloroform-d) δ 7.77 (d, J=1.8 Hz, 1H), 7.44 (dd, J=7.6, 1.8 Hz, 1H), 7.21 (td, J=7.8, 1.8 Hz, 1H), 7.11 (d, J=1.9 Hz, 1H), 6.99-6.78 (m, 2H), 4.62-4.53 (m, 1H), 4.26 (dd, J=10.1, 2.8 Hz, 1H), 4.12 (qd, J=6.7, 3.4 Hz, 1H), 3.77 (ddd, J=11.3, 9.4, 2.9 Hz, 2H), 3.69-3.53 (m, 2H), 3.56-3.34 (m, 3H), 3.19 (dd, J=11.9, 3.1 Hz, 3H), 3.07-2.77 (m, 5H), 2.44-2.11 (m, 2H), 1.34 (d, J=6.1 Hz, 3H), 1.29-1.22 (m, 3H), 1.12 (d, J=6.6 Hz, 3H).

Synthesis of 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2S)-2-(2-isopropoxyphenyl)-4-({5-methoxy-6-[(3R)-3-methylmorpholin-4-yl]pyridin-3-yl}methyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed): Into an 8 mL vial were added (3R)-4-(5-{[(3S)-3-(2-isopropoxyphenyl)piperazin-1-yl]methyl}-3-methoxypyridin-2-yl)-3-methylmorpholine (40 mg, 0.1 mmol, 1.0 eq) and 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-N-[3-nitro-4-({[(1r,4r)-4-hydroxy-4-methylcyclohexyl]methyl}amino)benzenesulfonyl]-4-{2-oxo-7-azaspiro[3.5]nonan-7-yl}benzamide (80 mg, 0.1 mmol, 1.1 eq), NaBH₃CN (29 mg, 0.5 mmol, 5.0 eq), ZnCl₂ (62 mg, 0.5 mmol, 5.0 eq) and MeOH (2 mL). The resulting mixture was stirred for 4 hours at 70° C. The reaction was quenched by the addition of water (2 mL) and then extracted with CH₂Cl₂ (3×5 mL). The combined organic layer was washed with brine (3 mL), dried over anhydrous Na₂SO₄. After filtration, the filtrate was concentrated under vacuum. The residue was purified by Prep-TLC (CH₂Cl₂/MeOH 15:1) to give 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2S)-2-(2-isopropoxyphenyl)-4-({5-methoxy-6-[(3R)-3-methylmorpholin-4-yl]pyridin-3-yl}methyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed) (25 mg, 22.2%) as a yellow solid. LC-MS (ES, m/z) M+1: 1239. ¹HNMR (300 MHz, Chloroform-d) δ 12.58 (s, 1H), 8.65 (s, 1H), 8.57-8.35 (m, 2H), 8.28-7.99 (m, 2H), 7.99-7.65 (m, 2H), 7.44 (d, J=7.5 Hz, 1H), 7.22-7.05 (m, 1H), 7.05-6.78 (m, 2H), 6.78-6.55 (m, 2H), 6.40-6.38 (m, 1H), 6.11 (s, 2H), 4.78-4.35 (m, 4H), 4.13 (s, 2H), 4.05-3.72 (m, 6H), 3.55 (d, J=10.4 Hz, 6H), 3.36-3.15 (m, 3H), 3.15-2.79 (m, 5H), 2.81-2.56 (m, 2H), 2.51-2.05 (m, 4H), 2.03 (s, 1H), 1.96-1.70 (m, 6H), 1.52-1.50 (m, 5H), 1.50-1.27 (m, 11H), 1.27-1.01 (m, 5H), 0.89 (d, J=7.5 Hz, 2H).

### Example 112 Preparation of 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2R)-2-(2-methylphenyl)-4-[(1r,4r)-4-hydroxy-4-methylcyclohexyl]piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed)

Synthesis of 1-methyl-4-[(3R)-3-(2-methylphenyl)piperazin-1-yl]cyclohexan-1-ol (assumed): Into an 8 mL vial, were added (2R)-2-(2-methylphenyl)piperazine (180 mg, 1.0 mmol, 1.0 eq), 4-hydroxy-4-methylcyclohexan-1-one (130 mg, 1.0 mmol, 1.0 eq), DCE (6 mL), NaBH(OAc)₃ (649 mg, 3.0 mmol, 3.0 eq). The resulting mixture was stirred for overnight at 25° C. The reaction was quenched with water (10 mL) and then extracted with dichloromethane (3×10 mL). The combined organic layer was dried over anhydrous Na₂SO₄. After filtration, the filtrate was concentrated under vacuum. The residue was purified by Prep-TLC (dichloromethane/methanol=10:1) to give 1-methyl-4-[(3R)-3-(2-methylphenyl)piperazin-1-yl]cyclohexan-1-ol as a colorless oil (190 mg, 64.5%). LC-MS (ES, m/z) M+1: 289.

Synthesis of (1R,4r)-1-methyl-4-((R)-3-(o-tolyl)piperazin-1-yl)cyclohexan-1-ol (assumed) &(1S,4s)-1-methyl-4-((R)-3-(o-tolyl)piperazin-1-yl)cyclohexan-1-ol (assumed): 1-methyl-4-[(3R)-3-(2-methylphenyl)piperazin-1-yl]cyclohexan-1-ol (800 mg) was purified by Chiral-SFC using the following conditions: CHIRAL ART Cellulose-SB, 3*25 cm, 5 um; Mobile Phase A: CO₂, Mobile Phase B: MeOH (0.1% 2 M NH₃-MeOH); Flow rate: 80 mL/min; Gradient: isocratic 30% B; Wave Length: 220 nm; RT(min): 2.52; RT(min): 3.74; Sample Solvent: MeOH—Preparative; Injection Volume: 2 mL; Number Of Runs: 15. Finally, (1R,4r)-1-methyl-4-((R)-3-(o-tolyl)piperazin-1-yl)cyclohexan-1-ol was obtained as a colorless oil (60 mg, 31.5%) and (1S,4s)-1-methyl-4-((R)-3-(o-tolyl)piperazin-1-yl)cyclohexan-1-ol was obtained as a colorless oil (20 mg, 10.5%).

Synthesis of 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2R)-2-(2-methylphenyl)-4-[(1r,4r)-4-hydroxy-4-methylcyclohexyl]piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed): Into an 8 mL vial, were added 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-N-[3-nitro-4-({[(1r,4r)-4-hydroxy-4-methylcyclohexyl]methyl}amino)benzenesulfonyl]-4-{2-oxo-7-azaspiro[3.5]nonan-7-yl}benzamide (70 mg, 0.08 mmol, 1.0 eq), MeOH (5 mL), (1r,4r)-1-methyl-4-[(3R)-3-(2-methylphenyl)piperazin-1-yl]cyclohexan-1-ol (25 mg, 0.08 mmol, 1.0 eq), ZnCl₂ (35.16 mg, 0.24 mmol, 3.0 eq), NaBH₃CN (16 mg, 0.24 mmol, 3.0 eq). The resulting mixture was stirred for 5 hours at 70° C. The reaction was quenched with water (10 mL) and then extracted with dichloromethane (3×10 mL). The combined organic layer was dried over anhydrous Na₂SO₄. After filtration, the filtrate was concentrated under vacuum. The residue was purified by Prep-TLC (dichloromethane/methanol=10:1) to give 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2R)-2-(2-methylphenyl)-4-[(1r,4r)-4-hydroxy-4-methylcyclohexyl]piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide as a yellow solid (8 mg, 8.5%). LC-MS (ES, m/z) M+1: 1086. ¹HNMR (400 MHz, Methanol-d₄) δ 8.63-8.47 (m, 1H), 7.81 (d, J=12.5 Hz, 1H), 7.52-7.42 (m, 2H), 7.27-7.10 (m, 3H), 7.08-6.75 (m, 2H), 6.75-6.56 (m, 2H), 6.52 (s, 1H), 6.03 (dd, J=13.4, 3.5 Hz, 1H), δ 4.23 (s, 1H), 4.11-4.02 (m, 1H), 3.99-3.86 (m, 1H), 3.79-3.54 (m, 2H), 3.54-3.40 (m, 4H), 3.25 (d, J=6.6 Hz, 2H), 3.19-2.77 (m, 10H), 2.69-2.26 (m, 7H), 2.19-1.91 (m, 1H), 1.89-1.10 (m, 27H), 1.09-0.87 (m, 1H).

### Example 113 Preparation of 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2R)-2-(2-methylphenyl)-4-[(1s,4s)-4-hydroxy-4-methylcyclohexyl]piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide hydrochloride (assumed)

Synthesis of 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2R)-2-(2-methylphenyl)-4-[(1s,4s)-4-hydroxy-4-methylcyclohexyl]piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide hydrochloride (assumed): Into a 5 mL vial, were added 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-N-[3-nitro-4-({[(1r,4r)-4-hydroxy-4-methylcyclohexyl]methyl}amino)benzenesulfonyl]-4-{2-oxo-7-azaspiro[3.5]nonan-7-yl}benzamide ((70 mg, 0.08 mmol, 1.0 eq), (1s,4s)-1-methyl-4-[(3R)-3-(2-methylphenyl)piperazin-1-yl]cyclohexan-1-ol (25 mg, 0.08 mmol, 1.0 eq), MeOH (5 mL), ZnCl₂ (35.16 mg, 0.24 mmol, 3.0 eq), NaBH₃CN (16 mg, 0.24 mmol, 3.0 eq). The resulting mixture was stirred for 5 hours at 70° C. The reaction was quenched with water (10 mL) and then extracted with dichloromethane (3×10 mL). The combined organic layer was dried over anhydrous Na₂SO₄. After filtration, the filtrate was concentrated under vacuum. The residue was purified by Prep-TLC (dichloromethane/methanol=10:1) to give 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2R)-2-(2-methylphenyl)-4-[(1s,4s)-4-hydroxy-4-methylcyclohexyl]piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide hydrochloride as a yellow solid (15 mg, 15.5%). LC-MS (ES, m/z) M+1-HCl: 1086. ¹HNMR (400 MHz, Methanol-d₄) δ 8.53 (dd, J=14.7, 2.2 Hz, 1H), 8.06 (t, J=9.3 Hz, 1H), 7.85-7.74 (m, 1H), 7.70 (dd, J=8.8, 3.1 Hz, 1H), 7.38 (dt, J=19.2, 6.6 Hz, 3H), 7.17 (d, J=12.1 Hz, 2H), 7.08-6.74 (m, 3H), 5.38 (d, J=10.7 Hz, 1H), 4.34-4.08 (m, 2H), 4.10-3.62 (m, 9H), 3.55-3.38 (m, 3H), 3.39-3.34 (m, 1H), 3.29-3.13 (m, 5H), 2.57 (d, J=5.3 Hz, 3H), 2.41-2.13 (m, 4H), 1.93-1.37 (m, 21H), 1.27 (dd, J=10.3, 2.2 Hz, 8H), 1.07 (s, 1H).

### Example 114 Preparation of 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2R)-2-(2-methylphenyl)-4-(oxan-4-yl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed)

Synthesis of (3R)-3-(2-methylphenyl)-1-(oxan-4-yl)piperazine: Into a 40 mL vial, were added (2R)-2-(2-methylphenyl)piperazine (85 mg, 0.5 mmol, 1.0 eq), tetrahydro-4H-pyran-4-one (48 mg, 0.5 mmol, 1.0 eq), DCE (4 mL), NaBH(OAc)₃ (306 mg, 1.5 mmol, 3.0 eq). The resulting mixture was stirred for overnight at 25° C. The reaction was quenched with water (10 mL) and then extracted with dichloromethane (3×10 mL). The combined organic layer was dried over anhydrous Na₂SO₄. After filtration, the filtrate was concentrated under vacuum. The residue was purified by Prep-TLC (dichloromethane/methanol=10:1) to give (3R)-3-(2-methylphenyl)-1-(oxan-4-yl)piperazine as a colorless oil (60 mg, 47.7%). LC-MS (ES, m/z) M+1: 261.

Synthesis of 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2R)-2-(2-methylphenyl)-4-(oxan-4-yl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed): Into an 8 mL vial, were added 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-N-[3-nitro-4-({[(1r,4r)-4-hydroxy- 4-methylcyclohexyl]methyl}amino)benzenesulfonyl]-4-{2-oxo-7-azaspiro[3.5]nonan-7-yl}benzamide (81 mg, 0.1 mmol, 1.0 eq), (3R)-3-(2-methylphenyl)-1-(oxan-4-yl)piperazine (26 mg, 0.1 mmol, 1.0 eq), MeOH (3 mL), ZnCl₂ (41 mg, 0.3 mmol, 3.0 eq), NaBH₃CN (19 mg, 0.3 mmol, 3.0 eq). The resulting mixture was stirred for 7 hours at 70° C. The reaction was quenched with water (5 mL) and then extracted with dichloromethane (3×10 mL). The combined organic layer was dried over anhydrous Na₂SO₄. After filtration, the filtrate was concentrated under vacuum. The residue was purified by Prep-TLC (dichloromethane/methanol=10:1) to give 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2R)-2-(2-methylphenyl)-4-(oxan-4-yl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide as a yellow solid (16 mg, 15.1%). LC-MS (ES, m/z) M+1: 1059. ¹HNMR (400 MHz, Methanol-d₄) δ 8.49 (d, J=2.2 Hz, 1H), 7.88 (d, J=9.3 Hz, 1H), 7.49 (dd, J=9.3, 2.2 Hz, 2H), 7.22-7.12 (m, 3H), 7.08 (d, J=3.5 Hz, 1H), 6.96 (d, J=7.9 Hz, 1H), 6.70 (d, J=3.0 Hz, 2H), 6.49 (s, 1H), 6.02 (d, J=3.5 Hz, 1H), 4.29 (d, J=7.5 Hz, 2H), 4.05-3.86 (m, 3H), 3.81-3.6 (m, 2H), 3.58-3.45 (m, 5H), 3.44-3.37 (m, 2H), 3.26 (d, J=7.1 Hz, 2H), 3.21-2.96 (m, 2H), 2.84 (d, J=11.6 Hz, 1H), 2.61-2.45 (m, 3H), 2.39 (s, 3H), 1.94-1.78 (m, 7H), 1.76-1.65 (d, J=10.2 Hz, 4H), 1.62-1.38 (m, 5H), 1.37-1.20 (m, 10H), 1.20-1.08 (m, 1H), 0.99-0.83 (m, 2H).

### Example 115 Preparation of 4-{2-[(2S)-4-[(3,4-difluorophenyl)methyl]-2-(2-isopropoxyphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-N-[3-nitro-4-({[(1r,4r)-4-hydroxy-4-methylcyclohexyl]methyl}amino)benzenesulfonyl]benzamide (assumed)

Synthesis of (3S)-1-[(3,4-difluorophenyl)methyl]-3-(2-isopropoxyphenyl)piperazine (assumed): Into an 8-mL sealed-tube, were placed (2S)-2-(2-isopropoxyphenyl)piperazine (assumed) (100 mg, 0.5 mmol, 1.0 eq), 3,4-difluorobenzaldehyde (65 mg, 0.5 mmol, 1.0 eq), MeOH (2 mL), ZnCl₂ (186 mg, 1.4 mmol, 3.0 eq), NaBH₃CN (86 mg, 1.4 mmol, 3.0 eq). The resulting solution was stirred for 4 hours at 25° C. The reaction was then quenched by the addition of water (20 mL) and then extracted with dichloromethane/methanol=10:1 (2×20 mL). The combined organic layer was washed with brine (2×40 mL) and dried over anhydrous Na₂SO₄. After filtration, the filtrate was concentrated under vacuum. The crude residue was purified onto a silica gel column and eluted with dichloromethane/methanol=10:1 to give (3S)-1-[(3,4-difluorophenyl)methyl]-3-(2-isopropoxyphenyl)piperazine (assumed) as an off-white solid (60 mg, 38.2%). LC-MS (ES, m/z) M+1: 347.

Synthesis of 4-{2-[(2S)-4-[(3,4-difluorophenyl)methyl]-2-(2-isopropoxyphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-N-[3-nitro-4-({[(1r,4r)-4-hydroxy-4-methylcyclohexyl]methyl}amino)benzenesulfonyl]benzamide (assumed): Into an 8-mL sealed-tube, were placed (3S)-1-[(3,4-difluorophenyl)methyl]-3-(2-isopropoxyphenyl)piperazine (assumed) (35 mg, 0.1 mmol, 1.0 eq), 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-N-[3-nitro-4-({[(1r,4r)-4-hydroxy-4-methylcyclohexyl]methyl}amino)benzenesulfonyl]-4-{2-oxo-7-azaspiro[3.5]nonan-7-yl}benzamide (82 mg, 0.1 mmol, 1.0 eq), MeOH (1 mL), ZnCl₂ (41 mg, 0.3 mmol, 3.0 eq), NaBH₃CN (19 mg, 0.3 mmol, 3.0 eq). The resulting solution was stirred for 3 hours at 70° C. The reaction was then quenched by the addition of water (20 mL) and then extracted with dichloromethane/methanol=10:1 (2×30 mL). The combined organic layer was washed with brine (2×20 mL) and dried over anhydrous Na₂SO₄. After filtration, the filtrate was concentrated under vacuum. The residue was purified by Prep-TLC (dichloromethane/methanol=15:1) to give 4-{2-[(2S)-4-[(3,4-difluorophenyl)methyl]-2-(2-isopropoxyphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-N-[3-nitro-4-({[(1r,4r)-4-hydroxy-4-methylcyclohexyl]methyl}amino)benzenesulfonyl]benzamide (assumed) as a light yellow solid (25 mg, 21.6%). LC-MS (ES, m/z) M+1: 1145. ¹HNMR (400 MHz, Methanol-d₄) δ 8.53-8.46 (m, 1H), 7.87 (d, J=9.0 Hz, 1H), 7.55-7.40 (m, 2H), 7.32-7.11 (m, 4H), 7.07 (d, J=3.6 Hz, 1H), 7.03-6.86 (m, 3H), 6.72-6.63 (m, 2H), 6.49 (s, 1H), 6.02 (d, J=3.4 Hz, 1H), 4.61 (p, J=6.7, 6.0 Hz, 1H), 4.31-4.26 (m, 2H), 4.16-4.12 (m, 1H), 3.93-3.89 (m, 1H), 3.73-3.67 (m, 1H), 3.59-3.55 (m, 2H), 3.52-3.45 (m, 2H), 3.27-3.09 (m, 6H), 2.98-2.92 (m, 1H), 2.68-2.63 (m, 1H), 2.48-2.28 (m, 3H), 2.04-1.93 (m, 1H), 1.87-1.81 (m, 2H), 1.76-1.65 (m, 5H), 1.57-1.49 (m, 5H), 1.44-1.40 (m, 2H), 1.39-1.37 (m, 2H), 1.34-1.31 (m, 4H), 1.29-1.22 (m, 9H).

### Example 116 Preparation of 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2S)-2-(2-isopropoxyphenyl)-4-({4-[(3R)-3-methylmorpholin-4-yl]phenyl}methyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed)

Synthesis of 4-[(3R)-3-methylmorpholin-4-yl]benzaldehyde: Into a 100 mL round-bottom flask, were placed 4-fluoro-benzaldehyde (5.0 g, 40.3 mmol, 1.0 eq), (3R)-3-methylmorpholine (8.2 g, 80.6 mmol, 2.0 eq), K₂CO₃ (11.1 g, 80.6 mmol, 2.0 eq), DMSO (50 mL). The resulting solution was stirred for 4 hours at 130° C. The reaction was then quenched by the addition of water (50 mL) and extracted with ethyl acetate (2×50 mL). The combined organic layer was washed with water (2×50 mL), brine (2×50 mL) and over anhydrous Na₂SO₄. After filtration, the filtrate was concentrated under vacuum. The crude residue was applied onto a silica gel column and eluted with ethyl acetate/petroleum ether=1:3 to give 4-[(3R)-3-methylmorpholin-4-yl]benzaldehyde as a yellow solid (1.5 g, 18.1%). LC-MS (ES, m/z) M+1: 206. ¹HNMR (300 MHz, DMSO-d₆) δ 9.72 (s, 1H), 7.86-7.62 (m, 2H), 7.13-6.85 (m, 2H), 4.17-4.06 (m, 1H), 3.99-3.91 (m, 1H), 3.74 (dt, J=11.4, 1.2 Hz, 1H), 3.67 (dd, J=11.4, 3.0 Hz, 1H), 3.56 (d, J=2.7 Hz, 1H), 3.54-3.51 (m, 1H), 3.10 (td, J=13.2, 4.1 Hz, 1H), 1.11 (d, J=6.6 Hz, 3H).

Synthesis of (3R)-4-(4-{[(3S)-3-(2-isopropoxyphenyl)piperazin-1-yl]methyl}phenyl)-3-methylmorpholine (assumed): Into an 8-mL sealed tube purged and maintained with an inert atmosphere of nitrogen, were placed 4-[(3R)-3-methylmorpholin-4-yl]benzaldehyde (75 mg, 0.4 mmol, 1.0 eq), (2S)-2-(2-isopropoxyphenyl)piperazine (assumed) (81 mg, 0.4 mmol, 1.0 eq), NaBH(OAc)₃ (232 mg, 1.1 mmol, 3.0 eq), DCE (1 mL). The resulting solution was stirred for 2 hours at 25° C. The reaction was then quenched by the addition of water (10 mL) and extracted with ethyl acetate (2×10 mL). The combined organic layer was washed with brine (2×10 mL) and dried over anhydrous Na₂SO₄. After filtration, the filtrate was concentrated under vacuum. The crude residue was applied onto a silica gel column and eluted with dichloromethane/methanol=10:1 to give (3R)-4-(4-{[(3S)-3-(2-isopropoxyphenyl)piperazin-1-yl]methyl}phenyl)-3-methylmorpholine (assumed) as a white solid (90 mg, 60.1%). LC-MS (ES, m/z) M+1: 410. ¹HNMR (300 MHz, Chloroform-d) δ 7.52 (d, J=7.5 Hz, 1H), 7.28-7.19 (m, 4H), 6.86 (dd, J=8.7, 2.1 Hz, 3H), 4.59 (dd, J=12.3, 6.0 Hz, 1H), 4.55-4.49 (m, 1H), 3.99 (dt, J=11.1, 3.3 Hz, 1H), 3.90-3.82 (m, 1H), 3.72 (dt, J=11.1, 4.2 Hz, 4H), 3.57 (d, J=12.9 Hz, 1H), 3.30-3.25 (m, 2H), 3.14 (dd, J=8.4, 3.9 Hz, 2H), 3.12-2.97 (m, 1H), 2.60 (t, J=13.2 Hz, 1H), 2.47 (t, J=11.1 Hz, 1H), 1.38 (d, J=6.0 Hz, 3H), 1.26 (d, J=6.0 Hz, 3H), 1.09 (d, J=6.3 Hz, 3H).

Synthesis of 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2S)-2-(2-isopropoxyphenyl)-4-({4-[(3R)-3-methylmorpholin-4-yl]phenyl}methyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed): Into an 8-mL sealed tube, were placed (3R)-4-(4-{[(3S)-3-(2-isopropoxyphenyl)piperazin-1-yl]methyl}phenyl)-3-methylmorpholine (assumed) (80 mg, 0.2 mmol, 1.0 eq), 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-N-[3-nitro-4-({[(1r,4r)-4-hydroxy-4-methylcyclohexyl]methyl}amino)benzenesulfonyl]-4-{2-oxo-7-azaspiro[3.5]nonan-7-yl}benzamide (159 mg, 0.2 mmol, 1.0 eq), ZnCl₂ (80 mg, 0.6 mmol, 3.0 eq), NaBH₃CN (37 mg, 0.6 mmol, 3.0 eq), MeOH (1 mL). The resulting solution was stirred for 6 hours at 70° C. The resulting mixture was filtered, the filtrate was concentrated under vacuum. The crude residue was applied onto a silica gel column and eluted with dichloromethane/methanol=15:1 to give 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2S)-2-(2-isopropoxyphenyl)-4-({4-[(3R)-3-methylmorpholin-4-yl]phenyl}methyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed) as a yellow solid (3.4 mg, 1.4%). LC-MS (ES, m/z) M+1: 1208. ¹HNMR (300 MHz, Chloroform-d) δ 12.59 (s, 1H), 8.65 (d, J=2.1 Hz, 1H), 8.45 (dd, J=12.3, 6.8 Hz, 2H), 8.11 (d, J=9.3 Hz, 1H), 7.85-7.76 (m, 1H), 7.43 (s, 1H), 7.20 (d, J=8.4 Hz, 2H), 7.10 (t, J=3.0 Hz, 1H), 6.83 (d, J=8.7 Hz, 5H), 6.71 (d, J=9.3 Hz, 1H), 6.64 (s, 1H), 6.43 (s, 1H), 6.11 (s, 1H), 4.52 (d, J=9.9 Hz, 3H), 4.45 (dd, J=11.1, 5.0 Hz, 1H), 3.98 (d, J=12.0 Hz, 4H), 3.86 (d, J=11.7 Hz, 2H), 3.72 (s, 4H), 3.48 (td, J=21.9, 21.3, 11.2 Hz, 6H), 3.21 (t, J=6.0 Hz, 2H), 3.08 (d, J=17.1 Hz, 9H), 2.98-2.91 (m, 3H), 2.68 (d, J=12.3 Hz, 1H), 2.29 (br, 1H), 2.16 (s, 1H), 1.87 (d, J=12.0 Hz, 2H), 1.77 (d, J=13.2 Hz, 4H), 1.56-1.43 (m, 2H), 1.43-1.30 (m, 6H), 1.28 (s, 5H), 1.19 (d, J=10.5 Hz, 1H), 1.07 (s, 4H).

### Example 117 Preparation of 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2S)-4-{2H,3H-[1,4]dioxino[2,3-b]pyridin-7-ylmethyl}-2-(2-isopropoxyphenyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed)

Synthesis of (3S)-1-{2H,3H-[1,4]dioxino[2,3-b]pyridin-7-ylmethyl}-3-(2-isopropoxyphenyl)piperazine (assumed): Into an 8 mL vial were added 2H,3H-[1,4]dioxino[2,3-b]pyridine-7-carbaldehyde (80 mg, 0.5 mmol, 1.0 eq), (2S)-2-(2-isopropoxyphenyl)piperazine (107 mg, 0.5 mmol, 1.0 eq), NaBH₃CN (152 mg, 2.4 mmol, 5.0 eq), ZnCl₂ (330 mg, 2.4 mmol, 5.0 eq) and MeOH (1 mL). The resulting mixture was stirred for 2 hours at 25° C. The crude residue was purified by Prep-TLC (CH₂Cl₂/MeOH=10:1) to give (3S)-1-{2H,3H-[1,4]dioxino[2,3-b]pyridin-7-ylmethyl}-3-(2-isopropoxyphenyl)piperazine (assumed) as a white solid (63 mg, 35.2%). ¹HNMR (300 MHz, Chloroform-d) δ 7.77 (d, J=16.4 Hz, 1H), 7.37 (d, J=7.6 Hz, 1H), 7.08-6.82 (m, 2H), 4.78-4.66 (m, 1H), 4.65 (s, 2H), 4.49-4.44 (m, 2H), 4.29 (d, J=4.3 Hz, 2H), 3.63-3.67 (m, 3H), 3.30 (s, 1H), 3.02-3.05 (m, 2H), 2.90-2.98 (m, 2H), 2.69 (s, 1H), 1.54-1.30 (m, 6H).

Synthesis of 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2S)-4-{2H,3H-[1,4]dioxino[2,3-b]pyridin-7-ylmethyl}-2-(2-isopropoxyphenyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed): Into an 8 mL vial were added (3S)-1-{2H,3H-[1,4]dioxino[2,3-b]pyridin-7-ylmethyl}-3-(2-isopropoxyphenyl)piperazine (assumed) (40 mg, 0.1 mmol, 1.0 eq), 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-N-[3-nitro-4-({[(1r,4r)-4-hydroxy-4-methylcyclohexyl]methyl}amino)benzenesulfonyl]-4-{2-oxo-7-azaspiro[3.5]nonan-7-yl}benzamide (88 mg, 0.1 mmol, 1.0 eq), NaBH₃CN (34 mg, 0.5 mmol, 5.0 eq), ZnCl₂ (74 mg, 0.5 mmol, 5.0 eq) and MeOH (2 mL) at 70° C. The resulting mixture was stirred for 4 hours. The reaction was quenched by the addition of water (2 mL) and extracted with CH₂Cl₂ (3×5 mL). The combined organic layer was washed with brine (5 mL), dried over anhydrous Na₂SO₄. After filtration, the filtrate was concentrated under vacuum. The residue was purified by Prep-TLC (CH₂Cl₂/MeOH=17:1) to give 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2S)-4-{2H,3H-[1,4]dioxino[2,3-b]pyridin-7-ylmethyl}-2-(2-isopropoxyphenyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed) as a yellow solid (8 mg, 6.3%). LC-MS (ES, m/z) M+1: 1168. ¹HNMR (300 MHz, Chloroform-d) δ 12.59 (s, 1H), 8.65 (d, J=2.2 Hz, 1H), 8.47 (s, 2H), 8.11 (d, J=10.6 Hz, 2H), 7.80 (d, J=9.1 Hz, 1H), 7.74 (s, 1H), 7.16 (s, 1H), 7.10 (t, J=2.9 Hz, 1H), 6.83 (d, J=9.2 Hz, 2H), 6.70 (d, J=9.3 Hz, 1H), 6.63 (s, 1H), 6.43 (s, 1H), 6.11 (s, 1H), 4.71 (s, 1H), 4.52 (d, J=11.4 Hz, 3H), 4.48-4.36 (m, 3H), 4.25 (s, 2H), 4.07-3.81 (m, 2H), 3.74-3.37 (m, 7H), 3.21 (t, J=6.1 Hz, 3H), 3.07-3.01 (m, 6H), 1.86 (d, J=13.3 Hz, 3H), 1.77 (d, J=13.4 Hz, 6H), 1.60-1.38 (m, 12H), 1.28 (d, J=2.0 Hz, 9H).

### Example 118 Preparation of 4-{2-[(2R)-4-[(5,6-dimethoxypyridin-3-yl)methyl]-2-(2-methylphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-N-[3-nitro-4-({[(1r,4r)-4-hydroxy-4-methylcyclohexyl]methyl}amino)benzenesulfonyl]benzamide (assumed)

Synthesis of 5-bromo-2,3-dimethoxypyridine: Into a 50-mL round-bottom flask, were placed 2,3-dimethoxypyridine (8.0 g, 57.5 mmol, 1.0 eq), Br₂ (10.1 g, 63.2 mmol, 1.1 eq), dichloromethane (80 mL). The resulting mixture was stirred for 2 hours at 0° C. The reaction was then quenched by the addition of water (50 mL) and extracted with ethyl acetate (50 mL). The combined organic layer was washed with water (50 mL×2), brine (50 mL×2) and dried over Na₂SO₄. After filtration, the resulting mixture was concentrated under vacuum. The crude residue was applied onto a silica gel column and eluted with ethyl acetate/petroleum ether=1:2 to give 5-bromo-2,3-dimethoxypyridine as an off-white oil (5.0 g, 40.0%). LC-MS (ES, m/z) M+1: 218. ¹HNMR (300 MHz, Chloroform-d) δ 7.73 (d, J=2.1 Hz, 1H), 7.09 (d, J=2.1 Hz, 1H), 3.95 (s, 3H), 3.83 (s, 3H).

Synthesis of 5,6-dimethoxypyridine-3-carbaldehyde: Into a 250 mL 3-necked round-bottom flask, were placed 5-bromo-2,3-dimethoxypyridine (4.7 g, 21.6 mmol, 1.0 eq), THE (50 mL). After that, n-BuLi (1.1 eq) was added at −78° C. The resulting mixture was stirred for 30 minutes at −78° C. To the above mixture was added DMF (2.2 g, 30.2 mmol, 1.4 eq) at −78° C. The resulting mixture was stirred for additional 30 minutes at −78° C. The reaction was then quenched by the addition of water (200 mL) and extracted with ethyl acetate (2×100 mL). The combined organic layer was washed with brine (2×100 mL), dried over anhydrous Na₂SO₄. After filtration, the filtrate was concentrated under vacuum. The crude residue was purified onto a silica gel column and eluted with ethyl acetate/petroleum ether=1:1 to give 5,6-dimethoxypyridine-3-carbaldehyde as a light pink solid (2.7 g, 74.9%). LC-MS (ES, m/z) M+1: 168. ¹HNMR (300 MHz, DMSO-d₆) δ 9.95 (s, 1H), 8.35 (d, J=1.8 Hz, 1H), 7.53 (d, J=1.8 Hz, 1H), 3.99 (s, 3H), 3.87 (s, 3H).

Synthesis of (3R)-1-[(5,6-dimethoxypyridin-3-yl)methyl]-3-(2-methylphenyl)piperazine (assumed): Into a 40 mL vial were added 5,6-dimethoxypyridine-3-carbaldehyde (100 mg, 0.6 mmol, 1.0 eq), (2R)-2-(2-methylphenyl)piperazine (assumed) (105 mg, 0.6 mmol, 1.0 eq), NaBH₃CN (1878 mg, 3.0 mmol, 5.0 eq), ZnCl₂ (196 mg, 3.0 mmol, 5.0 eq) and MeOH (2 mL). The resulting mixture was stirred for 4 hours at 25° C. The reaction was quenched by the addition of water (2 mL) and extracted with EtOAc (3×5 mL). The combined organic layer was washed with brine (5 mL), dried over anhydrous Na₂SO₄. After filtration, the filtrate was concentrated under vacuum. The crude residue was purified by Prep-TLC (CH₂Cl₂/MeOH=10:1) to give (3R)-1-[(5,6-dimethoxypyridin-3-yl)methyl]-3-(2-methylphenyl)piperazine (assumed) as an off-white solid (75 mg, 38.3%). ¹HNMR (300 MHz, Chloroform-d) δ 7.57 (s, 1H), 7.53-7.38 (m, 1H), 7.27-7.18 (m, 2H), 7.15 (d, J=5.2 Hz, 2H), 4.43-4.24 (m, 1H), 3.97 (s, 3H), 3.89 (s, 3H), 3.75-3.66 (m, 1H), 3.65-3.49 (m, 2H), 3.37 (s, 2H), 2.96 (dd, J=19.1, 12.3 Hz, 2H), 2.62 (s, 1H), 2.45 (t, J=11.6 Hz, 1H), 2.32 (s, 3H).

Synthesis of 4-{2-[(2R)-4-[(5,6-dimethoxypyridin-3-yl)methyl]-2-(2-methylphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-N-[3-nitro-4-({[(1r,4r)-4-hydroxy-4-methylcyclohexyl]methyl}amino)benzenesulfonyl]benzamide (assumed): Into an 8 mL vial were added (3R)-1-[(5,6-dimethoxypyridin-3-yl)methyl]-3-(2-methylphenyl)piperazine (assumed) (40 mg, 0.1 mmol, 1.0 eq), 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-N-[3-nitro-4-({[(1r,4r)-4-hydroxy-4-methylcyclohexyl]methyl}amino)benzenesulfonyl]-4-{2-oxo-7-azaspiro[3.5]nonan-7-yl}benzamide (99 mg, 0.1 mmol, 1.0 eq), NaBH₃CN (39 mg, 0.6 mmol, 5.0 eq), ZnCl₂ (83 mg, 0.6 mmol, 5.0 eq) and MeOH (2 mL). The resulting mixture was stirred for 4 hours at 70° C. The reaction was quenched by the addition of water (2 mL) and extracted with EtOAc (3×5 mL). The combined organic layer was washed with brine (5 mL), dried over anhydrous Na₂SO₄. After filtration, the filtrate was concentrated under vacuum. The crude residue was purified by Prep-TLC (CH₂Cl₂/MeOH=15:1) to give 4-{2-[(2R)-4-[(5,6-dimethoxypyridin-3-yl)methyl]-2-(2-methylphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-N-[3-nitro-4-({[(1r,4r)-4-hydroxy- 4-methylcyclohexyl]methyl}amino)benzenesulfonyl]benzamide (assumed)(16 mg, 11.6%) as a yellow solid. LC-MS (ES, m/z) M+1: 1126. ¹HNMR (300 MHz, Chloroform-d) δ 12.59 (s, 1H), 8.65 (d, J=2.2 Hz, 1H), 8.46 (d, J=5.4 Hz, 2H), 8.11 (d, J=9.1 Hz, 1H), 7.80 (d, J=9.6 Hz, 1H), 7.59 (s, 1H), 7.43 (s, 1H), 7.10 (t, J=3.0 Hz, 3H), 6.84 (d, J=9.3 Hz, 1H), 6.76-6.65 (m, 1H), 6.63 (s, 1H), 6.44 (d, J=2.4 Hz, 1H), 6.11 (s, 1H), 4.63-4.40 (m, 2H), 4.01 (s, 3H), 3.89 (s, 3H), 3.59-3.35 (m, 6H), 3.21 (t, J=6.1 Hz, 4H), 3.15-2.87 (m, 9H), 2.47-2.24 (m, 6H), 1.86 (d, J=13.7 Hz, 5H), 1.77 (d, J=13.1 Hz, 6H), 1.28 (s, 7H), 1.25-0.99 (m, 3H).

### Example 119 Preparation of N-((4-((((1r,4r)-4-hydroxy-4-methylcyclohexyl)methyl)amino)-3-nitrophenyl)sulfonyl)-4-(2-((S)-2-(2-isopropoxyphenyl)-4-((5-(methoxy-d3)-6-morpholinopyridin-3-yl)methyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed)

Synthesis of (S)-4-(5-((3-(2-isopropoxyphenyl)piperazin-1-yl)methyl)-3-(methoxy-d3)pyridin-2-yl)morpholine (assumed): Into a 40-mL sealed-tube, were placed (S)-2-(2-isopropoxyphenyl)piperazine (assumed) (150 mg, 0.5 mmol, 1.0 eq), 5-(methoxy-d3)-6-morpholinonicotinaldehyde (118 mg, 0.5 mmol, 1.1 eq), DCE (4 mL), NaBH(OAc)₃ (307 mg, 1.4 mmol, 3.0 eq). The resulting solution was stirred for 2 hours at 25° C. The reaction was then quenched by the addition of water (40 mL) and extracted with ethyl acetate (2×40 mL). The combined organic layer was washed with brine (2×40 mL) and dried over anhydrous Na₂SO₄. After filtration, the filtrate was concentrated under vacuum. The crude residue was purified onto a silica gel column and eluted with ethyl acetate/petroleum ether=1:2 to give (S)-4-(5-((3-(2-isopropoxyphenyl)piperazin-1-yl)methyl)-3-(methoxy-d3)pyridin-2-yl)morpholine (assumed) as a light yellow solid (110 mg, 44.1%). LC-MS (ES, m/z) M+1: 430.

Synthesis of N-((4-((((1r,4r)-4-hydroxy-4-methylcyclohexyl)methyl)amino)-3-nitrophenyl)sulfonyl)-4-(2-((S)-2-(2-isopropoxyphenyl)-4-((5-(methoxy-d3)-6-morpholinopyridin-3-yl)methyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed): Into an 8 mL vial, were added N-((4-((((1r,4r)-4-hydroxy-4-methylcyclohexyl)methyl)amino)-3-nitrophenyl)sulfonyl)-4-(2-oxo-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (87 mg, 0.1 mmol, 0.7 eq), (S)-4-(5-((3-(2-isopropoxyphenyl)piperazin-1-yl)methyl)-3-(methoxy-d3)pyridin-2-yl)morpholine (assumed) (45 mg, 0.1 mmol, 1.0 eq), NaBH₃CN (41 mg, 0.7 mmol, 5.0 eq), ZnCl₂ (90 mg, 0.7 mmol, 5.0 eq) and MeOH (2 mL). The resulting mixture was stirred for 5 hours at 70° C. The reaction was quenched by the addition of water (2 mL) and then extracted with CH₂Cl₂ (3×3 mL). The combined organic layer was washed with brine (3 mL), dried over anhydrous Na₂SO₄. After filtration, the filtrate was concentrated under vacuum. The residue was purified by Prep-TLC (CH₂Cl₂/MeOH=20:1) to give N-((4-((((1r,4r)-4-hydroxy-4-methylcyclohexyl)methyl)amino)-3-nitrophenyl)sulfonyl)-4-(2-((S)-2-(2-isopropoxyphenyl)-4-((5-(methoxy-d3)-6-morpholinopyridin-3-yl)methyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed) as a yellow solid (15 mg, 15.9%). LC-MS (ES, m/z) M+1: 1128. ¹HNMR (300 MHz, Chloroform-d) δ 12.57 (s, 1H), 8.65 (s, 2H), 8.46 (s, 2H), 8.40 (s, 1H), 8.11 (d, J=9.6 Hz, 2H), 7.80 (s, 2H), 7.07 (d, J=16.5 Hz, 3H), 6.98 (s, 1H), 6.85 (d, J=8.1 Hz, 4H), 6.71 (d, J=9.3 Hz, 2H), 6.62 (s, 1H), 6.43 (s, 2H), 6.11 (s, 2H), 4.61-4.50 (m, 1H), 4.00 (s, 1H), 3.86 (s, 8H), 3.58 (s, 2H), 3.51 (d, J=9.0 Hz, 4H), 3.38 (s, 8H), 3.21 (t, J=6.3 Hz, 3H), 3.06 (s, 11H), 1.75 (s, 2H), 1.51 (d, J=12.3 Hz, 1H), 1.37 (dd, J=14.7, 9.0 Hz, 9H), 0.89 (d, J=8.1 Hz, 3H).

### Example 120 Preparation of 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-[(1S,2R)-2-[(2S)-2-(2-isopropoxyphenyl)-4-{[5-methoxy-6-(morpholin-4-yl)pyridin-3-yl]methyl}piperazin-1-yl]-1-methyl-7-azaspiro[3.5]nonan-7-yl]-N-[3-nitro-4-({[(1r,4r)-4-hydroxy-4-methylcyclohexyl]methyl}amino)benzenesulfonyl]benzamide (assumed)

Synthesis of 2-bromo-4-{1-methyl-2-oxo-7-azaspiro[3.5]nonan-7-yl}benzoic acid: Into a 100 mL round-bottom flask, were placed methyl 2-bromo-4-{1-methyl-2-oxo-7-azaspiro[3.5]nonan-7-yl}benzoate (2.5 g, 6.8 mmol, 1.0 eq), methanol (12.5 mL), 1,4-dioxane (12.5 mL), NaOH (1.1 g, 27.3 mmol, 4.0 eq). The resulting mixture was stirred for 16 hours at 45° C. The resulting mixture was then diluted with water (30 mL) and acidified to pH=5 with HCl (1 M). The precipitated solids were collected by filtration and dried under infrared light to give 2-bromo-4-{1-methyl-2-oxo-7-azaspiro[3.5]nonan-7-yl}benzoic acid as a yellow solid (2.0 g, crude). LC-MS (ES, m/z) M+1: 352.

Synthesis of 2-bromo-4-{1-methyl-2-oxo-7-azaspiro[3.5]nonan-7-yl}-N-[3-nitro-4-({[(1r,4r)-4-hydroxy-4-methylcyclohexyl]methyl}amino)benzenesulfonyl]benzamide: Into a 40 mL vial, were placed 2-bromo-4-{1-methyl-2-oxo-7-azaspiro[3.5]nonan-7-yl}benzoic acid (1.0 g, 2.9 mmol, 1.0 eq), 3-nitro-4-({[(1r,4r)-4-hydroxy-4-methylcyclohexyl]methyl}amino)benzenesulfonamide (970 mg, 2.8 mmol, 1.0 eq), dichloromethane (10 mL), EDCl (1.1 g, 5.7 mmol, 2.0 eq), DMAP (1.4 g, 11.4 mmol, 4.0 eq). The resulting mixture was stirred overnight at 25° C. The resulting mixture was concentrated under vacuum. The crude residue was applied onto a silica gel column and eluted with dichloromethane/ethyl acetate (included 20% of methanol)=1:1 to give 2-bromo-4-{1-methyl-2-oxo-7-azaspiro[3.5]nonan-7-yl}-N-[3-nitro-4-({[(1r,4r)-4-hydroxy-4-methylcyclohexyl]methyl}amino)benzenesulfonyl]benzamide as a yellow solid (1.2 g, 62.4%). LC-MS (ES, m/z) M+1: 677.

Synthesis of 4-{1-methyl-2-oxo-7-azaspiro[3.5]nonan-7-yl}-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide: Into an 8-mL sealed tube purged and maintained with an inert atmosphere of nitrogen, were placed 2-bromo-4-{1-methyl-2-oxo-7-azaspiro[3.5]nonan-7-yl}-N-[3-nitro-4-({[(1r,4r)-4-hydroxy-4-methylcyclohexyl]methyl}amino)benzenesulfonyl]benzamide (800 mg, 1.2 mmol, 1.0 eq), (3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraene (427 mg, 1.2 mmol, 1.0 eq), N₁,N₂-di-o-tolyloxalamide (170 mg, 0.7 mmol, 0.6 eq), K₂CO₃ (490 mg, 3.5 mmol, 3.0 eq), CuI (135 mg, 0.7 mmol, 0.6 eq), DMSO (1 mL). The resulting solution was stirred for 2 hours at 100° C. The reaction was then quenched by the addition of water (30 mL) and extracted with ethyl acetate (2×30 mL). The combined organic layer was washed with brine (2×30 mL) and dried over anhydrous Na₂SO₄. After filtration, the filtrate was concentrated under vacuum. The crude residue was applied onto a silica gel column and eluted with dichloromethane/methanol=10:1 to give 4-{1-methyl-2-oxo-7-azaspiro[3.5]nonan-7-yl}-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide as a yellow solid (300 mg, 26.5%). LC-MS (ES, m/z) M+1: 958.

Synthesis of 4-[(1R,2R)-2-[(2S)-2-(2-isopropoxyphenyl)-4-{[5-methoxy-6-(morpholin-4-yl)pyridin-3-yl]methyl}piperazin-1-yl]-1-methyl-7-azaspiro[3.5]nonan-7-yl]-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide (assumed) & 4-[(1S,2R)-2-[(2S)-2-(2-isopropoxyphenyl)-4-{[5-methoxy-6-(morpholin-4-yl)pyridin-3-yl]methyl}piperazin-1-yl]-1-methyl-7-azaspiro[3.5]nonan-7-yl]-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide (assumed): Into an 8-mL sealed tube purged and maintained with an inert atmosphere of nitrogen, were placed 4-{1-methyl-2-oxo-7-azaspiro[3.5]nonan-7-yl}-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide (200 mg, 0.2 mmol, 1.0 eq), 4-(5-{[(3S)-3-(2-isopropoxyphenyl)piperazin-1-yl]methyl}-3-methoxypyridin-2-yl)morpholine (assumed) (89 mg, 0.2 mmol, 1.0 eq), ZnCl₂ (85 mg, 0.6 mmol, 3.0 eq), NaBH₃CN (39 mg, 0.6 mmol, 3.0 eq), MeOH (2 mL). The resulting solution was stirred for 16 hours at 70° C. The reaction was then quenched by the addition of water (30 mL) and extracted with ethyl acetate (2×30 mL). The combined organic layer was washed with brine (2×30 mL) and dried over anhydrous Na₂SO₄. After filtration, the filtrate was concentrated under vacuum. The crude residue was applied onto a silica gel column and eluted with dichloromethane/methanol=15:1 to give 4-[(1R,2R)-2-[(2S)-2-(2-isopropoxyphenyl)-4-{[5-methoxy-6-(morpholin-4-yl)pyridin-3-yl]methyl}piperazin-1-yl]-1-methyl-7-azaspiro[3.5]nonan-7-yl]-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide (assumed) as a yellow solid (50 mg, 17.5%) and 4-[(1S,2R)-2-[(2S)-2-(2-isopropoxyphenyl)-4-{[5-methoxy-6-(morpholin-4-yl)pyridin-3-yl]methyl}piperazin-1-yl]-1-methyl-7-azaspiro[3.5]nonan-7-yl]-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]benzamide (assumed) as a yellow solid (50 mg, 17.5%). LC-MS (ES, m/z) M+1: 1369.

Synthesis of 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-[(1S,2R)-2-[(2S)-2-(2-isopropoxyphenyl)-4-{[5-methoxy-6-(morpholin-4-yl)pyridin-3-yl]methyl}piperazin-1-yl]-1-methyl-7-azaspiro[3.5]nonan-7-yl]-N-[3-nitro-4-({[(1r,4r)-4-hydroxy-4-methylcyclohexyl]methyl}amino)benzenesulfonyl]benzamide (assumed): Into an 8 mL vial, were placed 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-[(1 S,2R)-2-[(2S)-2-(2-isopropoxyphenyl)-4-{[5-methoxy-6-(morpholin-4-yl)pyridin-3-yl]methyl}piperazin-1-yl]-1-methyl-7-azaspiro[3.5]nonan-7-yl]-N-[3-nitro-4-({[(1r,4r)-4-hydroxy-4-methylcyclohexyl]methyl}amino)benzenesulfonyl]benzamide (assumed) (45 mg, 0.04 mmol, 1.0 eq), ethylenediamine (44 mg, 0.7 mmol, 20.0 eq) and TBAF in THE (1 M, 1 mL). The resulting solution was stirred for 6 hours at 80° C. The reaction was then quenched by the addition of water (30 mL) and extracted with ethyl acetate (2×30 mL). The combined organic layer was washed with brine (2×30 mL) and dried over anhydrous Na₂SO₄. After filtration, the filtrate was concentrated under vacuum. The crude residue was applied onto a silica gel column and eluted with dichloromethane/methanol=15:1 to give 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-[(1S,2R)-2-[(2S)-2-(2-isopropoxyphenyl)-4-{[5-methoxy-6-(morpholin-4-yl)pyridin-3-yl]methyl}piperazin-1-yl]-1-methyl-7-azaspiro[3.5]nonan-7-yl]-N-[3-nitro-4-({[(1r,4r)-4-hydroxy-4-methylcyclohexyl]methyl}amino)benzenesulfonyl]benzamide (assumed) as a yellow solid (3.6 mg, 8.0%). LC-MS (ES, m/z) M+1: 1239. ¹HNMR (400 MHz, Methanol-d₄) δ 8.53-8.46 (m, 1H), 7.93-7.86 (m, 1H), 7.71-7.66 (m, 1H), 7.55-7.45 (m, 2H), 7.34-7.28 (m, 1H), 7.25-7.18 (m, 1H), 7.10-7.05 (m, 1H), 7.01-6.88 (m, 3H), 6.74-6.64 (m, 2H), 6.55-6.47 (m, 1H), 6.04-5.99 (m, 1H), 4.65-4.55 (m, 1H), 4.35-4.25 (m, 2H), 3.89-3.84 (m, 1H), 3.83-3.77 (m, 3H), 3.76-3.67 (m, 4H), 3.67-3.54 (m, 4H), 3.53-3.43 (m, 3H), 3.28-3.23 (m, 2H), 3.17-3.07 (m, 1H), 3.03-2.95 (m, 1H), 2.88-2.70 (m, 3H), 2.69-2.60 (m, 1H), 2.56-2.38 (m, 1H), 2.17-1.97 (m, 3H), 1.94-1.66 (m, 10H), 1.65-1.48 (m, 6H), 1.45-1.38 (m, 3H), 1.35-1.27 (m, 12H), 1.26-1.21 (m, 3H).

### Example 121 Preparation of 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-[(1R,2R)-2-[(2S)-2-(2-isopropoxyphenyl)-4-{[5-methoxy-6-(morpholin-4-yl)pyridin-3-yl]methyl}piperazin-1-yl]-1-methyl-7-azaspiro[3.5]nonan-7-yl]-N-[3-nitro-4-({[(1r,4r)-4-hydroxy-4-methylcyclohexyl]methyl}amino)benzenesulfonyl]benzamide (assumed)

Synthesis of 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-[(1R,2R)-2-[(2S)-2-(2-isopropoxyphenyl)-4-{[5-methoxy-6-(morpholin-4-yl)pyridin-3-yl]methyl}piperazin-1-yl]-1-methyl-7-azaspiro[3.5]nonan-7-yl]-N-[3-nitro-4-({[(1r,4r)-4-hydroxy-4-methylcyclohexyl]methyl}amino)benzenesulfonyl]benzamide (assumed): Into an 8 mL vial, were placed 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-[(1R,2R)-2-[(2S)-2-(2-isopropoxyphenyl)-4-{[5-methoxy-6-(morpholin-4-yl)pyridin-3-yl]methyl}piperazin-1-yl]-1-methyl-7-azaspiro[3.5]nonan-7-yl]-N-[3-nitro-4-({[(1r,4r)-4-hydroxy-4-methylcyclohexyl]methyl}amino)benzenesulfonyl]benzamide (assumed) (45 mg, 0.04 mmol, 1.0 eq), ethylenediamine (44 mg, 0.7 mmol, 20.0 eq) and TBAF in THE (1 M, 1 mL). The resulting solution was stirred for 6 hours at 80° C. The reaction was then quenched by the addition of water (30 mL) and extracted with ethyl acetate (2×30 mL). The combined organic layer was washed with brine (2×30 mL) and dried over anhydrous Na₂SO₄. After filtration, the filtrate was concentrated under vacuum. The crude residue was applied onto a silica gel column and eluted with dichloromethane/methanol=15:1 to give 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-[(1R,2R)-2-[(2S)-2-(2-isopropoxyphenyl)-4-{[5-methoxy-6-(morpholin-4-yl)pyridin-3-yl]methyl}piperazin-1-yl]-1-methyl-7-azaspiro[3.5]nonan-7-yl]-N-[3-nitro-4-({[(1r,4r)-4-hydroxy-4-methylcyclohexyl]methyl}amino)benzenesulfonyl]benzamide (assumed) as a yellow solid. (2.1 mg, 4.7%). LC-MS (ES, m/z) M+1: 1239. ¹HNMR (400 MHz, Methanol-d₄) δ 8.53-8.46 (m, 1H), 7.91-7.84 (m, 1H), 7.73-7.67 (m, 1H), 7.51-7.45 (m, 1H), 7.45-7.40 (m, 1H), 7.33-7.29 (m, 1H), 7.25-7.18 (m, 1H), 7.10-7.06 (m, 1H), 6.97-6.86 (m, 3H), 6.72-6.63 (m, 2H), 6.55-6.47 (m, 1H), 6.04-5.94 (m, 1H), 4.65-4.55 (m, 1H), 4.35-4.25 (m, 2H), 3.89-3.84 (m, 1H), 3.83-3.77 (m, 3H), 3.76-3.67 (m, 4H), 3.67-3.54 (m, 4H), 3.53-3.43 (m, 3H), 3.28-3.23 (m, 2H), 3.17-3.07 (m, 1H), 3.03-2.95 (m, 1H), 2.88-2.70 (m, 3H), 2.69-2.60 (m, 1H), 2.56-2.38 (m, 1H), 2.17-1.97 (m, 3H), 1.94-1.66 (m, 10H), 1.65-1.48 (m, 6H), 1.45-1.38 (m, 3H), 1.35-1.27 (m, 12H), 1.26-1.21 (m, 3H).

### Example 122 Preparation of 2-[(3R,8S)-13-fluoro-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2S)-2-(2-isopropoxyphenyl)-4-{[5-methoxy-6-(morpholin-4-yl)pyridin-3-yl]methyl}piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed)

Synthesis of 2-[(3R,8S)-13-fluoro-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2S)-2-(2-isopropoxyphenyl)-4-{[5-methoxy-6-(morpholin-4-yl)pyridin-3-yl]methyl}piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed): Into an 8-mL sealed tube purged and maintained with an inert atmosphere of nitrogen, were placed 2-[(3R,8S)-13-fluoro-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]-N-[3-nitro-4-({[(1r,4r)-4-hydroxy-4-methylcyclohexyl]methyl}amino)benzenesulfonyl]-4-{2-oxo-7-azaspiro[3.5]nonan-7-yl}benzamide (100 mg, 0.1 mmol, 1.0 eq), 4-(5-{[(3S)-3-(2-isopropoxyphenyl)piperazin-1-yl]methyl}-3-methoxypyridin-2-yl)morpholine (assumed) (44 mg, 0.1 mmol, 1.0 eq), ZnCl₂ (42 mg, 0.3 mmol, 3.0 eq), NaBH₃CN (20 mg, 0.3 mmol, 3.0 eq), MeOH (1 mL). The resulting solution was stirred for 5 hours at 70° C. The reaction was then quenched by the addition of water (30 mL) and extracted with ethyl acetate (2×30 mL). The combined organic layer was washed with brine (2×30 mL) and dried over anhydrous Na₂SO₄. After filtration, the filtrate was concentrated under vacuum. The crude residue was applied onto a silica gel column and eluted with dichloromethane/methanol=10:1 to give 2-[(3R, 8S)-13-fluoro-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2S)-2-(2-isopropoxyphenyl)-4-{[5-methoxy-6-(morpholin-4-yl)pyridin-3-yl]methyl }piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed) as a yellow solid (60 mg, 42.1%). LC-MS (ES, m/z) M+1: 1373.

Synthesis of 2-[(3R,8S)-13-fluoro-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2S)-2-(2-isopropoxyphenyl)-4-{[5-methoxy-6-(morpholin-4-yl)pyridin-3-yl]methyl}piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed): Into an 8-mL sealed tube, were placed 2-[(3R,8S)-13-fluoro-15-{[2-(trimethylsilyl)ethoxy]methyl}-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2S)-2-(2-isopropoxyphenyl)-4-{[5-methoxy-6-(morpholin-4-yl)pyridin-3-yl]methyl}piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed) (60 mg, 0.1 mmol, 1.0 eq), ethylenediamine (53 mg, 0.9 mmol, 20.0 eq) and TBAF in THF (1 M, 1 mL). The resulting solution was stirred for 2 hours at 50° C. The reaction was then quenched by the addition of water (30 mL) and extracted with ethyl acetate (2×30 mL). The combined organic layer was washed with brine (2×30 mL) and dried over anhydrous Na₂SO₄. After filtration, the filtrate was concentrated under vacuum. The crude residue was applied onto a silica gel column and eluted with dichloromethane/methanol=15:1 to give 2-[(3R,8S)-13-fluoro-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2S)-2-(2- isopropoxyphenyl)-4-{[5-methoxy-6-(morpholin-4-yl)pyridin-3-yl]methyl}piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed) as a yellow solid (3.0 mg, 5.5%). LC-MS (ES, m/z) M+1: 1243. ¹HNMR (400 MHz, Methanol-d₄) δ 8.48 (d, J=2.3 Hz, 1H), 7.84-7.78 (m, 1H), 7.73-7.68 (m, 1H), 7.71-7.65 (m, 1H), 7.63-7.56 (m, 1H), 7.51-7.42 (m, 2H), 7.00-6.86 (m, 3H), 6.87-6.78 (m, 1H), 6.77-6.62 (m, 2H), 6.40-6.34 (m, 1H), 4.69-4.58 (m, 1H), 4.23 (t, J=9.3 Hz, 3H), 3.89-3.87 (m, 3H), 3.80 (t, J=4.6 Hz, 4H), 3.72-3.65 (m, 1H), 3.58-3.56 (m, 2H), 3.50-3.44 (m, 2H), 3.29-3.26 (m, 2H), 3.19-3.08 (m, 6H), 3.02-2.96 (m, 1H), 2.51-2.39 (m, 1H), 2.30-2.17 (m, 2H), 2.09-1.92 (m, 2H), 1.90-1.82 (m, 2H), 1.81-1.68 (m, 5H), 1.65-1.40 (m, 10H), 1.40-1.23 (m, 15H).

### Example 123 Preparation of N-((4-((((1r,4r)-4-hydroxy-4-methylcyclohexyl)methyl)amino)-3-nitrophenyl)sulfonyl)-4-(2-((R)-4-((5-methoxy-6-morpholinopyridin-3-yl)methyl)-2-(o-tolyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed)

Synthesis of 4-(3-methoxy-5-{[(3R)-3-(2-methylphenyl)piperazin-1-yl]methyl}pyridin-2-yl)morpholine(assumed): Into an 8 mL vial were added (2R)-2-(2-methylphenyl)piperazine (70 mg, 0.4 mmol, 1.0 eq), 5-methoxy-6-(morpholin-4-yl)pyridine-3-carbaldehyde (88 mg, 0.4 mmol, 1.0 eq), DCE (5 mL), NaBH(OAc)₃ (252 mg, 1.2 mmol, 3.0 eq). The resulting mixture was stirred for 4 hours at 25° C. The reaction was quenched with water (10 mL) and then extracted with dichloromethane (3×10 mL). The combined organic layer was dried over anhydrous Na₂SO₄. After filtration, the filtrate was concentrated under vacuum. The residue was purified by Prep-TLC (dichloromethane/methanol=10:1) to give 4-(3-methoxy-5-{[(3R)-3-(2-methylphenyl)piperazin-1-yl]methyl}pyridin-2-yl)morpholine as a colorless oil (70 mg, 46.0%). LC-MS (ES, m/z) M+1: 383.

Synthesis of N-((4-((((1r,4r)-4-hydroxy-4-methylcyclohexyl)methyl)amino)-3-nitrophenyl)sulfonyl)-4-(2-((R)-4-((5-methoxy-6-morpholinopyridin-3-yl)methyl)-2-(o-tolyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed): Into an 8 mL vial, were added 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-N-[3-nitro-4-({[(1r,4r)-4-hydroxy- 4-methylcyclohexyl]methyl}amino)benzenesulfonyl]-4-{2-oxo-7-azaspiro[3.5]nonan-7-yl}benzamide (70 mg, 0.08 mmol, 1.0 eq), 4-(3-methoxy-5-{[(3R)-3-(2-methylphenyl)piperazin-1-yl]methyl}pyridin-2-yl)morpholine (33 mg, 0.08 mmol, 1.0 eq), MeOH (4 mL), ZnCl₂ (35 mg, 0.24 mmol, 3.0 eq), NaBH₃CN (16 mg, 0.24 mmol, 3.0 eq). The resulting mixture was stirred for 7 hours at 70° C. The reaction was quenched with water (5 mL) and extracted with dichloromethane (3×10 mL). The combined organic layer was dried over anhydrous Na₂SO₄. After filtration, the filtrate was concentrated under vacuum. The residue was purified by Prep-TLC (dichloromethane/methanol=16:1) to give N-((4-((((1r,4r)-4-hydroxy-4-methylcyclohexyl)methyl)amino)-3-nitrophenyl)sulfonyl)-4-(2-((R)-4-((5-methoxy-6-morpholinopyridin-3-yl)methyl)-2-(o-tolyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide as a yellow solid (15 mg, 14.7%). LC-MS (ES, m/z) M+1: 1180. ¹HNMR (400 MHz, Methanol-d₄) δ 8.46 (dd, J=6.6, 2.6 Hz, 2H), 7.86 (d, J=9.3 Hz, 1H), 7.67 (d, J=1.7 Hz, 1H), 7.46 (dd, J=9.3, 2.2 Hz, 1H), 7.26 (d, J=1.7 Hz, 1H), 7.11 (q, J=5.3, 3.9 Hz, 3H), 7.05 (d, J=3.5 Hz, 1H), 6.95-6.87 (m, 1H), 6.66 (d, J=9.7 Hz, 2H), 6.46 (s, 1H), 6.00 (d, J=3.5 Hz, 1H), 4.27 (d, J=7.5 Hz, 2H), 3.85 (s, 1H), 3.78 (t, J=4.8 Hz, 7H), 3.65 (d, J=13.4 Hz, 2H), 3.59-3.40 (m, 4H), 3.24 (t, J=6.2 Hz, 2H), 3.17-2.90 (m, 8H), 2.67-2.64 (m, 3H), 2.33 (s, 6H), 1.82 (d, J=13.8 Hz, 3H), 1.71 (d, J=12.1 Hz, 5H), 1.59-1.08 (m, 13H), 0.99-0.83 (m, 2H).

### Example 124 Preparation of N-((4-((((1r,4r)-4-hydroxy-4-methylcyclohexyl)methyl)amino)-3-nitrophenyl)sulfonyl)-4-(2-((R)-4-((5-(methoxy-d3)-6-morpholinopyridin-3-yl)methyl)-2-(o-tolyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed)

Synthesis of (R)-4-(3-(methoxy-d3)-5-((3-(o-tolyl)piperazin-1-yl)methyl)pyridin-2-yl)morpholine (assumed): Into a 40-mL sealed-tube, were placed (R)-2-(o-tolyl)piperazine (assumed) (150 mg, 0.5 mmol, 1.0 eq), 5-(methoxy-d3)-6-morpholinonicotinaldehyde (118 mg, 0.5 mmol, 1.1 eq), DCE (4 mL), NaBH(OAc)₃ (307 mg, 1.4 mmol, 3.0 eq). The resulting solution was stirred for 2 hours at 25° C. The reaction was then quenched by the addition of water (40 mL) and extracted with ethyl acetate (2×40 mL). The combined organic layer was washed with brine (2×40 mL) and dried over anhydrous Na₂SO₄. After filtration, the filtrate was concentrated under vacuum. The crude residue was purified onto a silica gel column and eluted with ethyl acetate/petroleum ether=1:2 to give (R)-4-(3-(methoxy-d3)-5-((3-(o-tolyl)piperazin-1-yl)methyl)pyridin-2-yl)morpholine as a light yellow solid (100 mg, 40.1%). LC-MS (ES, m/z) M+1: 386.

Synthesis of N-((4-((((1r,4r)-4-hydroxy-4-methylcyclohexyl)methyl)amino)-3-nitrophenyl)sulfonyl)-4-(2-((R)-4-((5-(methoxy-d3)-6-morpholinopyridin-3-yl)methyl)-2-(o-tolyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed): Into an 8 mL vial, were added N-((4-((((1r,4r)-4-hydroxy-4-methylcyclohexyl)methyl)amino)-3-nitrophenyl)sulfonyl)-4-(2-oxo-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (87 mg, 0.1 mmol, 0.7 eq), (R)-4-(3-(methoxy-d3)-5-((3-(o-tolyl)piperazin-1-yl)methyl)pyridin-2-yl)morpholine (assumed) (45 mg, 0.1 mmol, 1.0 eq), NaBH₃CN (41 mg, 0.7 mmol, 5.0 eq), ZnCl₂ (90 mg, 0.7 mmol, 5.0 eq) and MeOH (2 mL). The resulting mixture was stirred for 5 hours at 70° C. The reaction was quenched by the addition of water (2 mL) and extracted with CH₂Cl₂ (3×3 mL). The combined organic layer was washed with brine (3 mL), and dried over anhydrous Na₂SO₄. After filtration, the filtrate was concentrated under vacuum. The residue was purified by Prep-TLC (CH₂Cl₂/MeOH=20:1) to give N-((4-((((1r,4r)-4-hydroxy-4-methylcyclohexyl)methyl)amino)-3-nitrophenyl)sulfonyl)-4-(2-((R)-4-((5-(methoxy-d3)-6-morpholinopyridin-3-yl)methyl)-2-(o-tolyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed) as a yellow solid (18 mg, 18.9%). LC-MS (ES, m/z) M+1: 1184. ¹HNMR (300 MHz, Chloroform-d) δ 12.58 (s, 1H), 8.65 (d, J=2.2 Hz, 1H), 8.45 (d, J=6.0 Hz, 2H), 8.11 (d, J=9.3 Hz, 1H), 7.81 (d, J=10.2 Hz, 1H), 7.17 (s, 2H), 7.10 (s, 1H), 6.84 (d, J=8.7 Hz, 1H), 6.71 (d, J=9.3 Hz, 1H), 6.63 (s, 1H), 6.43 (s, 1H), 6.11 (s, 1H), 4.61-4.39 (m, 1H), 3.98 (d, J=11.7 Hz, 1H), 3.86 (s, 4H), 3.55 (d, J=11.4 Hz, 1H), 3.48 (d, J=10.2 Hz, 4H), 3.39 (s, 4H), 3.21 (t, J=6.3 Hz, 2H), 3.07 (s, 4H), 2.38 (s, 3H), 1.88-1.72 (m, 4H), 1.28 (s, 6H), 1.23 (d, J=12.3 Hz, 12H), 0.89 (d, J=7.5 Hz, 6H), 0.09 (s, 8H).

### Example 125 Preparation of 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2R)-4-{2H,3H-[1,4]dioxino[2,3-b]pyridin-7-ylmethyl}-2-(2-methylphenyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed)

Synthesis of (3R)-1-{2H,3H-[1,4]dioxino[2,3-b]pyridin-7-ylmethyl}-3-(2-methylphenyl)piperazine (assumed): Into an 8 mL vial were added 2H,3H-[1,4]dioxino[2,3-b]pyridine-7-carbaldehyde (60 mg, 0.4 mmol, 1.0 eq), (2R)-2-(2-methylphenyl)piperazine (6 mg, 0.4 mmol, 1.0 eq), NaBH₃CN (114 mg, 1.8 mmol, 5.0 eq), ZnCl₂ (248 mg, 1.8 mmol, 5.0 eq) and MeOH (1 mL). The resulting mixture was stirred for 2 hours at 25° C. The crude residue was purified by Prep-TLC (CH₂Cl₂/MeOH=10:1) to give (3R)-1-{2H,3H-[1,4]dioxino[2,3-b]pyridin-7-ylmethyl}-3-(2-methylphenyl)piperazine as a white solid (28 mg, 23.7%). ¹HNMR (300 MHz, Chloroform-d) δ 7.73 (d, J=2.0 Hz, 1H), 7.57-7.49 (m, 1H), 7.26-7.20 (m, 1H), 7.16 (tq, J=6.4, 3.9, 3.1 Hz, 1H), 4.47-4.38 (m, 3H), 4.27 (ddq, J=8.3, 4.1, 2.2 Hz, 3H), 4.19-4.08 (m, 1H), 3.48 (d, J=2.0 Hz, 2H), 3.10 (td, J=10.6, 5.2 Hz, 2H), 2.94-2.83 (m, 2H), 2.35 (s, 3H), 2.27 (td, J=10.8, 4.0 Hz, 1H), 2.13-2.03 (m, 1H).

Synthesis of 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2R)-4-{2H,3H-[1,4]dioxino[2,3-b]pyridin-7-ylmethyl}-2-(2-methylphenyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed): Into an 8 mL vial were added (3R)-1-{2H,3H-[1,4]dioxino[2,3-b]pyridin-7-ylmethyl}-3-(2-methylphenyl)piperazine (28 mg, 0.1 mmol, 1.0 eq), 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-N-[3-nitro-4-({[(1r,4r)-4-hydroxy-4-methylcyclohexyl]methyl}amino)benzenesulfonyl]-4-{2-oxo-7-azaspiro[3.5]nonan-7-yl}benzamide (70 mg, 0.1 mmol, 1.0 eq), NaBH₃CN (27 mg, 0.4 mmol, 5.0 eq), ZnCl₂ (59 mg, 0.4 mmol, 5.0 eq) and MeOH (2 mL). The resulting mixture was stirred for 2 hours at 70° C. The reaction was quenched by the addition of water (2 mL) and extracted with CH₂Cl₂ (3×5 mL). The combined organic layer was washed with brine (5 mL), dried over anhydrous Na₂SO₄. After filtration, the filtrate was concentrated under vacuum. The residue was purified by Prep-TLC (CH₂Cl₂/MeOH=17:1) to give 2-[(3R,8S)-2,5-dioxa-9,15,17-triazatetracyclo[8.7.0.0\hat{ }{3,8}0.0\hat{ }{12,16}]heptadeca-1(10),11,13,16-tetraen-9-yl]-4-{2-[(2R)-4-{2H,3H-[1,4]dioxino[2,3-b]pyridin-7-ylmethyl}-2-(2-methylphenyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide as a yellow solid (7 mg, 7.2%). LC-MS (ES, m/z) M+1: 1124. ¹HNMR (300 MHz, Chloroform-d) δ 12.58 (s, 1H), 8.65 (s, 1H), 8.46 (s, 1H), 8.38 (s, 1H), 8.11 (d, J=9.4 Hz, 1H), 7.78 (dd, J=19.6, 8.2 Hz, 2H), 7.21-7.06 (m, 3H), 6.98 (s, 1H), 6.84 (d, J=8.9 Hz, 1H), 6.71 (d, J=9.2 Hz, 1H), 6.64 (s, 1H), 6.43 (s, 1H), 6.11 (s, 1H), 4.71 (s, 1H), 4.60-4.39 (m, 3H), 4.25 (s, 2H), 4.05-3.88 (m, 2H), 3.67 (s, 2H), 3.60-3.37 (m, 4H), 3.21 (t, J=6.0 Hz, 2H), 3.06 (s, 4H), 2.43-2.21 (m, 4H), 1.86 (dd, J=17.1, 11.8 Hz, 8H), 1.44 (d, J=18.5 Hz, 6H), 1.37-1.16 (m, 14H).

### Biological Example 1: Bcl-2 Competition Binding Assay

The fluorescence-labeled 23 amino acid peptide BH3 was purchased from CalBiochem (NLWAAQRYGRELRRMSDKFVD). An unbound Fluorescein labeled BH3 peptide emits random light with respect to the plane of polarization plane of excited light, resulting in a lower polarization degree (mP) value. When the peptide is bound to Bcl-2, the complex tumble slower and the emitted light can have a higher level of polarization, resulting in a higher mP value. This binding assay was performed in 96-well plate and with each assay contained 15 and 30 nM of labeled peptide and purified Bcl-2 protein (purchased from R&D Systems, Inc). The assay buffer contained 20 mM Hepes (pH 7.0), 50 mM KCl, 5 mM MgCl₂, 20 mM Na₂MoO₄, 0.1 mg/ml Bovine Gamma Globulin and 0.01% NP40.

Compounds were diluted in DMSO and added to the final assay with concentration range from 20 μM to 2 nM. The polarization degree (mP) value was determined by BioTek Synergy II with background subtraction after 3 hours of incubation at room temperature. IC₅₀ was calculated using Prism software with sigmoidal dose-response curve fitting. ABT-737 was used as reference compound. Such assays, carried out with a range of doses of test compounds, allowed the determination of an approximate IC₅₀ value. Although the inhibitory properties of the compounds of the present invention vary with structural change as expected, the activity generally exhibited by these agents was in the range of IC₅₀=0.1-1000 nM. The following table lists the IC₅₀ values of certain compounds of the invention.

### Biological Example 2: In Vitro Anti-Proliferation Assay in BCL-2-Dependent Acute Lymphoblastic Leukemia (ALL) Cell Line RS4;11 with G101V Mutation

Cell antiproliferation was assayed by PerkinElmer ATPlite™ Luminescence Assay System. Briefly, the various test cancer cell lines were plated at a density of about 1×10⁴ cells per well in Costar 96-well plates, and were incubated with different concentrations of compounds for about 72 hours in medium supplemented with 5% FBS or 10% normal human serum(NHS). One lyophilized substrate solution vial was then reconstituted by adding 5 mL of substrate buffer solution, and was agitated gently until the solution was homogeneous. About 50 μL of mammalian cell lysis solution was added to 100 μL of cell suspension per well of a microplate, and the plate was shaken for about five minutes in an orbital shaker at −700 rpm. This procedure was used to lyse the cells and to stabilize the ATP. Next, 50 μL substrate solution was added to the wells and microplate was shaken for five minutes in an orbital shaker at ˜700 rpm. Finally, the luminescence was measured by a PerkinElmer TopCount® Microplate Scintillation Counter. Such assays, carried out with a range of doses of test compounds, allowed the determination of the cellular anti-antiproliferative IC₅₀ of the compounds of the present invention.

### Biological Example 3: Mice PK Study

The pharmacokinetics of compounds were evaluated in CD-1 mouse via Intravenous and Oral Administration. The IV dose was administered as a slow bolus in the Jugular vein, and oral doses were administered by gavage. The formulation for IV dosing was 5% DMSO in 20% HPBCD in water, and the PO formulation was 2.5% DMSO, 10% EtOH, 20% Cremphor EL, 67.5% D5W. The PK time point for the IV arm was 5, 15, 30 min, 1, 2, 4, 6, 8, 12, 24 hours post dose, and for PO arm was 15, 30 min, 1, 2, 4, 6, 8, 12, 24 hours post dose. Approximately 0.03 mL blood was collected at each time point. Blood of each sample was transferred into plastic micro centrifuge tubes containing EDTA-K2 and collect plasma within 15 min by centrifugation at 4000 g for 5 minutes in a 4° C. centrifuge. Plasma samples were stored in polypropylene tubes. The samples were stored in a freezer at −75±15° C. prior to analysis. Concentrations of compounds in the plasma samples were analyzed using a LC-MS/MS method. WinNonlin (Phoenix™, version 6.1) or other similar software was used for pharmacokinetic calculations. The following pharmacokinetic parameters were calculated, whenever possible from the plasma concentration versus time data: IV administration: C₀, CL, V_{d}, T_{1/2}, AUC_{inf}, AUCₗₐₛₜ, MRT, Number of Points for Regression; PO administration: Cₘₐₓ, Tₘₐₓ, T_{1/2}, AUC_{inf}, AUCₗₐₛₜ, F %, Number of Points for Regression. The pharmacokinetic data was described using descriptive statistics such as mean, standard deviation. Additional pharmacokinetic or statistical analysis was performed at the discretion of the contributing scientist, and was documented in the data summary.

### Biological Example 4: In Vivo Xenograft Studies

Compound of Example 3 is selected for in vivo studies in the BCL-2-dependent acute lymphoblastic leukemia (ALL) RS4;11 xenograft model. The CB.17 SCID mice are obtained at age 6-8 weeks from vendors and acclimated for a minimum 7-day period. The cancer cells are then implanted into the nude mice. Depending on the specific tumor type, tumors are typically detectable about two weeks following implantation. When tumor sizes reach ˜100-200 mm³, the animals with appreciable tumor size and shape are randomly assigned into groups of 8 mice each, including one vehicle control group and treatment groups. Dosing varies depending on the purpose and length of each study, which typically proceeds for about 3-4 weeks. Tumor sizes and body weight are typically measured three times per week. In addition to the determination of tumor size changes, the last tumor measurement is used to generate the tumor size change ratio (T/C value), a standard metric developed by the National Cancer Institute for xenograft tumor evaluation. In most cases, % T/C values are calculated using the following formula: % T/C=100×ΔT/ΔC if ΔT>0. When tumor regression occurred (ΔT<0), however, the following formula is used: % T/T0=100×ΔT/T0. Values of <42% are considered significant.

What is claimed is:

1. A compound of Formula (1), or an N-oxide thereof, or a pharmaceutically acceptable salt, solvate, polymorph, tautomer, stereoisomer, an isotopic form, or a prodrug of said compound of Formula (1) or N-oxide thereof: 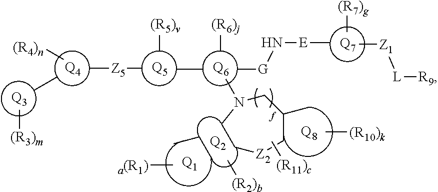
wherein
G is C(O), S(O₂), P(O)(Rₐ), or S(O)(NRₐ);
E is S(O₂), P(O)(Rₐ), S(O)(NRₐ) or C(O);
each of Q₁, Q₂, Q₃, Q₄, Q₅, Q₆, Q₇, and Q₈, independently, is cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is independently optionally substituted with one or more R_{d};
each of R₁, R₂, R₃, R₄, R₅, R₆, R₇, R₉, R₁₀, and R₁₁, independently, is absent, H, D, alkyl, alkenyl, alkynyl, halo, nitro, oxo, cyano, ORₐ, SRₐ, alkyl-Rₐ, NH(CH₂)ₚRₐ, C(O)Rₐ, S(O)Rₐ, SO₂Rₐ, C(O)ORₐ, OC(O)Rₐ, NR_{b}R_{c}, C(O)N(R_{b})R_{c}, N(R_{b})C(O)R_{c}, —P(O)R_{b}R_{c}, -alkyl-P(O)R_{b}R_{c}, —S(O)(=N(R_{b}))R_{c}, —N═S(O)R_{b}R_{c}, ═NR_{b}, SO₂N(R_{b})R_{c}, N(R_{b})SO₂R_{c}, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is independently optionally substituted with one or more R_{d};
each of Z₁, Z₂, and Z₅, independently, is a bond, (CRₐR_{b})ₚ, a bivalent alkenyl group, or a bivalent alkynyl group, N(Rₐ), O, S, C(O), S(O₂), —O(CRₐR_{b})ₚ—, —N(Rₐ)(CRₐR_{b})ₚ—, OC(O), C(O)O, OSO₂, S(O₂)O, C(O)S, SC(O), C(O)C(O), C(O)N(Rₐ), N(Rₐ)C(O), S(O₂)N(Rₐ), N(Rₐ)S(O₂), OC(O)O, OC(O)S, OC(O)N(Rₐ), N(Rₐ)C(O)O, N(Rₐ)C(O)S, N(Rₐ)C(O)N(Rₐ), (CRₐR_{b})ₚN(Rₐ)(CRₐR_{b})_{q}, (CRₐR_{b})ₚN(Rₐ)C(O)(CRₐR_{b})_{q}, OC(O)N(R_{b})(CRₐR_{b})ₚ₊₁N(R_{b})(CRₐR_{b})_{q}, or (CRₐR_{b})PC(O)N(Rₐ)(CRₐR_{b})_{q};
L is absent, a bond, (CRₐR_{b})ₚ, N(R_{c}), O, S, C(O), S(O₂), —O(CRₐR_{b})ₚ—, —N(R_{c})(CRₐR_{b})ₚ—, OC(O), C(O)O, OSO₂, S(O₂)O, C(O)S, SC(O), C(O)C(O), C(O)N(R_{c}), N(R_{c})C(O), S(O₂)N(R_{c}), N(R_{c})S(O₂), OC(O)O, OC(O)S, OC(O)N(R_{c}), N(R_{c})C(O)O, N(R_{c})C(O)S, N(R_{c})C(O)N(R_{c}), (CRₐR_{b})ₚN(R_{c})(CRₐR_{b})_{q}, (CRₐR_{b})_{P}N(R_{c})C(O)(CRₐR_{b})_{q}, OC(O)N(R_{c})(CRₐR_{b})ₚ₊₁N(R_{c})(CRₐR_{b})_{q}, (CRₐR_{b})PC(O)N(R_{c})(CRₐR_{b})_{q}, bivalent alkenyl, bivalent alkynyl, bivalent cycloalkyl, bivalent cycloalkenyl, bivalent spirocycloalkyl, bivalent fused-carbocyclic, bivalent bridged-carbocyclic, bivalent heterocycloalkyl, bivalent heterocycloalkenyl, bivalent spiro-heterocyclic, bivalent fused-heterocyclic, bivalent bridged-heterocyclic, bivalent aryl, or bivalent heteroaryl, each of the aforementioned is independently optionally substituted with one or more R_{d};
each of Rₐ, R_{b}, R_{c} and R_{d}, independently, is H, D, alkyl, spiroalkyl, alkenyl, alkynyl, halo, cyano, amine, nitro, hydroxy, ═O, -alkyl-O—P(O)(OH)(OH), C(O)NHOH, C(O)OH, C(O)NH₂, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is independently optionally substituted with one or more Rₑ;
each Rₑ is independently H, D, alkyl, alkenyl, alkynyl, halo, cyano, amine, nitro, hydroxy, ═O, -alkyl-O—P(O)(OH)(OH), C(O)NHOH, alkoxy, alkoxyalkyl, haloalkyl, hydroxyalkyl, aminoalkyl, alkylcarbonyl, alkoxycarbonyl, alkylcarbonylamino, alkylamino, halo-alkylamino, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is independently optionally substituted with one or more R_{f};
R_{f} is absent, H, D, alkyl, alkenyl, alkynyl, halo, cyano, amine, nitro, hydroxy, ═O, -alkyl-O—P(O)(OH)(OH), C(O)NHOH, alkoxy, alkoxyalkyl, haloalkyl, hydroxyalkyl, aminoalkyl, alkylcarbonyl, alkoxycarbonyl, alkylcarbonylamino, alkylamino, halo-alkylamino, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is independently optionally substituted with one or more R_{g};
R_{g} is absent, H, D, alkyl, alkenyl, alkynyl, halo, cyano, amine, nitro, hydroxy, ═O, -alkyl-O—P(O)(OH)(OH), C(O)NHOH, alkoxy, alkoxyalkyl, haloalkyl, hydroxyalkyl, aminoalkyl, alkylcarbonyl, alkoxycarbonyl, alkylcarbonylamino, alkylamino, halo-alkylamino, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl;
Rₐ and R_{b}, groups, taken together with the atom to which they are attached, may optionally form a cycloalkyl, heterocycloalkyl, aryl, or heteroaryl, each of the aforementioned is independently optionally substituted with one or more R_{d};
R_{b} and R_{c} groups, taken together with the atom to which they are attached, may optionally form a cycloalkyl, heterocycloalkyl, aryl, or heteroaryl, each of the aforementioned is independently optionally substituted with one or more R_{d};
two of R_{d} group, taken together with the atom to which they are attached, may optionally form a cycloalkyl, heterocycloalkyl, aryl, or heteroaryl, each of the aforementioned is independently optionally substituted with one or more Rₑ;
two of Rₑ groups, taken together with the atom to which they are attached, may optionally form a cycloalkyl, heterocycloalkyl, aryl, or heteroaryl, each of the aforementioned is independently optionally substituted with one or more R_{f};
two of R_{f} groups, taken together with the atom to which they are attached, may optionally form a cycloalkyl, heterocycloalkyl, aryl, or heteroaryl, each of the aforementioned is independently optionally substituted with one or more R_{g};
two of R₁ group, taken together with the atom to which they are attached, may optionally form a cycloalkyl, heterocycloalkyl, aryl, or heteroaryl, each of the aforementioned is independently optionally substituted with one or more R_{d};
two of R₂ group, taken together with the atom to which they are attached, may optionally form a cycloalkyl, heterocycloalkyl, aryl, or heteroaryl, each of the aforementioned is independently optionally substituted with one or more R_{d};
two of R₃ group, taken together with the atom to which they are attached, may optionally form a cycloalkyl, heterocycloalkyl, aryl, or heteroaryl, each of the aforementioned is independently optionally substituted with one or more R_{d};
two of R₄ group, taken together with the atom to which they are attached, may optionally form a cycloalkyl, heterocycloalkyl, aryl, or heteroaryl, each of the aforementioned is independently optionally substituted with one or more R_{d};
two of R₅ group, taken together with the atom to which they are attached, may optionally form a cycloalkyl, heterocycloalkyl, aryl, or heteroaryl, each of the aforementioned is independently optionally substituted with one or more R_{d};
two of R₆ group, taken together with the atom to which they are attached, may optionally form a cycloalkyl, heterocycloalkyl, aryl, or heteroaryl, each of the aforementioned is independently optionally substituted with one or more R_{d};
two of R₇ group, taken together with the atom to which they are attached, may optionally form a cycloalkyl, heterocycloalkyl, aryl, or heteroaryl, each of the aforementioned is independently optionally substituted with one or more R_{d};
two of R₁₀ group, taken together with the atom to which they are attached, may optionally form a cycloalkyl, heterocycloalkyl, aryl, or heteroaryl, each of the aforementioned is independently optionally substituted with one or more R_{d};
two of R₁₁ group, taken together with the atom to which they are attached, may optionally form a cycloalkyl, heterocycloalkyl, aryl, or heteroaryl, each of the aforementioned is independently optionally substituted with one or more R_{d};
R₃ and R₄ group, taken together with the atom to which they are attached, may optionally form a cycloalkyl, heterocycloalkyl, aryl, or heteroaryl, each of the aforementioned is independently optionally substituted with one or more R_{d};
R₄ and R₅ group, taken together with the atom to which they are attached, may optionally form a cycloalkyl, heterocycloalkyl, aryl, or heteroaryl, each of the aforementioned is independently optionally substituted with one or more R_{d};
R₅ and R₆ group, taken together with the atom to which they are attached, may optionally form a cycloalkyl, heterocycloalkyl, aryl, or heteroaryl, each of the aforementioned is independently optionally substituted with one or more R_{d};
R₁₀ and R₁₁ group, taken together with the atom to which they are attached, may optionally form a cycloalkyl, heterocycloalkyl, aryl, or heteroaryl, each of the aforementioned is independently optionally substituted with one or more R_{d};
R₇ and —Z₁-L-R₉ group, taken together with the atom to which they are attached, may optionally form a cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is independently optionally substituted with one or more R_{d};
one or more (e.g., both) border ring atom(s) between ring Q₈ and the ring with Z₂ can be carbon or heteroatom(s);
each of a, b, c, g, j, k, m, n, and v is, independently, 0, 1, 2, 3, 4, 5, 6, 7, or 8;
f is 0, 1, or 2; and
each of p, and q is, independently, 0, 1, 2, 3, 4, or 5.

2. The compound according to claim 1 or an N-oxide thereof, or a pharmaceutically acceptable salt, solvate, polymorph, tautomer, stereoisomer, an isotopic form, or a prodrug thereof, wherein the compound is represented by Formula (2): 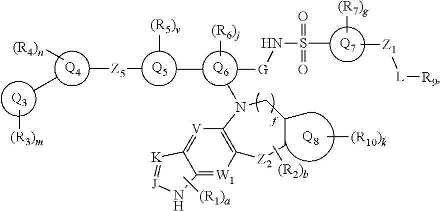
wherein
each of W₁, V, K, J, independently, is C(Rₐ) or N.

3. The compound according to claim 2 or an N-oxide thereof, or a pharmaceutically acceptable salt, solvate, polymorph, tautomer, stereoisomer, an isotopic form, or a prodrug thereof, wherein the compound is represented by Formula (3):

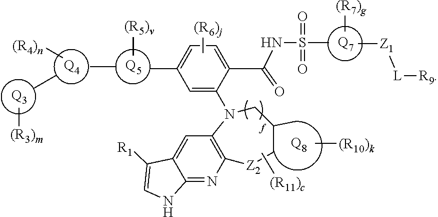

4. The compound according to claim 3 or an N-oxide thereof, or a pharmaceutically acceptable salt, solvate, polymorph, tautomer, stereoisomer, an isotopic form, or a prodrug thereof, wherein the compound is represented by Formula (4): 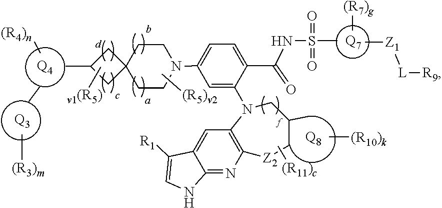
wherein
each of a, b, c, and d, independently, is 0, 1, or 2; and
v1 and v2 are both integers including 0, and v1+v2=v.

5. The compound according to claim 4 or an N-oxide thereof, or a pharmaceutically acceptable salt, solvate, polymorph, tautomer, stereoisomer, an isotopic form, or a prodrug thereof, wherein the compound is represented by Formula (5): 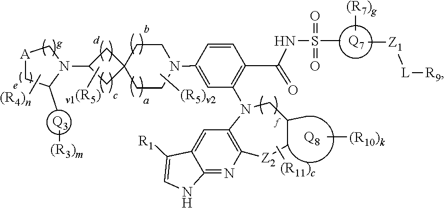
wherein
each of e and g, independently is 0, 1, or 2; and
A is O, S, SO₂, N(Rₐ), or C(RₐR^{b}).

6. The compound according to claim 5 or an N-oxide thereof, or a pharmaceutically acceptable salt, solvate, polymorph, tautomer, stereoisomer, an isotopic form, or a prodrug thereof, wherein the compound is represented by Formula (6): 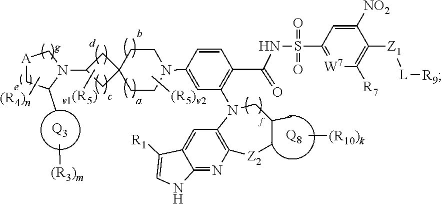
wherein
W⁷ is N, or C(Rₐ).

7. The compound according to claim 5 or an N-oxide thereof, or a pharmaceutically acceptable salt, solvate, polymorph, tautomer, stereoisomer, an isotopic form, or a prodrug thereof, wherein the compound is represented by Formula (7): 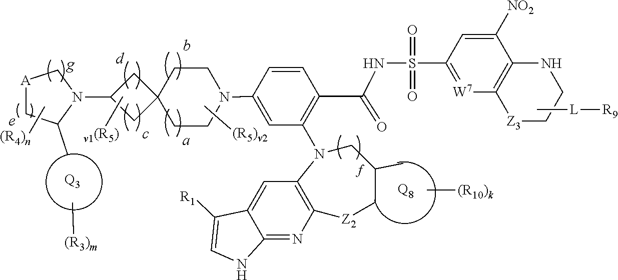
wherein
W⁷ is N, or C(Rₐ); and
Z₃ is a bond, (CH₂)ₚ, N(H), O, S, C(O), S(O₂), OC(O), C(O)O, OSO₂, S(O₂)O, C(O)S, SC(O), C(O)C(O), C(O)N(H), N(H)C(O), S(O₂)N(H), N(H)S(O₂), OC(O)O, OC(O)S, OC(O)N(H), N(H)C(O)O, N(H)C(O)S, N(H)C(O)N(H), (CH₂)ₚN(H)(CH₂)_{q}, (CH₂)ₚN(H)C(O)(CH₂)_{q}, (CH₂)PC(O)N(H)(CH₂)_{q}, or OC(O)N(H)(CH₂)ₚ₊₁N(H)(CH₂)_{q}.

8. The compound according to claim 1 or an N-oxide thereof, or a pharmaceutically acceptable salt, solvate, polymorph, tautomer, stereoisomer, an isotopic form, or a prodrug thereof, wherein the compound is represented by Formula (A): 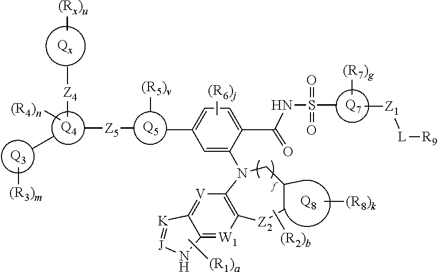
wherein
each of W₁, V, K, J, independently, is C(Rₐ) or N;
u is 0, 1, 2, 3, 4, or 5;
Z₄ is a bond, (CRₐR_{b})ₚ, a bivalent alkenyl group, or a bivalent alkynyl group, N(Rₐ), O, S, C(O), S(O₂), —O(CRₐR_{b})ₚ—, —N(Rₐ)(CRₐR_{b})ₚ—, OC(O), C(O)O, OSO₂, S(O₂)O, C(O)S, SC(O), C(O)C(O), C(O)N(Rₐ), N(Rₐ)C(O), S(O₂)N(Rₐ), N(Rₐ)S(O₂), OC(O)O, OC(O)S, OC(O)N(Rₐ), N(Rₐ)C(O)O, N(Rₐ)C(O)S, N(Rₐ)C(O)N(Rₐ), (CRₐR_{b})ₚN(Rₐ)(CRₐR_{b})_{q}, (CRₐR_{b})ₚN(Rₐ)C(O)(CRₐR_{b})_{q}, OC(O)N(R_{b})(CRₐR_{b})ₚ₊₁N(R_{b})(CRₐR_{b})_{q}, or (CRₐR_{b})PC(O)N(Rₐ)(CRₐR_{b})_{q};
Qₓ is cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is independently optionally substituted with one or more R_{d}; and
Rₓ is absent, H, D, alkyl, alkenyl, alkynyl, halo, nitro, oxo, cyano, ORₐ, SRₐ, alkyl-Rₐ, NH(CH₂)ₚRₐ, C(O)Rₐ, S(O)Rₐ, SO₂Rₐ, C(O)ORₐ, OC(O)Rₐ, NR_{b}R_{c}, C(O)N(R_{b})R_{c}, N(R_{b})C(O)R_{c}, —P(O)R_{b}R_{c}, -alkyl-P(O)R_{b}R_{c}, —S(O)(=N(R_{b}))R_{c}, —N═S(O)R_{b}R_{c}, ═NR_{b}, SO₂N(R_{b})R_{c}, or N(R_{b})SO₂R_{c}, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is independently optionally substituted with one or more R_{d}.

9. The compound according to claim 8 or an N-oxide thereof, or a pharmaceutically acceptable salt, solvate, polymorph, tautomer, stereoisomer, an isotopic form, or a prodrug thereof, wherein the compound is represented by Formula (B):

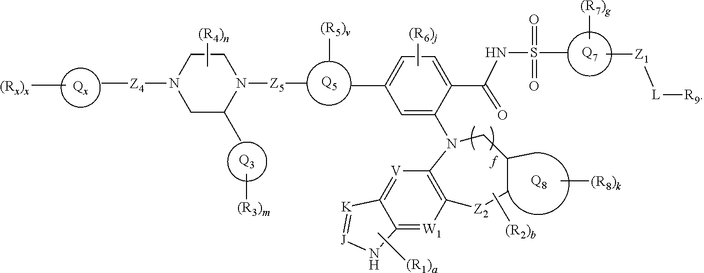

10. The compound according to claim 9 or an N-oxide thereof, or a pharmaceutically acceptable salt, solvate, polymorph, tautomer, stereoisomer, an isotopic form, or a prodrug thereof, wherein the compound is represented by Formula (C):

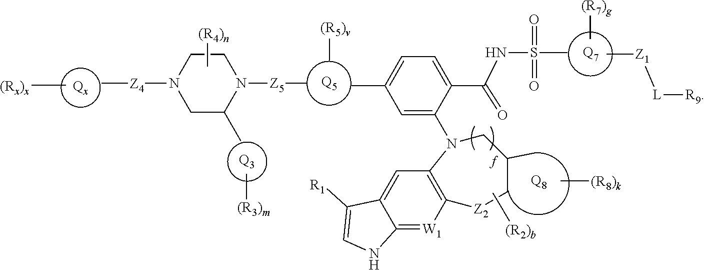

11. The compound according to claim 10 or an N-oxide thereof, or a pharmaceutically acceptable salt, solvate, polymorph, tautomer, stereoisomer, an isotopic form, or a prodrug thereof, wherein the compound is represented by Formula (D): 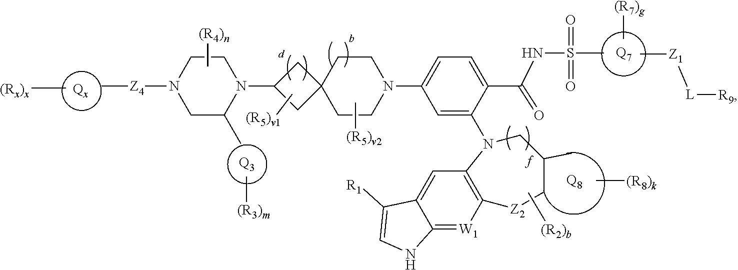
wherein
each of b, and d, independently, is 0, 1, or 2; and
v1 and v2 are both integers including 0, and v1+v2=v.

12. The compound according to claim 11 or an N-oxide thereof, or a pharmaceutically acceptable salt, solvate, polymorph, tautomer, stereoisomer, an isotopic form, or a prodrug thereof, wherein the compound is represented by Formula (E): 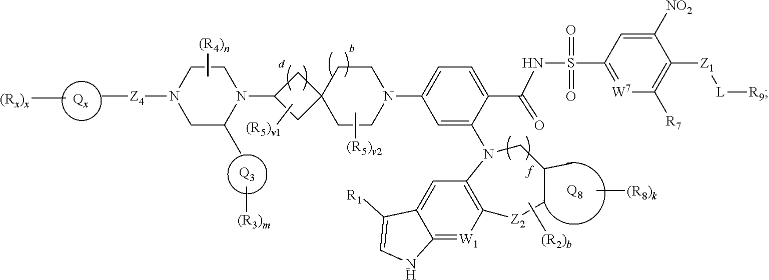
wherein
W⁷ is N, or C(Rₐ).

13. The compound according to claim 11 or an N-oxide thereof, or a pharmaceutically acceptable salt, solvate, polymorph, tautomer, stereoisomer, an isotopic form, or a prodrug thereof, wherein the compound is represented by Formula (F): 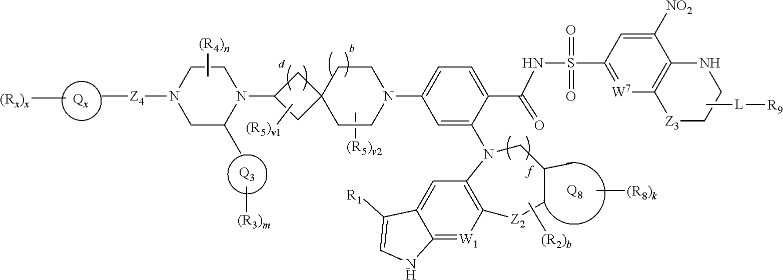
wherein
W⁷ is N, or C(Rₐ); and
Z₃ is a bond, (CH₂)ₚ, N(H), O, S, C(O), S(O₂), OC(O), C(O)O, OSO₂, S(O₂)O, C(O)S, SC(O), C(O)C(O), C(O)N(H), N(H)C(O), S(O₂)N(H), N(H)S(O₂), OC(O)O, OC(O)S, OC(O)N(H), N(H)C(O)O, N(H)C(O)S, N(H)C(O)N(H), (CH₂)ₚN(H)(CH₂)_{q}, (CH₂)ₚN(H)C(O)(CH₂)_{q}, (CH₂)PC(O)N(H)(CH₂)_{q}, or OC(O)N(H)(CH₂)ₚ₊₁N(H)(CH₂)_{q}.

14. A pharmaceutical composition comprising a compound of any one of Formulae (1), or an N-oxide thereof as defined in any one of claims 1-13, or a pharmaceutically acceptable salt, solvate, polymorph, tautomer, stereoisomer, an isotopic form, or a prodrug of said compound of any one of Formulae (1) or N-oxide thereof, and a pharmaceutically acceptable diluent or carrier.

15. A method of treating a neoplastic disease, an autoimmune disease, or a neurodegenerative disease, comprising administering to a subject in need thereof an effective amount of a compound of any one of Formulas (1) or an N-oxide thereof as defined in any one of claims 1-13, or a pharmaceutically acceptable salt, solvate, polymorph, tautomer, stereoisomer, an isotopic form, or a prodrug of said compound of any one of Formulae (1) or N-oxide thereof.
